(19) United States
(12) Patent Application Publication
(10) Pub. No.: US 2023/0355813 A1
(43) Pub. Date: Nov. 9, 2023

(54) Theranostic Silicon-Fluoride Heteroaromatic Systems and Methods Thereof

(71) Applicant: Fuzionaire Diagnostics, Inc., The Woodlands, TX (US)

(72) Inventors: Christopher Martin Waldmann, The Woodlands, TX (US); Thomas A. Singleton, Montreal (CA); Anton A. Toutov, Magnolia, TX (US); Carl Daniel Estrada, The Woodlands, TX (US); Prashant Suresh Deore, The Woodlands, TX (US); Julie Delecueillerie, The Woodlands, TX (US); Patrick DeRoy, The Woodlands, TX (US); Alexandre Larivée, The Woodlands, TX (US); Alexey Kostikov, Montreal (CA); Jean-Philip George Lumb, Montreal (CA); HyunJune Jun, Montreal (CA); En Zhao, Montreal (CA); Ryan Christopher Clark, Santa Barbara, CA (US); Burhan Ahmed Hussein, The Woodlands, TX (US); Bren Jordan Perez Atienza, The Woodlands, TX (US)

(73) Assignee: Fuzionaire Diagnostics, Inc., The Woodlands, TX (US)

(21) Appl. No.: 18/304,302

(22) Filed: Apr. 20, 2023

Related U.S. Application Data
(60) Provisional application No. 63/363,309, filed on Apr. 20, 2022.
(60) Provisional application No. 63/363,841, filed on Apr. 29, 2022.

(51) Int. Cl. A61K 51/04; A61K 51/08

(52) U.S. Cl. CPC A61K 51/0482; A61K 51/088

(57) ABSTRACT

Systems and methods for heteroaromatic silicon fluoride compounds as radiopharmaceuticals with theranostic properties are described. The theranostic heteroaromatic silicon fluoride compounds can be conjugated with various disease binding ligands and/or chelators for imaging and therapeutic applications.

### CROSS-REFERENCE TO RELATED APPLICATIONS

The current application claims the benefit of and priority to U.S. Provisional Patent Application No. 63/363,309 entitled “Silicon-Fluoride Heteroaromatic Systems and Methods Thereof” filed Apr. 20, 2022, U.S. Provisional Patent Application No. 63/363,841 entitled “Silicon-Fluoride Heteroaromatic Systems and Methods Thereof” filed Apr. 29, 2022. The disclosures of U.S. Provisional Patent Application No. 63/363,309 and U.S. Provisional Patent Application No. 63/363,841 are hereby incorporated by reference in their entireties for all purposes.

### FIELD OF THE INVENTION

The present invention generally relates to systems and methods for heteroaromatic silicon-fluoride acceptors. More particularly, the invention relates to systems and methods for heteroaromatic silicon-fluoride acceptor systems with theranostic properties.

### BACKGROUND OF THE INVENTION

Nuclear medicine is a field of radiology that takes advantage of the radiation produced by the decay of medical radioisotopes typically administered to a patient. Nuclear medicine has distinctive therapeutic and diagnostic branches, which differ by the type of radiation produced in the decay of the medical radioisotopes.

In diagnostic nuclear medicine, the scintigraphic techniques single-photon emission computed tomography (SPECT) and positron emission tomography (PET) are used for the molecular imaging of biological processes implicated in various diseases at the molecular and cellular level. These techniques rely on radiolabeled compounds, referred to as tracers or radiopharmaceuticals, that detect disease-specific biological targets or biochemical processes. SPECT and PET cameras often include computer tomography (CT) capabilities, resulting in high-end hybrid tomographic imaging devices such as SPECT/CT or PET/CT. Alternatively, PET is combined with magnetic resonance imaging (PET/MRI), which is effective in brain imaging applications.

In therapeutic nuclear medicine, radiopharmaceutical agents target pathological tissues to deliver cytotoxic radiation at the cellular level. Nuclides emitting cytotoxic α- and β⁻-radiation are used to destroy malignant cells to which the radiopharmaceutical is bound. The α-particles have higher energy and much greater mass. Actinium-225 is a commonly used α-emitter, whereas lutetium-177 is a commonly used β⁻-emitter. Therapeutic nuclear medicine therapy can be repeated multiple times to achieve the most benefit. Therapeutic nuclear medicine might be used in combination with or as an alternative to other treatment options, such as chemotherapy, radiotherapy, and surgery. It may improve the quality of life and shrink or stabilize the tumors for many patients.

Theranostics is a combination of the terms therapeutics and diagnostics. Theranostics can be used to describe the combination of using one radioactive drug to identify (diagnose) and a second radioactive drug to deliver therapy to treat the main tumor and any metastatic tumors.

Theranostic methods refer to the pairing of molecular imaging with therapeutic nuclear medicine. The ‘see it, treat it’ approach allows selecting patients amenable to therapeutic nuclear medicine with PET imaging. A pair of radiopharmaceuticals targeting the same disease is needed to image and treat the condition, for example, the theranostic pairs targeting somatostatin receptor type 2 (SSTR2) on neuroendocrine tumors (NETs). Octapeptide TATE is a somatostatin analog (SSA) which has high affinity to SSTR2. Therapeutic nuclear medicine with the ¹⁷⁷Lu-labeled [¹⁷⁷Lu]Lu-DOTA-TATE can be used. On the diagnostics side, ⁶⁸Ga-labeled SSAs are used for the molecular imaging of NETs with PET/CT. PET/CT imaging with [⁶⁸Ga]Ga-DOTA-TATE allows for NET staging with high accuracy and qualifies patients for therapeutic nuclear medicine. FIG. 1 illustrates the structures of ⁶⁸Ga-DOTA-TATE (PET imaging agent) and ¹⁷⁷Lu-DOTA-TATE (therapeutics). Another example includes the diagnosis of metastatic prostate cancer using PSMA PET. One PSMA targeting theranostic pair is ⁶⁸Ga-PSMA-617 for PET and ¹⁷⁷Lu-PSMA-617 for therapy. FIG. 2 illustrates the structures of the precursor for ⁶⁸Ga-PSMA-617 and the precursor for ¹⁷⁷Lu-PSMA-617. ⁶⁸Ga-PSMA-617 can be used as a PET imaging agent. ¹⁷⁷Lu-PSMA-617 can be used for therapeutics. The Glu-ureido-based peptidomimetic is a high-affinity PSMA binder.

Fluorine-18 is a common PET-radionuclide in clinical applications. The half-life of fluorine-18 (t_{1/2}=109.8 min, 97% β⁺-decay) is long enough to enable radiopharmaceutical production and shipment to off-site imaging facilities. The output of the cyclotron-produced nuclide is scalable, and up to several hundred patient doses can be produced in a single production run. Its comparably low positron energy (maximum β⁺ energy=633 keV) allows imaging with high spatial resolution and few artifacts.

Conventional ¹⁸F incorporation into radiotracers used for diagnostic PET imaging uses nucleophilic or electrophilic substitution chemistry, which involves the formation of C-¹⁸F bonds. Harsh reaction conditions including high temperatures, aprotic organic solvents, strong basic conditions during radiolabeling, and strong acidic conditions during the removal of protecting groups, are typically needed. Conventional ¹⁸F incorporation can also include complicated and lengthy multi-step production procedures and the generation of radioactive and non-radioactive byproducts that need a time-intensive and costly purification process, such as semi-preparative high-performance liquid chromatography.

Compared to the conventional radiolabeling methods, silicon-fluoride-acceptor (SiFA) building blocks enable rapid Si—¹⁸F bond formation, yielding ¹⁸F-SiFA-conjugated PET tracers in high radiochemical yields (RCY) and high molar activities (Aₘ). The formation of the Si—¹⁸F bond can occur under mild reaction conditions such as at room temperature. Protecting groups masking sensitive functional groups may not be required. Moreover, the absence of side products allows for a simple cartridge-based purification, resulting in a total synthesis time of about 20 minutes. However, the inherent shortcoming of traditional phenyl-SiFA technology is its high lipophilicity. The high lipophilicity can be caused by the two tert-butyl substituents on silicon and the phenyl core which has a log P of about 2.13. The two tert-butyl substituents on silicon are needed for steric shielding of the Si—¹⁸F bond to prevent its hydrolysis in vivo. (See, e.g., S. Niedermoser, et al., The Journal of Nuclear Medicine, 2015, 56, 7, 1100-1105; the disclosure of which is incorporated herein by reference in its entirety.) Consequently, polar (carboxylic acids, carbohydrates, polyethylene glycol) and charged (tertiary ammonium salts) linkers are necessary to mask the high lipophilicity of the phenyl-SiFA moiety.

SiFA with heteroaromatic rings (HetSiFA) technology uses more hydrophilic heteroaromatic rings to substitute the phenyl ring of SiFA. (See, e.g., PCT Application No. WO 2020/220020 A1 to J. M. Murphy et al., U.S. patent Ser. No. 10/800,797 B2 to C. M. Waldmann et al.; the disclosures of which are incorporated herein by references in their entirety.) The fast and reliable ¹⁸F-fluorination of HetSiFA is straightforward, and production can be scalable, which increases the accessibility of the drug for imaging centers and patients. The mild reaction conditions allow the labeling of sensitive substrates, including (but not limited to) peptides and peptidomimetics. Despite the advances, existing SiFA systems have inherit disadvantages for therapeutics, diagnostics, and/or theranostics applications.

### BRIEF SUMMARY OF THE INVENTION

Many embodiments are directed to systems and methods for heteroaromatic silicon-fluoride acceptor systems. In several embodiments, systems and methods for heteroaromatic silicon-fluoride acceptor systems with theranostic properties are described. Some embodiments are directed to systems and methods for heteroaromatic silicon-fluoride acceptor systems with theranostic capabilities targeting various types of metastatic cancers including (but not limited to) prostate cancers.

One embodiment of the invention includes a compound comprising: a silicon-fluoride acceptor; and a heteroaromatic ring; wherein the heteroaromatic ring is selected from the group consisting of: pyridine, pyridine oxide, pyridinium, pyrazole, fused pyrazole derivative, benzofuran, benzothiophene, indole, azaindole, imidazole, and pyrimidine;
wherein the pyridine, pyridine oxide, or pyridinium compound has a formula I of:

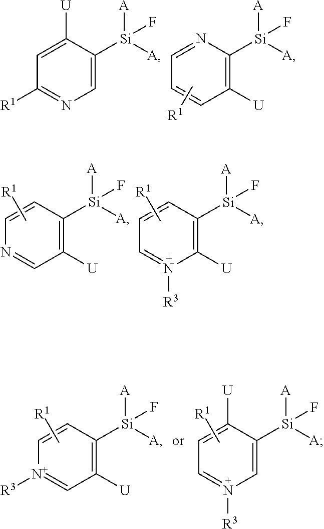

wherein the pyrazole compound has a formula II of:

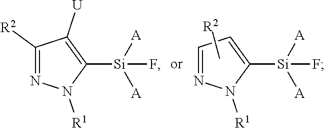

wherein the fused pyrazole derivative compound has a formula III of:

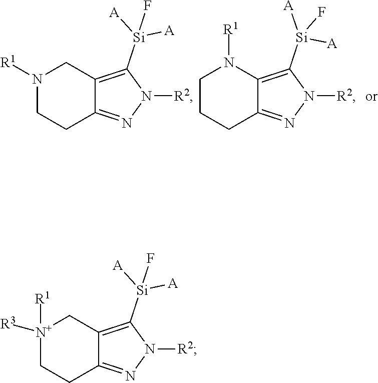

wherein the benzofuran, benzothiophene, indole, or azaindole compound has a formula IV of:

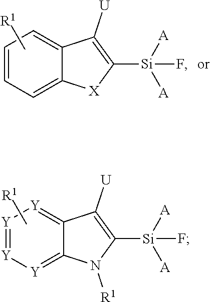

wherein the imidazole compound has a formula V of:

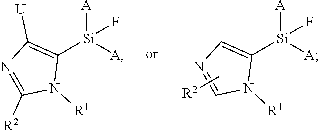

wherein the pyrimidine compound has a formula VI of:

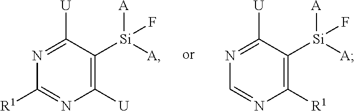

wherein:
each F is independently: F, or ¹⁸F, or ¹⁹F;
each A is independently: H, CH₃, CH₂—CH₃, CH₃—CH₂—CH(CH₃), CH(CH₃)₂, and C(CH₃)₃;
each U is independently: O—CH₃, CH₃, CH₂CH₃, H, I, Br, Cl, F, N(CH₃)₂ and CH₂CH(NH₂)CO₂H;
each X is independently: O, S, and N;
each Y is independently: C and N;
each R¹ or R² or R³ is independently: CH₃, CH₂—CH₃, H, L¹-CH₂—C≡C, L¹-CH₃, L¹-G, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²-L³-L⁴-G, L¹-L²-L³-L⁴-H, L¹-L²-L³-L⁴-Q-L⁵-G, L¹-L²-L³-Q, L¹-L²-L³-Q-L⁴-G, L¹-L²-N₃, L¹-L²-OH, L¹-L²-Q, L¹-L²-Q-G, L¹-L²-Q-L³-G, L¹-L²-Q-L³-L⁴-G, L¹-L²-Q-L³-L⁴-H, L¹-OH, L¹-Q, L¹-Q-L²-L³-L⁴-G, L¹-Q-L²-G, NH₂, O⁻, O—CH₃, OH,

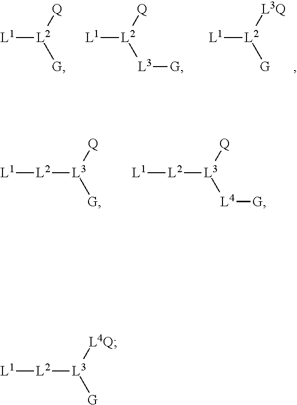

each L¹ or L² or L³ or L⁴ or L⁵ is independently: —(O—CH₂—CH₂)ₚ—, —(CH₂—CH₂—O)ₚ—, -(Glu-His)ₚ-, -(His-Glu)ₚ-, -(Glu-Trp)ₚ-, -(Trp-Glu)ₚ-, —NH—CH₂—C₆H₄—NH—(C═O)—CH₂—O—CH₂—(C═O)—, —(C═O)—CH₂—O—CH₂—(C═O)—NH—C₆H₄—CH₂—NH—, —(C═O)—CH₂—CH₂—(C═O)—, —NH—C₅H₉N—CH₂—(C═O)—, —(C═O)—CH₂—NC₅H₉—NH—, -(Gly)ₚ-, —CH₂—CH₂—NH—, —NH—CH₂—(C═O)—, -(Glu)-, —NH—CH₂—CH₂—, —(C═O)—, NH—CH₂—CH₂—(O—CH₂—CH₂)ₚ—(C═O)—, —(C═O)—(CH₂—CH₂—O)ₚ—CH₂—CH₂—NH—, —NH—(C═O)—CH₂—, —NH—(C═O)—NH—CH₂—, —NH—(C═S)—NH—CH₂—, —NH—(CH₂—CH₂—O)ₚ—CH₂—(C═O)—, —(C═O)—CH₂—(O—CH₂—CH₂)ₚ—NH—, -Asp-, —NH—, —NH—(CH₂—CH₂—O)ₚ—CH₂—CH₂—(C═O)—, —(C═O)—CH₂—CH₂—(O—CH₂—CH₂)ₚ—NH—, —CH₂—(C═O)—, —(C═O)—CH₂—, —O—(C═O)—, —(C═O)—O—, —CH₂—O—, —NH—(C═O)—O—, —O—(C═O)—NH—, —(C═O)—CH₂—(O—CH₂—CH₂)ₚ—O—CH₂—(C═O)—, —NH—(C═O)—NH—, —NH—(C═S)—NH—, —CH₂—N(CH₃)—CH₂—, —N(CH₃)—, —(C═O)—C₆H₄—(C═O)—, —C₆H₄—(C═O)—, —(C═O)—C₆H₄—, —O—CH₂—(C═O)—, —(C═O)—CH₂—O—, —CH₂—C₂N₃—CH₂—, —CH₂—CH₂—(C═O)—, —(C═O)—CH₂—CH₂—, —CH₂—N⁺(CH₃)₂—CH₂—, —CH₂—N(CH₃)—, —(C═O)—CH₂—(O—CH₂—CH₂)ₚ—, —(CH₂—CH₂—O)ₚ—CH₂—CH₂—NH—, or

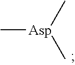

p=0 to 12;
each G is a disease binding ligand, wherein G is independently: a somatostatin receptor type 2 (SSTR2) binding ligand, a gastrin releasing peptide receptor (GRPR) binding ligand, a prostate specific membrane antigen (PSMA) binding ligand, a fibroblast activation protein (FAP) binding ligand, or a C-X-C chemokine receptor type 4 (CXCR-4) binding ligand; and
each Q is a chelator.

In a further embodiment, each Q is independently: -DOTA, -DOTAGA, -Dap(DOTA), -Lys(DOTA), -3p-C-NETA, -bis-thioseminarabazones, -EDTA, -CHX-A″-EDTA, -DTPA, -p-SCN-DPTA, -CHX-A″-DTPA, -p-SCN-Bz-Mx-DTPA, -NOTA, -TETA, -CB-TE2A, -p-SCN-NOTA (cNOTA), -nNOTA, -NODAGA, -p-SCN-DOTA (cDOTA), -2-cTETA, -6-cTETA, -BAT, -Diamsa, -SarAr, -PCTA, -NODIA-Me, -TRAP, -pycup1A1B, -p-SCN-DTPA, -Desferrioxamine B(DFO) Mesylate, -Desferrioxamine-p-SCN, -DFO-Star (DFO*), -L5, -Orn3hx-NCS, -Orn4hx-NCS, -p-SCN-Bn-HOPO, -2,3-HOPO-p-Bn-NCS, -YM103, -Tc(V)oxo, -Tc(V)nitride, -Tc(V)HYNIC, -Tc(I)-fac-tricarbonyl, -Tc(VII)trioxo, -3p-C-NETA-NCS, -3p-C-DEPA-NCS, -TCMC, -p-SCN-Bn-H₄octapa, -HEHA-NCS, or -Macropa-NCS.

In another embodiment, Q is unchelated or optionally chelated with M, wherein M is a cation of a metal selected from the group consisting of: ⁴³Sc, ⁴⁴Sc, ⁴⁵Sc, ⁴⁷SC, ⁵¹Cr, ^{52m}Mn, ⁶⁸Co, ⁵²Fe, ⁵⁶Ni, ⁵⁷Ni, ⁶¹Cu, ⁶²Cu, ⁶³Cu, ⁶⁴Cu, ⁶⁵Cu, ⁶⁷Cu, ⁶⁶Ga, ⁶⁷Ga, ⁶⁸Ga, ⁶⁹Ga, ⁷¹Ga, ^{nat}Ga, ⁹⁰Zr, ⁹¹Zr, ⁹²Zr, ⁸⁹Zr, ⁸⁶Y, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹I, ¹¹³In, ^{133m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵⁵Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁶Lu, ¹⁷⁷Lu, ^{177m}Lu, ^{nat}Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²⁰⁴Pb, ²⁰⁶Pb, ²⁰⁷Pb, ²⁰⁸Pb, ²¹¹At, ²⁰⁹Bi, ²¹²Bi, ²¹³Bi, ²²³Ra, ²²⁵Ac, ²²⁷Th, ²³²Th, and a cationic molecule comprising ¹⁸F, and ¹⁸F[AlF]²⁺.

In an additional embodiment, A is C(CH₃)₃.

In another further embodiment, G has a formula selected from the group consisting of:

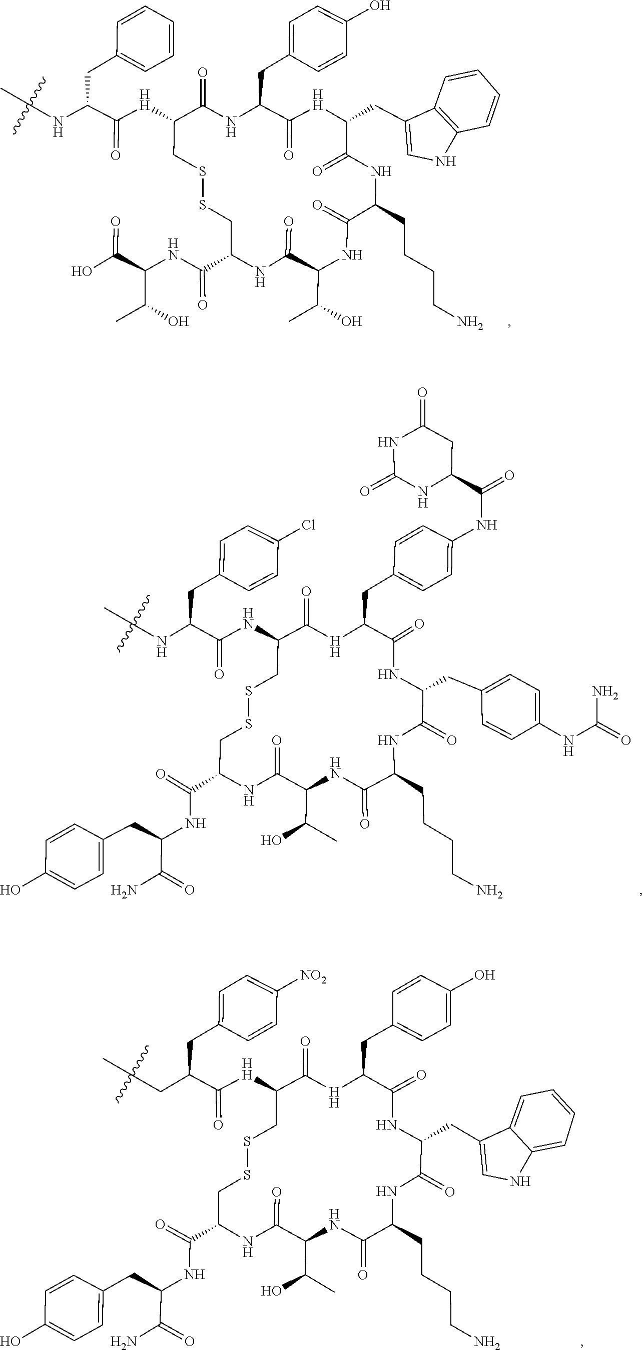

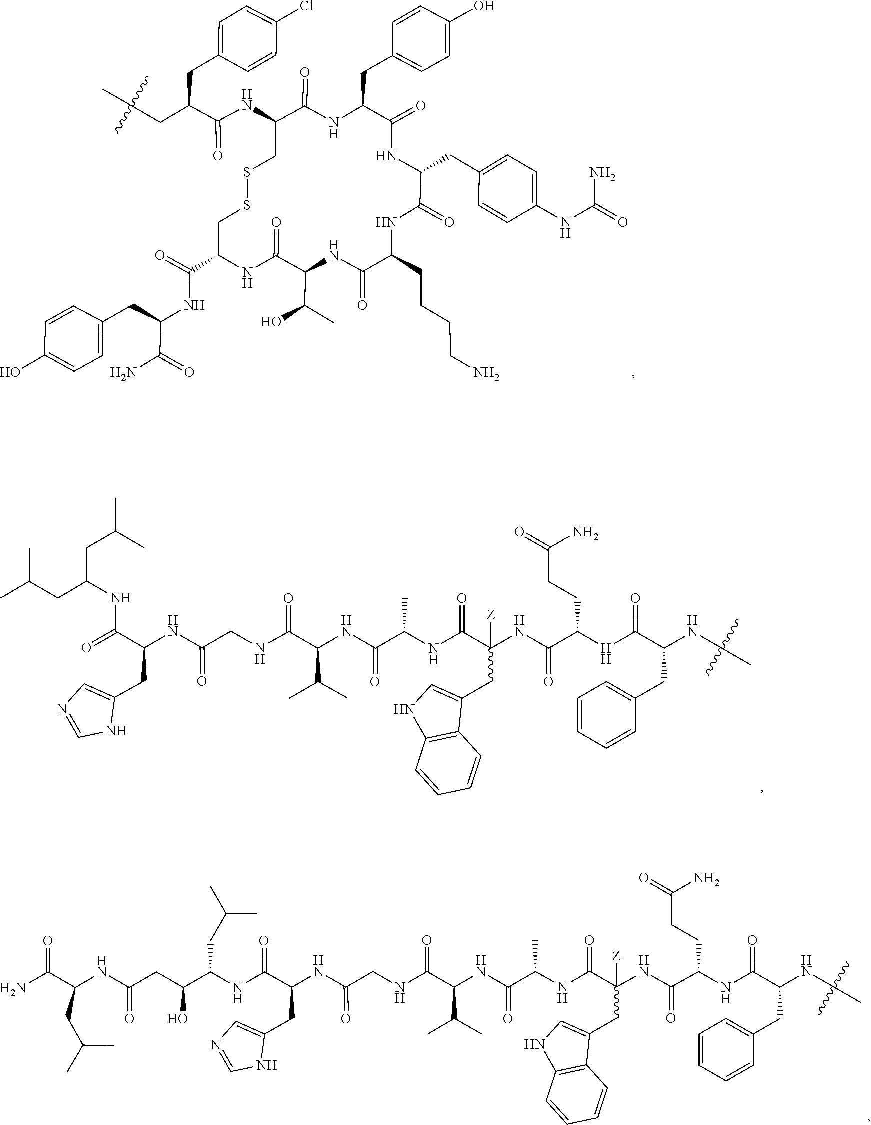

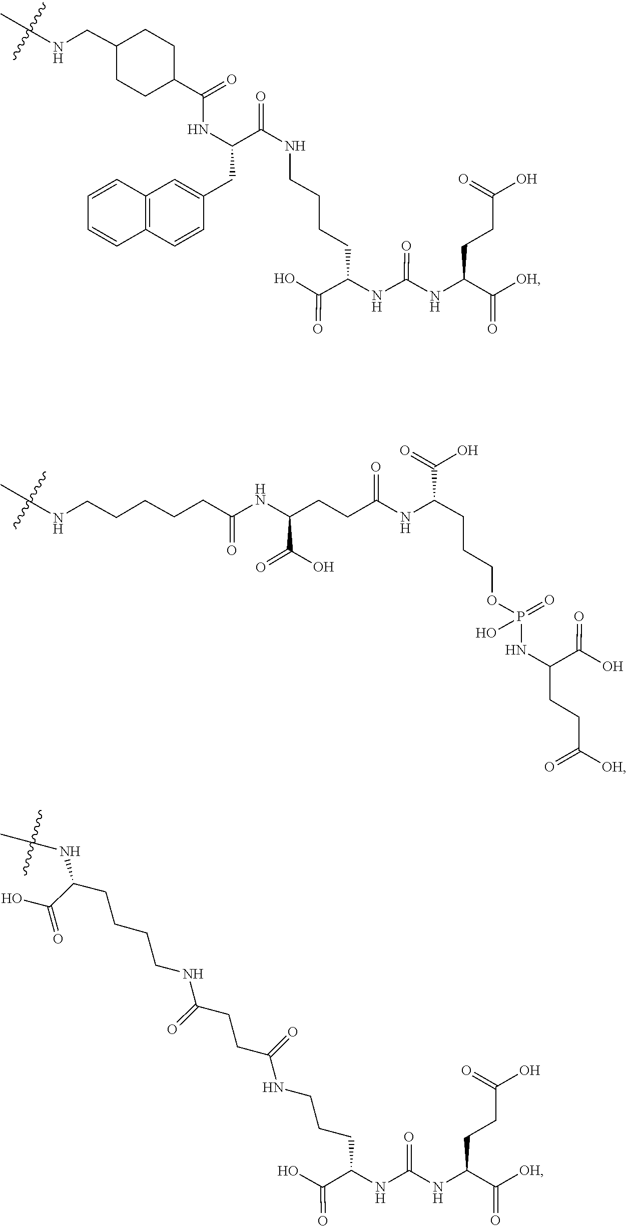

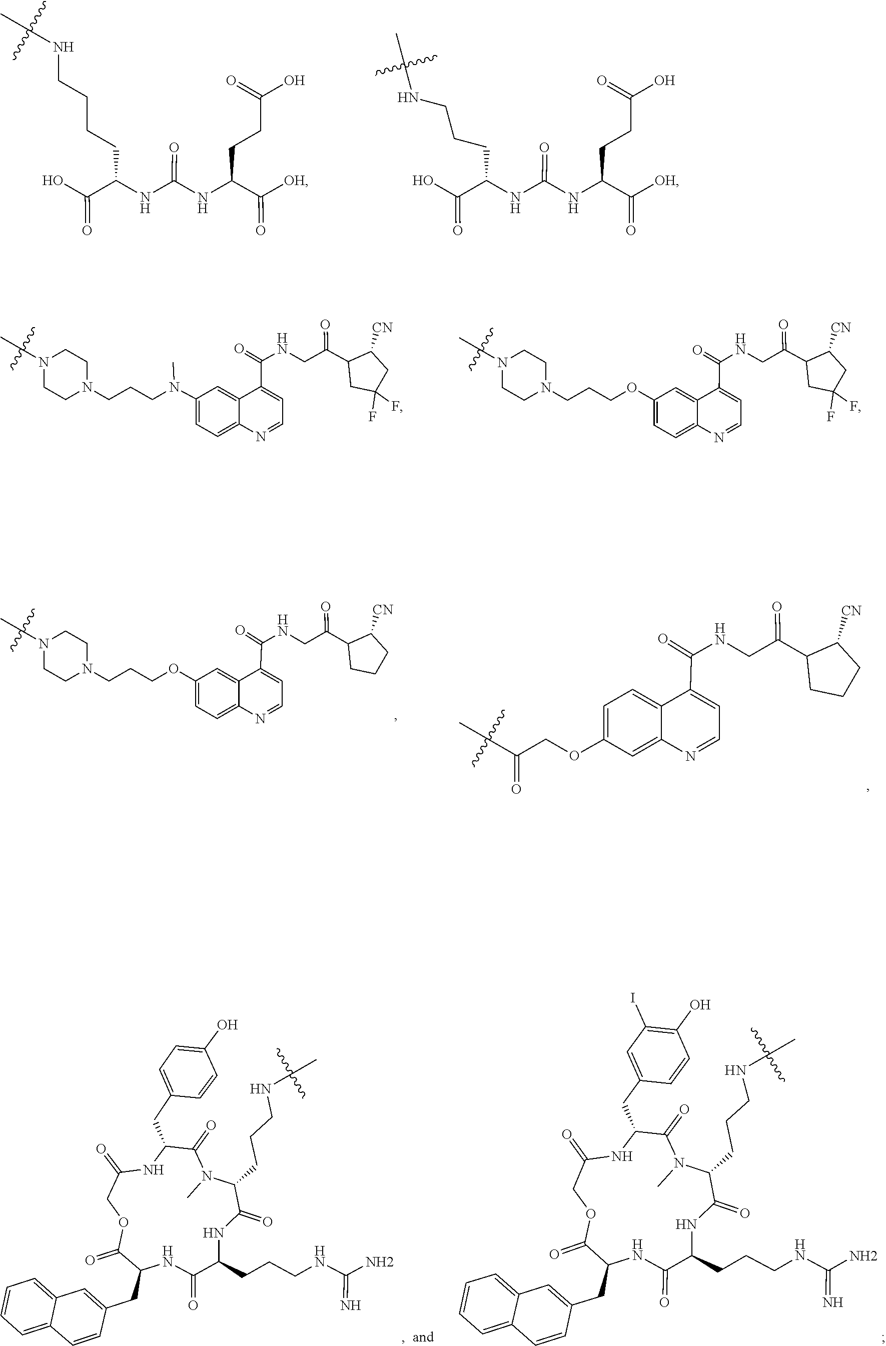

wherein Z is H, CHs or CF₃.

In a further yet embodiment,
R¹ is independently: CH₃, H, L¹-CH₂—C≡C, L¹-CH₃, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²-L³-L⁴-Q-L⁵-G, L¹-L²-L³- Q, L¹-L²-L³-Q-L⁴-G, L¹-L²-N₃, L¹-L²-OH, L¹-L²-Q, L¹-L²-Q-L³-L⁴-G, L¹-OH, L¹-Q, L¹-Q-L²-L³-

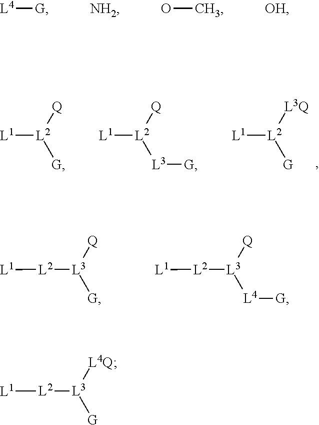

R² is independently: CH₃, H, L¹-CH₂—C≡C, L¹-G, L¹-H, L¹-L²-CH₂—C≡C, L¹-L²-G, L¹-L²-L³-G, L¹-L²-L³-L⁴-G, L¹-L²-L³-Q, L¹-L²-L³-Q-L⁴-G, L¹-L²-N₃, L¹-L²-Q, L¹-L²-Q-G, L¹-L²-Q-L³-G, L¹-OH, L¹-Q, L¹-Q-L²-G, NH₂, O⁻, or O—CH₃; and
R³ is independently: CH₃, CH₂—CH₃, or O⁻.

In another further yet embodiment, the compound is a small molecule with a formula selected from the group consisting of:

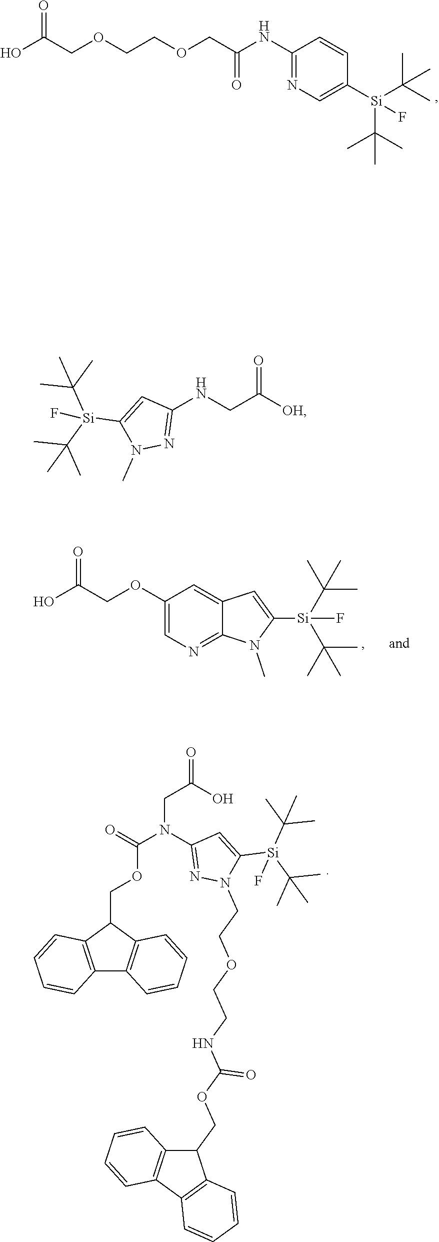

In an additional embodiment again, the compound is for positron emission tomography (PET) imaging and has a formula of:

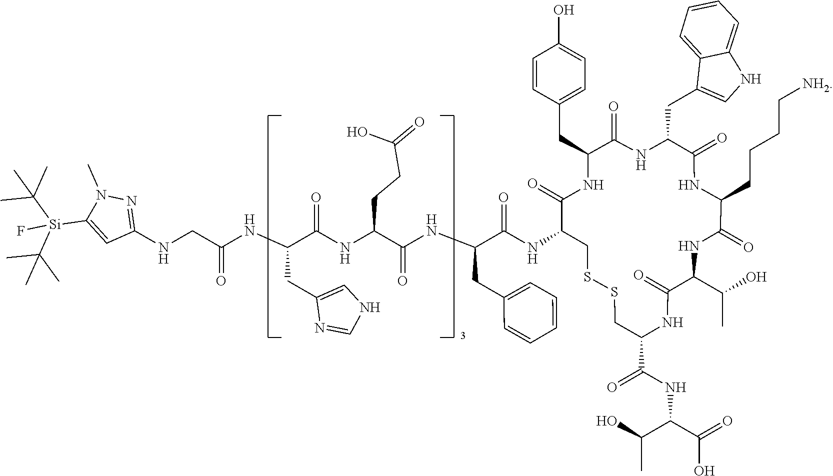

In a yet further embodiment, the compound is a theranostic compound with a formula selected from the group consisting of:

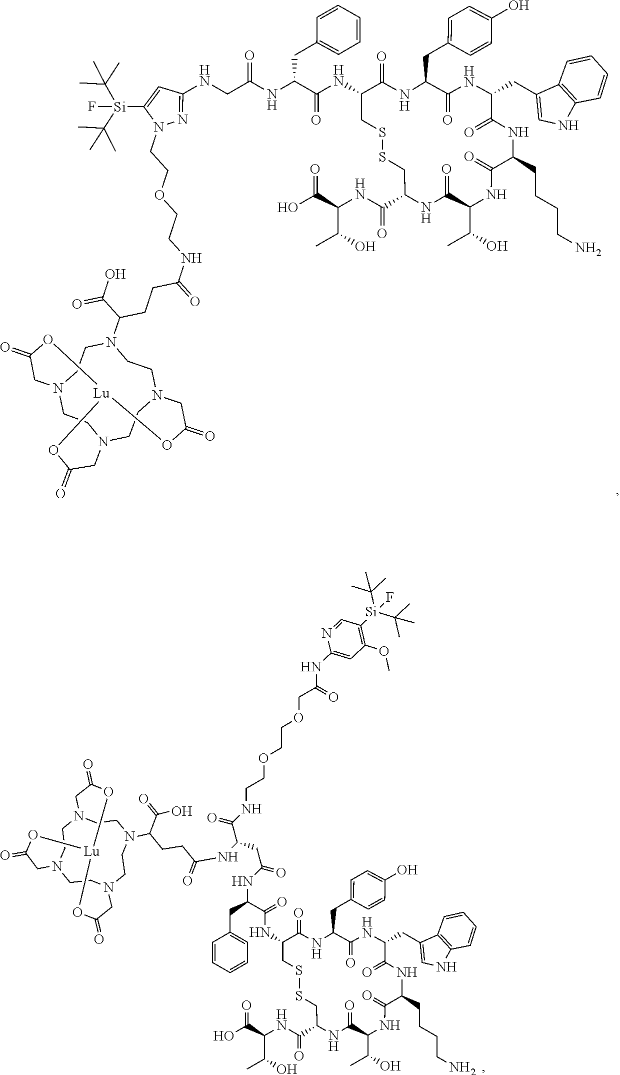

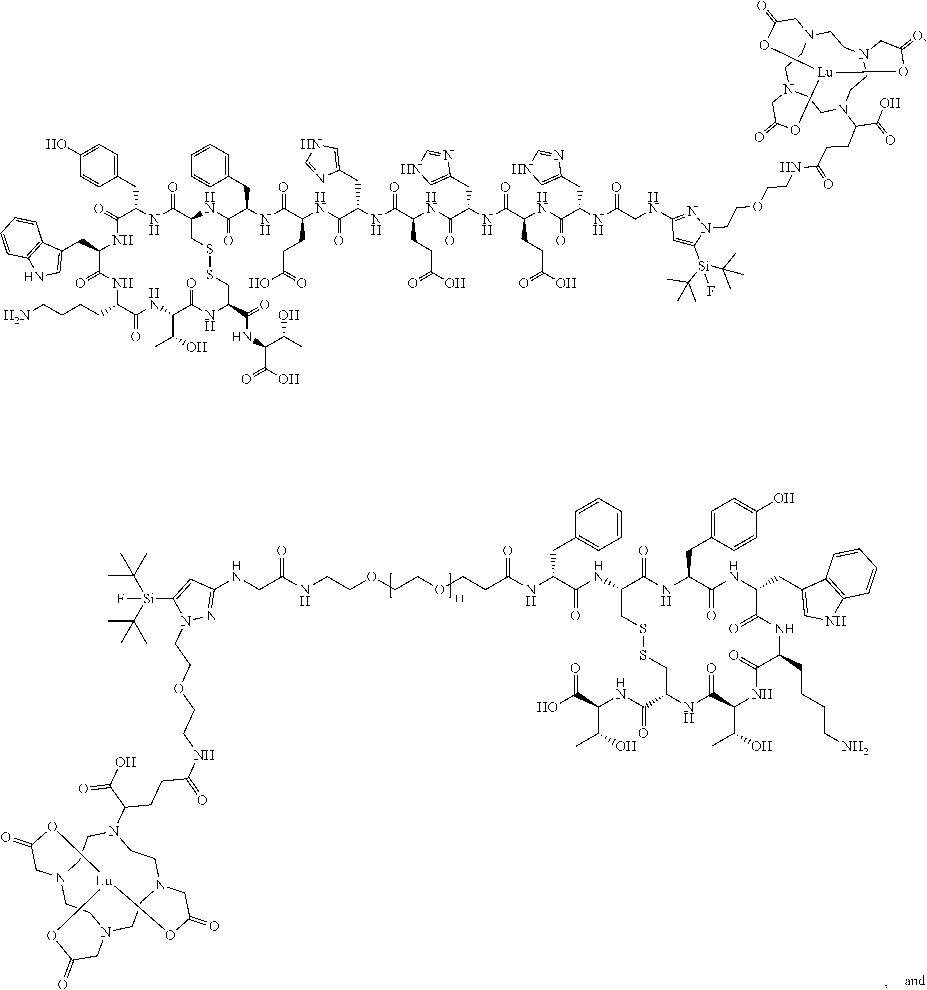

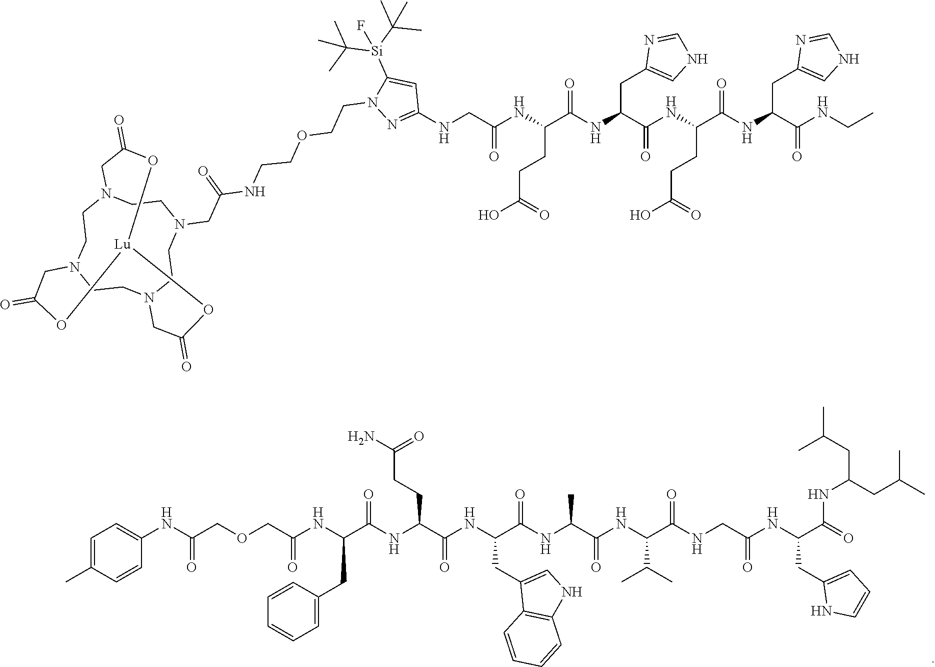

In another further yet embodiment again, the compound is a dual targeting theranostic compound with a formula of:

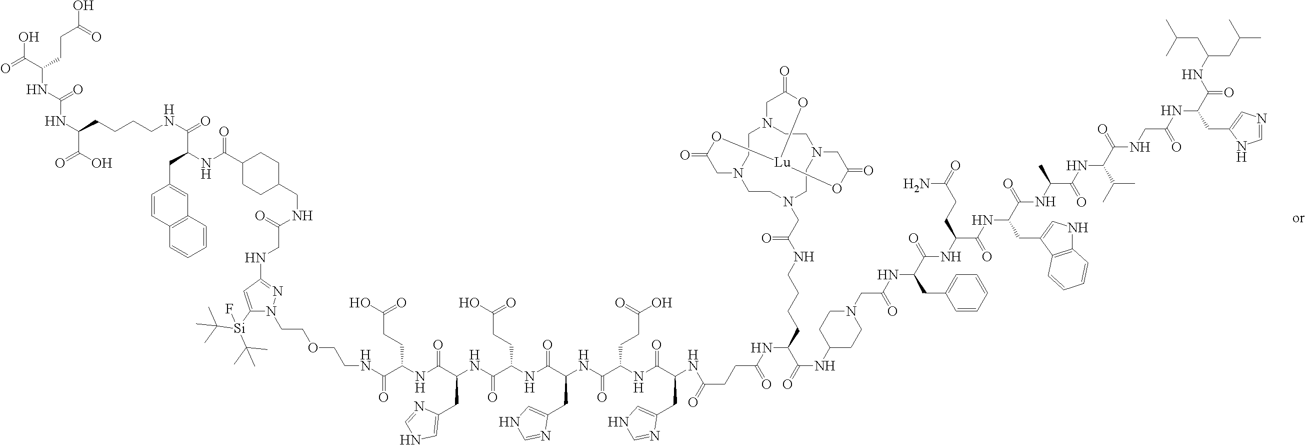

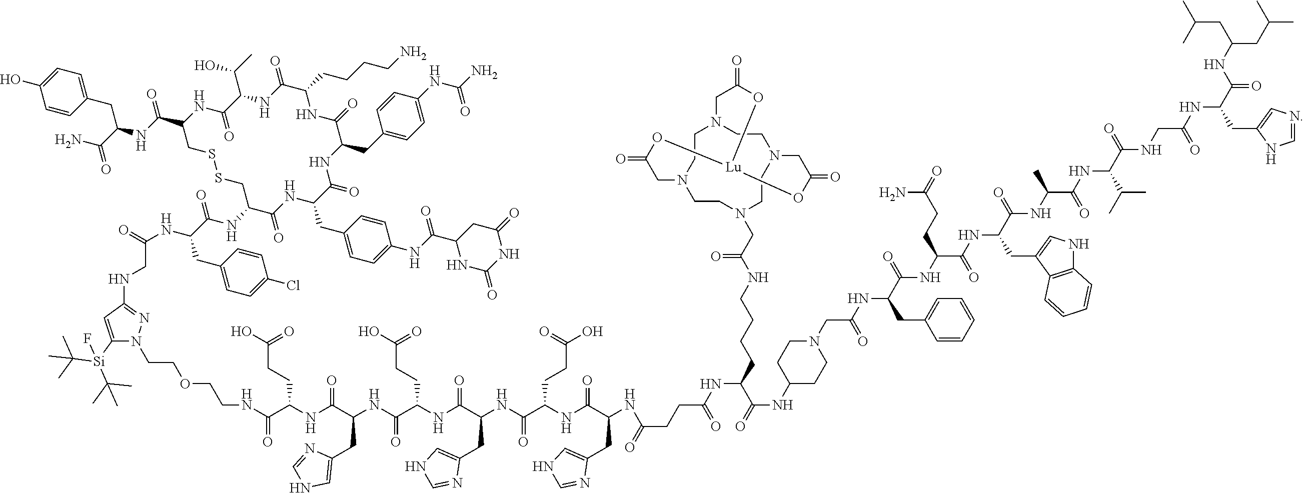

In an additional embodiment again, the compound is configured for PET imaging and therapeutic radioligand therapy.

In a yet further embodiment, the disease binding ligand G is conjugated to the compound via a process selected from the group consisting of: coupling chemistry, peptide coupling chemistry, copper-catalyzed azide-alkyne cycloaddition (CuAAC), and solid-phase peptide synthesis (SPPS).

In yet another embodiment, 3p-C-NETA is configured to chelate with ¹⁷⁷Lu for therapeutics; DOTA and DOTAGA are configured to chelate with ¹⁷⁷Lu or ²²⁵Ac for therapeutics; and DOTA and DOTAGA are configured to chelate with ⁶⁸Ga, ⁸⁹Zr, or ⁶⁴Cu for PET imaging.

Another embodiment includes a method for synthesizing a radiopharmaceutical compound comprising: providing a silicon fluoride compound comprising a heteroaromatic ring; and conjugating a disease binding ligand G to the silicon fluoride compound to form the radiopharmaceutical compound; wherein the heteroaromatic ring is selected from the group consisting of: pyridine, pyridine oxide, pyridinium, pyrazole, fused pyrazole derivative, benzofuran, benzothiophene, indole, azaindole, imidazole, and pyrimidine;
wherein the pyridine, pyridine oxide, or pyridinium compound has a formula I of:

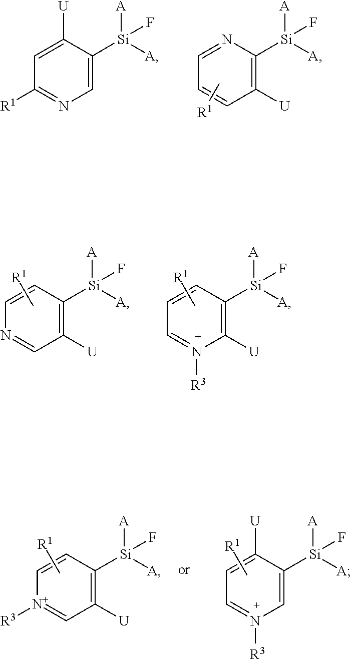

wherein the pyrazole compound has a formula II of:

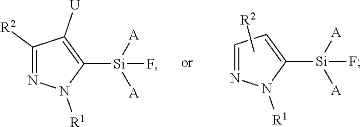

wherein the fused pyrazole derivative compound has a formula III of:

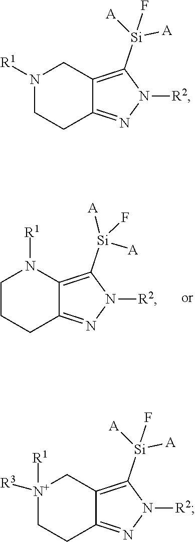

wherein the benzofuran, benzothiophene, indole, or azaindole compound has a formula IV of:

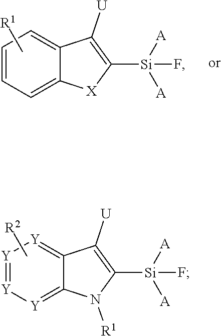

wherein the imidazole compound has a formula V of:

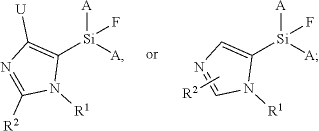

wherein the pyrimidine compound has a formula VI of:

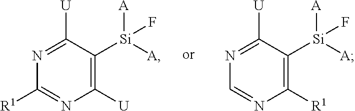

wherein:
each F is independently: F, or ¹⁸F, or ¹⁹F;
each A is independently: H, CH₃, CH₂—CH₃, CH₃—CH₂—CH(CH₃), CH(CH₃)₂, and C(CH₃)₃;
each U is independently: O—CH₃, CH₃, CH₂CH₃, H, I, Br, Cl, F, N(CH₃)₂ and CH₂CH(NH₂)CO₂H;
each X is independently: O, S, and N;
each Y is independently: C and N;
each R¹ or R² or R³ is independently: CH₃, CH₂—CH₃, H, L¹-CH₂—C≡C, L¹-CH₃, L¹-G, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²-L³-L⁴-G, L¹-L²-L³-L⁴-H, L¹-L²-L³-L⁴-Q-L⁵-G, L¹-L²-L³-Q, L¹-L²-L³-Q-L⁴-G, L¹-L²-N₃, L¹-L²-OH, L¹-L²-Q, L¹-L²-Q-G, L¹-L²-Q-L³-G, L¹-L²-Q-L³-L⁴-G, L¹-L²-Q-L³-L⁴-H, L¹-OH, L¹-Q, L¹-Q-L²-L³-L⁴-G, L¹-Q-L²-G, NH₂, O⁻, O—CH₃, OH,

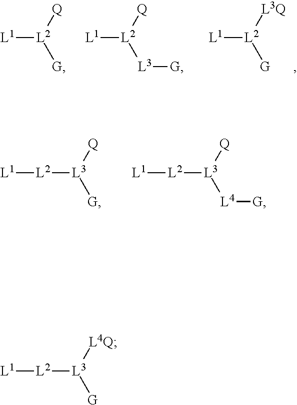

each L¹ or L² or L³ or L⁴ or L⁵ is independently: —(O—CH₂—CH₂)ₚ—, —(CH₂—CH₂—O)ₚ—, -(Glu-His)ₚ-, -(His-Glu)ₚ-, -(Glu-Trp)ₚ-, -(Trp-Glu)ₚ-, —NH—CH₂—C₆H₄—NH—(C═O)—CH₂—O—CH₂—(C═O)—, —(C═O)—CH₂—O—CH₂—(C═O)—NH—C₆H₄—CH₂—NH—, —(C═O)—CH₂—CH₂—(C═O)—, —NH—C₅H₉N—CH₂—(C═O)—, —(C═O)—CH₂—NC₅H₉—NH—, -(Gly)ₚ-, —CH₂—CH₂—NH—, —NH—CH₂—(C═O)—, -(Glu)-, —NH—CH₂—CH₂—, —(C═O)—, NH—CH₂—CH₂—(O—CH₂—CH₂)ₚ—(C═O)—, —(C═O)—(CH₂—CH₂—O)ₚ—CH₂—CH₂—NH—, —NH—(C═O)—CH₂—, —NH—(C═O)—NH—CH₂—, —NH—(C═S)—NH—CH₂—, —NH—(CH₂—CH₂—O)ₚ—CH₂—(C═O)—, —(C═O)—CH₂—(O—CH₂—CH₂)ₚ—NH—, -Asp-, —NH—, —NH—(CH₂—CH₂—O)ₚ—CH₂—CH₂—(C═O)—, —(C═O)—CH₂—CH₂—(O—CH₂—CH₂)ₚ—NH—, —CH₂—(C═O)—, —(C═O)—CH₂—, —O—(C═O)—, —(C═O)—O—, —CH₂—O—, —NH—(C═O)—O—, —O—(C═O)—NH—, —(C═O)—CH₂—(O—CH₂—CH₂)ₚ—O—CH₂—(C═O)—, —NH—(C═O)—NH—, —NH—(C═S)—NH—, —CH₂—N(CH₃)—CH₂—, —N(CH₃)—, —(C═O)—C₆H₄—(C═O)—, —C₆H₄—(C═O)—, —(C═O)—C₆H₄—, —O—CH₂—(C═O)—, —(C═O)—CH₂—O—, —CH₂—C₂N₃—CH₂—, —CH₂—CH₂—(C═O)—, —(C═O)—CH₂—CH₂—, —CH₂—N⁺(CH₃)₂—CH₂—, —CH₂—N(CH₃)—, —(C═O)—CH₂—(O—CH₂—CH₂)ₚ—, —(CH₂—CH₂—O)ₚ—CH₂—CH₂—NH—, or

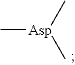

p=0 to 12;
each G is independently: a somatostatin receptor type 2 (SSTR2) binding ligand, a gastrin releasing peptide receptor (GRPR) binding ligand, a prostate specific membrane antigen (PSMA) binding ligand, a fibroblast activation protein (FAP) binding ligand, or a C-X-C chemokine receptor type 4 (CXCR-4) binding ligand; and each Q is a chelator.

In a further yet embodiment, each Q is independently: -DOTA, -DOTAGA, -Dap(DOTA), -Lys(DOTA), -3p-C-NETA, -bis-thioseminarabazones, -EDTA, -CHX-A″-EDTA, -DTPA, -p-SCN-DPTA, -CHX-A″-DTPA, -p-SCN-Bz-Mx-DTPA, -NOTA, -TETA, -CB-TE2A, -p-SCN-NOTA (cNOTA), -nNOTA, -NODAGA, -p-SCN-DOTA (cDOTA), -2-cTETA, -6-cTETA, -BAT, -Diamsa, -SarAr, -PCTA, -NODIA-Me, -TRAP, -pycup1A1B, -p-SCN-DTPA, -Desferrioxamine B(DFO) Mesylate, -Desferrioxamine-p-SCN, -DFO-Star (DFO*), -L5, -Orn3hx-NCS, -Orn4hx-NCS, -p-SCN-Bn-HOPO, -2,3-HOPO-p-Bn-NCS, -YM103, -Tc(V)oxo, -Tc(V)nitride, -Tc(V)HYNIC, -Tc(I)-fac-tricarbonyl, -Tc(VII)trioxo, -3p-C-NETA-NCS, -3p-C-DEPA-NCS, -TCMC, -p-SCN-Bn-H₄octapa, -HEHA-NCS, or -Macropa-NCS.

Another additional embodiment, further comprises chelating Q with M, wherein M is a cation of a metal selected from the group consisting of: ⁴³Sc, ⁴⁴Sc, ⁴⁵Sc, ⁴⁷SC, ⁵¹Cr, ^{52m}Mn, ⁶⁸Co, ⁵²Fe, ⁵⁶Ni, ⁵⁷Ni, ⁶¹Cu, ⁶²Cu, ⁶³Cu, ⁶⁴Cu, ⁶⁵Cu, ⁶⁷Cu, ⁶⁶Ga, ⁶⁷Ga, ⁶⁸Ga, ⁶⁹Ga, ⁷¹Ga, ^{nat}Ga, ⁹⁰Zr, ⁹¹Zr, ⁹²Zr, ⁸⁹Zr, ⁸⁶Y, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹I, ¹¹³In, ^{133m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵⁵Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁶Lu, ¹⁷⁷Lu, ^{177m}Lu, ^{nat}Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²⁰⁴Pb, ²⁰⁶Pb, ²⁰⁷Pb, ²⁰⁸Pb, ²¹¹At, ²⁰⁹Bi, ²¹²Bi, ²¹³Bi, ²²³Ra, ²²⁵Ac, ²²⁷Th, ²³²Th, and a cationic molecule comprising ¹⁸F, and ¹⁸F[AlF]²⁺.

In an additional embodiment again, A is C(CH₃)₃.

In another yet embodiment, G has a formula selected from the group consisting of:

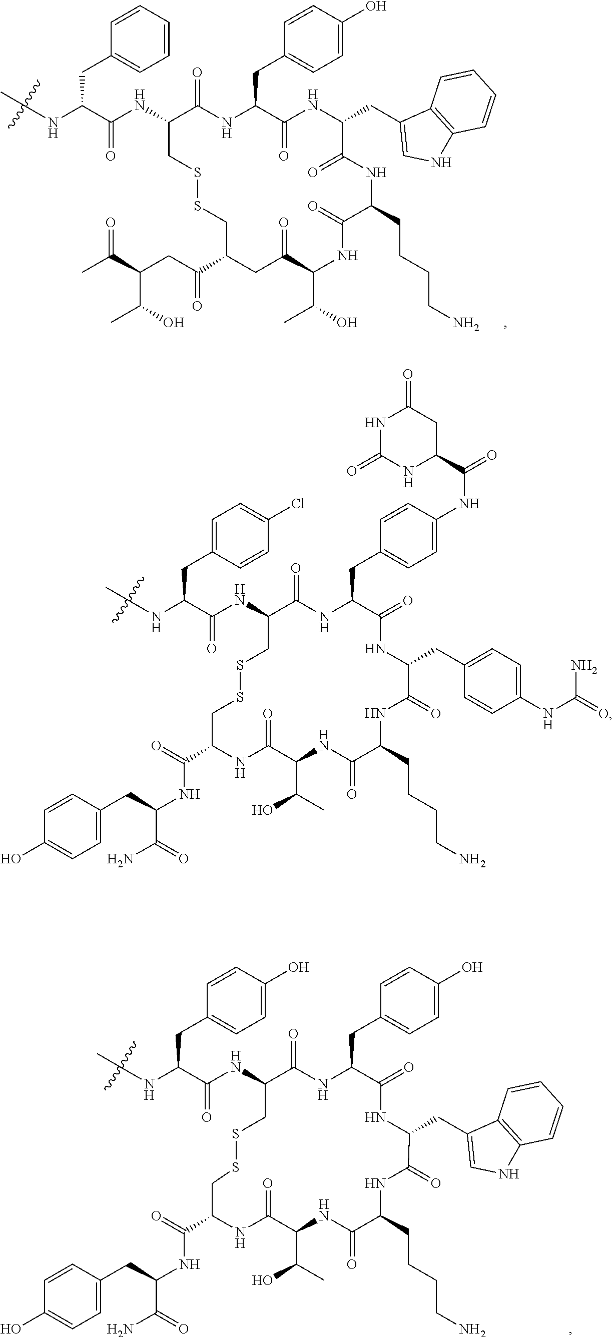

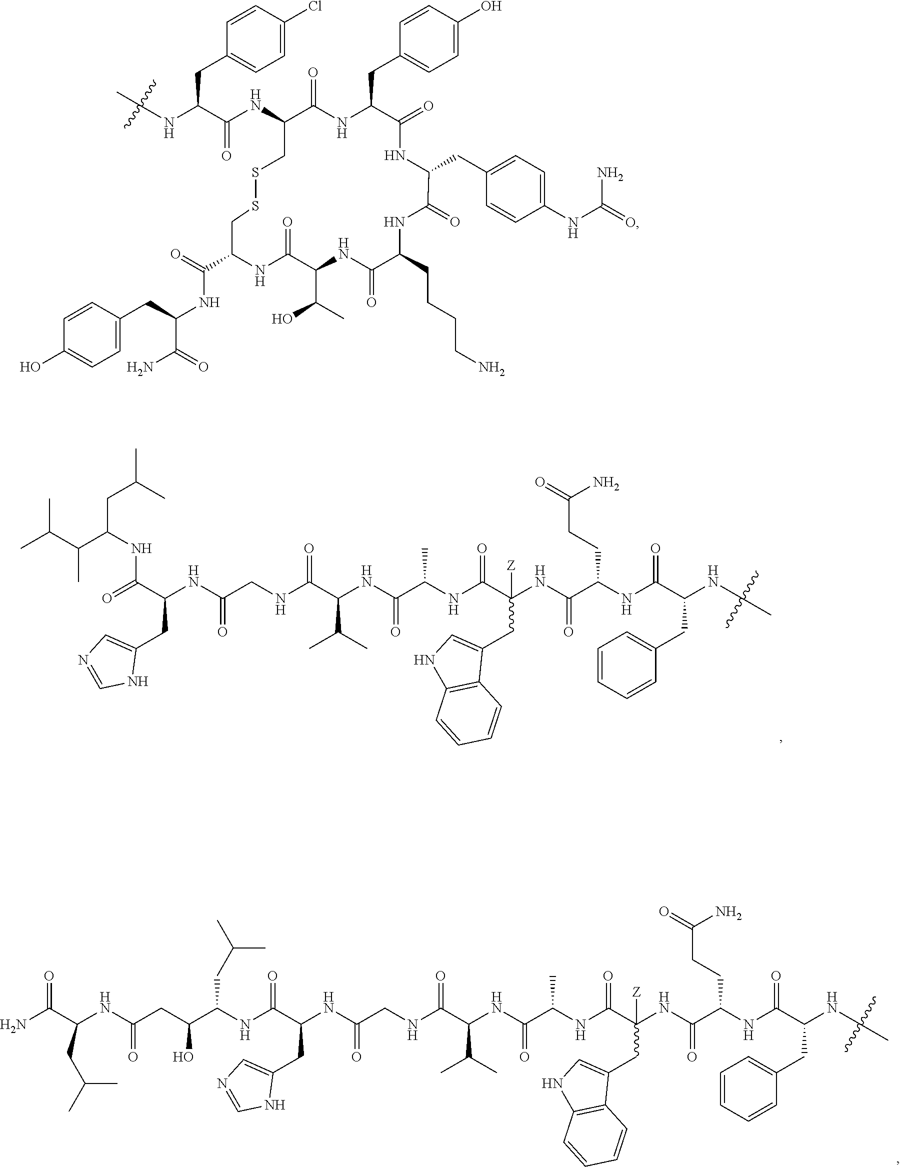

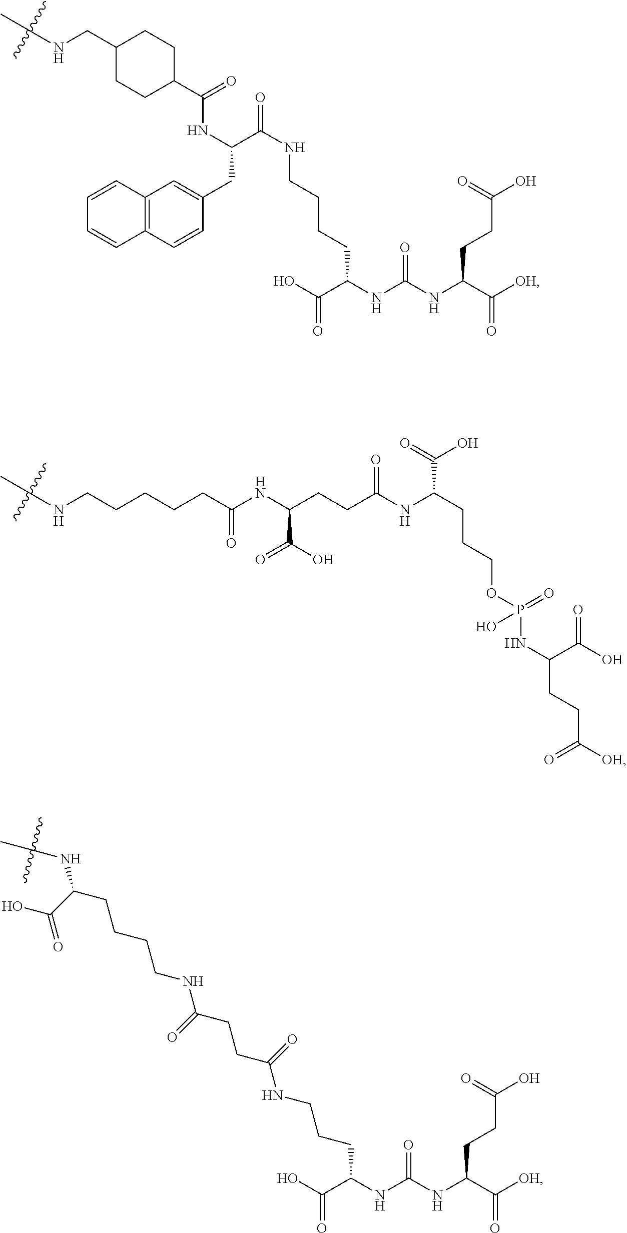

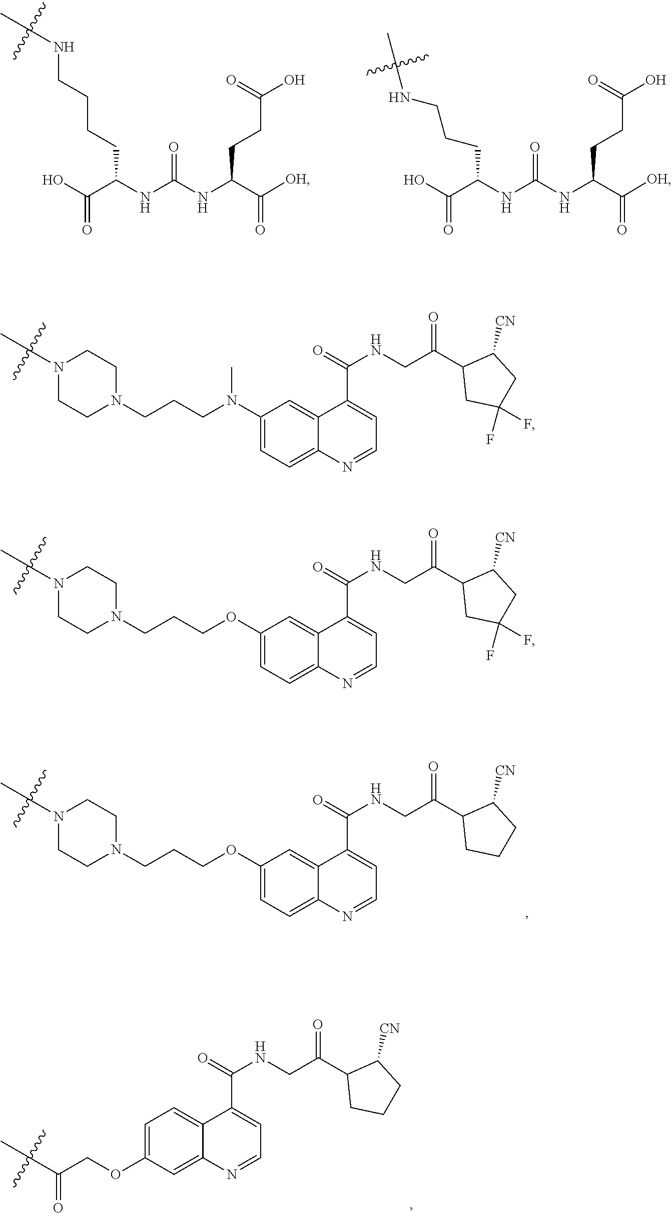

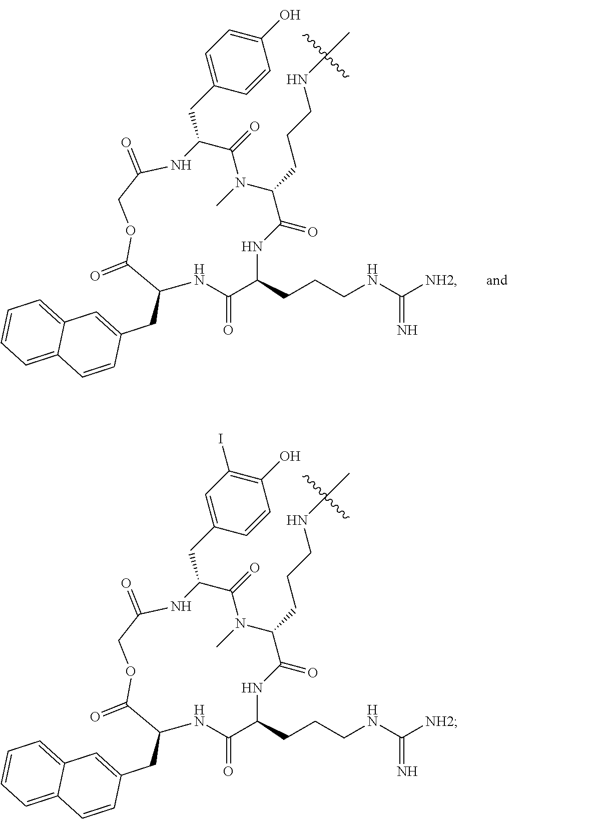

wherein Z is H, CHs or CF₃.

In another yet embodiment,
R¹ is independently: CH₃, H, L¹-CH₂—C≡C, L¹-CH₃, L¹-H, L¹-L²-CH₂—C≡C, L¹-L2-CH₃, L¹-L²-G, L¹-L²-H, L¹-L²-H₂, L¹-L²-L³-G, L¹-L²-L³-H, L¹-L²-L³-L⁴-Q-L⁵-G, L¹-L²-L³- Q, L¹-L²-L³-Q-L⁴-G, L¹-L²-N₃, L¹-L²-OH, L¹-L²-Q, L¹-L²-Q-L³-L⁴-G, L¹-OH, L¹-Q, L¹-Q-L²-L³-L⁴- G, NH₂, O-CH₃, OH,

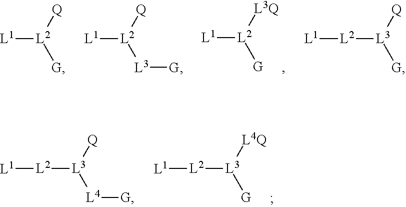

R² is independently: CH₃, H, L¹-CH₂—C≡C, L¹-G, L¹-H, L¹-L²-CH₂—C≡C, L¹-L²-G, L¹-L²-L³-G, L¹-L²-L³-L⁴-G, L¹-L²-L³-Q, L¹-L²-L³-Q-L⁴-G, L¹-L²-N₃, L¹-L²-Q, L¹-L²-Q-G, L¹-L²-Q-L³-G, L¹-OH, L¹-Q, L¹-Q-L²-G, NH₂, O⁻, or O—CH₃; and
R³ is independently: CH₃, CH₂—CH₃, or O⁻.

In a further embodiment again, the radiopharmaceutical compound is a small molecule with a formula selected from the group consisting of:

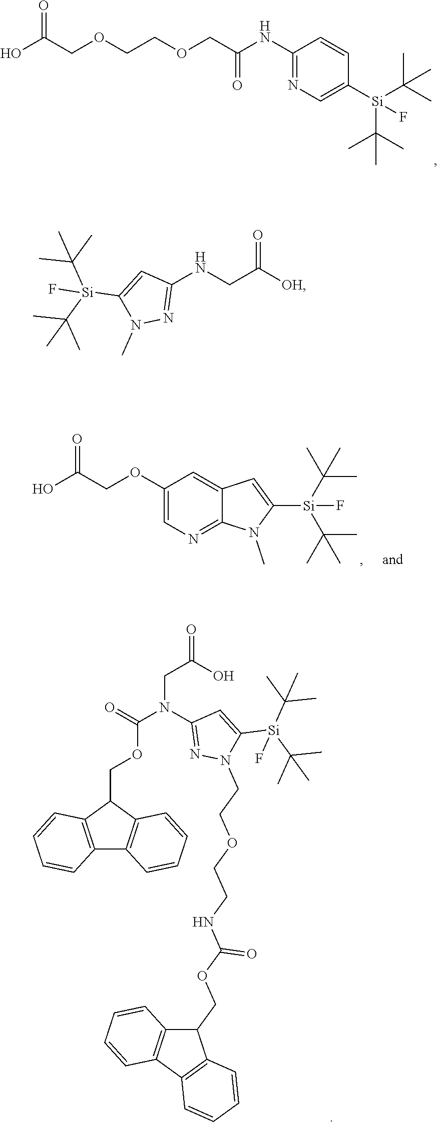

In a yet another embodiment again, the radiopharmaceutical compound is for positron emission tomography (PET) imaging and has a formula of:

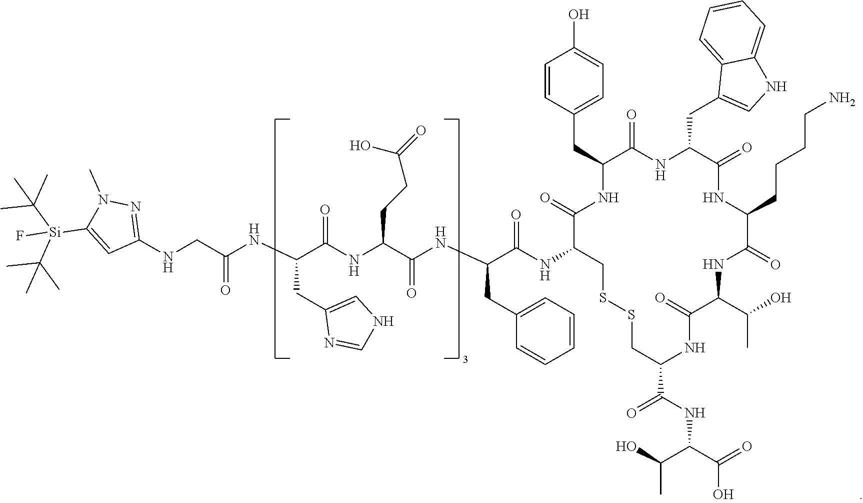

In yet another embodiment, the radiopharmaceutical compound is a theranostic compound with a formula selected from the group consisting of:

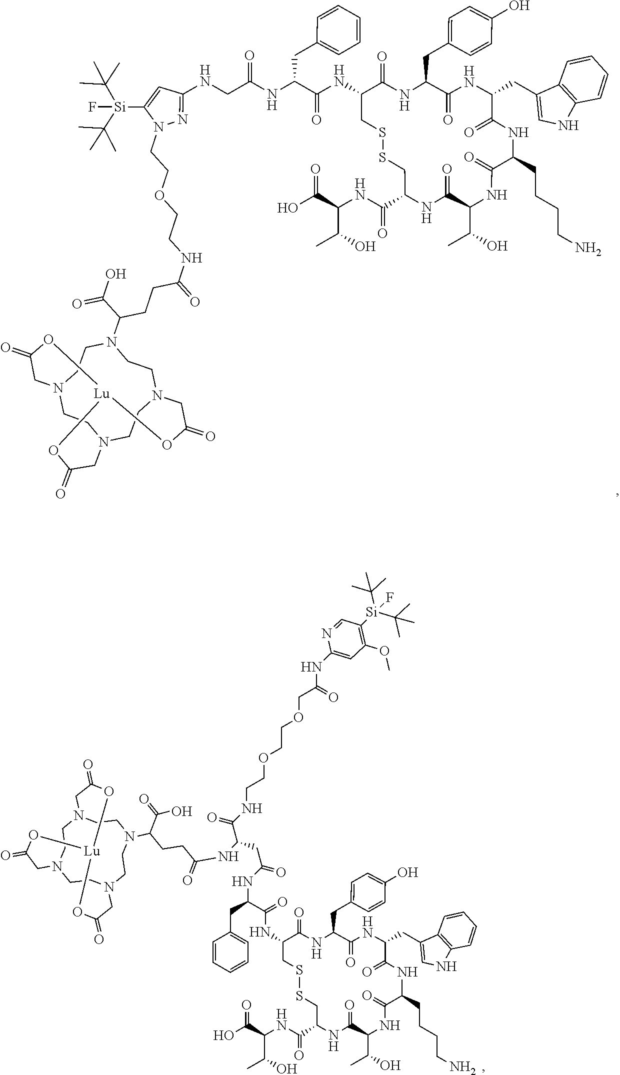

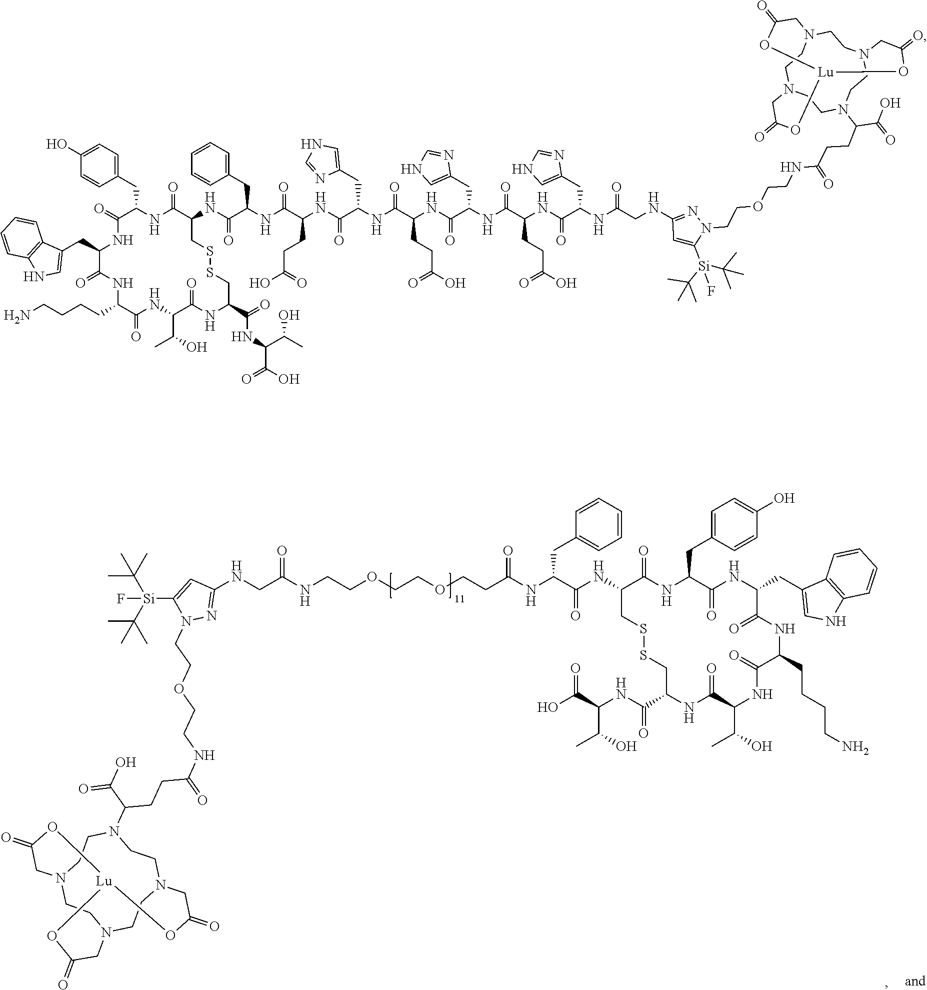

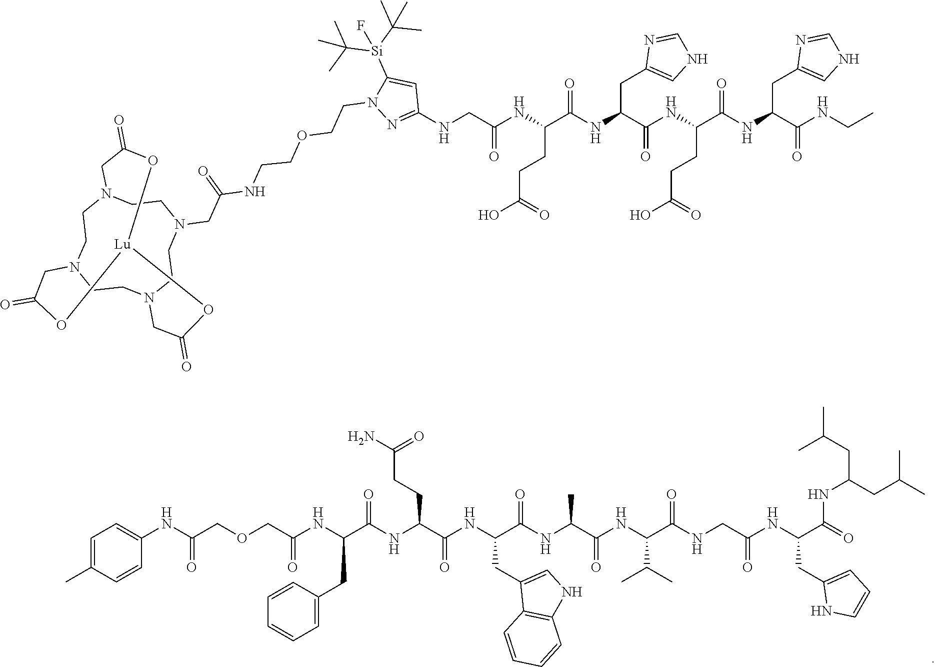

In an additional further embodiment, the radiopharmaceutical compound is a dual targeting theranostic compound with a formula of:

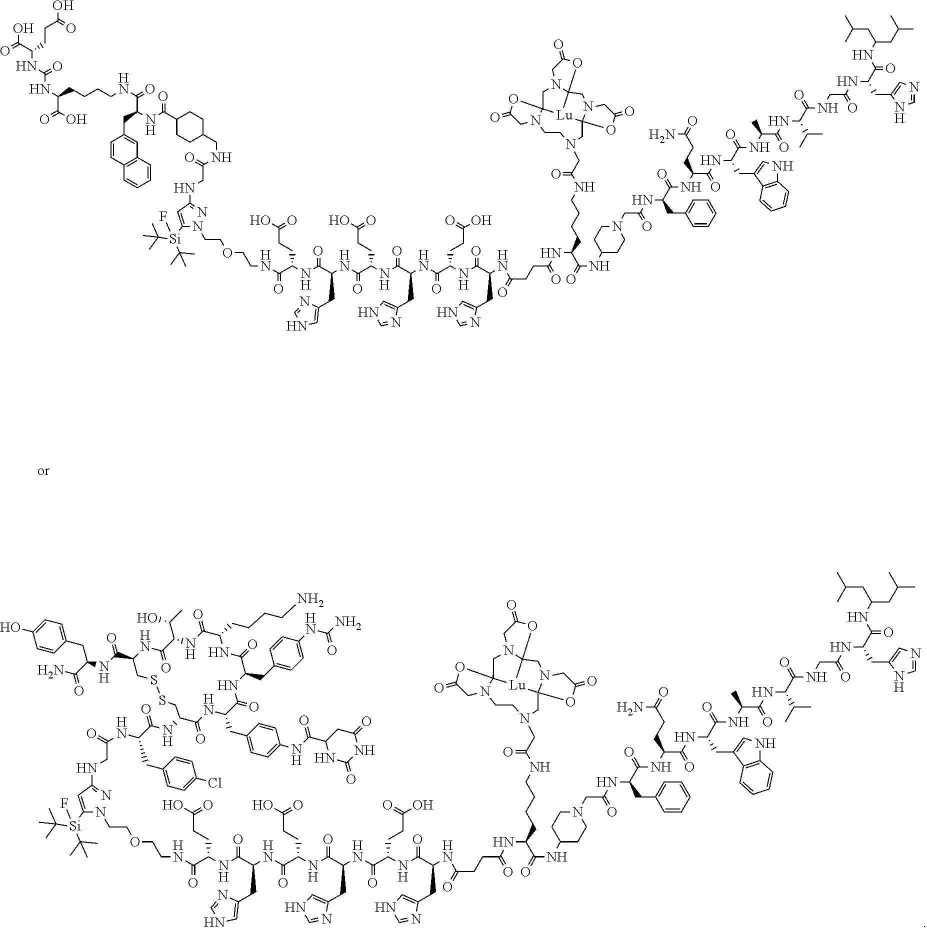

In a further embodiment again, the radiopharmaceutical compound is configured for PET imaging and therapeutic radioligand therapy.

In an additional further yet embodiment, the conjugation is via a process selected from the group consisting of: coupling chemistry, peptide coupling chemistry, copper-catalyzed azide-alkyne cycloaddition (CuAAC), and solid-phase peptide synthesis (SPPS).

Another yet further embodiment further comprises chelating 3p-C-NETA with ¹⁷⁷Lu for therapeutics; chelating DOTA and DOTAGA with ¹⁷⁷Lu or ²²⁵Ac for therapeutics; and chelating DOTA and DOTAGA with one of: ⁶⁸Ga, ⁸⁹Zr, and ⁶⁴Cu for PET imaging.

Additional embodiments and features are set forth in part in the description that follows and, in part, will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 illustrates the structures of ⁶⁸Ga-DOTA-TATE and ¹⁷⁷Lu-DOTA-TATE in accordance with prior art.

FIG. 2 illustrates the structures of ⁶⁸Ga-PSMA-617 and ¹⁷⁷Lu-PSMA-617 in accordance with prior art.

FIG. 3 illustrates an example HPLC chromatogram in a HetSiFA hydrolytic stability assay in accordance with an embodiment of the invention.

FIG. 4 illustrates structures of pyridine-HetSiFAs and their properties in accordance with an embodiment of the invention.

FIG. 5 illustrates hydrolytic stabilities and radiochemical conversions (RCC) of pyridyl-HetSiFAs in accordance with an embodiment of the invention.

FIG. 6 illustrates hydrolytic stabilities and radiochemical conversions (RCC) of pyridyl-HetSiFAs in accordance with an embodiment of the invention.

FIG. 7 illustrates structures of N-modified pyridine-HetSiFAs and their properties in accordance with an embodiment of the invention.

FIG. 8 illustrates structures of pyrazole-HetSiFAs and their properties in accordance with an embodiment of the invention.

FIG. 9 illustrates hydrolytic stabilities and radiochemical conversions (RCC) of pyrazole-HetSiFAs in accordance with an embodiment of the invention.

FIG. 10 illustrates structures of [6+5]-fused-HetSiFAs and their properties in accordance with an embodiment of the invention.

FIG. 11 illustrates structures of fused-HetSiFAs, furan-HetSiFAs, and pyrrole-HetSiFAs and their properties in accordance with an embodiment of the invention.

FIG. 12 illustrates a schematic of synthesizing a ¹⁸F-labeled PET imaging tracer comprising HetSiFAs and disease-binding ligands in accordance with an embodiment of the invention.

FIG. 13 illustrates a synthesis scheme of a pyrazole-HetSiFA building block for conjugation to a disease-binding ligand on resin in accordance with an embodiment of the invention.

FIG. 14 illustrates structures of ¹⁸F-labeled HetSiFAs conjugated with TATE for PET imaging of SSTR2 in cancer in accordance with an embodiment of the invention.

FIG. 15 illustrates structures of ¹⁸F-labeled HetSiFAs conjugated with JR11 for PET imaging of SSTR2 in cancer in accordance with an embodiment of the invention.

FIG. 16 illustrates a scheme of synthesizing theranostic pairs comprising HetSiFA-chelator constructs and disease-binding ligands in accordance with an embodiment of the invention.

FIG. 17 illustrates structures of HetSiFA-chelator constructs that are capped with a biologically inactive moiety for testing in accordance with an embodiment of the invention.

FIG. 18 illustrates a synthesis scheme for the HetSiFA theranostic FTX-165 in accordance with an embodiment of the invention.

FIG. 19 illustrates a HetSiFA-chelator-ligand construct forming chemically identical PET diagnostics and RLT therapeutics in accordance with an embodiment of the invention.

FIG. 20 illustrates radiolabeling results of ¹⁸F-FTX-165 in accordance with an embodiment of the invention.

FIG. 21 illustrates ex vivo biodistribution data of ¹⁸F-FTX-165 in healthy mice in accordance with an embodiment of the invention.

FIG. 22 illustrates radiolabeling results of ¹⁸F-FTX-164 indicating radiolysis in accordance with an embodiment of the invention.

FIG. 23 illustrates radiolabeling results of ¹⁸F-FTX-181 in accordance with an embodiment of the invention.

FIG. 24 illustrates ex vivo biodistribution data of ¹⁸F-FTX-181 in healthy mice in accordance with an embodiment of the invention.

FIG. 25 illustrates PET images of ¹⁸F-FTX-181 in healthy mice in accordance with an embodiment of the invention.

FIG. 26 illustrates HSA binding assay results of HetSiFA compounds in accordance with an embodiment of the invention.

FIGS. 27A-27N illustrate various theranostic HetSiFA pairs in accordance with an embodiment of the invention.

FIG. 28 illustrates a dual-targeting theranostic HetSiFA pair in accordance with an embodiment of the invention.

FIG. 29 illustrates a synthesis scheme of PPI-24069/FTX-175 in accordance with an embodiment of the invention.

FIG. 30 illustrates a synthesis scheme of PPI-24073/FTX-181 in accordance with an embodiment of the invention.

FIG. 31 illustrates a synthesis scheme of PPI-24082/FTX-219 in accordance with an embodiment of the invention.

### DETAILED DESCRIPTION OF THE INVENTION

In the description of embodiments, reference may be made to the accompanying figures which form a part hereof: and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those skilled in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following provides illustrative embodiments of the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (e.g., to at least one) of the grammatical object of the article. By way of example, “an element” means one element or more than one element.

“About” and “approximately” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained. For compositions suitable for administration to humans, the term “pharmaceutically acceptable” is meant to include, but is not limited to, those ingredients described in Remington: The Science and Practice of Pharmacy, Lippincott Williams & Wilkins, 21st ed. (2006).

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, and hexafluorophosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the terms “imaging agent”, “imaging probe”, or “imaging compound”, means, unless otherwise stated, a molecule which can be detected by its emitted signal, such as positron emission, autofluorescence emission, or optical properties. The method of detection of the compounds may include, but are not limited to, nuclear scintigraphy, positron emission tomography (PET), single photon emission computed tomography (SPECT), magnetic resonance imaging (MRI), magnetic resonance spectroscopy, computed tomography (CT), or a combination thereof depending on the intended use and the imaging methodology available to the medical or research personnel.

As used herein, the term “biomolecule” refers to any molecule produced by a living organism and may be selected from the group consisting of proteins, peptides, polysaccharides, carbohydrates, lipids, as well as analogs and fragments thereof.

As used herein, the terms “bioconjugation” and “conjugation”, unless otherwise stated, refer to the chemical derivatization of a macromolecule with another molecular entity. The molecular entity can be any molecule and can include a small molecule or another macromolecule. Examples of molecular entities include, but are not limited to, compounds of the invention, other macromolecules, polymers or resins, such as polyethylene glycol (PEG) or polystyrene, non-immunogenic high molecular weight compounds, fluorescent, chemiluminescent radioisotope and bioluminescent marker compounds, antibodies, biotin, diagnostic detector molecules, such as a maleimide derivatized fluorescein, coumarin, a metal chelator or any other modifying group. The term bioconjugation and conjugation are used interchangeably throughout the Specification.

As used herein, the term “alkyl”, by itself or as part of another substituent means, unless otherwise stated, a straight or branched chain hydrocarbon having the number of carbon atoms designated (e.g. C₁₋₆ means one to six carbon atoms) and including straight, branched chain, or cyclic substituent groups. Examples include methyl, ethyl, propyl, isopropyl, n-butyl, isobutyl, tert-butyl, sec-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, and —C(═NH)NH₂. Examples of substituted alkyls include, but are not limited to, 2,2-difluoropropyl, 2-carboxycydopentyl, and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “aromatic” refers to a carbocycle or heterocycle with one or more polyunsaturated rings and having aromatic character, e.g. having (4n+2) delocalized π (pi) electrons, where n is an integer.

As used herein, the term “aryl”, employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, for example, the referenced group is unsubstituted. In another embodiment, the referenced group is optionally substituted with one or more additional group(s) individually and independently selected from groups described herein.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Systems and methods for heteroaromatic silicon-fluoride acceptor (HetSiFA) systems with theranostic properties are described. Many embodiments include systems and methods for HetSiFA systems with theranostic capabilities to provide conjugates that bind to biological targets overexpressed in cancer. Theranostics has the combined capabilities of diagnostic PET imaging and therapeutic radioligand therapy (RLT). The theranostic pairs in accordance with several embodiments can have identical chemical structures and contain a heteroaromatic silicon-fluoride acceptor system for ¹⁸F incorporation for PET diagnostics as well as a chelator for incorporation with radiometals for RLT. In a number of embodiments, the theranostic pairs can allow diagnosis and treatment of patients with various types of metastatic cancers including (but not limited to) metastatic prostate cancer.

In many embodiments, various HetSiFA compounds can be used for imaging. In several embodiments, the HetSiFA compounds can be used for PET imaging. The HetSiFA compounds can include heteroaryl rings substituted with various side chains, linkers, and disease targeting ligands for PET imaging.

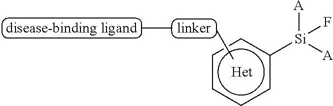

wherein: F is F, ¹⁸F, or ¹⁹F; each A is independently: H, methyl, ethyl, sec-butyl, isopropyl, and tert-butyl.

In many embodiments, various HetSiFA compounds can be used for theranostics. In several embodiments, the HetSiFA compounds can include a single disease binding ligand that targets receptors that are overexpressed on cancer cells. The single-target theranostic compounds can include heteroaryl rings substituted with various side chains, linkers, disease targeting ligands, and chelators for radiometalation.

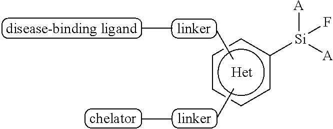

wherein: F is F, ¹⁸F, or ¹⁹F; each A is independently: H, methyl, ethyl, sec-butyl, isopropyl, and tert-butyl.

In many embodiments, various HetSiFA compounds can be used for theranostics. In several embodiments, the HetSiFA compounds can include two disease binding ligands as double-target theranostic compounds. The two disease binding ligands can target different overexpressed receptors on cancer cells. The dual-target theranostic compounds can include heteroaryl rings substituted with various side chains, linkers, disease targeting ligands, and chelators for radiometalation.

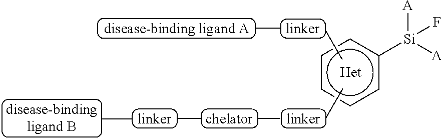

wherein: F is F, ¹⁸F, or ¹⁹F; each A is independently: H, methyl, ethyl, sec-butyl, isopropyl, and tert-butyl.

In many embodiments, the HetSiFA compounds can include heteroaryl rings including (but not limited to) pyridine, pyridinium, pyrazole, fused pyrazole, benzofuran, benzothiophene, indole, azaindole, imidazole, and pyrimidine. As can be readily appreciated, any type of a heteroaryl ring can be utilized as appropriate to the requirements of specific applications in accordance with various embodiments of the invention.

In some embodiments, the HetSiFA compounds can include heteroaryl rings such as pyridine or pyridinium, or a salt thereof:

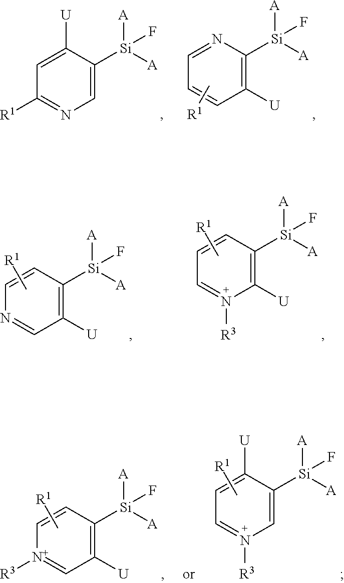

or the HetSiFA compounds can include heteroaryl rings such as pyrazole or a salt thereof:

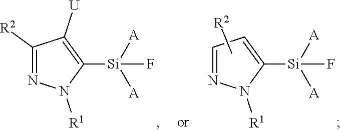

or the HetSiFA compounds can include heteroaryl rings such as fused pyrazole derivatives or a salt thereof:

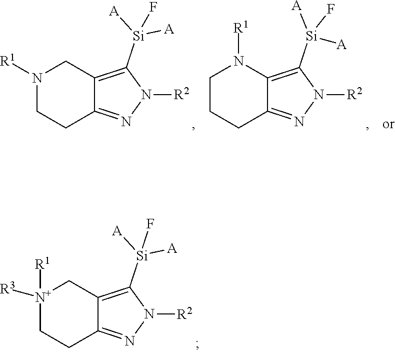

or the HetSiFA compounds can include heteroaryl rings such as benzofuran, benzothiophene, indole, azaindole or a salt thereof:

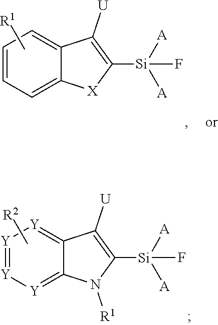

or the HetSiFA compounds can include heteroaryl rings such as imidazole or a salt thereof:

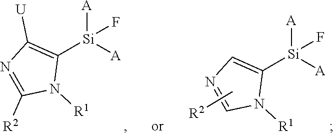

or the HetSiFA compounds can include heteroaryl rings such as pyrimidine or a salt thereof:

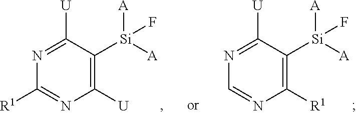

wherein:
F is independently: F, or ¹⁸F, or ¹⁹F;
A is independently: H, CH₃, CH₂—CH₃, CH₃—CH₂—CH(CH₃), CH(CH₃)₂, and C(CH₃)₃;
U is independently: O—CH₃, CH₃, CH₂CH₃, H, I, Br, Cl, F, N(CH₃)₂, and CH₂CH(NH₂)CO₂H;
X is independently: O, S, and N;
Y is independently: C and N;
R¹ or R² or R³ is independently: CH₃, CH₂—CH₃, H, L¹-CH₂—C≡C, L¹-CH₃, L¹-G, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²- L³-L⁴-G, L¹-L²-L³-L⁴-H, L¹-L²-L³-L⁴-Q-L⁵-G, L¹-L²-L³-Q, L¹-L²-L³-Q-L⁴-G, L¹-L²-N₃, L¹-L²-OH, L¹-L²-Q, L¹-L²-Q-G, L¹-L²-Q-L³-G, L¹-L²-Q-L³-L⁴-G, L¹-L²-Q-L³-L⁴-H, L¹-OH, L¹-Q, L¹-Q-L²-L³-L⁴-G, L¹-Q-L²-G, NH₂, O⁻, O—CH₃, OH,

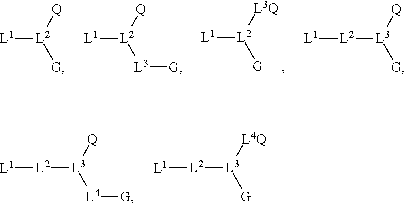

L¹ or L² or L³ or L⁴ or L⁵ is independently: —(O—CH₂—CH₂)ₚ—, —(CH₂—CH₂—O)ₚ—, -(Glu-His)ₚ-, -(His-Glu)ₚ-, -(Glu-Trp)ₚ-, -(Trp-Glu)ₚ-, —NH—CH₂—C₆H₄—NH—(C═O)—CH₂—O—CH₂—(C═O)—, —(C═O)—CH₂—O—CH₂—(C═O)—NH—C₆H₄—CH₂—NH—, —(C═O)—CH₂—CH₂—(C═O)—, —NH—C₅H₉N—CH₂—(C═O)—, —(C═O)—CH₂—NC₅H₉—NH—, -(Gly)ₚ-, —CH₂—CH₂—NH—, —NH—CH₂—(C═O)—, -(Glu)-, —NH—CH₂—CH₂—, —(C═O)—, NH—CH₂—CH₂—(O—CH₂—CH₂)ₚ—(C═O)—, —(C═O)—(CH₂—CH₂—O)ₚ—CH₂—CH₂—NH—, —NH—(C═O)—CH₂—, —NH—(C═O)—NH—CH₂—, —NH—(C═S)—NH—CH₂—, —NH—(CH₂—CH₂—O)ₚ—CH₂—(C═O)—, —(C═O)—CH₂—(O—CH₂—CH₂)ₚ—NH—, -Asp-, —NH—, —NH—(CH₂—CH₂—O)ₚ—CH₂—CH₂—(C═O)—, —(C═O)—CH₂—CH₂—(O—CH₂—CH₂)ₚ—NH—, —CH₂—(C═O)—, —(C═O)—CH₂—, —O—(C═O)—, —(C═O)—O—, —CH₂—O—, —NH—(C═O)—O—, —O—(C═O)—NH—, —(C═O)—CH₂—(O—CH₂—CH₂)ₚ—O—CH₂—(C═O)—, —NH—(C═O)—NH—, —NH—(C═S)—NH—, —CH₂—N(CH₃)—CH₂—, —N(CH₃)—, —(C═O)—C₆H₄—(C═O)—, —C₆H₄—(C═O)—, —(C═O)—C₆H₄—, —O—CH₂—(C═O)—, —(C═O)—CH₂—O—, —CH₂—C₂N₃—CH₂—, —CH₂—CH₂—(C═O)—, —(C═O)—CH₂—CH₂—, —CH₂—N⁺(CH₃)₂—CH₂—, —CH₂—N(CH₃)—, —(C═O)—CH₂—(O—CH₂—CH₂)ₚ—, —(CH₂—CH₂—O)ₚ—CH₂—CH₂—NH—,

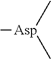

p=0 to 12;
G is a disease binding ligand, wherein each G is independently:
a somatostatin receptor type 2 (SSTR2) binding ligand G¹

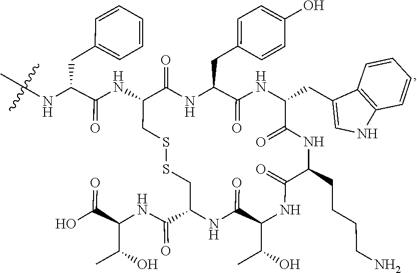 [SMILES: *N[C@H](Cc1ccccc1)C(=O)N[C@H]1CSSC[C@@H](C(=O)N[C@H](C(=O)O)[C@@H](C)O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O]

a SSTR2 binding ligand G²

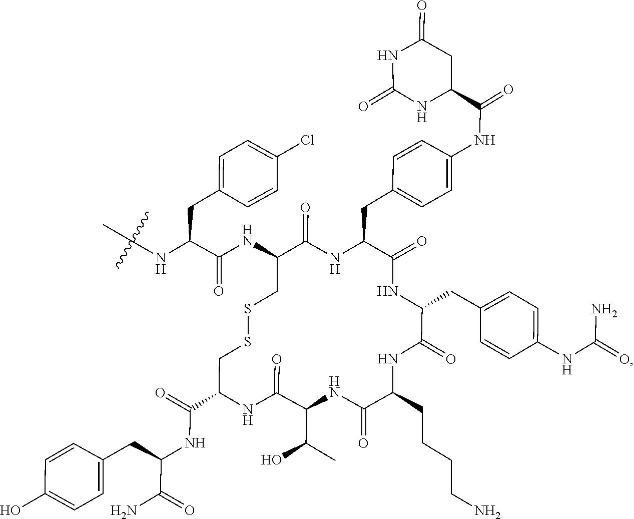 [SMILES: *N[C@@H](Cc1ccc(Cl)cc1)C(=O)N[C@@H]1CSSC[C@@H](C(=O)N[C@H](Cc2ccc(O)cc2)C(N)=O)NC(=O)[C@H]([C@@H](C)O)NC(=O)[C@H](CCCCN)NC(=O)[C@@H](Cc2ccc(NC(N)=O)cc2)NC(=O)[C@H](Cc2ccc(NC(=O)[C@@H]3CC(=O)NC(=O)N3)cc2)NC1=O]

a SSTR2 binding ligand G³

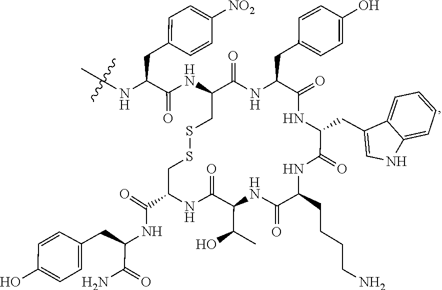 [SMILES: *N[C@@H](Cc1ccc([N+](=O)[O-])cc1)C(=O)N[C@@H]1CSSC[C@@H](C(=O)N[C@H](Cc2ccc(O)cc2)C(N)=O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O]

a SSTR2 binding ligand G⁴

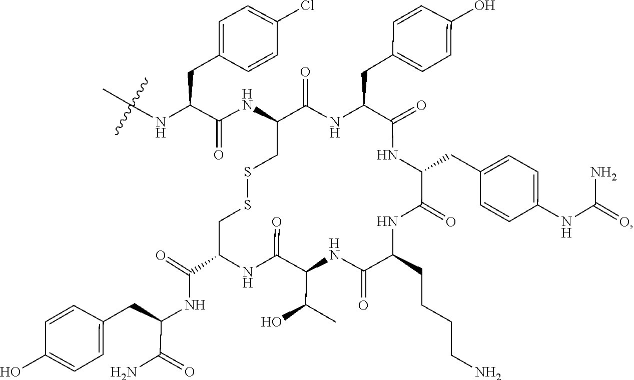 [SMILES: *N[C@@H](Cc1ccc(Cl)cc1)C(=O)N[C@@H]1CSSC[C@@H](C(=O)N[C@H](Cc2ccc(O)cc2)C(N)=O)NC(=O)[C@H]([C@@H](C)O)NC(=O)[C@H](CCCCN)NC(=O)[C@@H](Cc2ccc(NC(N)=O)cc2)NC(=O)[C@H](Cc2ccc(O)cc2)NC1=O]

a gastrin releasing peptide receptor (GRPR) binding ligand G⁵

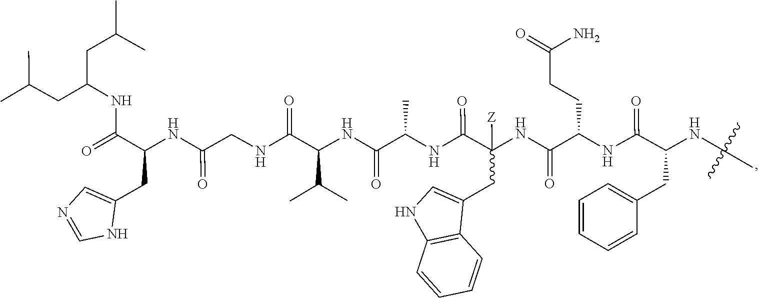

wherein Z is H, CH₃, or CF₃;
a GRPR binding ligand G⁶

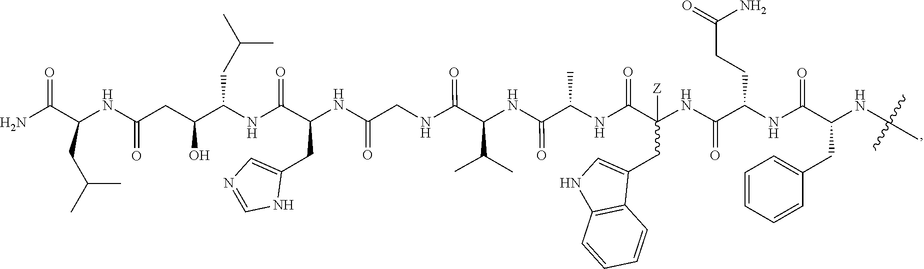

wherein Z is H, CH₃, or CF₃;
a prostate specific membrane antigen (PSMA) binding ligand G⁷

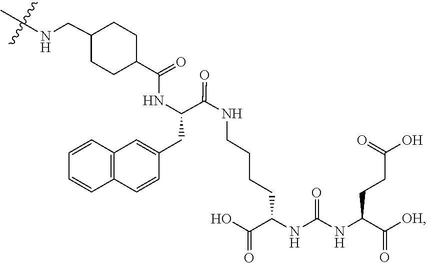 [SMILES: *NCC1CCC(C(=O)N[C@@H](Cc2ccc3ccccc3c2)C(=O)NCCCC[C@H](NC(=O)N[C@@H](CCC(=O)O)C(=O)O)C(=O)O)CC1]

a PSMA binding ligand G⁸

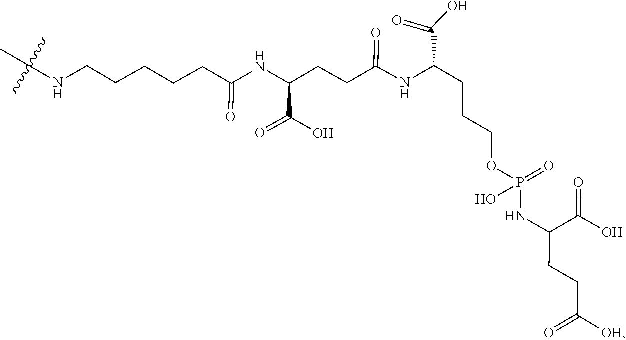

a PSMA binding ligand G⁹

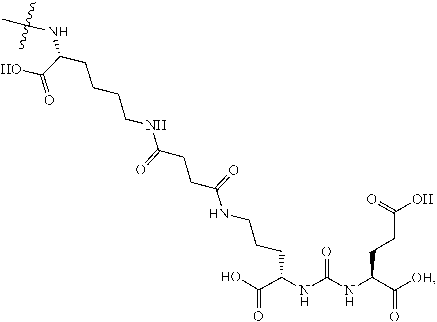 [SMILES: *N[C@H](CCCCNC(=O)CCC(=O)NCCC[C@H](NC(=O)N[C@@H](CCC(=O)O)C(=O)O)C(=O)O)C(=O)O]

a PSMA binding ligand G¹⁰

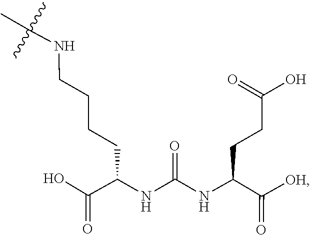 [SMILES: *NCCCC[C@H](NC(=O)N[C@@H](CCC(=O)O)C(=O)O)C(=O)O]

a PSMA binding ligand G¹¹

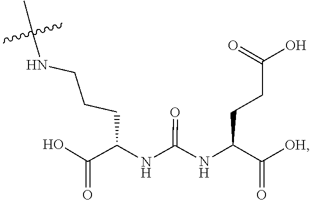 [SMILES: *NCCC[C@H](NC(=O)N[C@@H](CCC(=O)O)C(=O)O)C(=O)O]

a fibroblast activation protein (FAP) binding ligand G¹²

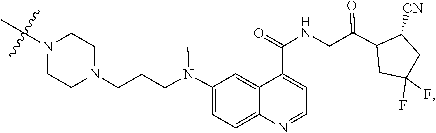 [SMILES: *N1CCN(CCCN(C)c2ccc3nccc(C(=O)NCC(=O)C4CC(F)(F)C[C@H]4C#N)c3c2)CC1]

a FAP binding ligand G¹³

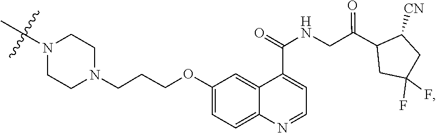 [SMILES: *N1CCN(CCCOc2ccc3nccc(C(=O)NCC(=O)C4CC(F)(F)C[C@H]4C#N)c3c2)CC1]

a FAP binding ligand G¹⁴

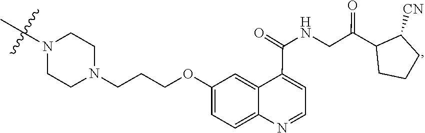 [SMILES: *N1CCN(CCCOc2ccc3nccc(C(=O)NCC(=O)C4CCC[C@H]4C#N)c3c2)CC1]

a FAP binding ligand G¹⁵

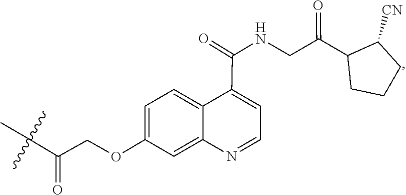 [SMILES: *C(=O)COc1ccc2c(C(=O)NCC(=O)C3CCC[C@H]3C#N)ccnc2c1]

a C-X-C chemokine receptor type 4 (CXCR-4) binding ligand G¹⁶

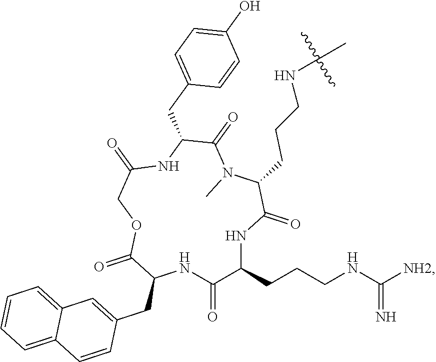 [SMILES: *NCCC[C@@H]1C(=O)N[C@@H](CCCNC(=N)N)C(=O)N[C@@H](Cc2ccc3ccccc3c2)C(=O)OCC(=O)N[C@H](Cc2ccc(O)cc2)C(=O)N1C]

a CXCR-4 binding ligand G¹⁷

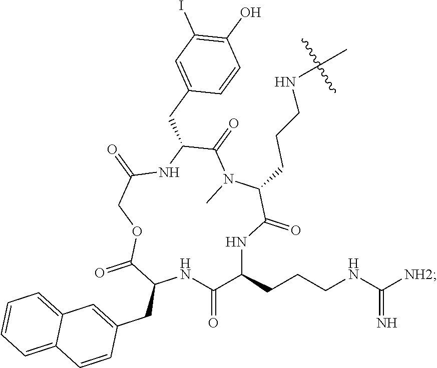 [SMILES: *NCCC[C@@H]1C(=O)N[C@@H](CCCNC(=N)N)C(=O)N[C@@H](Cc2ccc3ccccc3c2)C(=O)OCC(=O)N[C@H](Cc2ccc(O)c(I)c2)C(=O)N1C]

Q is a chelator, wherein each Q is independently:

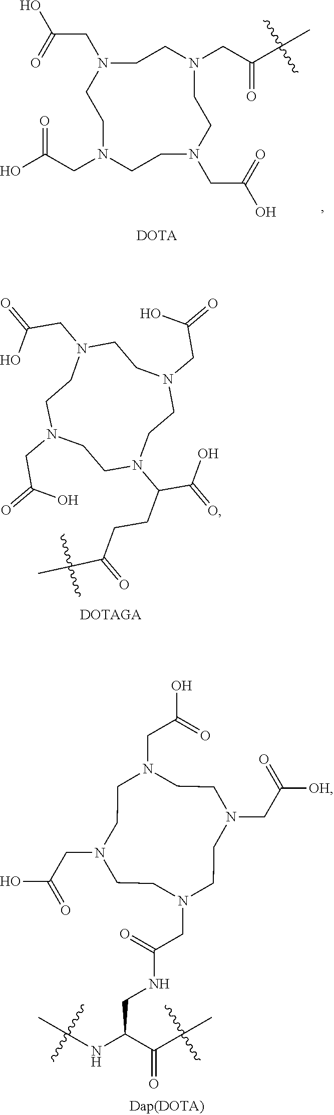 [SMILES: *C(=O)CN1CCN(CC(=O)O)CCN(CC(=O)O)CCN(CC(=O)O)CC1 ; "DOTA" ; *C(=O)CCC(C(=O)O)N1CCN(CC(=O)O)CCN(CC(=O)O)CCN(CC(=O)O)CC1 ; *N[C@@H](CNC(=O)CN1CCN(CC(=O)O)CCN(CC(=O)O)CCN(CC(=O)O)CC1)C(*)=O]

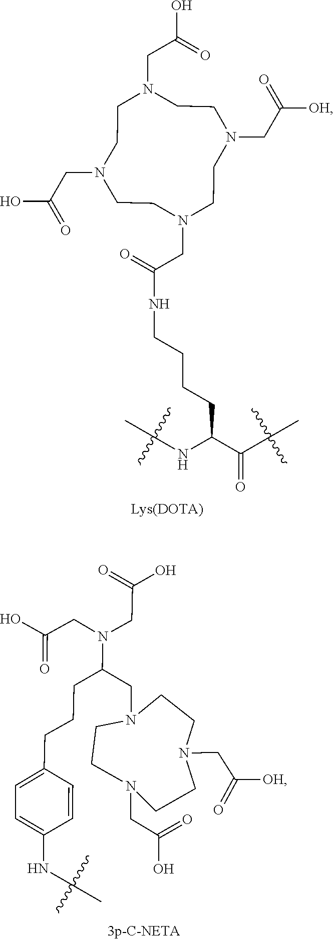 [SMILES: *N[C@@H](CCCCNC(=O)CN1CCN(CC(=O)O)CCN(CC(=O)O)CCN(CC(=O)O)CC1)C(*)=O ; *Nc1ccc(CCCC(CN2CCN(CC(=O)O)CCN(CC(=O)O)CC2)N(CC(=O)O)CC(=O)O)cc1]

bis-thioseminarabazones

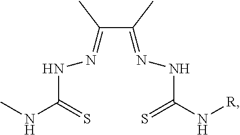

wherein R can be methyl (H₂ATSM), or R can be NH₂ (H₂ATSM/A),

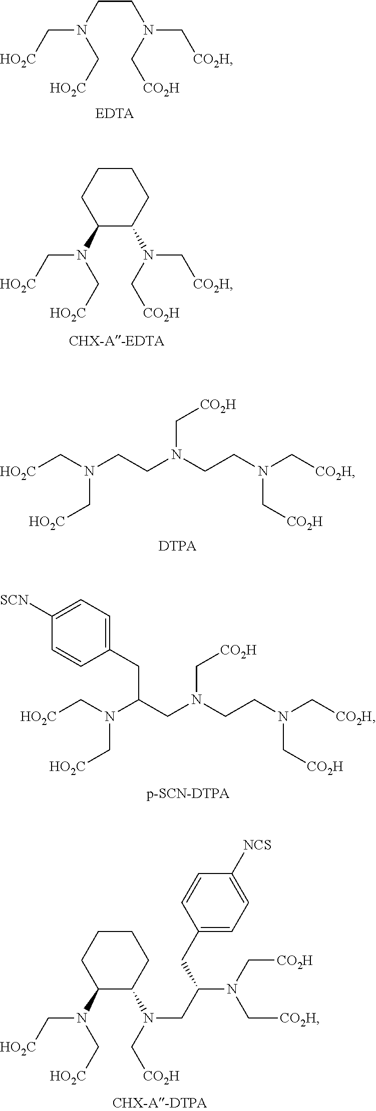

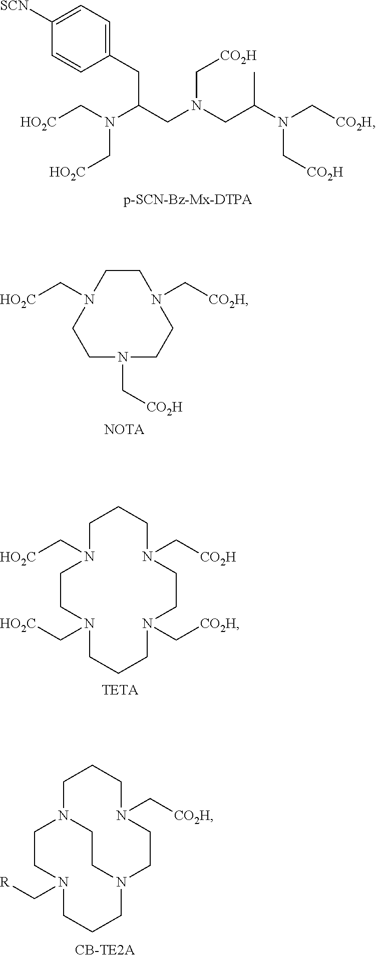

wherein R can be (C═O)—OH (CB-TE2A), or R can be P(O)(OH)₂ (CB-TE1A1P),

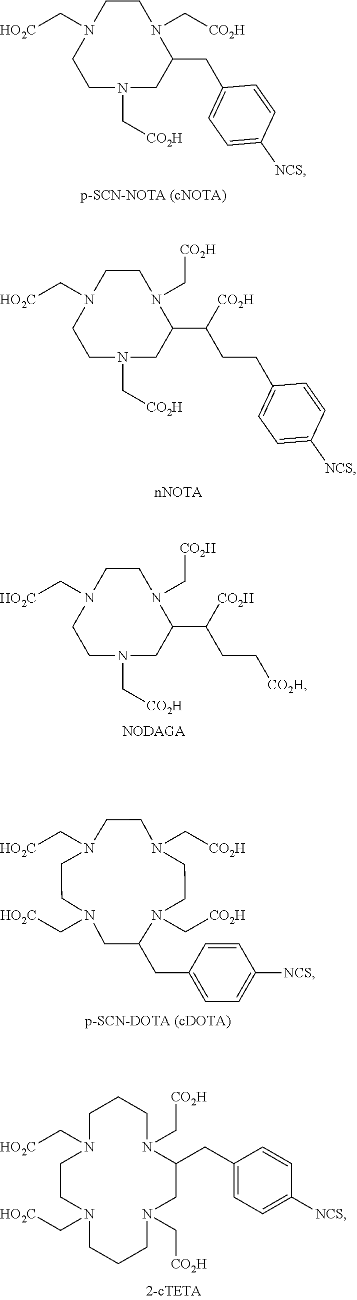

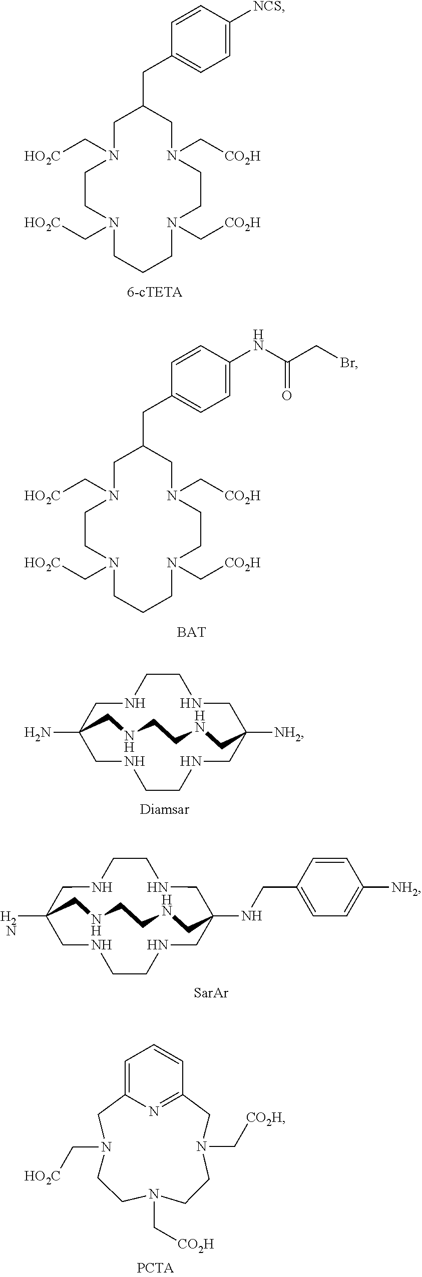

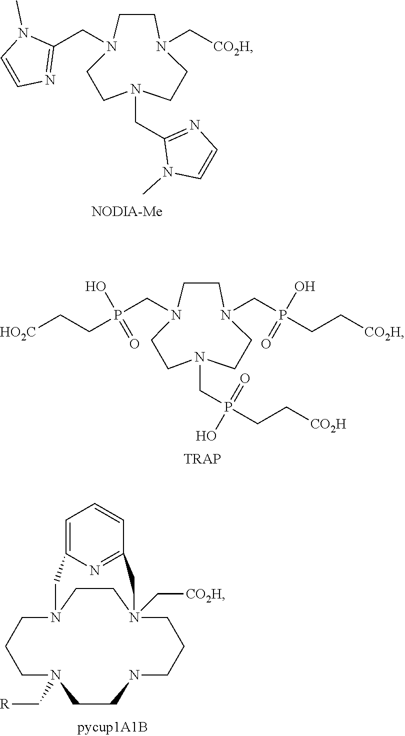

wherein R can be phenyl (pycup1A1B), or R can be (C═O)OH (pycup2A),

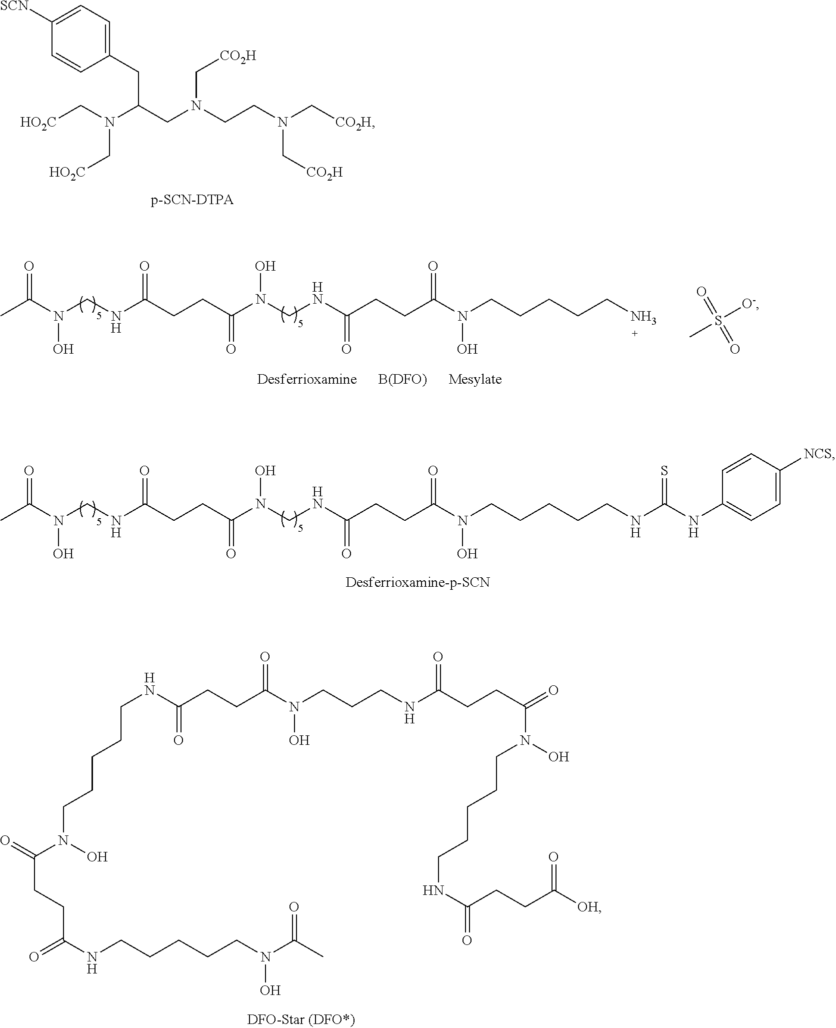

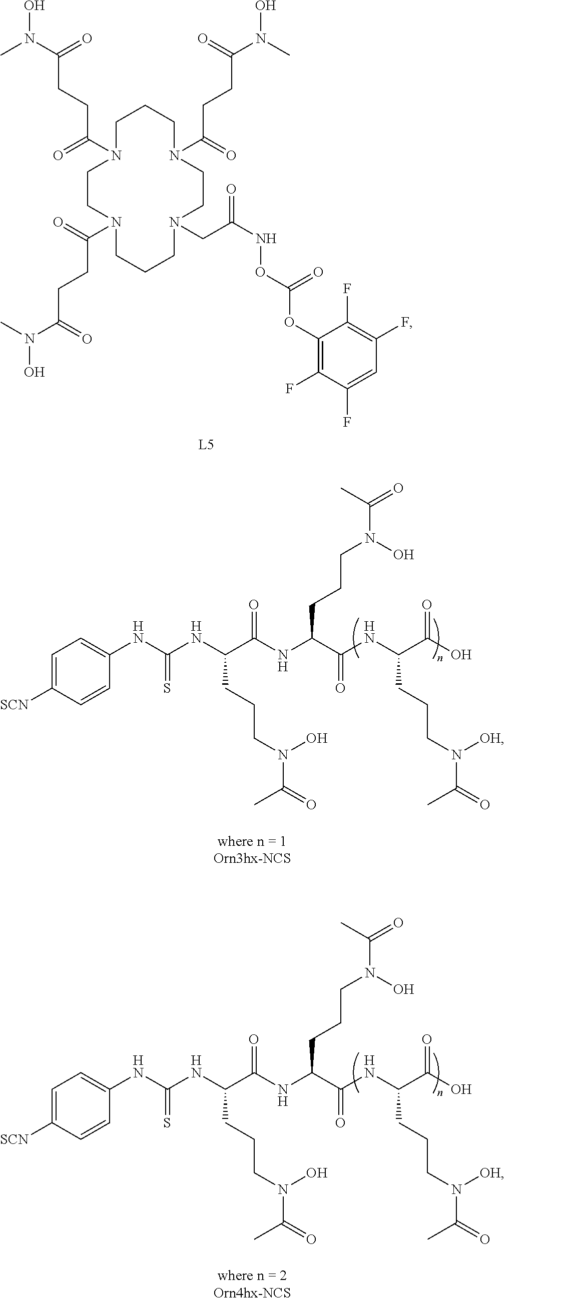

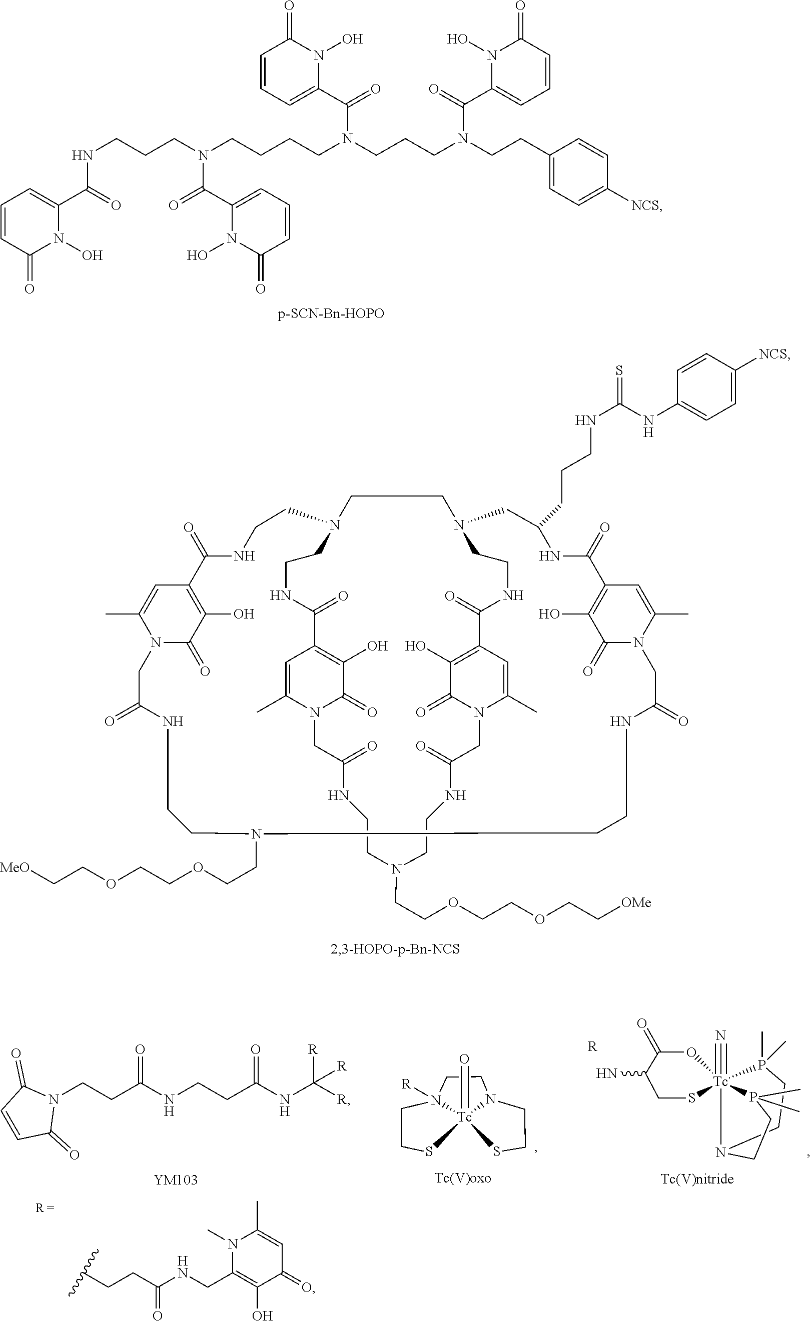

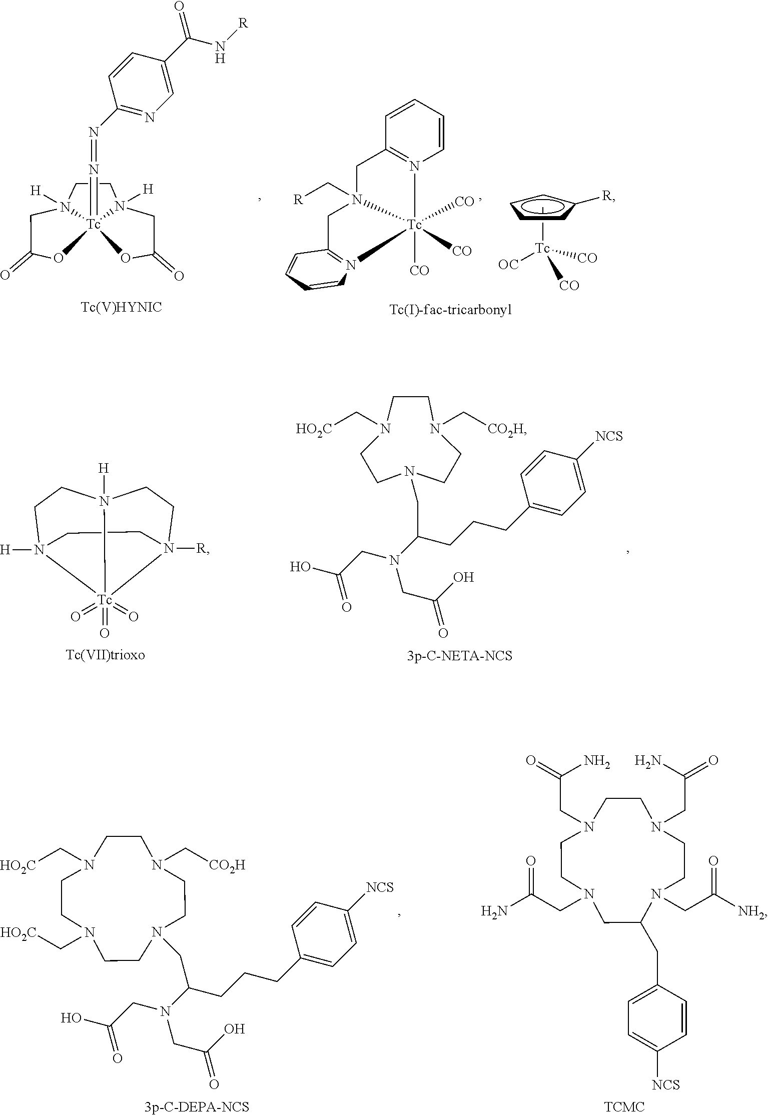

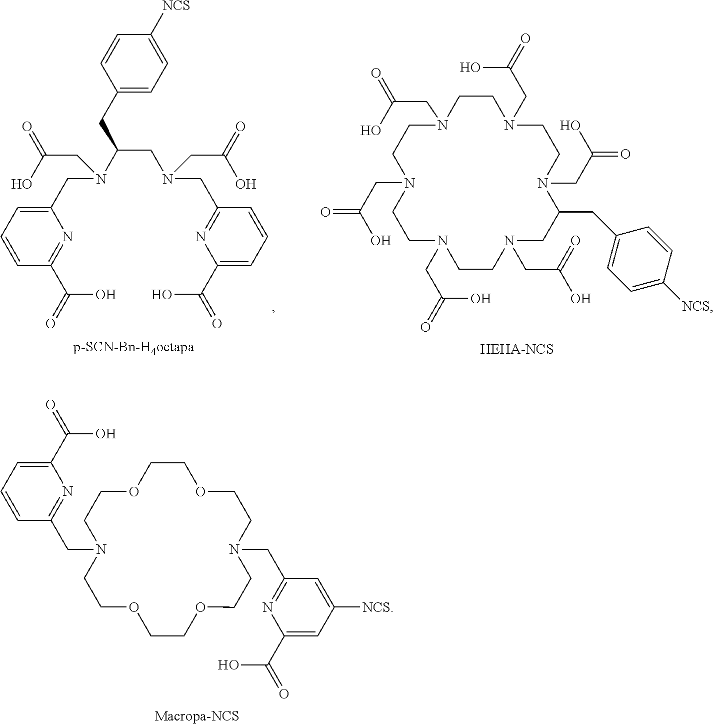

In some embodiments, Q can be unchelated. In several embodiments, Q can be optionally chelated with a metal cation M, wherein M is a cation of: ⁴³SC, ⁴⁴SC, ⁴⁵SC, ⁴⁷SC, ⁵¹Cr, ^{52m}Mn, ⁶⁸Co, ⁵²Fe, ⁵⁶Ni, ⁵⁷Ni, ⁶¹Cu, ⁶²Cu, ⁶³Cu, ⁶⁴Cu, ⁶⁵Cu, ⁶⁷Cu, ⁶⁶Ga, ⁶⁷Ga, ⁶⁸Ga, ⁶⁹Ga, ⁷¹Ga, ^{nat}Ga, ⁹⁰Zr, ⁹¹Zr, ⁹²Zr, ⁸⁹Zr, ⁸⁶Y, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹I, ¹¹³In, ^{133m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵⁵Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁶Lu, ¹⁷⁷Lu, ^{177m}Lu, ^{nat}Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²⁰⁴Pb, ²⁰⁶Pb, ²⁰⁷Pb, ²⁰⁸Pb, ²¹¹At, ²⁰⁹Bi, ²¹²Bi, ²¹³Bi, ²²³Ra, ²²⁵Ac, ²²⁷Th, ²³²Th, and a cationic molecule comprising ¹⁸F such as ¹⁸F[AlF]²⁺. The following unlimiting examples illustrate metal cation chelated chelators. As can be readily appreciated, any of the chelators described above can be chelated in a similar fashion. The chelated Q can be:

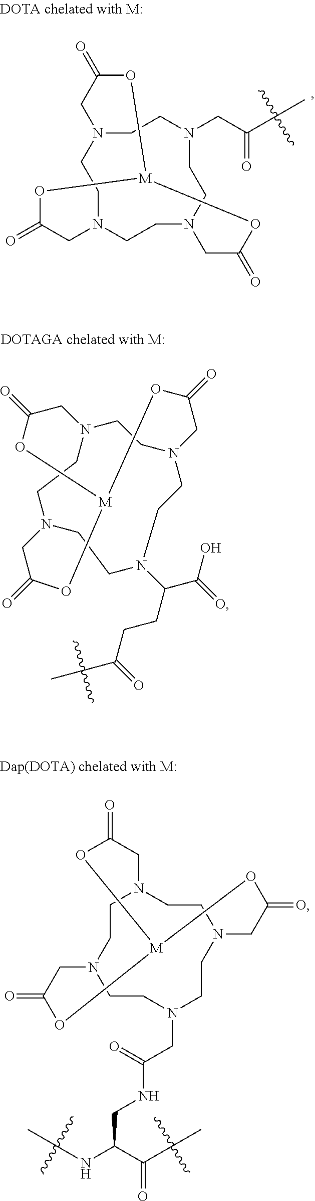

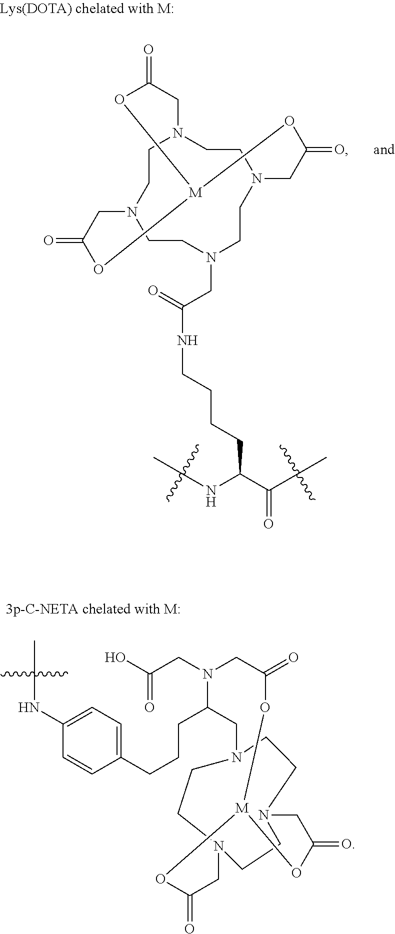

In several embodiments, various small molecule HetSiFA compounds can be used for synthesizing radiopharmaceuticals. Different types of disease-binding ligands and/or chelators can be attached to the small molecule HetSiFA compounds during the synthesis for their respective applications. In some embodiments, the small molecule HetSiFA compounds can have a formula:

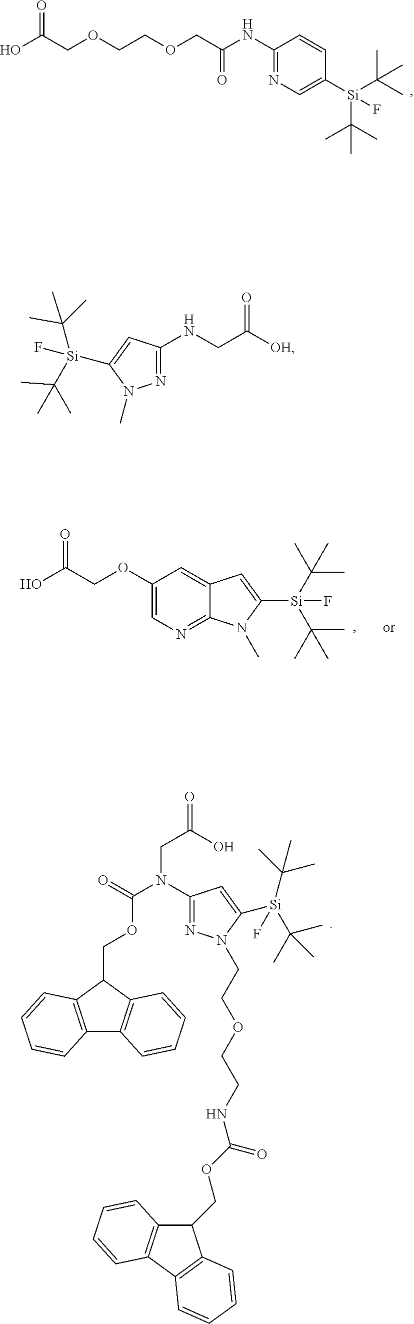

Various HetSiFA compounds can be used for PET imaging. Different types of disease-binding ligands are conjugated on the radiolabeled HetSiFA compounds. In several embodiments, the HetSiFA compounds for PET imaging can have a formula:

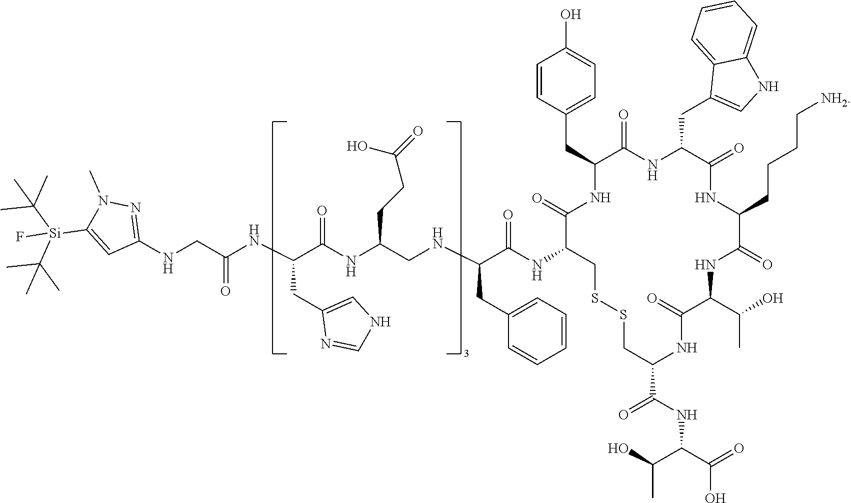

Various HetSiFA compounds are theranostic compounds. Different types of disease-binding ligands are conjugated to the radiolabeled HetSiFA compounds. The theranostic compounds have identical chemical structures but differ in nuclides. The theranostic compounds can be used for PET imaging and/or as therapeutics. In several embodiments, the theranostic HetSiFA compounds can have a formula:

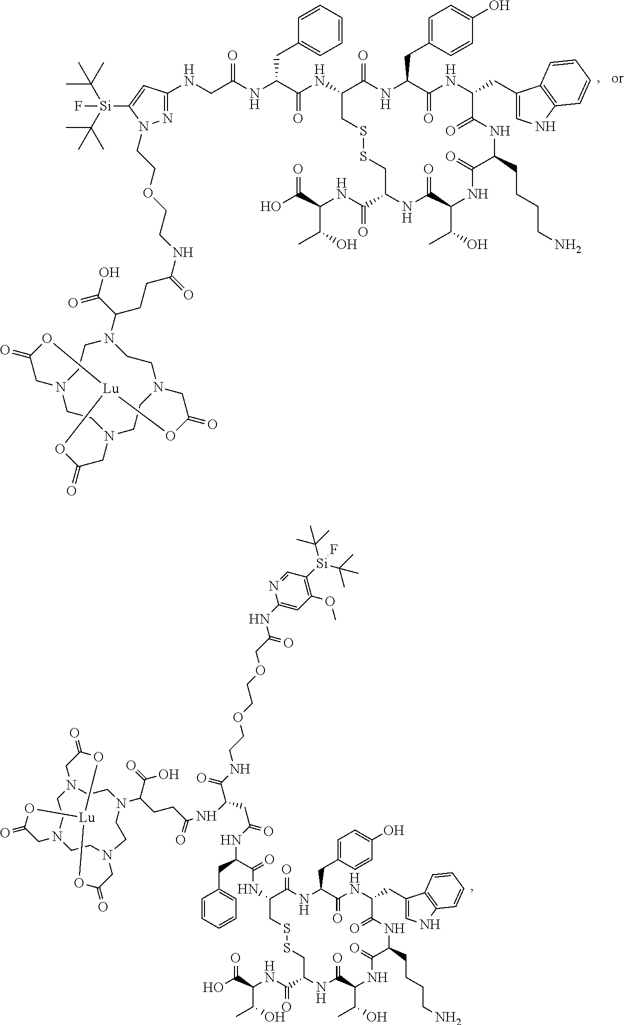

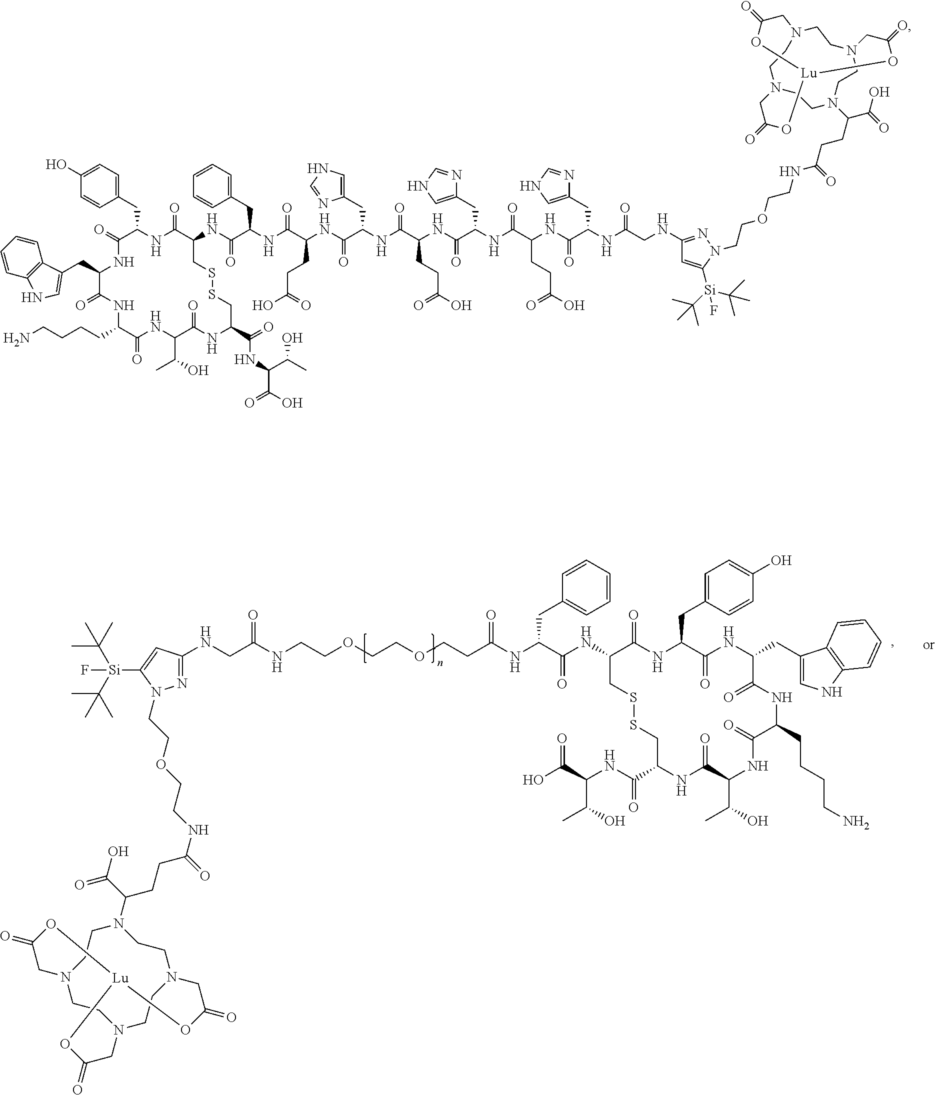

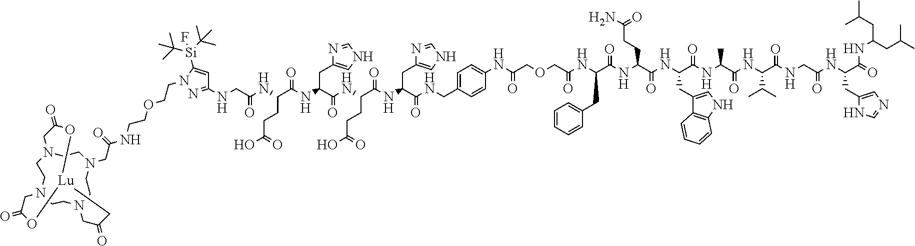

Various HetSiFA compounds are dual-targeting theranostic compounds. Two types of disease-binding ligands are conjugated to a radiolabeled HetSiFA compound. The theranostic compounds can be used for PET imaging and/or as therapeutics. In several embodiments, the theranostic HetSiFA compounds can have a formula:

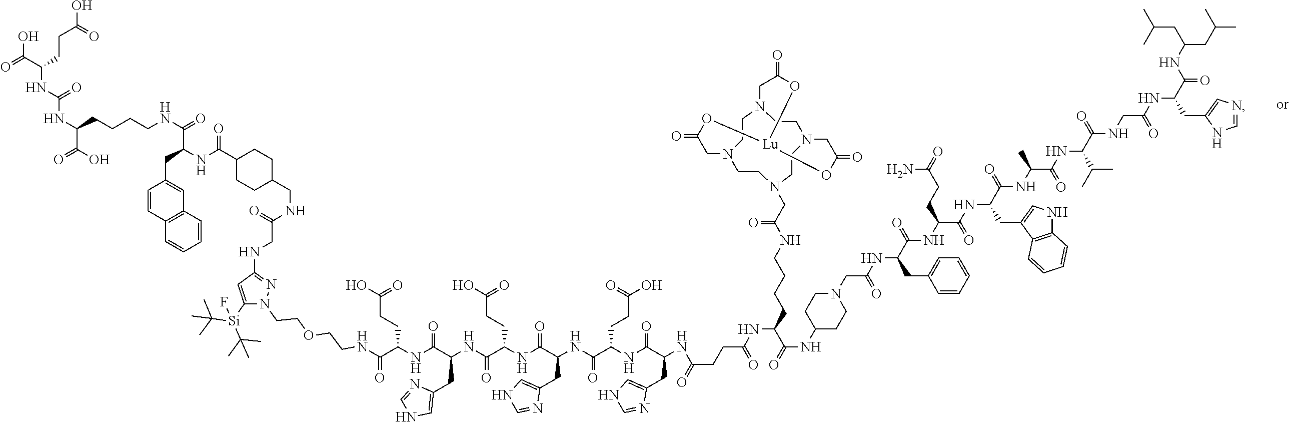

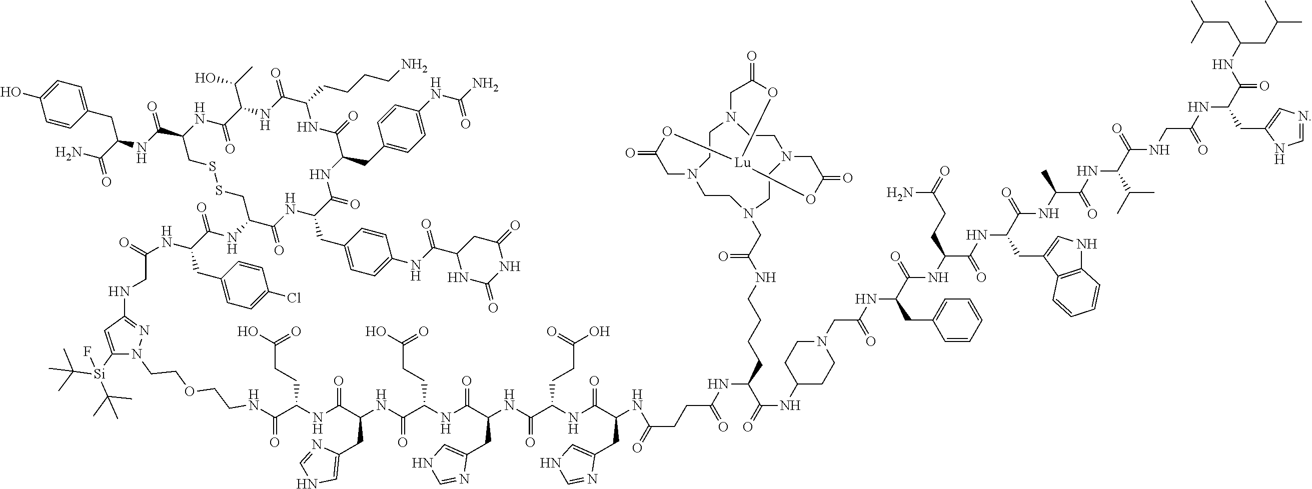

Many embodiments provide HetSiFA systems with desired properties including (but not limited to) radiolabeling efficiency, hydrophilicity, chemical versatility, hydrolytic stability, plasma protein binding ability, kinetic solubility, target binding affinity, and biodistribution in mice for making radiopharmaceuticals. In several embodiments, HetSiFAs with heteroaromatics including (but not limited to) pyrazoles, pyridines, pyridine oxides, and azaindoles show better properties for making radiopharmaceuticals than phenyl-SiFAs. Various HetSiFAs with heteroaromatics including (but not limited to) indoles, benzothiophenes, benzofurans, furans, and pyrroles show less favorable properties than pyrazoles, pyridines, pyridine oxides, and azaindoles.

HetSiFAs including (but not limited to) pyrazole-HetSiFAs, pyridine-HetSiFAs, pyridine oxide-HetSiFAs, and azaindole-HetSiFAs, can be conjugated to the disease binding ligands. Examples of disease binding ligands include (but are not limited to) somatostatin receptor type 2 (SSTR2) binding ligands (such as G¹, G², G³, and G⁴), gastrin releasing peptide receptor (GRPR) binding ligands (such as G⁵ and G⁶), prostate specific membrane antigen (PSMA) binding ligands (such as G⁷, G⁸, G⁹, G¹⁰, and G¹¹), fibroblast activation protein (FAP) binding ligands (such as G¹², G¹³, G¹⁴, and G¹⁵), and C-X-C chemokine receptor type 4 (CXCR-4) binding ligands (such as G¹⁶ and G¹⁷), to form precursors for ¹⁸F-PET tracers. Various HetSiFA compounds can be conjugated to disease-binding ligands via a variety of processes including (but not limited to) coupling chemistry, peptide coupling chemistry, copper-catalyzed azide-alkyne cycloaddition (CuAAC), and solid-phase peptide synthesis (SPPS).

Prostate cancer cells may become PSMA-negative during the malignancy in the stage of metastatic castration-resistant prostate cancer (mCRPC). The GRPR is a member of the G-protein coupled receptor superfamily and part of the family of bombesin receptors. The GRPR can be a potentially specific target for different malignancies as it is being overexpressed in various malignant entities, including neuroblastoma, lung, pancreatic, gastric, colorectal, esophageal, breast, and prostate cancer. This overexpression of GRPR and its rather low expression in normal organs and healthy tissue makes the receptor a promising diagnostic and therapeutic target for PET and RLT, respectively. Given the different expression patterns of PSMA and GRPR and GRPR overexpression in cell membranes of prostate cancer and prostatic intraepithelial neoplasms in contrast to healthy prostate tissue and benign prostate hyperplasia, targeting GRPR with theranostic radiopharmaceuticals can be suitable in patients who do not respond to PSMA theranostics and might be PSMA-negative.

In many embodiments, systems and methods for theranostic HetSiFA systems with dual targeting capabilities are described. HetSiFA systems can be conjugated with two different disease binding ligands for targeting receptors that are overexpressed on cancer cells. The dual targeting capabilities can improve the accuracy in imaging and/or treatment. In some embodiments, HetSiFA systems can be conjugated with dual disease binding ligands for receptors PSMA and GRPR for imaging and treatment of metastatic prostate cancer. In certain embodiments, HetSiFA systems can be conjugated with dual disease binding ligands for receptors GRPR and SSTR2 for imaging and treatment of metastatic breast or lung cancer.

Various HetSiFA compounds can be conjugated to small molecules with no biological function (or known as caps) in accordance with some embodiments. The resulting compounds can be used for radiolabeling testing and/or other assays.

HetSiFAs combined with metal chelators can be made as theranostic HetSiFAs. Many embodiments provide various HetSiFA compounds can be conjugated with chelators including (but not limited to) DOTA, DOTAGA, Dap(DOTA), Lys(DOTA), 3p-C-NETA, AAZTA, bis-thioseminarabazones, H₂ATSM, H₂ATSM/A, EDTA, CHX-A″-EDTA, DTPA, p-SCN-DPTA, CHX-A″-DTPA, p-SCN-Bz-Mx-DTPA, NOTA, TETA, CB-TE2A, CB-TE1A1P, p-SCN-NOTA, nNOTA, NODAGA, p-SCN-DOTA, 2-cTETA, 6-cTETA, BAT, Diamsar, SarAr, PCTA, NODIA-Me, TRAP, pycup1A1B, pycup2A, p-SCN-DTPA, Desferrioxamine, Desferrioxamine B, Desferrioxamine B (DFO) Mesylate, Desferrioxamine-p-SCN, DFO-Star (DFO*), L5, Orn3hx-NCS, Orn4hx-NCS, p-SCN-Bn-HOPO, 2,3-HOPO-p-Bn-NCS, YM103, Tc(V)oxo, Tc(V)nitride, Tc(V)HYNIC, Tc(I)-fac-tricarbonyl, Tc(VII)trioxo, 3p-C-NETA-NCS, 3p-C-DEPA-NCS, TCMC, p-SCN-Bn-H₄octapa, HEHA-NCS, Macropa-NCS.

The chelators may increase the lipophilicity of the ¹⁸F-PET-radiopharmaceuticals. The compounds of HetSiFAs with metal chelators can be used in a non-theranostic ¹⁸F-PET imaging radiopharmaceutical in accordance with certain embodiments. The chelator can be added to increase the overall polarity of the HetSiFA moiety. In some embodiments, the chelator can be left free without any metal ions attached. In several embodiments, the chelators can be chelated with a non-radioactive metal including (but not limited to) ^{nat}Ga and ^{nat}Lu.

In many embodiments, the chelators can be chelated with a metal cation. Each of the chelators can be chelated with a cation selected from the group consisting of: ⁴³Sc, ⁴⁴Sc, ⁴⁵Sc, ⁴⁷Sc, ⁵¹Cr, ^{52m}Mn, ⁶⁸Co, ⁵²Fe, ⁵⁶Ni, ⁵⁷Ni, ⁶¹Cu, ⁶²Cu, ⁶³Cu, ⁶⁴Cu, ⁶⁵Cu, ⁶⁷Cu, ⁶⁶Ga, ⁶⁷Ga, ⁶⁸Ga, ⁶⁹Ga, ⁷¹Ga, ⁹⁰Zr, ⁹¹Zr, ⁹²Zr, ⁸⁹Zr, ⁸⁶Y, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹I, ¹¹³In, ^{133m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵⁵Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁶Lu, ¹⁷⁷Lu, ^{177m}Lu ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²⁰⁴Pb, ²⁰⁶Pb, ²⁰⁷Pb, ²⁰⁸Pb, ²¹¹At, ²⁰⁹Bi, ²¹²Bi, ²¹³Bi, ²²³Ra, ²²⁵Ac, ²²⁷Th, ²³²Th, and a cationic molecule comprising ¹⁸F such as ¹⁸F[AlF]²⁺.

In a number of embodiments, 3p-C-NETA chelators can be labeled with nuclides including (but not limited to)¹⁷⁷Lu for therapeutic applications. 3p-C-NETA can be efficiently labeled with ¹⁷⁷Lu, to form ¹⁷⁷Lu-3p-C-NETA complex at room temperature in about 5 minutes. In a number of embodiments, DOTA and DOTAGA chelators can be labeled with nuclides including (but not limited to)⁶⁸Ga, ⁸⁹Zr, or ⁶⁴Cu for PET imaging applications. In several embodiments, DOTA and DOTAGA chelators can be labeled with nuclides including (but not limited to)¹⁷⁷Lu or ²²⁵Ac for therapeutic applications. DOTA- and DOTAGA-¹⁷⁷Lu-labeling may occur at about 95° C. for about 15-45 min.

In many embodiments, amino acid spacers can be added to HetSiFA tracers. In several embodiments, amino acid spacers including (but not limited to) histidine (H or His)/glutamic acid (E or Glu) and/or tryptophan (W or Trp)/glutamic acid (E or Glu) can enhance the affinity of the HetSiFA tracers to target receptors and to obtain better in vivo imaging properties.

In many embodiments, HetSiFA-chelator constructs can be conjugated to disease-binding ligands to form precursors for theranostic application. HetSiFA-derived theranostic pairs in accordance with some embodiments can be identical twins with a genuine ‘see it, treat it’ capability. The diagnostic and therapeutic pairs have the same chemical composition but differ in the nuclides.

### HetSiFA Compounds Properties

Many embodiments provide theranostic HetSiFA compounds with unexpected properties, including (but not limited to) radiolabeling efficiency, hydrophilicity, chemical versatility, hydrolytic stability, human serum albumin (HSA) binding ability, plasma protein binding ability, kinetic solubility, target binding affinity, and biodistribution properties in mice.

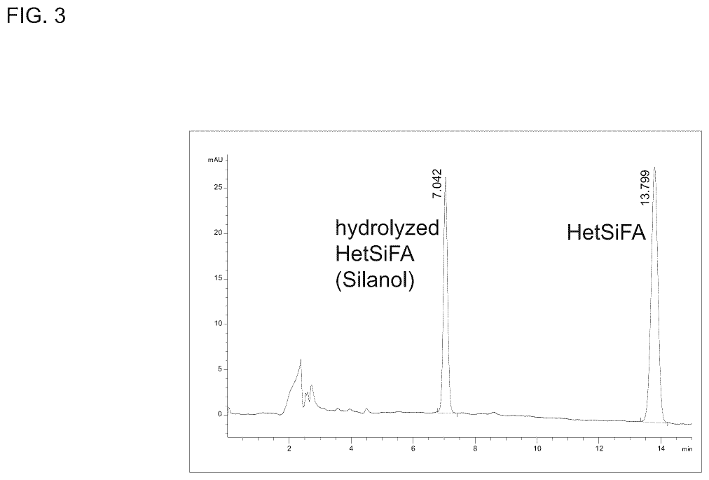

In many embodiments, pyrazole-HetSiFAs compounds have desired radiolabeling properties, kinetic solubility, chemical versatility, hydrophilicity, and in vivo stability. Pyridine-HetSiFAs in accordance with some embodiments have desired hydrophilicity and chemical versatility. In certain embodiments, azaindole-HetSiFAs have desired radiolabeling properties, are hydrolytically stable, and chemically versatile. Brief descriptions of each property and the characterization of each property are stated below.
a) Hydrophilicity. Hydrophilicity relates to the strength of being dissolved in or absorbed in aqueous solutions. It denotes a preference for liquids, solutions, or surfaces with a high dipole moment, like water. Lipophilicity denotes a preference for liquids, solutions, or surfaces with a low dipole moment, such as lipid solutions. The more polar the HetSiFA, the less masking of the lipophilicity may be needed in the radiopharmaceutical via introducing cumbersome and lengthy linkers. The hydrophilicity of HetSiFAs is assessed by calculating and/or measuring the octanol/water partition coefficient log P. Log D measures the lipophilicity of ionizable compounds, where the partition is a function of the pH. The reversed-phase HPLC retention times t_{R} can be used to qualitatively compare the polarity of HetSiFAs.
b) Hydrolytic stability. The HetSiFA Si—¹⁸F bond may be susceptible to hydrolysis in aqueous media resulting in the formation of silanol. The HetSiFA molecular design can influence the hydrolytic stability of the Si—¹⁸F bond. The hydrolytic stability of synthesized HetSiFAs can be examined in carbonate buffer (0.1 M aqueous, 0.05 M in mixture) at pH 10. The HetSiFA substrate (100 μM in 1:1 MeCN/pH 10 carbonate buffer) is aged at room temperature (25° C.). At different time points, the substrate is quantified by HPLC UV monitoring. HetSiFA is dissolved in acetonitrile—aqueous buffer (pH 7.4 or 10) 1:1 mixture at 100 μM. The hydrolysis is monitored by HPLC (˜7 timepoints). Hydrolytic stability can be referred to as pH10 stability and reported as the half-life of the Si—F bond at pH10 (t_{1/2(pH10)}). FIG. 3 illustrates an HPLC chromatogram of HetSiFA and hydrolyzed HetSiFA is illustrated in accordance with an embodiment. Typical HPLC conditions: Phenomenex Prodigy ODS-3 column (250×4.6 mm, 10 pm), mobile phase: MeCN/H₂O (0.1% HCO₂H), gradient, 20:80 to 50:50 (15 min), hold (5 min), 0.8 mL/min. T=25° C. In addition, the stability of compounds can be assessed in rat and human plasma and in microsomes.
c) Radiolabeling. For HetSiFA-based PET tracers to be clinically useful, they need to be radiolabeled in high radiochemical yields (RCY). The radiolabeling properties of synthesized HetSiFAs can be assessed using low amounts of radioactivity. Radio-thin-layer chromatography (radio-TLC) is used to measure the ¹⁸F-fluoride incorporation or radiochemical conversion (RCC).
d) Chemical versatility. The availability of aromatic heterocycles enables HetSiFAs with different ring sizes, heteroatom cores, and substitution patterns. Modifying the HetSiFA core enables fine-tuning of steric and electronic properties and the addition of linkers and other functionalities.
e) Human serum albumin and plasma protein binding. Binding to plasma proteins such as human serum albumin (HSA) influences the blood-circulation times of radiopharmaceuticals and impacts their ability to bind to the biological target. HSA binding properties of HetSiFAs can be assessed with an HPLC method using a Chiralpak HSA column. For the calibration of the HSA column, nine reference substances displaying a range from 13% to 99% HSA binding are measured, and a standard curve is established. Reference substances are p-benzyl alcohol (lit. HSA %=13.15), aniline (lit. HSA %=14.06), phenol (lit. HSA %=20.69), benzoic acid (lit. HSA %=34.27), carbamazepine (lit. HSA %=75.00), p-nitrophenol (lit. HSA %=77.65), estradiol (lit. HSA %=64.81), probenecid (95.00), and glibenclamide (lit. HSA %=99.00). HSA % binding of compounds was derived from the standard curve. The plasma protein binding properties of HetSiFAs can be assessed with mouse and human plasma proteins.
f) Kinetic solubility. Solubility is an important parameter that influences pharmacokinetic behavior. Kinetic solubility can be assessed in phosphate-buffered saline (PBS) at pH 7.4.
g) Target binding affinity. Binding affinity indicates the strength of drug-target interactions. It can be measured in cells that stably express the target of interest by displacing iodine-125 labeled ligands in a dose-depended manner.
h) Biodistribution in mice. Ex vivo biodistribution studies in mice reveal the distribution of the injected tracer in certain organs after a pre-defined time post-injection (p.i.). In a typical ex vivo biodistribution experiment, four healthy mice are injected with 3.7-10 MBq of ¹⁸F-labeled HetSiFA tracer. One mouse is PET imaged 10-90 min p.i. and then euthanized. Three mice are PET imaged 70-90 min p.i. and then euthanized. Organs are weighed, and the activity is measured in a gamma counter.

### HetSiFA Compounds for ¹⁸F Radiolabeling

Many embodiments provide an array of HetSiFAs for ¹⁸F radiolabeling. In several embodiments, HetSiFAs with heteroaromatics including (but not limited to) pyridines, pyridiniums, pyridine oxides, pyrazoles, fused pyrazoles, imidazoles, pyrimidines, and azaindoles show better properties for making radiopharmaceuticals than phenyl-SiFA. Some embodiments provide that HetSiFAs with heteroaromatics, including (but not limited to) indoles, benzothiophenes, benzofurans, furans, and pyrroles, show less favorable radiolabeling properties than phenyl-SiFA.

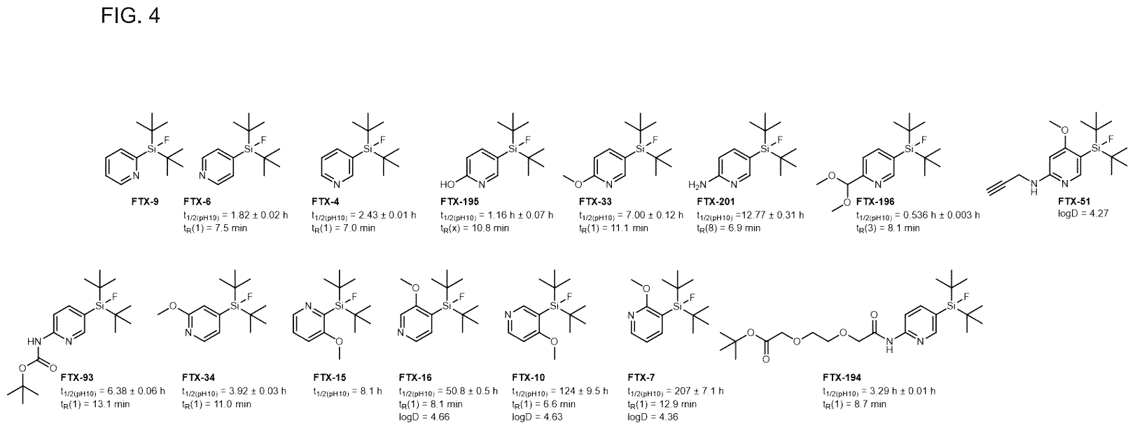

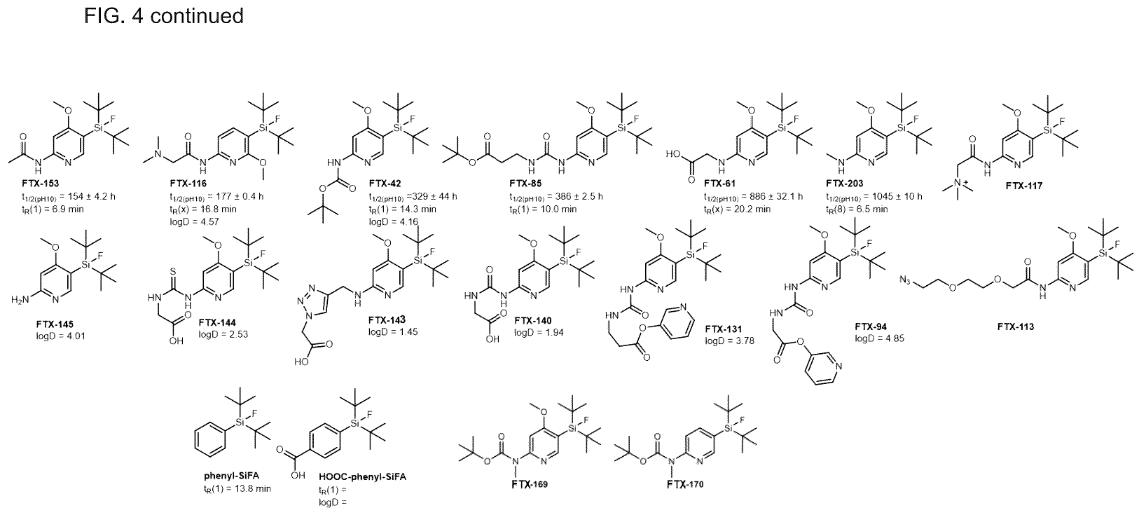

FIG. 4 illustrates structures of pyridine-HetSiFA compounds in accordance with an embodiment of the invention. Hydrolytic stability at pH10 is reported as the half-life of the Si—F bond at pH10 (t_{1/2(pH10)}). The reversed-phase HPLC retention times t_{R} can be used to qualitatively compare the polarity of HetSiFAs. Pyridyl-HetSiFAs are more polar than phenyl-SiFA, as evidenced by the shorter t_{R} on reversed-phase HPLC. This is in accordance with the log P of the aromatic cores (log P_{pyridine}=0.65; log P_{benzene}=2.13). The incorporation percentage of ¹⁸F via ¹⁸F-for-¹⁹F isotopic exchange on silicon (SiFEx) can be reported as the radiochemical conversion percentage (RCC).

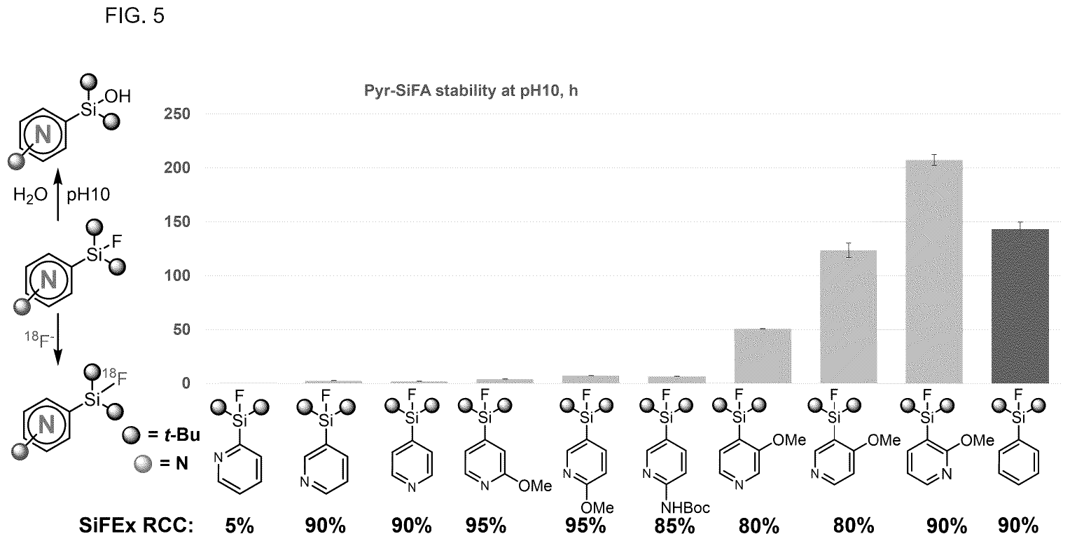

Many embodiments provide that substituent effects can increase Si—F bond stability. A methoxy group in ortho-position to the silicon increases Si—F stability. 3-(Di-tert-butylfluorosilyl)-4-methoxypyridine has slightly lower hydrolytic stability than phenyl-SiFA. 5-(Di-tert-butylfluorosilyl)-6-methoxypyridine has higher hydrolytic stability than phenyl-SiFA. FIG. 5 illustrates the hydrolytic stabilities and radiochemical conversions (RCC) of pyridyl-HetSiFAs in accordance with an embodiment.

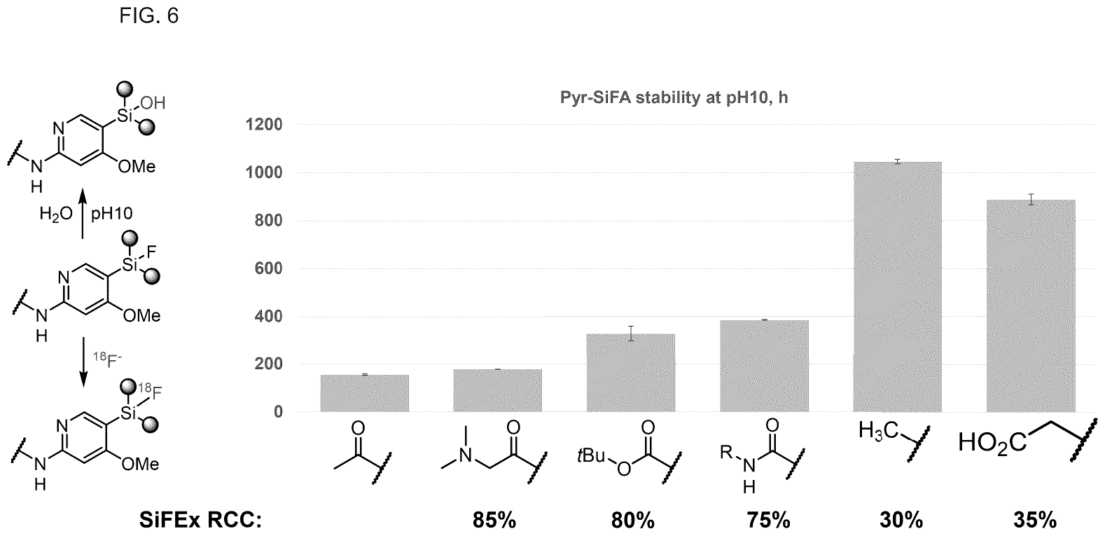

FIG. 6 illustrates the hydrolytic stabilities and radiochemical conversions (RCC) of pyridyl-HetSiFAs in accordance with an embodiment. FIG. 6 shows that substituents in para-position to silicon influence Si—F stability. Pyridine-HetSiFAs are efficiently radiolabeled with ¹⁸F. Radiochemical conversions (RCC) are comparable to phenyl-SiFAs, with RCC often exceeding 80%.

Pyridine-HetSiFAs are chemically versatile and allow extensive regioselective modification of the pyridine core, also via substitutions on both oxygen and nitrogen in accordance with some embodiments. Sidechains with functional groups can be added to provide a point of attachment for the disease-binding ligand.

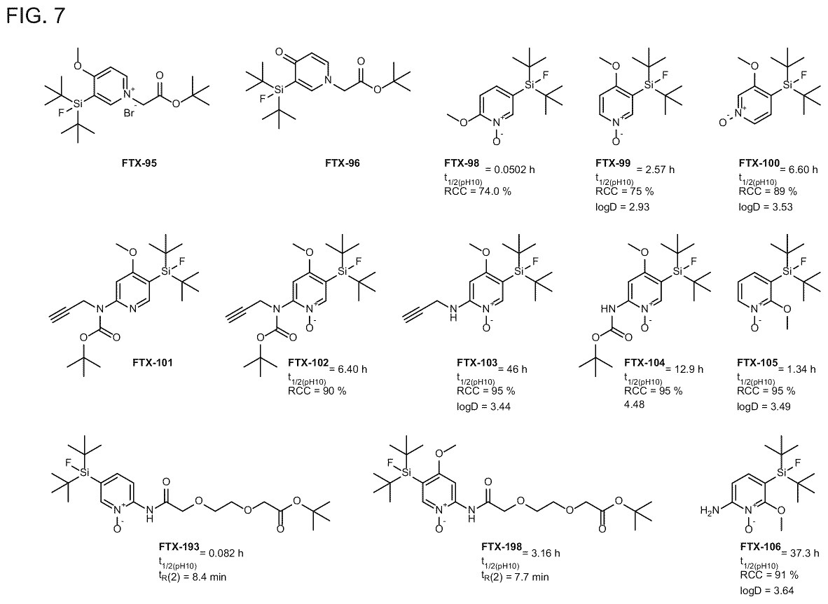

In many embodiments, pyridine-HetSiFAs are converted into the corresponding N-modified derivatives to increase their polarity. FIG. 7 illustrates structures of pyridyl N-modified HetSiFA compounds in accordance with an embodiment. In many embodiments, N-modified pyridine-HetSiFAs provide a point of attachment at nitrogen. Pyridine-oxide and pyridinium HetSiFAs are more polar than their pyridine counterparts. They are radiolabeled efficiently but tend to decompose in solution over time.

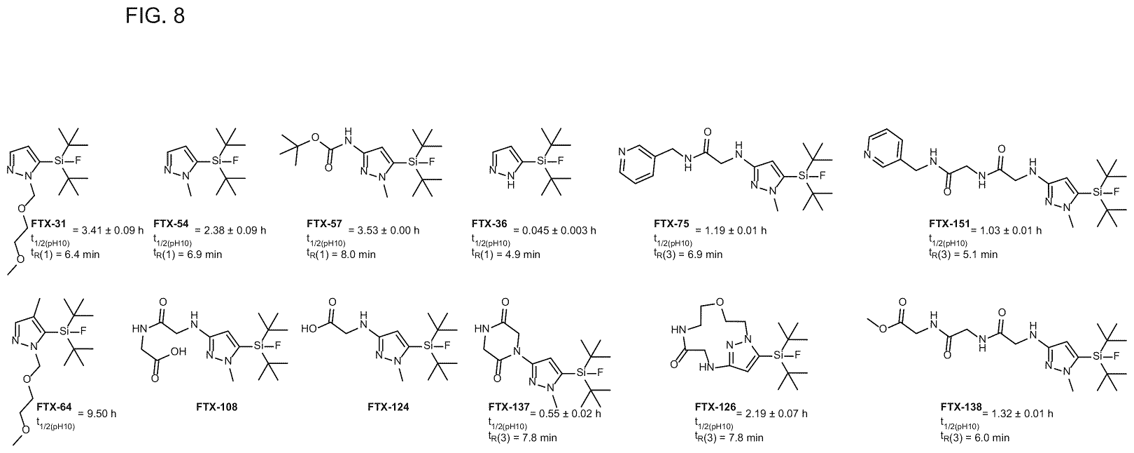

Many embodiments provide pyrazole-HetSiFA compounds with better radiolabeling properties, kinetic solubility, and hydrophilicity. Compared to phenyl-SiFAs, pyrazole-HetSiFA compounds are smaller and more polar (log P_{pyrazole}=0.26; log P_{benzene}=2.13). FIG. 8 illustrates structures of pyrazole-HetSiFA compounds in accordance with an embodiment of the invention.

Up to three side chains can be attached to pyrazole-HetSiFAs, thus providing access to a wide variety of distinct molecules. Pyrazole-HetSiFAs provide good chemical versatility.

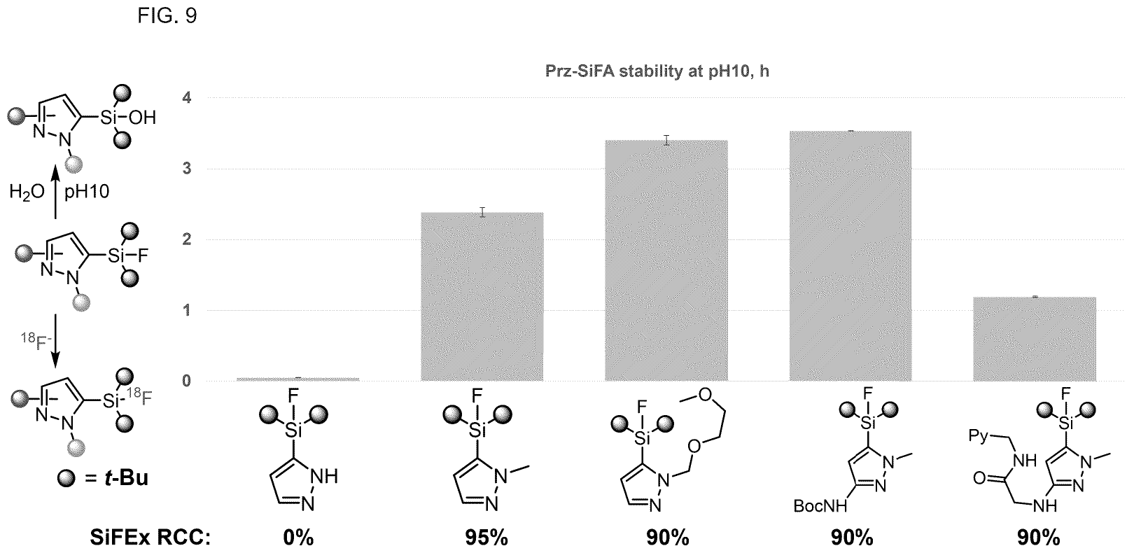

The hydrolytic stabilities of pyrazole-HetSiFA assessed in the pH10 stability assay are comparably low. However, a pH10 environment may not be reached in a physiological environment. FIG. 9 illustrates hydrolytic stabilities and radiochemical conversions (RCC) of pyrazole-HetSiFAs. FIG. 9 shows that substituents of pyrazole-HetSiFA can influence hydrolytic stability.

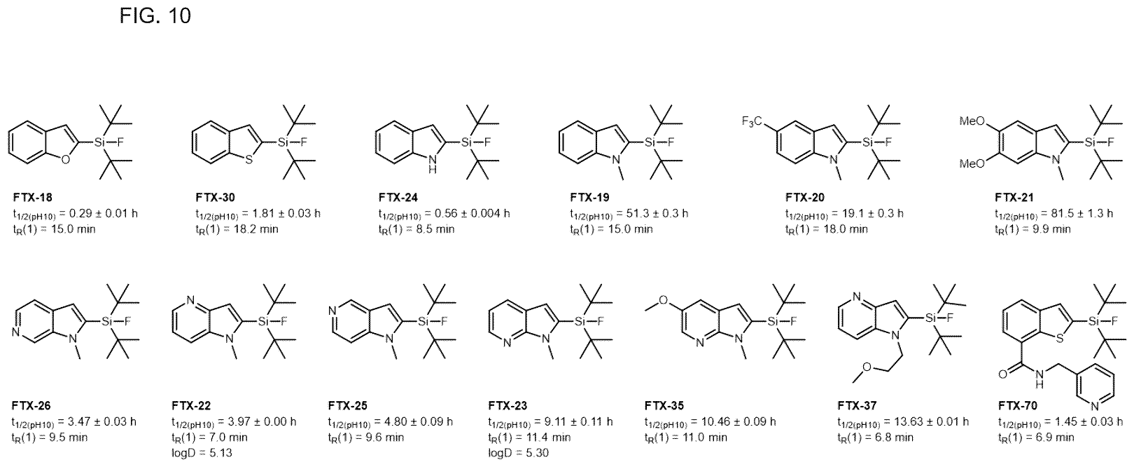

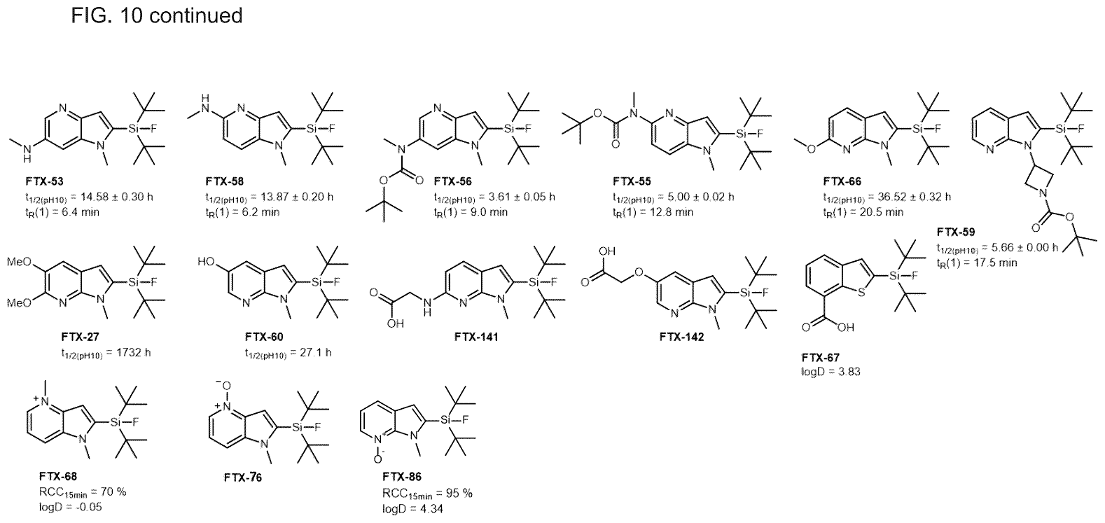

Many embodiments provide [6+5]-fused-HetSiFA compounds with favorable radiolabeling properties, hydrolytic stability, kinetic solubility, and hydrophilicity. FIG. 10 illustrates structures of [6+5]-fused-HetSiFA compounds in accordance with an embodiment of the invention.

Several embodiments provide that some [6+5]-fused-HetSiFAs radiolabel efficiently, are hydrolytically stable, and chemically versatile. [6+5]-Fused-HetSiFAs are generally larger than pyrazole- and pyridine-HetSiFAs but some remain more polar than phenyl-SiFA.

Many [6+5]-fused-HetSiFAs have an azaindole core. Azaindole-HetSiFAs in accordance with some embodiments can radiolabel well and show high hydrolytic stability, depending on how the azaindole core is modified.

Some embodiments provide that the nitrogen of the pyridine-moiety in azaindoles can be in the 7-, 6-, 5-, or 4-position. Azaindole-HetSiFAs provide high chemical versatility.

In some embodiments, azaindoles are converted into their N-oxide or N-substituted forms. The modifications increase the polarity of the core. They are often radiolabeled efficiently at the cost of hydrolytic stability. Due to their large size and comparably low hydrophilicity, azaindoles have a minor priority as HetSiFA cores for radiopharmaceutical development.

Many embodiments provide that indole, benzothiophene, and benzofurane are less polar than phenyl-SiFA, and their chemical versatility may be low. Some furan- and pyrrole-HetSiFAs lack chemical versatility and have high lipophilicity. Some furan-HetSiFAs radiolabel efficiently, but the stability of the Si—F-bond is low. Some pyrrole-HetSiFA are ¹⁸F-labeled less efficiently. These compounds may not be ideal for HetSiFA radiopharmaceutical development.

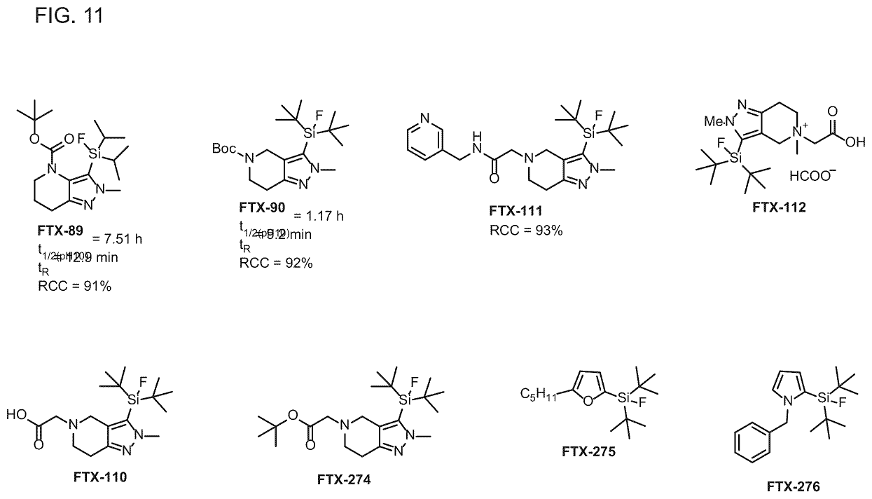

FIG. 11 illustrates structures of fused-HetSiFAs, furan-HetSiFAs, and pyrrole-HetSiFAs and their properties in accordance with an embodiment of the invention.

### HetSiFA Constructs for PET Imaging

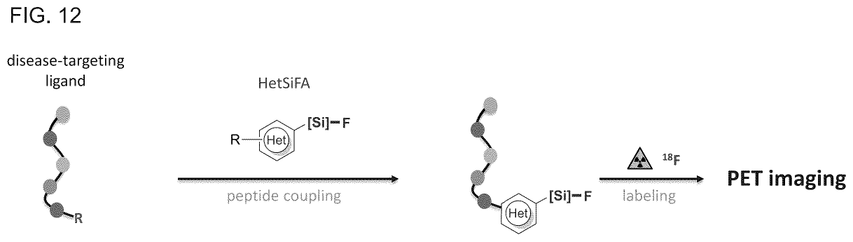

HetSiFAs with varying physicochemical and labeling properties can be conjugated with disease-targeting ligands to generate precursors labeled with ¹⁸F for PET imaging. FIG. 12 illustrates a schematic of synthesizing a HetSiFA PET tracer in accordance with an embodiment. The disease-targeting ligand can be conjugated via the N-terminus of a peptide on resin. Any type of a disease-targeting ligand such as (but not limited to) peptide or peptidomimetic, that can target cancer cells can be implemented. Examples of targets for disease-binding ligands include (but are not limited to): GRPR, PSMA, SSTR2, FAP, and CXCR4. HetSiFA compounds with various aromatic cores such as (but not limited to) pyrazole, pyridine, pyridinium, azaindole, imidazole, and pyrimidine, and polar linkers can be conjugated with the disease-targeting ligand. The aromatic cores of the HetSiFA compounds may affect the pharmacological properties of the HetSiFA-ligand constructs. The conjugated candidates may vary in physicochemical and labeling characteristics and can be screened for their radiolabeling, hydrolytic stability, binding affinity, and other properties. HetSiFA-ligand constructs can then be radiolabeled with ¹⁸F for PET imaging yielding up to hundreds of patient doses.

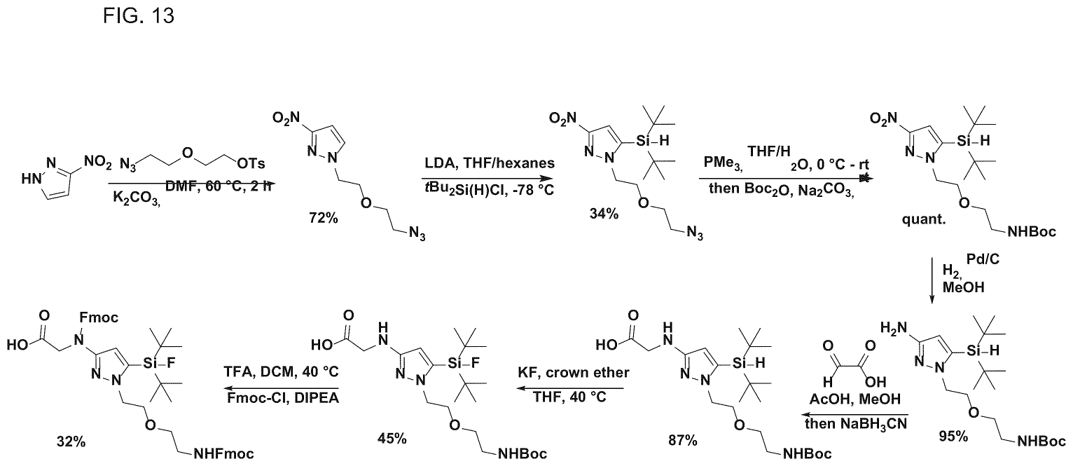

FIG. 13 shows the synthetic scheme of a pyrazole-HetSiFA building block that can be conjugated to disease-binding ligands in accordance with an embodiment of the invention.

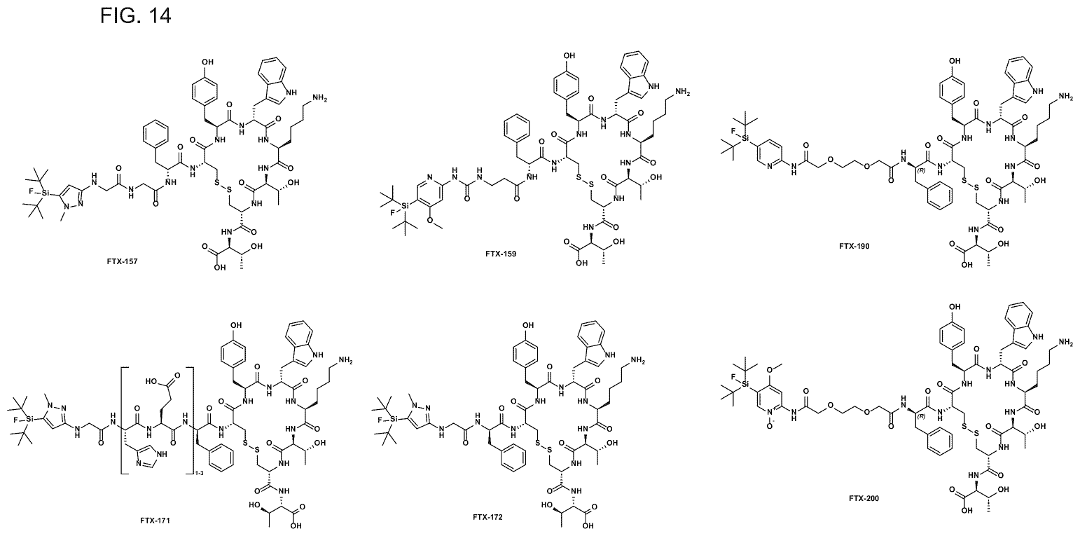

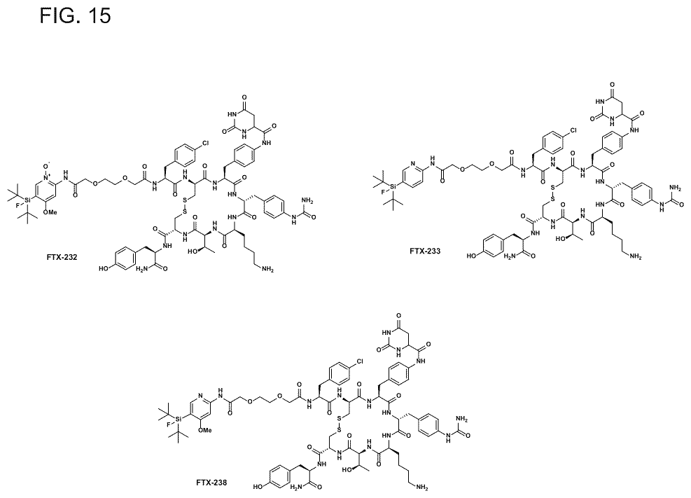

FIG. 14 illustrates structures of HetSiFAs conjugated to TATE for PET imaging of SSTR2 in accordance with an embodiment of the invention. FIG. 15 illustrates HetSiFAs conjugated to JR11 for PET imaging of SSTR2 in accordance with an embodiment of the invention.

### HetSiFA Constructs for Theranostics

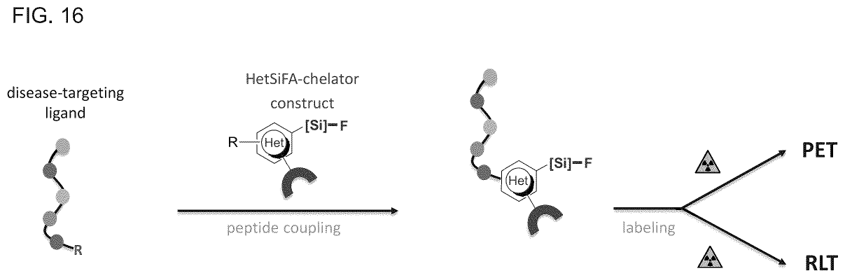

For theranostic applications, HetSiFAs can be combined with chelators for (radio)metal complexation. FIG. 16 illustrates a schematic of the conjugation of HetSiFA-chelator construct with disease-targeting ligands in accordance with an embodiment. The HetSiFA-chelator construct can be conjugated with disease-targeting ligands to generate precursors for PET imaging and RLT as theranostic pairs. The disease-targeting ligand can be conjugated via the N-terminus of a peptide on resin. Any type of a disease-targeting ligand such as (but not limited to) peptide or peptidomimetic, that can target cancer cells can be implemented. Examples of targets for disease-binding ligands include (but are not limited to): GRPR, PSMA, SSTR2, FAP, and CXCR4. HetSiFA with various aromatic cores including (but not limited to) pyrazole, pyridine, pyridinium, azaindole, imidazole, and pyrimidine can be applied. The HetSiFA-chelator constructs may vary in aromatic cores, polar linkers, and chelators. The HetSiFA-chelator constructs may affect the pharmacological properties of the HetSiFA-chelator-ligand constructs. The conjugated candidates may vary in physicochemical and labeling characteristics and can be screened for their radiolabeling, hydrolytic stability, binding affinity, and other properties. The conjugated candidates can be labeled with either ¹⁸F for PET imaging or ¹⁷⁷Lu/²²⁵Ac for RLT. Furthermore, the chelator enables labeling with other PET isotopes, including (but not limited to) ⁶⁸Ga, ⁶⁴Cu, or ⁸⁹Zr.

Development of theranostic pairs historically needed separate and lengthy development processes for both the imaging and therapeutic companions. The fact that only one precursor is needed to manufacture some ⁶⁸Ga PET/¹⁷⁷Lu theranostic pairs has likely contributed to their popularity. ¹⁸F/¹⁷⁷Lu pairs may need to be more accessible to increase the use of ¹⁸F in theranostics. Substituting ¹⁸F for ⁶⁸Ga can improve patient care (through imaging with higher resolution) and increase the availability of radiopharmaceuticals (through scaling). HetSiFA-chelator-derived precursors allow labeling with nuclides which include but are not limited to ¹⁸F, ¹⁷⁷Lu, or ²²⁵Ac. Labeling with ⁶⁸Ga or ⁶⁴Cu for PET imaging is also possible if required.

In some embodiments, HetSiFAs combined with metal chelators can be made as theranostic HetSiFAs. The compounds of HetSiFAs with metal chelators can also be used as non-theranostic ¹⁸F-PET imaging radiopharmaceuticals in accordance with several embodiments. In such embodiments, the chelator can be added to reduce the overall polarity of the HetSiFA moiety. The chelator can be left empty, or a non-radioactive metal can be chelated.

Chelators that may be conjugated to the HetSiFA moieties to obtain HetSiFA-chelator conjugates include (but are not limited to) DOTA, DOTAGA, NOTA, and NODAGA. DOTA and DOTAGA chelators can be labeled with nuclides for PET (⁶⁸Ga or ⁶⁴Cu) or therapy (¹⁷⁷Lu or ²²⁵Ac). 3p-C-NETA may also be conjugated to HetSiFAs. Examples of chelators include (but are not limited to) EDTA, CHX-A″-EDTA, DTPA, p-SCN-DTPA, CHX-A″-DTPA, pSCN-Bz-Mx-DTPA, TETA, CB-TE2A, CB-TE1A1P, p-SCN-NOTA, nNOTA, NODAGA, p-SCN-DOTA, DOTAGA, 2-cTETA, 6-cTETA, BAT, Diamsar, SarAr, PCTA, NODIA-Me, TRAP, pycup1A1B, pycup2A, p-SCN-DTPA, DFO, DFO B, DFO B Mesylate, DFO-p-SCN, DFO-Star, L5, Orn3hx-NCS, Orn4hx-NCS, p-SCN-Bn-HOPO, YM103, 2,3-HOPO-p-Bn-NCS, oxo, nitride, HYNIC, fac-tricarbonyl, trioxo, 3p-C-NETA-NCS, 3p-C-DEPA-NCS, TCMC, p-SCN-Bn-H₄octapa, HEHA-NCS, and Macropa-NCS.

Cations that the chelating group may chelate are the cations of ⁴³Sc, ⁴⁴Sc, ⁴⁵Sc, ⁴⁷Sc, ⁵¹Cr, ^{52m}Mn, ⁶⁸Co, ⁵²Fe, ⁵⁶Ni, ⁵⁷Ni, ⁶¹Cu, ⁶²Cu, ⁶³Cu, ⁶⁴Cu, ⁶⁵Cu, ⁶⁷Cu, ⁶⁶Ga, ⁶⁷Ga, ⁶⁸Ga, ⁶⁹Ga, ⁷¹Ga, ⁹⁰Zr, ⁹¹Zr, ⁹²Zr, ⁸⁹Zr, ⁸⁶Y, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹I, ¹¹³In, ^{133m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵⁵Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁶Lu, ¹⁷⁷Lu, ^{177m}Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²⁰⁴Pb, ²⁰⁶Pb, ²⁰⁷Pb, ²⁰⁸Pb, ²¹¹At, ²⁰⁹Bi, ²¹²Bi, ²¹³Bi, ²²³Ra, ²²⁵Ac, ²²⁷Th, ²³²Th, or a cationic molecule comprising ¹⁸F such as ¹⁸F[AlF]²⁺.

FIG. 17 illustrates the structures of HetSiFA theranostic compounds with no biological function for radiolabeling testing in accordance with an embodiment of the invention.

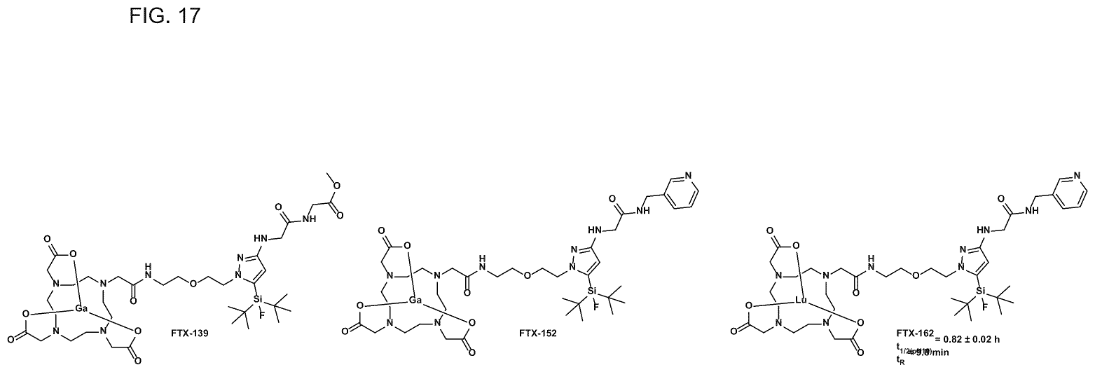

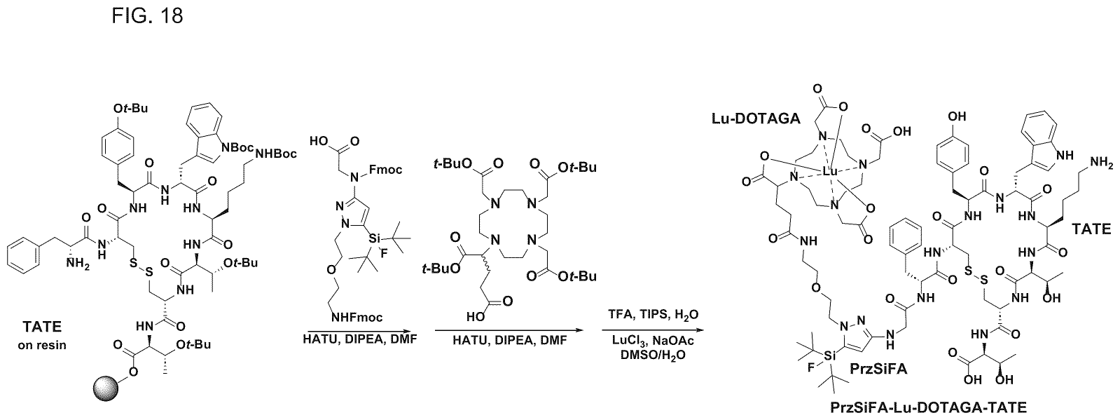

FIG. 18 illustrates a synthetic scheme for a theranostic HetSiFA targeting SSTR2 in accordance with an embodiment of the invention.

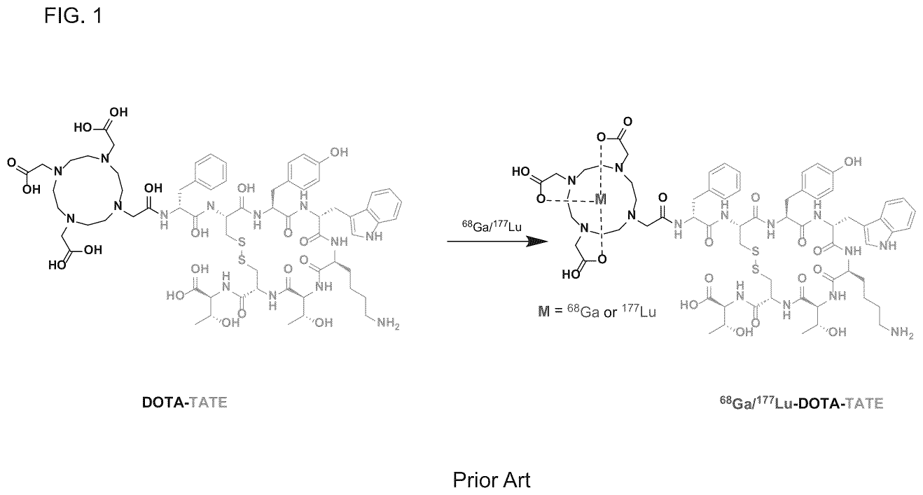

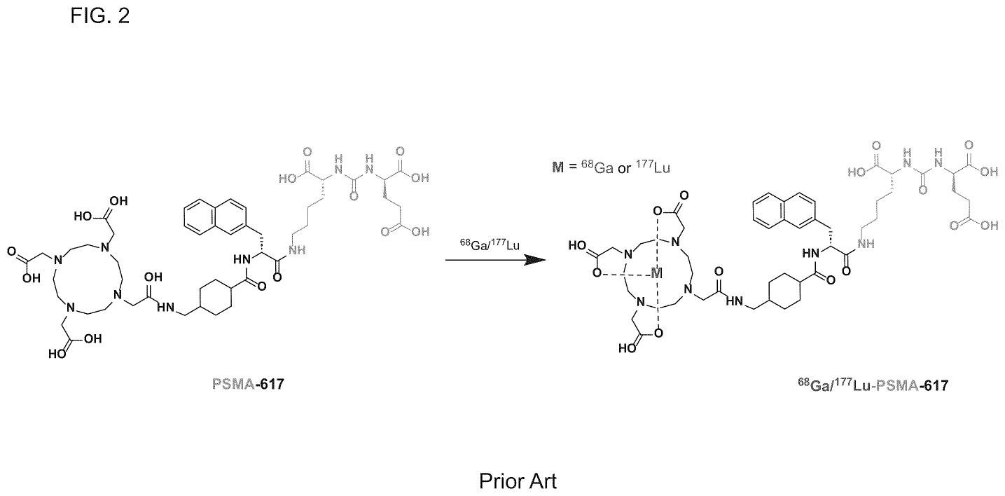

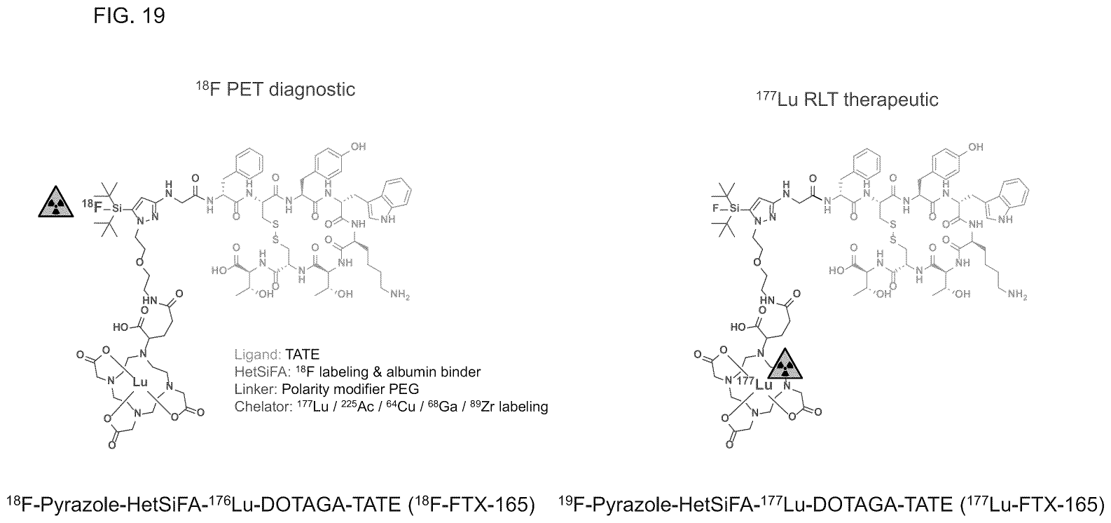

FIG. 19 illustrates an SSTR2-ligand conjugated with a HetSiFA-chelator construct forming an SSTR2-targeting HetSiFA-theranostic in accordance with an embodiment of the invention. With a ^{nat}Lu complexed in the chelator, the compound can be labeled with ¹⁸F for PET diagnostics (¹⁸F-Pyrazole-HetSiFA-¹⁷⁶Lu-DOTAGA-TATE (¹⁸F-FTX-165)). When the chelator is empty, the compound can be labeled with ¹⁷⁷Lu for RLT (¹⁹F-Pyrazole-HetSiFA-¹⁷⁷Lu-DOTAGA-TATE (¹⁷⁷Lu-FTX-165)). HetSiFA-derived theranostic pairs in accordance with some embodiments, can be identical twins with a genuine ‘see it, treat it’ capability. The diagnostic and therapeutic pairs differ in the nuclides but not the chemical composition. FIG. 19 illustrates that the ¹⁸F PET diagnostic and the ¹⁷⁷Lu therapeutic differ only in the radioactive labels. Other theranostic pairs, such as ⁶⁸Ga-DOTA-TATE and ¹⁷⁷Lu-DOTA-TATE (FIG. 1), are not chemically identical and differ in the radiometal. Replacement of the radiometal may impact biodistribution, target affinity, metabolic stability, and other properties.

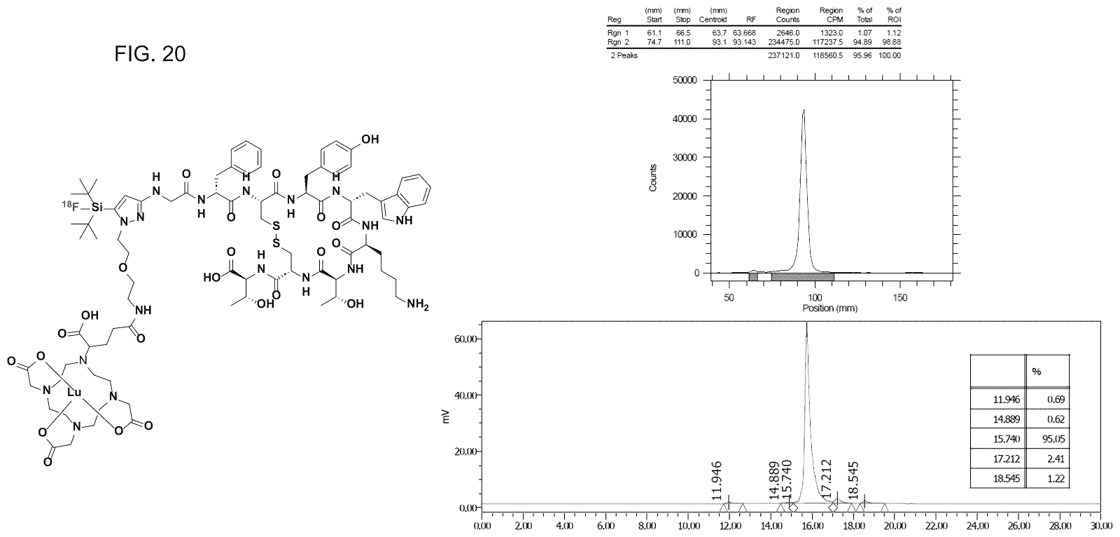

FIG. 20 illustrates a ¹⁸F labeled pyrazole-HetSiFA compound ¹⁸F-FTX-165 in accordance with an embodiment. FIG. 20 shows the radio-HPLC and radio-TLC chromatograms of the compound ¹⁸F-Pyrazole-HetSiFA-Lu-DOTAGA-TATE. RCC (low activity) equals to about 80% (rTLC, 5 min). RCY (medium activity) equals to about 54% (isolated). RCY (high activity) equals to about 47% (isolated). Max isolated activity equals to about 23.9 GBq (645 mCi). Aₘ is greater than about 159 GBq/μmol (4.3 Ci/μmol).

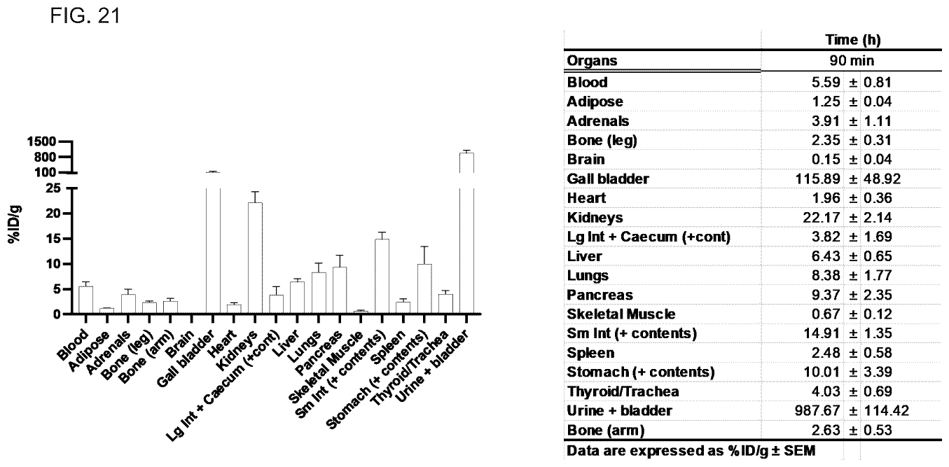

FIG. 21 illustrates ex vivo biodistribution data of ¹⁸F-Pyrazole-HetSiFA-Lu-DOTAGA-TATE (¹⁸F-FTX-165) in healthy mice in accordance with an embodiment. The chemical structure of the compound is shown in FIG. 20. The ex vivo biodistribution data is taken in 3 Balb/c female mice. Little bone uptake is observed, indicating sufficient stability of the Si—¹⁸F bond in vivo.

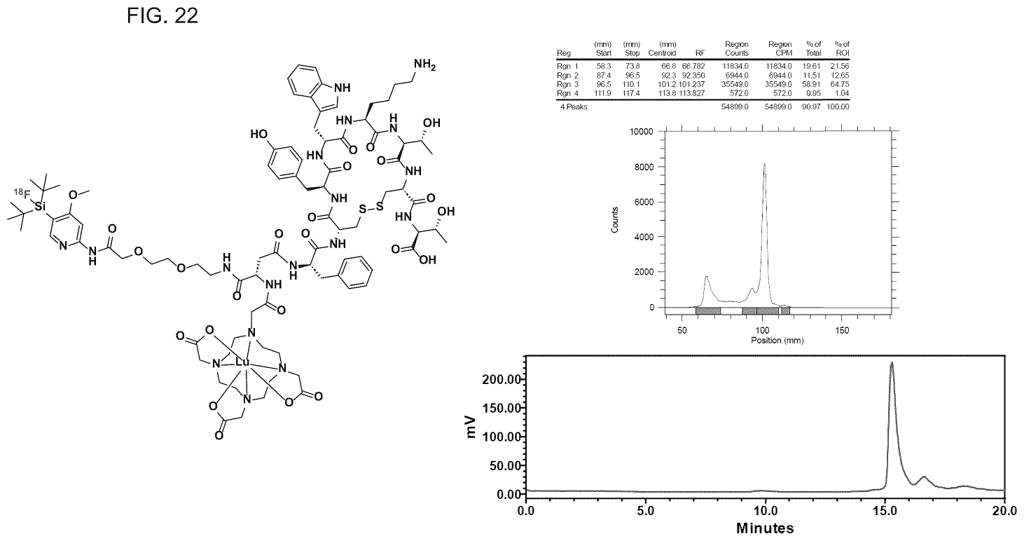

FIG. 22 illustrates a ¹⁸F labeled pyridine-HetSiFA compound ¹⁸F-FTX-164 in accordance with an embodiment. FIG. 22 shows the radio-HPLC and radio-TLC chromatograms of the compound ¹⁸F-Pyridine-HetSiFA-Lu-DOTAGA-TATE. The compound is decomposed when radiolabeled with about 77 GBq of starting activity. RCC (low activity) equals to about 65%. RCY (medium activity) equals to about 15%. RCY (high activity) is less than about 1%. Radiolysis may have happened.

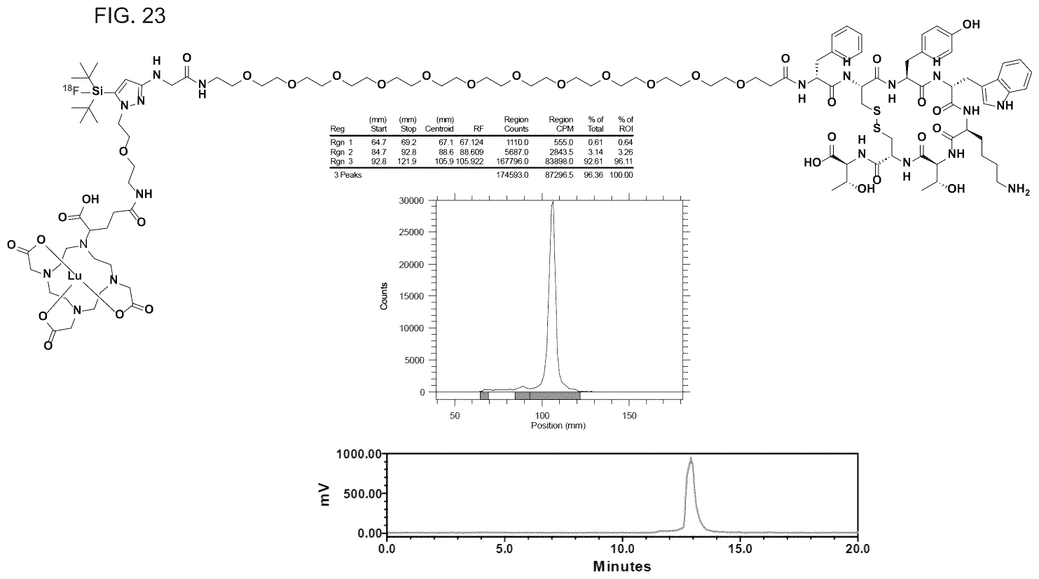

Countering lipophilicity of HetSiFAs in radiopharmaceuticals may be needed. FIG. 23 illustrates a ¹⁸F labeled pyrazole-HetSiFA compound in accordance with an embodiment. FIG. 23 shows the radio-HPLC and radio-TLC chromatograms of the compound Lu-DOTAGA-¹⁸F-Pyrazole-HetSiFA-PEG₁₂-TATE (¹⁸F-FTX-181). A polyethylene glycol (PEG) chain is added to increase solubility and hydrophilicity. The ¹⁸F-labeled compound can be isolated in high activity yields of up to 41 GBq. RCC (low activity) equals to about 75% (rTLC, 5 min). RCY (medium activity) equals to about 31% (isolated). RCY (high activity) equals to about 51% (isolated). Max isolated activity equals to about 41 GBq (1.1 Ci). Aₘ is greater than about 273 GBq/μmol (7.33 Ci/μmol).

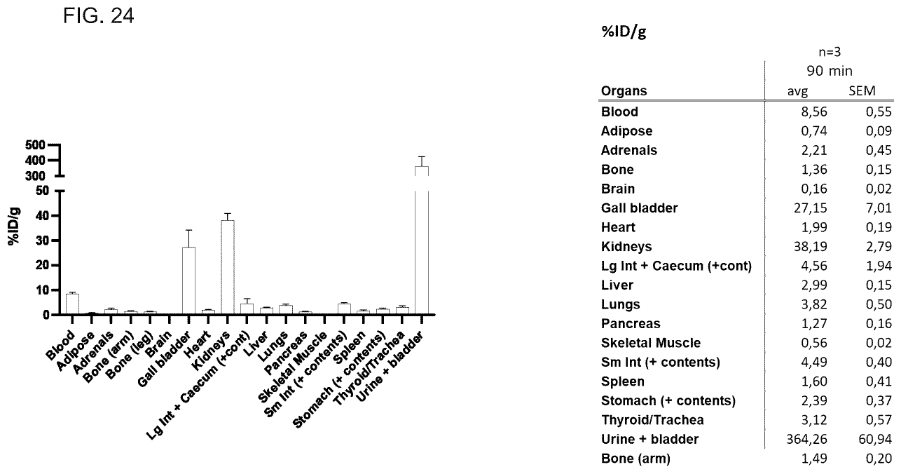

FIG. 24 illustrates ex vivo biodistribution data of Lu-DOTAGA-¹⁸F-Pyrazole-HetSiFA-PEG₁₂-TATE in healthy mice in accordance with an embodiment. The chemical structure of the compound ¹⁸F-FTX-181 is shown in FIG. 23. The ex vivo biodistribution data is taken in 3 Balb/c female mice. Compared to ¹⁸F-FTX-165, less bone uptake is observed, indicating sufficient stability of the Si—¹⁸F bond in vivo. This suggests that factors other than the hydrolytic stability of the Si—¹⁸F bond influence the Si—¹⁸F bond stability in vivo. Enzymatic metabolism in the liver may contribute to the defluorination of lipophilic HetSiFAs, even the ones stable towards hydrolysis. (See, e.g., S. Otaru, et al., Mol. Pharmaceutics, 2020, 17, 3106-3115; S. Otaru, et al., Molecules, 2020, 25, 5, 1208; the disclosures of which are herein incorporated by references.) Favoring renal elimination of the HetSiFA radiopharmaceutical likely helps to reduce hepatic defluorination. Accumulation of ¹⁸F-FTX-181 in the liver is low, and it washes out from non-targeted organs except for the gallbladder. ¹⁸F-FTX-181 shows a promising biodistribution that warrants further development.

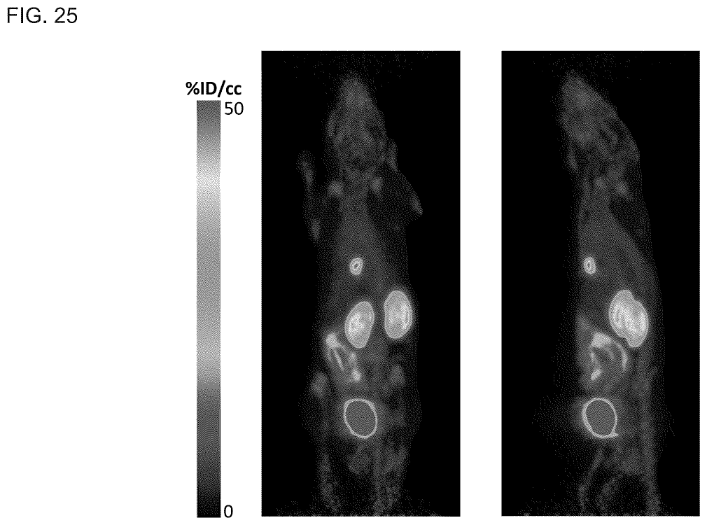

FIG. 25 illustrates a maximum-intensity PET image of ¹⁸F-FTX-181 in a healthy mouse 70-90 min post-injection.

(Het)SiFAs are known to bind to plasma proteins. Binding to plasma proteins may influence the blood-circulation times of radiopharmaceuticals and impact their ability to bind to the biological target. HetSiFAs may alter plasma protein-binding properties in two ways: 1) The large variety of heterocycles in the chemical space enables the search for candidates with desired plasma protein-binding properties. 2) The plasma protein-binding HetSiFA moiety can be placed in different parts of the radiopharmaceutical. The HetSiFA can either stick out from the radiopharmaceutical to make it more accessible to plasma proteins or be “sandwiched” by other parts of the molecule to make it less accessible. The phenyl in phenyl-SiFAs has not been chemically modified to influence their plasma protein binding. Phenyl-SiFAs are generally connected to the radiopharmaceutical via a side chain going off from the para-position relative to the silicon substituent. Consequently, all phenyl-SiFAs stick out of the molecular framework, which renders the “sandwiching” construct not possible.

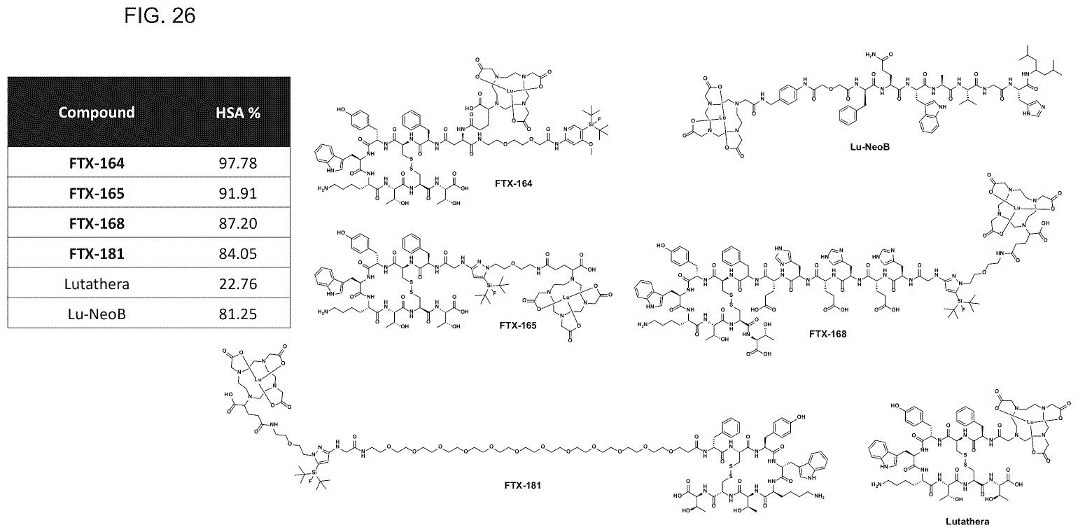

FIG. 26 illustrates human serum albumin (HSA) binding assay results of HetSiFA compounds in accordance with an embodiment. A standard curve can be first established using reference compounds (See, e.g., Valko, K. et al., J. Pharm. Sci. 2003, 92 (11), 2236-2248; Wurzer, A. J. et al., PSMA binding dual mode radiotracer and therapeutic. 2020, WO2020157177A1; the disclosures of which are herein incorporated by reference.) The HSA binding assay results include four HetSiFA theranostic compounds, FTX-164, FTX-165, FTX-168, and FTX-181 (structures shown in FIG. 26), and two therapeutics Lutathera and Lu-NeoB. As shown in FIG. 26, the four HetSiFA compounds have HSA % ranges from about 84% to about 98%. Test condition of the HSA binding assay includes: Column: Chiralpak HSA (100×2.1 mm, 5 μm, Cat. No. HSA5LM-C0004), flow rate: 0.3 mL/min. Mobile phase: A—NH₄₀Ac buffer (50 mM, pH=7), freshly prepared each day; B—isopropanol (HPLC grade); 0-3 min: isocratic 100% A, 0% B; 3-40 min: gradient to 80% A, 20% B; 40-60 min: isocratic 80% A, 20% B. Samples: 9 reference compounds, concentration 0.5 mg/mL in i-PrOH/NH₄OAc (50 mM) pH=7, 50:50. All measurements of the retention time are done in triplicates for each sample).

### Exemplary Embodiments

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1: HetSiFA Compounds

In certain embodiments, HetSiFA compounds can include pyridine rings with various side chains, linkers, and/or chelators. Examples of HetSiFA compounds can include: di(tert-butyl)(fluoro)(2-pyridyl)silane, with the following structure:

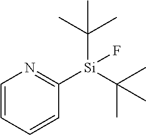 [SMILES: CC(C)(C)[Si](F)(c1ccccn1)C(C)(C)C]

di(tert-butyl)(fluoro)(2-methoxy-3-pyridyl)silane, with the following structure:

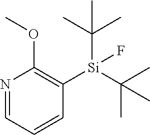 [SMILES: COc1ncccc1[Si](F)(C(C)(C)C)C(C)(C)C]

di(tert-butyl)(fluoro)(4-pyridyl)silane, with the following structure:

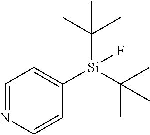 [SMILES: CC(C)(C)[Si](F)(c1ccncc1)C(C)(C)C]

di(tert-butyl)(fluoro)(2-methoxy-4-pyridyl)silane, with the following structure:

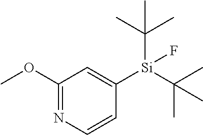 [SMILES: COc1cc([Si](F)(C(C)(C)C)C(C)(C)C)ccn1]

di(tert-butyl)(fluoro)(6-methoxy-3-pyridyl)silane, with the following structure:

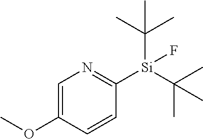 [SMILES: COc1ccc([Si](F)(C(C)(C)C)C(C)(C)C)nc1]

di(tert-butyl)(fluoro)(3-methoxy-4-pyridyl)silane, with the following structure:

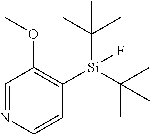 [SMILES: COc1cnccc1[Si](F)(C(C)(C)C)C(C)(C)C]

di(tert-butyl)(fluoro)(3-methoxy-2-pyridyl)silane, with the following structure, with the following structure:

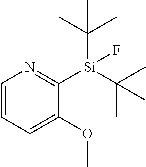 [SMILES: COc1cccnc1[Si](F)(C(C)(C)C)C(C)(C)C]

di(tert-butyl)(fluoro)(4-methoxy-3-pyridyl)silane, with the following structure:

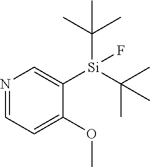 [SMILES: COc1ccncc1[Si](F)(C(C)(C)C)C(C)(C)C]

di(tert-butyl)(fluoro)(3-pyridyl)silane, with the following structure, with the following structure:

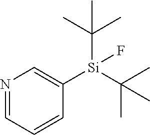 [SMILES: CC(C)(C)[Si](F)(c1cccnc1)C(C)(C)C]

[2-({N-5-[di(tert-butyl)(fluoro)silyl]-4-methoxy-2-pyridylcarbamoyl}methoxy)ethoxy]acetic acid, with the following structure:

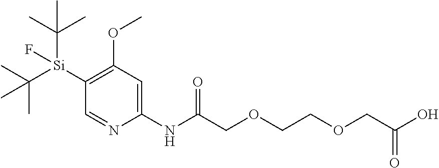 [SMILES: COc1cc(NC(=O)COCCOCC(=O)O)ncc1[Si](F)(C(C)(C)C)C(C)(C)C]

tert-butyl{[(3-{5-[di(tert-butyl)(fluoro)silyl]-4-methoxy-2-pyridyl}ureido)methyl]carbonylamino}acetate, with the following structure:

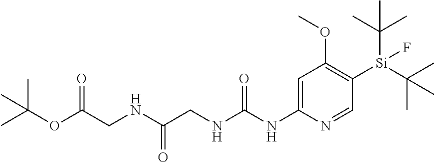 [SMILES: COc1cc(NC(=O)NCC(=O)NCC(=O)OC(C)(C)C)ncc1[Si](F)(C(C)(C)C)C(C)(C)C]

N-methyl{5-[di(tert-butyl)(fluoro)silyl]-4-methoxy-2-pyridyl}amine, with the following structure:

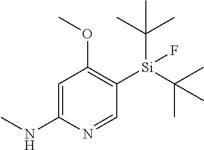 [SMILES: CNc1cc(OC)c([Si](F)(C(C)(C)C)C(C)(C)C)cn1]

5-[di(tert-butyl)(fluoro)silyl]-2-pyridylamine, with the following structure:

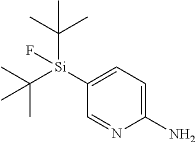 [SMILES: CC(C)(C)[Si](F)(c1ccc(N)nc1)C(C)(C)C]

5-[di(tert-butyl)(fluoro)silyl]-2-{[(2-carboxymethoxyethoxy)methyl]carbonylamino}-4-methoxy-1-pyridinium-1-olate, with the following structure:

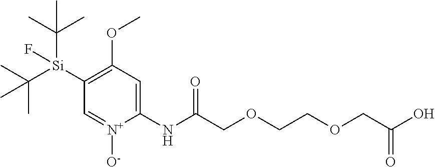 [SMILES: COc1cc(NC(=O)COCCOCC(=O)O)[n+]([O-])cc1[Si](F)(C(C)(C)C)C(C)(C)C]

5-[di(tert-butyl)(fluoro)silyl]-2-({[2-(tert-butoxycarbonylmethoxy)ethoxy]methyl}carbonylamino)-4-methoxy-1-pyridinium-1-olate, with the following structure:

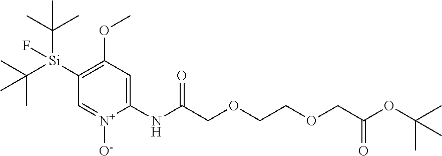 [SMILES: COc1cc(NC(=O)COCCOCC(=O)OC(C)(C)C)[n+]([O-])cc1[Si](F)(C(C)(C)C)C(C)(C)C]

di(tert-butyl)(fluoro)(4-methoxy-6-methyl-3-pyridyl)silane, with the following structure:

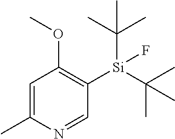 [SMILES: COc1cc(C)ncc1[Si](F)(C(C)(C)C)C(C)(C)C]

di(tert-butyl)[6-(dimethoxymethyl)-3-pyridyl](fluoro)silane, with the following structure:

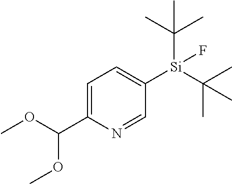 [SMILES: COC(OC)c1ccc([Si](F)(C(C)(C)C)C(C)(C)C)cn1]

5-[di(tert-butyl)(fluoro)silyl]-2-pyridinol, with the following structure:

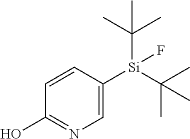 [SMILES: CC(C)(C)[Si](F)(c1ccc(O)nc1)C(C)(C)C]

tert-butyl[2-({N-5-[di(tert-butyl)(fluoro)silyl]-2-pyridylcarbamoyl}methoxy)ethoxy]acetate, with the following structure:

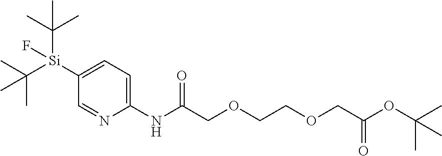 [SMILES: CC(C)(C)OC(=O)COCCOCC(=O)Nc1ccc([Si](F)(C(C)(C)C)C(C)(C)C)cn1]

3-[di(tert-butyl)(fluoro)silyl]-1-methyl-1-pyridinium, with the following structure:

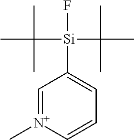 [SMILES: C[n+]1cccc([Si](F)(C(C)(C)C)C(C)(C)C)c1]

4-[di(tert-butyl)(fluoro)silyl]-1-methyl-1-pyridinium, with the following structure:

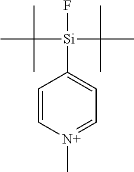 [SMILES: C[n+]1ccc([Si](F)(C(C)(C)C)C(C)(C)C)cc1]

5-[di(tert-butyl)(fluoro)silyl]-4-methoxy-2-pyridylamine, with the following structure:

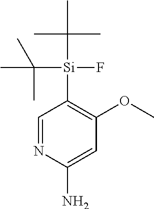 [SMILES: COc1cc(N)ncc1[Si](F)(C(C)(C)C)C(C)(C)C]

5-[di(tert-butyl)(fluoro)silyl]-2-({[2-(tert-butoxycarbonylmethoxy)ethoxy]methyl}carbonylamino)-1-pyridinium-1-olate, with the following structure:

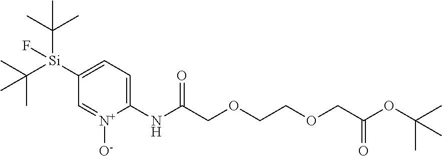 [SMILES: CC(C)(C)OC(=O)COCCOCC(=O)Nc1ccc([Si](F)(C(C)(C)C)C(C)(C)C)c[n+]1[O-]]

[2-({N-5-[di(tert-butyl)(fluoro)silyl]-2-pyridylcarbamoyl}methoxy)ethoxy]acetic acid, with the following structure:

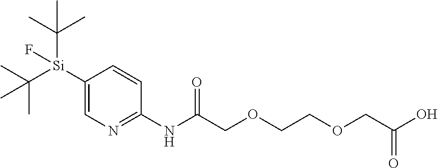 [SMILES: CC(C)(C)[Si](F)(c1ccc(NC(=O)COCCOCC(=O)O)nc1)C(C)(C)C]

{5-[di(tert-butyl)(fluoro)silyl]-4-methoxy-2-pyridyl}-N-methylamino-tert-butylformylate, with the following structure:

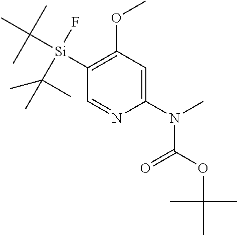 [SMILES: COc1cc(N(C)C(=O)OC(C)(C)C)ncc1[Si](F)(C(C)(C)C)C(C)(C)C]

N-{5-[di(tert-butyl)(fluoro)silyl]-4-methoxy-2-pyridyl}acetamide, with the following structure:

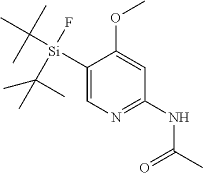 [SMILES: COc1cc(NC(C)=O)ncc1[Si](F)(C(C)(C)C)C(C)(C)C]

(3-{5-[di(tert-butyl)(fluoro)silyl]-4-methoxy-2-pyridyl}thioureido)acetic acid, with the following structure:

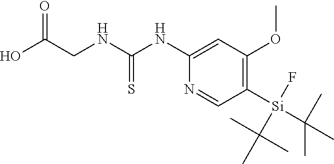 [SMILES: COc1cc(NC(=S)NCC(=O)O)ncc1[Si](F)(C(C)(C)C)C(C)(C)C]

[4-({5-[di(tert-butyl)(fluoro)silyl]-4-methoxy-2-pyridylamino}methyl)-1H-1,2,3-triazol-1-yl]acetic acid, with the following structure:

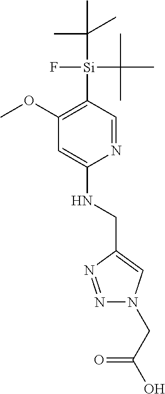 [SMILES: COc1cc(NCc2cn(CC(=O)O)nn2)ncc1[Si](F)(C(C)(C)C)C(C)(C)C]

((5-(di-tert-butylfluorosilyl)-4-methoxypyridin-2-yl)carbamoyl)glycine, with the following structure:

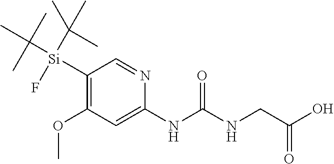 [SMILES: COc1cc(NC(=O)NCC(=O)O)ncc1[Si](F)(C(C)(C)C)C(C)(C)C]

tert-butyl 4-[N—(S)-1,2-bis{N-2-[2-({N-5-[di(tert-butyl)(fluoro)silyl]-4-methoxy-2-pyridylcarbamoyl}methoxy)ethoxy]ethylcarbamoyl}ethylcarbamoyl]-2-[4,7,10-tris(tert-butoxycarbonylmethyl)-1,4,7,10-tetraaza-1-cyclododecyl]butyrate, with the following structure:

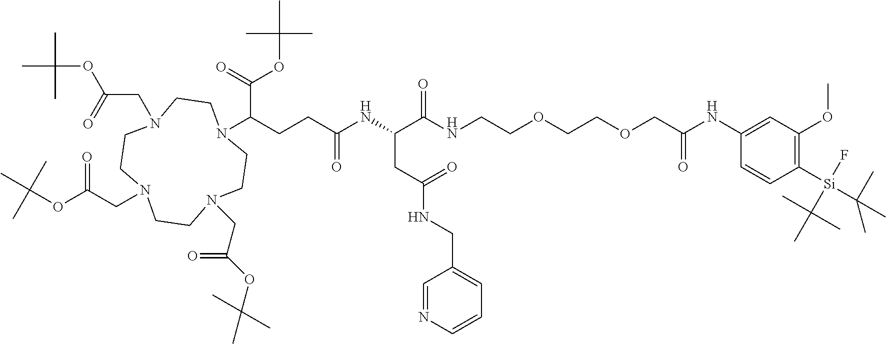 [SMILES: COc1cc(NC(=O)COCCOCCNC(=O)[C@H](CC(=O)NCc2cccnc2)NC(=O)CCC(C(=O)OC(C)(C)C)N2CCN(CC(=O)OC(C)(C)C)CCN(CC(=O)OC(C)(C)C)CCN(CC(=O)OC(C)(C)C)CC2)ccc1[Si](F)(C(C)(C)C)C(C)(C)C]

(S)-3-{N-2-[2-({N-5-[di(tert-butyl)(fluoro)silyl]-4-methoxy-2-pyridylcarbamoyl}methoxy)ethoxy]ethylcarbamoyl}-3-{4-tert-butoxycarbonyl-4-[4,7,10-tris(tert-butoxycarbonylmethyl)-1,4,7,10-tetraaza-1-cyclododecyl]butyrylamino}propionic acid, with the following structure:

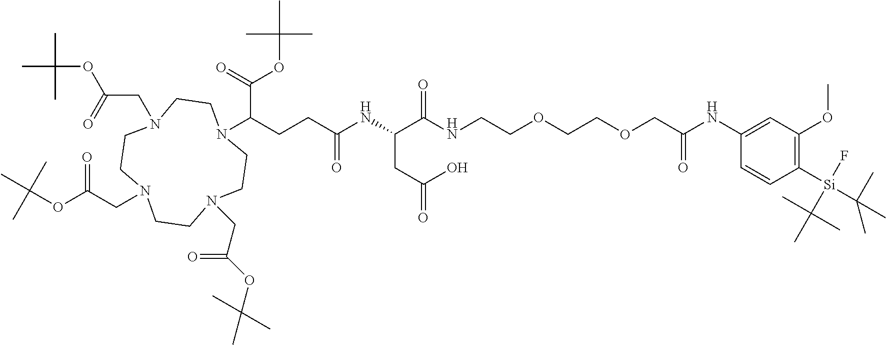 [SMILES: COc1cc(NC(=O)COCCOCCNC(=O)[C@H](CC(=O)O)NC(=O)CCC(C(=O)OC(C)(C)C)N2CCN(CC(=O)OC(C)(C)C)CCN(CC(=O)OC(C)(C)C)CCN(CC(=O)OC(C)(C)C)CC2)ccc1[Si](F)(C(C)(C)C)C(C)(C)C]

(4-{[N—(S)-1,2-bis{N-2-[2-({N-5-[di(tert-butyl)(fluoro)silyl]-4-methoxy-2-pyridylcarbamoyl}methoxy)ethoxy]ethylcarbamoyl}ethylcarbamoyl]methyl}-7,10-bis(carboxymethyl)-1,4,7,10-tetraaza-1-cyclododecyl)acetic acid, with the following structure:

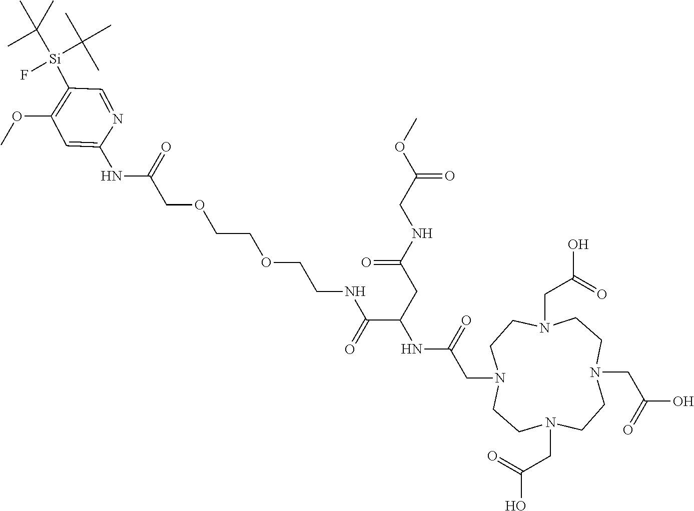 [SMILES: COC(=O)CNC(=O)CC(NC(=O)CN1CCN(CC(=O)O)CCN(CC(=O)O)CCN(CC(=O)O)CC1)C(=O)NCCOCCOCC(=O)Nc1cc(OC)c([Si](F)(C(C)(C)C)C(C)(C)C)cn1]

(S)-3-[N—(S)-2-carboxy-1-{[(3-pyridyl)methyl]carbamoyl}ethylcarbamoyl]-3-[3-(3-{5-[di(tert-butyl)(fluoro)silyl]-4-methoxy-2-pyridyl}ureido)propionylamino]propionic acid, with the following structure:

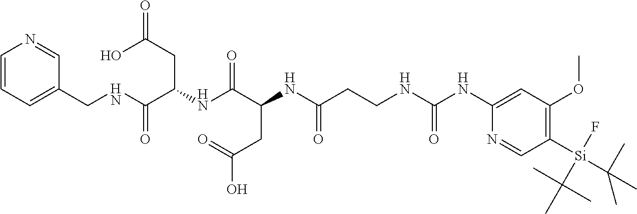 [SMILES: COc1cc(NC(=O)NCCC(=O)N[C@@H](CC(=O)O)C(=O)N[C@@H](CC(=O)O)C(=O)NCc2cccnc2)ncc1[Si](F)(C(C)(C)C)C(C)(C)C]

{5-[di(tert-butyl)(fluoro)silyl]-2-pyridyl}-N-methylamino-tert-butylformylate, with the following structure:

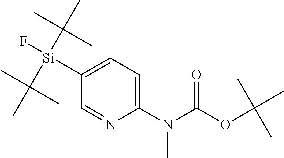 [SMILES: CN(C(=O)OC(C)(C)C)c1ccc([Si](F)(C(C)(C)C)C(C)(C)C)cn1]

N-(3-pyridyl)methyl3-(3-{5-[di(tert-butyl)(fluoro)silyl]-4-methoxy-2-pyridyl}ureido)propionamide, with the following structure:

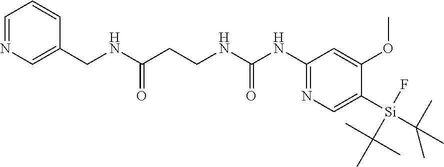 [SMILES: COc1cc(NC(=O)NCCC(=O)NCc2cccnc2)ncc1[Si](F)(C(C)(C)C)C(C)(C)C]

{N-5-[di(tert-butyl)(fluoro)silyl]-4-methoxy-2-pyridylcarbamoyl}(trimethylammonio)methane iodide, with the following structure:

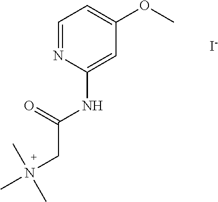

N-{5-[di(tert-butyl)(fluoro)silyl]-4-methoxy-2-pyridyl}(dimethylamino)acetamide, with the following structure:

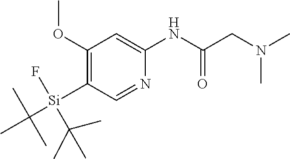 [SMILES: COc1cc(NC(=O)CN(C)C)ncc1[Si](F)(C(C)(C)C)C(C)(C)C]

3-{N-2-[2-({N-5-[di(tert-butyl)(fluoro)silyl]-4-methoxy-2-pyridylcarbamoyl}methoxy)ethoxy]ethylcarbamoyl}-3-({[4,7,10-tris(tert-butoxycarbonylmethyl)-1,4,7,10-tetraaza-1-cyclododecyl]methyl}carbonylamino)propionic acid, with the following structure:

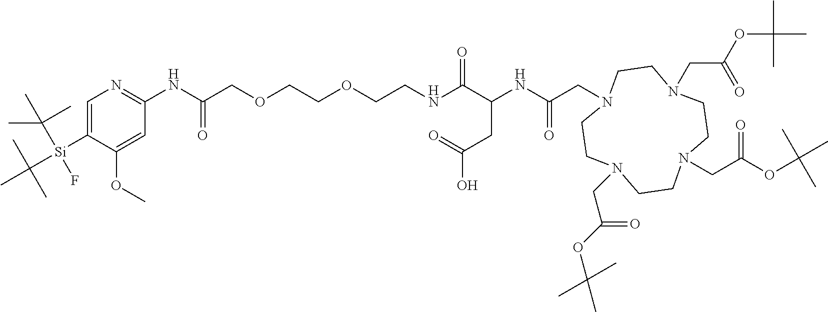 [SMILES: COc1cc(NC(=O)COCCOCCNC(=O)C(CC(=O)O)NC(=O)CN2CCN(CC(=O)OC(C)(C)C)CCN(CC(=O)OC(C)(C)C)CCN(CC(=O)OC(C)(C)C)CC2)ncc1[Si](F)(C(C)(C)C)C(C)(C)C]

benzyl 3-{N-2-[2-({N-5-[di(tert-butyl)(fluoro)silyl]-4-methoxy-2-pyridylcarbamoyl}methoxy)ethoxy]ethylcarbamoyl}-3-({[4,7,10-tris(tert-butoxycarbonylmethyl)-1,4,7,10-tetraaza-1-cyclododecyl]methyl}carbonylamino)propionate, with the following structure:

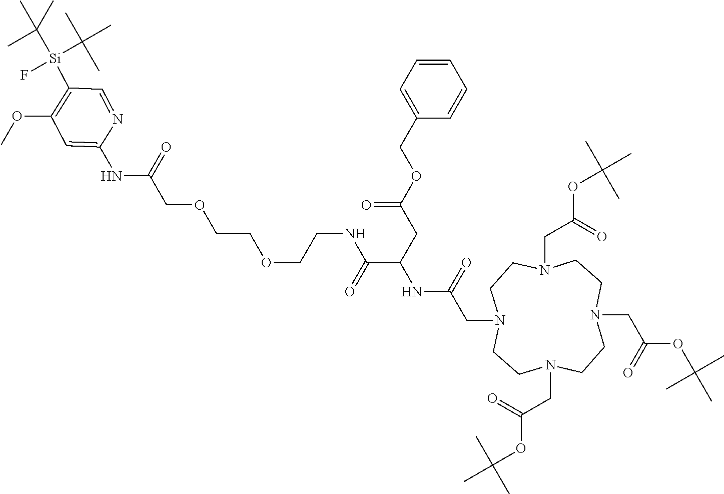 [SMILES: COc1cc(NC(=O)COCCOCCNC(=O)C(CC(=O)OCc2ccccc2)NC(=O)CN2CCN(CC(=O)OC(C)(C)C)CCN(CC(=O)OC(C)(C)C)CCN(CC(=O)OC(C)(C)C)CC2)ncc1[Si](F)(C(C)(C)C)C(C)(C)C]

N-{5-[di(tert-butyl)(fluoro)silyl]-4-methoxy-2-pyridyl}[2-(2-azidoethoxy)ethoxy]acetamide, with the following structure:

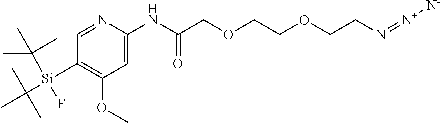 [SMILES: COc1cc(NC(=O)COCCOCCN=[NH+][NH-])ncc1[Si](F)(C(C)(C)C)C(C)(C)C]

3-(3-{5-[di(tert-butyl)(fluoro)silyl]-4-methoxy-2-pyridyl}ureido)propionic acid, with the following structure:

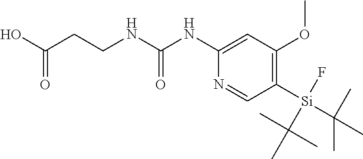 [SMILES: COc1cc(NC(=O)NCCC(=O)O)ncc1[Si](F)(C(C)(C)C)C(C)(C)C]

2-amino-5-[di(tert-butyl)(fluoro)silyl]-4-methoxy-1-pyridinium-1-olate, with the following structure

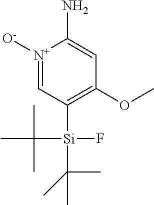 [SMILES: COc1cc(N)[n+]([O-])cc1[Si](F)(C(C)(C)C)C(C)(C)C]

3-[di(tert-butyl)(fluoro)silyl]-2-methoxy-1-pyridinium-1-olate, with the following structure:

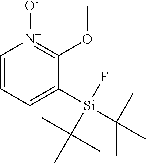 [SMILES: COc1c([Si](F)(C(C)(C)C)C(C)(C)C)ccc[n+]1[O-]]

5-[di(tert-butyl)(fluoro)silyl]-2-(tert-butoxycarbonylamino)-4-methoxy-1-pyridinium-1-olate, with the following structure:

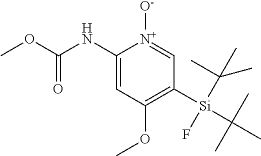 [SMILES: COC(=O)Nc1cc(OC)c([Si](F)(C(C)(C)C)C(C)(C)C)c[n+]1[O-]]

5-[di(tert-butyl)(fluoro)silyl]-4-methoxy-2-(2-propynylamino)-1-pyridinium-1-olate, with the following structure:

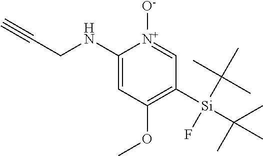 [SMILES: C#CCNc1cc(OC)c([Si](F)(C(C)(C)C)C(C)(C)C)c[n+]1[O-]]

2-(N-tert-butoxycarbonyl-N-2-propynylamino)-5-[di(tert-butyl)(fluoro)silyl]-4-methoxy-1-pyridinium-1-olate, with the following structure:

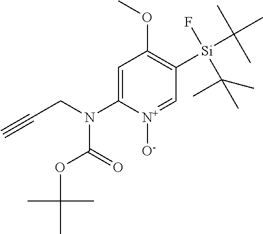 [SMILES: C#CCN(C(=O)OC(C)(C)C)c1cc(OC)c([Si](F)(C(C)(C)C)C(C)(C)C)c[n+]1[O-]]

{5-[di(tert-butyl)(fluoro)silyl]-4-methoxy-2-pyridyl}-N-2-propynylamino-tert-butylformylate, with the following structure:

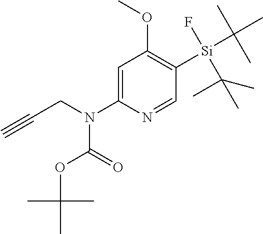 [SMILES: C#CCN(C(=O)OC(C)(C)C)c1cc(OC)c([Si](F)(C(C)(C)C)C(C)(C)C)cn1]

4-[di(tert-butyl)(fluoro)silyl]-3-methoxy-1-pyridinium-1-olate, with the following structure:

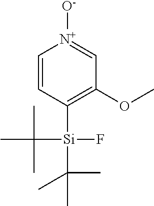 [SMILES: COc1c[n+]([O-])ccc1[Si](F)(C(C)(C)C)C(C)(C)C]

3-[di(tert-butyl)(fluoro)silyl]-4-methoxy-1-pyridinium-1-olate, with the following structure:

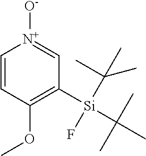 [SMILES: COc1cc[n+]([O-])cc1[Si](F)(C(C)(C)C)C(C)(C)C]

5-[di(tert-butyl)(fluoro)silyl]-2-methoxy-1-pyridinium-1-olate, with the following structure:

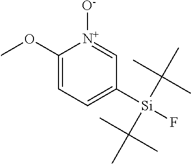 [SMILES: COc1ccc([Si](F)(C(C)(C)C)C(C)(C)C)c[n+]1[O-]]

4-[di(tert-butyl)(fluoro)silyl]-1-(tert-butoxycarbonylmethyl)-3-methoxy-1-pyridinium, with the following structure:

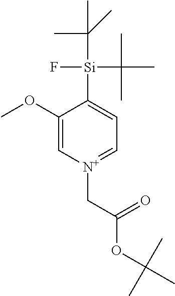 [SMILES: COc1c[n+](CC(=O)OC(C)(C)C)ccc1[Si](F)(C(C)(C)C)C(C)(C)C]

tert-butyl {3-[di(tert-butyl)(fluoro)silyl]-4-oxo-1-pyridyl}acetate, with the following structure:

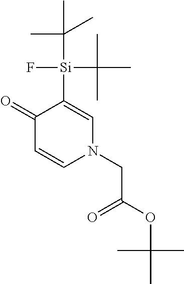 [SMILES: CC(C)(C)OC(=O)Cn1ccc(=O)c([Si](F)(C(C)(C)C)C(C)(C)C)c1]

3-[di(tert-butyl)(fluoro)silyl]-1-(tert-butoxycarbonylmethyl)-4-methoxy-1-pyridinium bromide, with the following structure:

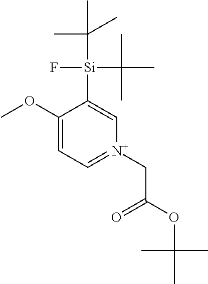 [SMILES: COc1cc[n+](CC(=O)OC(C)(C)C)cc1[Si](F)(C(C)(C)C)C(C)(C)C]

N-(3-pyridyl)methyl(3-{5-[di(tert-butyl)(fluoro)silyl]-4-methoxy-2-pyridyl}ureido)acetamide, with the following structure:

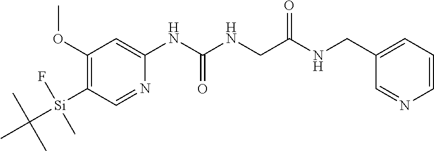 [SMILES: COc1cc(NC(=O)NCC(=O)NCc2cccnc2)ncc1[Si](C)(F)C(C)(C)C]

5-[di(tert-butyl)(fluoro)silyl]-2-pyridylamino-tert-butylformylate, with the following structure:

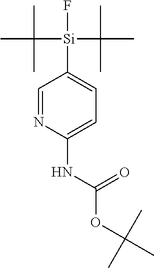 [SMILES: CC(C)(C)OC(=O)Nc1ccc([Si](F)(C(C)(C)C)C(C)(C)C)cn1]

N-(3-pyridyl)methyl[4-({5-[di(tert-butyl)(fluoro)silyl]-4-methoxy-2-pyridylamino}methyl)-1H-1,2,3-triazol-1-yl]acetamide, with the following structure:

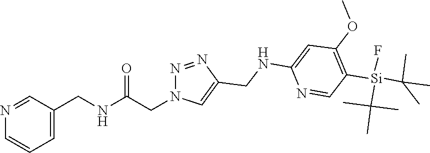 [SMILES: COc1cc(NCc2cn(CC(=O)NCc3cccnc3)nn2)ncc1[Si](F)(C(C)(C)C)C(C)(C)C]

N-(3-pyridyl)methyl(3-{5-[di(tert-butyl)(fluoro)silyl]-4-methoxy-2-pyridyl}thioureido)acetamide, with the following structure:

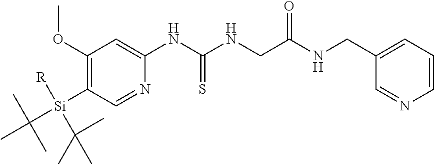

tert-butyl 3-(3-{5-[di(tert-butyl)(fluoro)silyl]-4-methoxy-2-pyridyl}ureido)propionate, with the following structure:

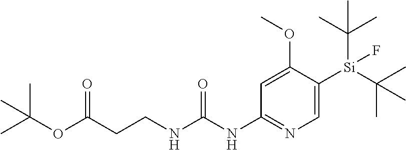 [SMILES: COc1cc(NC(=O)NCCC(=O)OC(C)(C)C)ncc1[Si](F)(C(C)(C)C)C(C)(C)C]

tert-butyl (3-{5-[di(tert-butyl)(fluoro)silyl]-4-methoxy-2-pyridyl}thioureido)acetate, with the following structure:

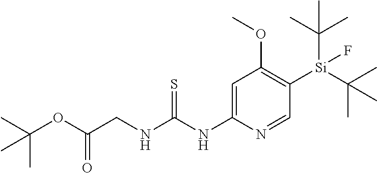 [SMILES: COc1cc(NC(=S)NCC(=O)OC(C)(C)C)ncc1[Si](F)(C(C)(C)C)C(C)(C)C]

N-(3-pyridyl)methyl{5-[di(tert-butyl)(fluoro)silyl]-4-methoxy-2-pyridylamino}acetamide, with the following structure:

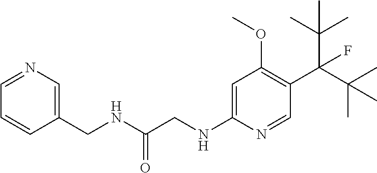 [SMILES: COc1cc(NCC(=O)NCc2cccnc2)ncc1C(F)(C(C)(C)C)C(C)(C)C]

{5-[di(tert-butyl)(fluoro)silyl]-4-methoxy-2-pyridylamino}acetic acid, with the following structure:

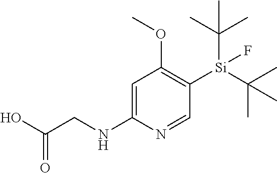 [SMILES: COc1cc(NCC(=O)O)ncc1[Si](F)(C(C)(C)C)C(C)(C)C]

tert-butyl-5-[fluorobis(isopropyl)silyl]-4-methoxy-2-pyridylaminoformylate, with the following structure:

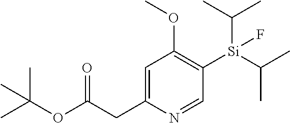 [SMILES: COc1cc(CC(=O)OC(C)(C)C)ncc1[Si](F)(C(C)C)C(C)C]

N-2-propynyl{5-[di(tert-butyl)(fluoro)silyl]-4-methoxy-2-pyridyl}amine, with the following structure:

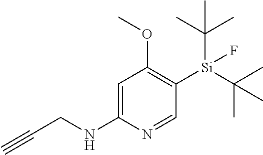 [SMILES: C#CCNc1cc(OC)c([Si](F)(C(C)(C)C)C(C)(C)C)cn1]

tert-butyl (3-{5-[di(tert-butyl)(fluoro)silyl]-4-methoxy-2-pyridyl}ureido)acetate, with the following structure:

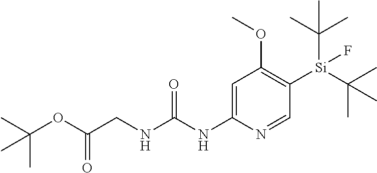 [SMILES: COc1cc(NC(=O)NCC(=O)OC(C)(C)C)ncc1[Si](F)(C(C)(C)C)C(C)(C)C]

N-benzyl{5-[di(tert-butyl)(fluoro)silyl]-4-methoxy-2-pyridylamino}acetamide, with the following structure:

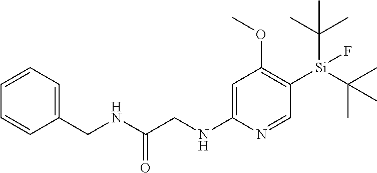 [SMILES: COc1cc(NCC(=O)NCc2ccccc2)ncc1[Si](F)(C(C)(C)C)C(C)(C)C]

N-{5-[di(tert-butyl)(fluoro)silyl]-4-methoxy-2-pyridyl}-N-butylsuccinamide, with the following structure:

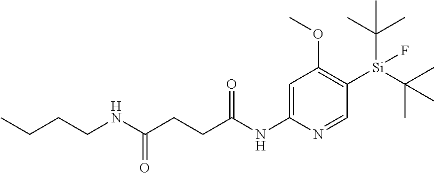 [SMILES: CCCCNC(=O)CCC(=O)Nc1cc(OC)c([Si](F)(C(C)(C)C)C(C)(C)C)cn1]

N-benzyl-N-{5-[di(tert-butyl)(fluoro)silyl]-4-methoxy-2-pyridyl}succinamide, with the following structure:

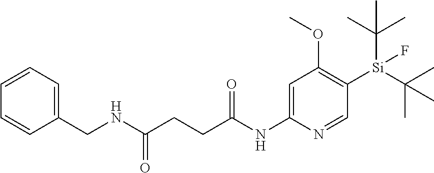 [SMILES: COc1cc(NC(=O)CCC(=O)NCc2ccccc2)ncc1[Si](F)(C(C)(C)C)C(C)(C)C]

tert-butyl 3-{N-5-[di(tert-butyl)(fluoro)silyl]-4-methoxy-2-pyridylcarbamoyl}propionate, with the following structure:

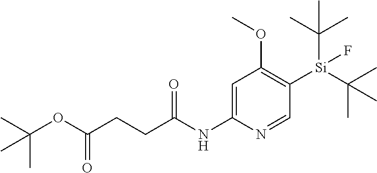 [SMILES: COc1cc(NC(=O)CCC(=O)OC(C)(C)C)ncc1[Si](F)(C(C)(C)C)C(C)(C)C]

5-[di(tert-butyl)(fluoro)silyl]-4-methoxy-2-pyridylamino-tert-butylformylate, with the following structure:

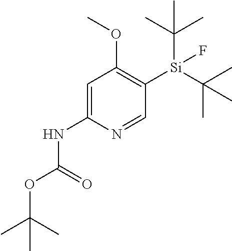 [SMILES: COc1cc(NC(=O)OC(C)(C)C)ncc1[Si](F)(C(C)(C)C)C(C)(C)C]

In certain embodiments, HetSiFA compounds can include pyrazole rings with various side chains, linkers, and/or chelators. Examples of HetSiFA compounds can include: di(tert-butyl)(fluoro)(1-methyl-5-pyrazolyl)silane, with the following structure:

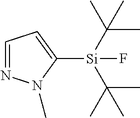 [SMILES: Cn1nccc1[Si](F)(C(C)(C)C)C(C)(C)C]

di(tert-butyl)(fluoro)(5-pyrazolyl)silane, with the following structure:

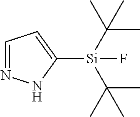

di(tert-butyl)(fluoro){1-[(2-methoxyethoxy)methyl]-5-pyrazolyl}silane with the following structure:

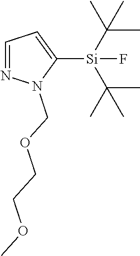 [SMILES: COCCOCn1nccc1[Si](F)(C(C)(C)C)C(C)(C)C]

di(tert-butyl)(fluoro){1-[(2-methoxyethoxy)methyl]-4-methyl-5-pyrazolyl}silane, with the following structure:

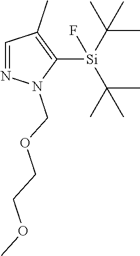 [SMILES: COCCOCn1ncc(C)c1[Si](F)(C(C)(C)C)C(C)(C)C]

{5-[di(tert-butyl)(fluoro)silyl]-1-methyl-3-pyrazolylamino}acetic acid, with the following structure:

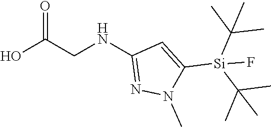 [SMILES: Cn1nc(NCC(=O)O)cc1[Si](F)(C(C)(C)C)C(C)(C)C]

N-(3-pyridyl)methyl{5-[di(tert-butyl)(fluoro)silyl]-1-methyl-3-pyrazolylamino}acetamide, with the following structure:

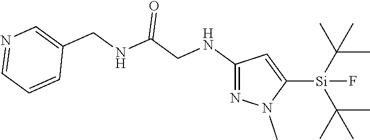 [SMILES: Cn1nc(NCC(=O)NCc2cccnc2)cc1[Si](F)(C(C)(C)C)C(C)(C)C]

{5-[di(tert-butyl)(fluoro)silyl]-1-{2-[2-(tert-butoxycarbonylamino)ethoxy]ethyl}-3-pyrazolylamino}acetic acid, with the following structure:

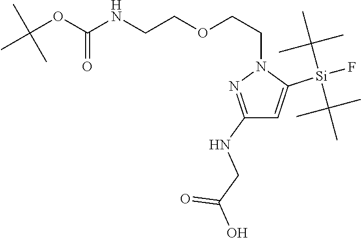 [SMILES: CC(C)(C)OC(=O)NCCOCCn1nc(NCC(=O)O)cc1[Si](F)(C(C)(C)C)C(C)(C)C]

3-[di(tert-butyl)(fluoro)silyl]-5-(carboxymethyl)-2,5-dimethyl-4,5,6,7-tetrahydro-2H-1,2,5-triazainden-5-ium formate, with the following structure:

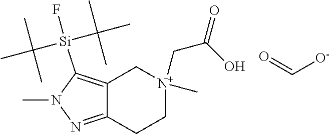

N-(3-pyridyl)methyl{3-[di(tert-butyl)(fluoro)silyl]-2-methyl-4,5,6,7-tetrahydro-2H-1,2,5-triazainden-5-yl}acetamide, with the following structure:

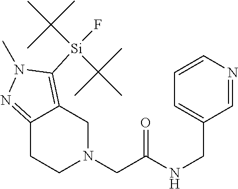 [SMILES: Cn1nc2c(c1[Si](F)(C(C)(C)C)C(C)(C)C)CN(CC(=O)NCc1cccnc1)CC2]

{3-[di(tert-butyl)(fluoro)silyl]-2-methyl-4,5,6,7-tetrahydro-2H-1,2,5-triazainden-5-yl}acetic acid, with the following structure:

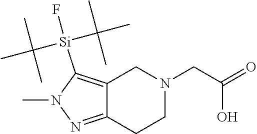 [SMILES: Cn1nc2c(c1[Si](F)(C(C)(C)C)C(C)(C)C)CN(CC(=O)O)CC2]

[({5-[di(tert-butyl)(fluoro)silyl]-1-methyl-3-pyrazolylamino}methyl)carbonylamino]acetic acid, with the following structure:

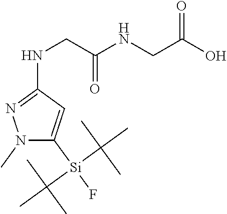 [SMILES: Cn1nc(NCC(=O)NCC(=O)O)cc1[Si](F)(C(C)(C)C)C(C)(C)C]

tert-butyl 3-[di(tert-butyl)(fluoro)silyl]-2-methyl-4,5,6,7-tetrahydro-2H-1,2,5-triazaindene-5-carboxylate, with the following structure:

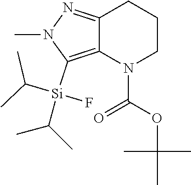 [SMILES: CC(C)[Si](F)(c1c2c(nn1C)CCCN2C(=O)OC(C)(C)C)C(C)C]

tert-butyl 3-[fluorobis(isopropyl)silyl]-2-methyl-4,5,6,7-tetrahydro-2H-1,2,4-triazaindene-4-carboxylate, with the following structure:

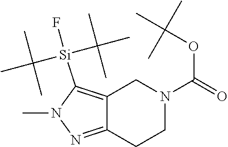 [SMILES: Cn1nc2c(c1[Si](F)(C(C)(C)C)C(C)(C)C)CN(C(=O)OC(C)(C)C)CC2]

5-[di(tert-butyl)(fluoro)silyl]-3-(tert-butoxycarbonylamino)-1-methylpyrazole, with the following structure:

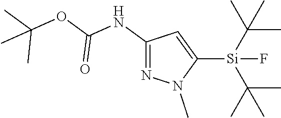 [SMILES: Cn1nc(NC(=O)OC(C)(C)C)cc1[Si](F)(C(C)(C)C)C(C)(C)C]

({5-[di(tert-butyl)(fluoro)silyl]-1-(2-{2-[(9H-fluoren-9-yl)methoxycarbonylamino]ethoxy}ethyl)-3-pyrazolyl}[(9H-fluoren-9-yl)methoxycarbonyl]amino)acetic acid, with the following structure:

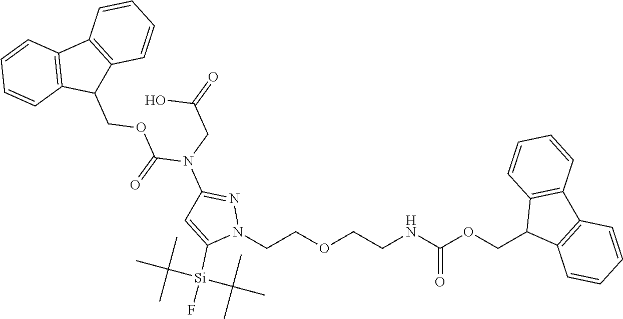 [SMILES: CC(C)(C)[Si](F)(c1cc(N(CC(=O)O)C(=O)OCC2c3ccccc3-c3ccccc32)nn1CCOCCNC(=O)OCC1c2ccccc2-c2ccccc21)C(C)(C)C]

N-[2-(2-{5-[di(tert-butyl)(fluoro)silyl]-3-[({[(3-pyridyl)methyl]carbamoyl}methyl)amino]-1-pyrazolyl}ethoxy)ethyl](3,16,19-trioxo-2,17,18-trioxa-5,8,11,14-tetraaza-1-lutetatricyclo[9.6.3.2^{5,14}]docos-8-yl)acetamide, with the following structure:

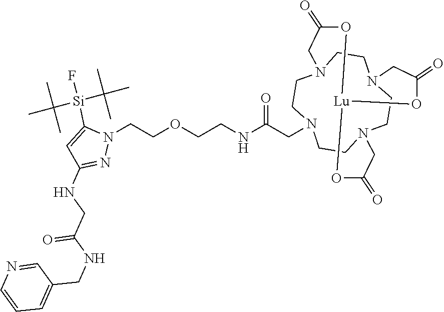

4-[N-2-(2-{5-[di(tert-butyl)(fluoro)silyl]-3-[({[(3-pyridyl)methyl]carbamoyl}methyl)amino]-1-pyrazolyl}ethoxy)ethylcarbamoyl]-2-[4,7,10-tris(carboxymethyl)-1,4,7,10-tetraaza-1-cyclododecyl]butyric acid, with the following structure:

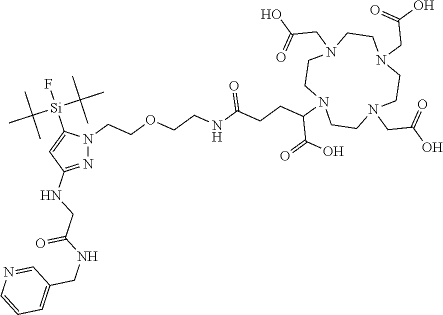 [SMILES: CC(C)(C)[Si](F)(c1cc(NCC(=O)NCc2cccnc2)nn1CCOCCNC(=O)CCC(C(=O)O)N1CCN(CC(=O)O)CCN(CC(=O)O)CCN(CC(=O)O)CC1)C(C)(C)C]

4-[N-2-(2-{5-[di(tert-butyl)(fluoro)silyl]-3-[({[(3-pyridyl)methyl]carbamoyl}methyl)amino]-1-pyrazolyl}ethoxy)ethylcarbamoyl]-2-(3,16,19-trioxo-2,17,18-trioxa-5,8,11,14-tetraaza-1-gallatricyclo[9.6.3.2^{5,14}]docos-8-yl)butyric acid, with the following structure:

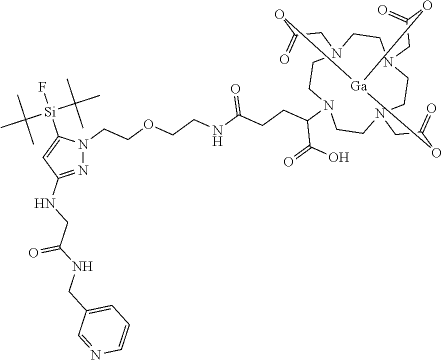

N-(3-pyridyl)methyl[({5-[di(tert-butyl)(fluoro)silyl]-1-methyl-3-pyrazolylamino}methyl) carbonylamino]acetamide, with the following structure:

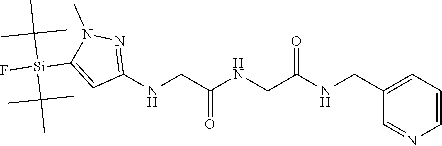 [SMILES: Cn1nc(NCC(=O)NCC(=O)NCc2cccnc2)cc1[Si](F)(C(C)(C)C)C(C)(C)C]

methyl [({5-[di(tert-butyl)(fluoro)silyl]-1-{2-[2-({(3,16,19-trioxo-2,17,18-trioxa-5,8,11,14-tetraaza-1-gallatricyclo[9.6.3.2^{5,14}]docos-8-yl)methyl}carbonylamino)ethoxy]ethyl}-3-pyrazolylamino}methyl)carbonylamino]acetate, with the following structure:

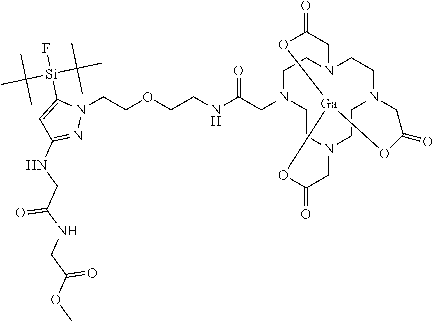

methyl({[({5-[di(tert-butyl)(fluoro)silyl]-1-methyl-3-pyrazolylamino}methyl)carbonylamino]methyl}carbonylamino)acetate, with the following structure:

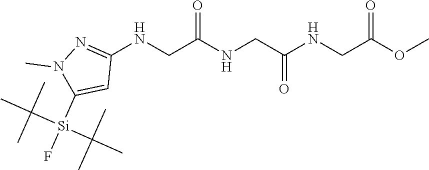 [SMILES: COC(=O)CNC(=O)CNC(=O)CNc1cc([Si](F)(C(C)(C)C)C(C)(C)C)n(C)n1]

1-{5-[di(tert-butyl)(fluoro)silyl]-1-methyl-3-pyrazolyl}-2,5-piperazinedione, with the following structure:

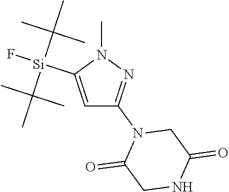 [SMILES: Cn1nc(N2CC(=O)NCC2=O)cc1[Si](F)(C(C)(C)C)C(C)(C)C]

(4-{[N-2-(2-{5-[di(tert-butyl)(fluoro)silyl]-3-({[(methoxycarbonylmethyl)carbamoyl]methyl}amino)-1-pyrazolyl}ethoxy)ethylcarbamoyl]methyl}-7,10-bis(carboxymethyl)-1,4,7,10-tetraaza-1-cyclododecyl)acetic acid, with the following structure:

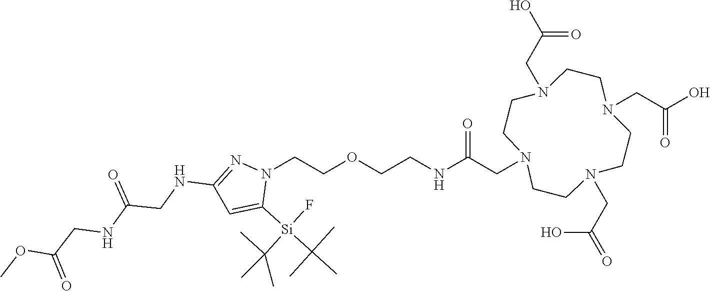 [SMILES: COC(=O)CNC(=O)CNc1cc([Si](F)(C(C)(C)C)C(C)(C)C)n(CCOCCNC(=O)CN2CCN(CC(=O)O)CCN(CC(=O)O)CCN(CC(=O)O)CC2)n1]

methyl [({5-[di(tert-butyl)(fluoro)silyl]-1-{2-[2-({[4,7,10-tris(tert-butoxycarbonylmethyl)-1,4,7,10-tetraaza-1-cyclododecyl]methyl}carbonylamino)ethoxy]ethyl}-3-pyrazolylamino}methyl)carbonylamino]acetate, with the following structure:

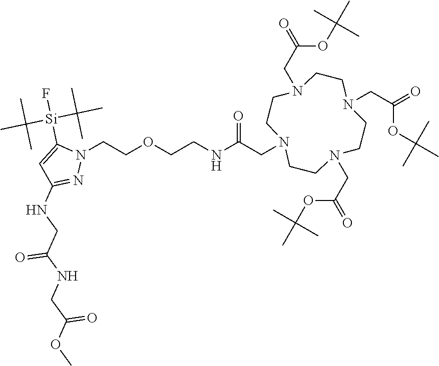 [SMILES: COC(=O)CNC(=O)CNc1cc([Si](F)(C(C)(C)C)C(C)(C)C)n(CCOCCNC(=O)CN2CCN(CC(=O)OC(C)(C)C)CCN(CC(=O)OC(C)(C)C)CCN(CC(=O)OC(C)(C)C)CC2)n1]

N-[2-(2-{5-[di(tert-butyl)(fluoro)silyl]-3-[({[(3-pyridyl)methyl]carbamoyl}methyl)amino]-1-pyrazolyl}ethoxy)ethyl](3,16,19-trioxo-2,17,18-trioxa-5,8,11,14-tetraaza-1-gallatricyclo[9.6.3.2^{5,14}]docos-8-yl)acetamide, with the following structure:

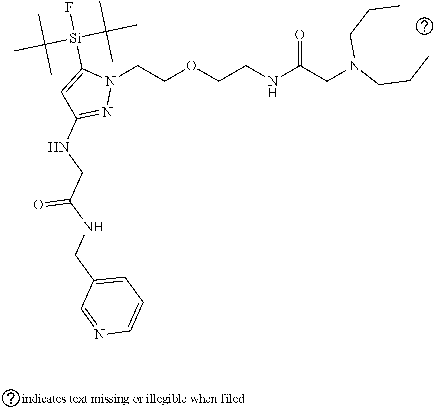 [SMILES: CCCN(CCC)CC(=O)NCCOCCn1nc(NCC(=O)NCc2cccnc2)cc1[Si](F)(C(C)(C)C)C(C)(C)C]

(4-{[N-2-(2-{5-[di(tert-butyl)(fluoro)silyl]-3-[({[(3-pyridyl)methyl]carbamoyl}methyl)amino]-1-pyrazolyl}ethoxy)ethylcarbamoyl]methyl}-7,10-bis(carboxymethyl)-1,4,7,10-tetraaza-1-cyclododecyl)acetic acid, with the following structure:

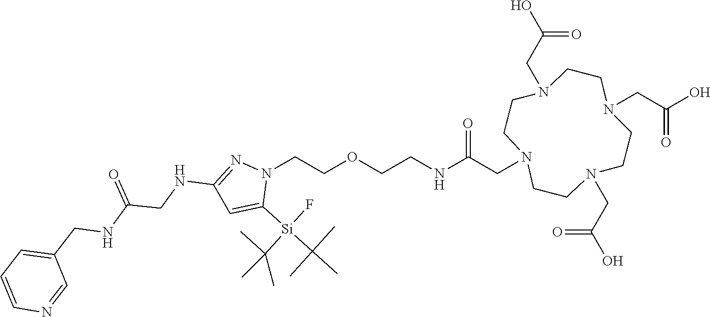 [SMILES: CC(C)(C)[Si](F)(c1cc(NCC(=O)NCc2cccnc2)nn1CCOCCNC(=O)CN1CCN(CC(=O)O)CCN(CC(=O)O)CCN(CC(=O)O)CC1)C(C)(C)C]

13-[di(tert-butyl)(fluoro)silyl]-4-oxa-1,7,10,14-tetraazabicyclo[9.2.1]tetradeca-11(14),12-dien-8-one, with the following structure:

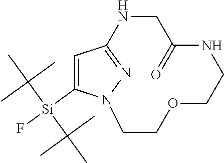 [SMILES: CC(C)(C)[Si](F)(c1cc2nn1CCOCCNC(=O)CN2)C(C)(C)C]

tert-butyl (4-{[N-2-(2-{5-[di(tert-butyl)(fluoro)silyl]-3-[({[(3-pyridyl)methyl]carbamoyl}methyl)amino]-1-pyrazolyl}ethoxy)ethylcarbamoyl]methyl}-7,10-bis(tert-butoxycarbonylmethyl)-1,4,7,10-tetraaza-1-cyclododecyl)acetate, with the following structure:

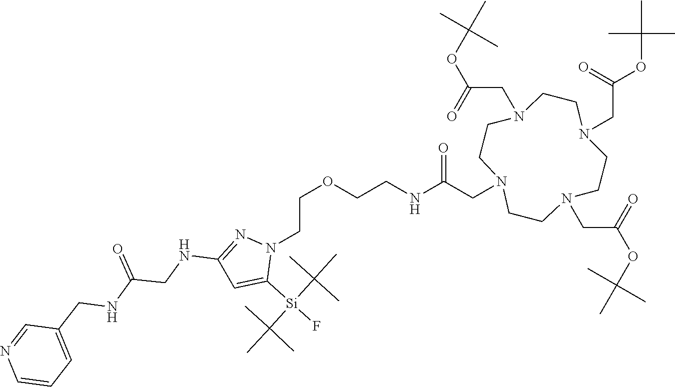 [SMILES: CC(C)(C)OC(=O)CN1CCN(CC(=O)NCCOCCn2nc(NCC(=O)NCc3cccnc3)cc2[Si](F)(C(C)(C)C)C(C)(C)C)CCN(CC(=O)OC(C)(C)C)CCN(CC(=O)OC(C)(C)C)CC1]

{5-[di(tert-butyl)(fluoro)silyl]-1-{2-[2-({[4,7,10-tris(tert-butoxycarbonylmethyl)-1,4,7,10-tetraaza-1-cyclododecyl]methyl}carbonylamino)ethoxy]ethyl}-3-pyrazolylamino}acetic acid, with the following structure:

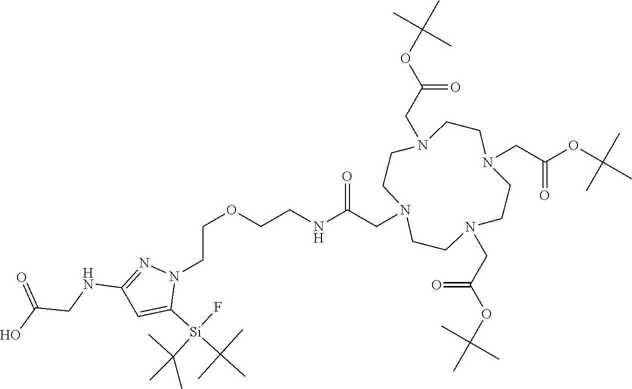 [SMILES: CC(C)(C)OC(=O)CN1CCN(CC(=O)NCCOCCn2nc(NCC(=O)O)cc2[Si](F)(C(C)(C)C)C(C)(C)C)CCN(CC(=O)OC(C)(C)C)CCN(CC(=O)OC(C)(C)C)CC1]

In certain embodiments, HetSiFA compounds can include azaindole rings with various side chains, linkers, and/or chelators. Examples of HetSiFA compounds can include: {2-[di(tert-butyl)(fluoro)silyl]-1-methyl-1H-1,7-diazainden-5-yloxy}acetic acid, with the following structure:

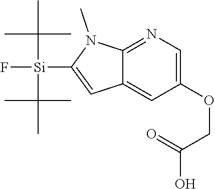 [SMILES: Cn1c([Si](F)(C(C)(C)C)C(C)(C)C)cc2cc(OCC(=O)O)cnc21]

{{2-[di(tert-butyl)(fluoro)silyl]-1-methyl-1H-1,7-diazainden-6-ylamino}acetic acid, with the following structure:

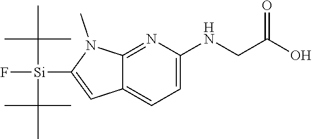 [SMILES: Cn1c([Si](F)(C(C)(C)C)C(C)(C)C)cc2ccc(NCC(=O)O)nc21]

di(tert-butyl)(fluoro)(1-{1-[(p-methoxyphenyl)methyl]-3-azetidinyl}-1H-1,7-diazainden-2-yl)silane, with the following structure:

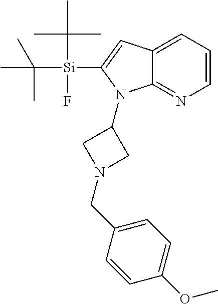 [SMILES: COc1ccc(CN2CC(n3c([Si](F)(C(C)(C)C)C(C)(C)C)cc4cccnc43)C2)cc1]

2-[di(tert-butyl)(fluoro)silyl]-1-methyl-1H-1,7-diazainden-7-ium-7-olate, with the following structure:

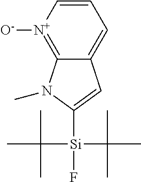 [SMILES: Cn1c([Si](F)(C(C)(C)C)C(C)(C)C)cc2ccc[n+]([O-])c21]

2-[di(tert-butyl)(fluoro)silyl]-1-methyl-1H-1,4-diazainden-5-ium-4-olate, with the following structure:

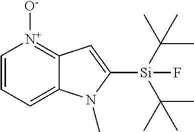 [SMILES: Cn1c([Si](F)(C(C)(C)C)C(C)(C)C)cc2c1ccc[n+]2[O-]]

N-(3-pyridyl)methyl{2-[di(tert-butyl)(fluoro)silyl]-1-methyl-1H-1,7-diazainden-5-yloxy}acetamide, with the following structure:

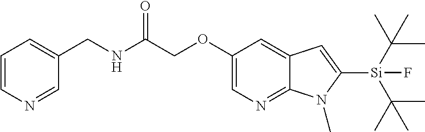 [SMILES: Cn1c([Si](F)(C(C)(C)C)C(C)(C)C)cc2cc(OCC(=O)NCc3cccnc3)cnc21]

2-[di(tert-butyl)(fluoro)silyl]-1,4-dimethyl-1H-1,4-diazainden-4-ium, with the following structure:

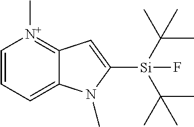 [SMILES: Cn1c([Si](F)(C(C)(C)C)C(C)(C)C)cc2c1ccc[n+]2C]

di(tert-butyl)(fluoro)(6-methoxy-1-methyl-1H-1,7-diazainden-2-yl)silane, with the following structure:

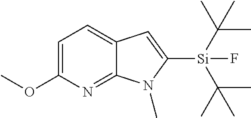 [SMILES: COc1ccc2cc([Si](F)(C(C)(C)C)C(C)(C)C)n(C)c2n1]

tert-butyl {2-[di(tert-butyl)(fluoro)silyl]-1-methyl-1H-1,7-diazainden-5-yloxy}acetate, with the following structure:

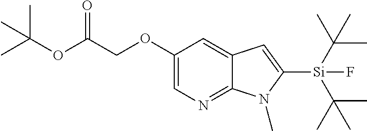 [SMILES: Cn1c([Si](F)(C(C)(C)C)C(C)(C)C)cc2cc(OCC(=O)OC(C)(C)C)cnc21]

2-[di(tert-butyl)(fluoro)silyl]-1-methyl-1H-1,7-diazainden-5-ol, with the following structure:

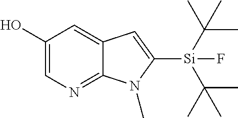 [SMILES: Cn1c([Si](F)(C(C)(C)C)C(C)(C)C)cc2cc(O)cnc21]

tert-butyl 3-{2-[di(tert-butyl)(fluoro)silyl]-1H-1,7-diazainden-1-yl}-1-azetidinecarboxylate, with the following structure:

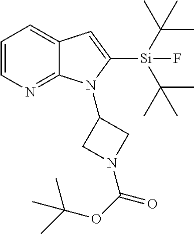 [SMILES: CC(C)(C)OC(=O)N1CC(n2c([Si](F)(C(C)(C)C)C(C)(C)C)cc3cccnc32)C1]

N-methyl{2-[di(tert-butyl)(fluoro)silyl]-1-methyl-1H-1,4-diazainden-5-yl}amine, with the following structure:

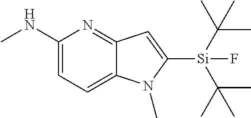 [SMILES: CNc1ccc2c(cc([Si](F)(C(C)(C)C)C(C)(C)C)n2C)n1]

{2-[di(tert-butyl)(fluoro)silyl]-1-methyl-1H-1,4-diazainden-6-yl}-N-methylamino-tert-butylformylate, with the following structure:

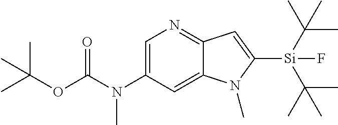 [SMILES: CN(C(=O)OC(C)(C)C)c1cnc2cc([Si](F)(C(C)(C)C)C(C)(C)C)n(C)c2c1]

{2-[di(tert-butyl)(fluoro)silyl]-1-methyl-1H-1,4-diazainden-5-yl}-N-methylamino-tert-butylformylate, with the following structure:

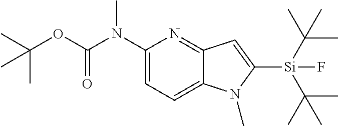 [SMILES: CN(C(=O)OC(C)(C)C)c1ccc2c(cc([Si](F)(C(C)(C)C)C(C)(C)C)n2C)n1]

N-methyl{2-[di(tert-butyl)(fluoro)silyl]-1-methyl-1H-1,4-diazainden-6-yl}amine, with the following structure:

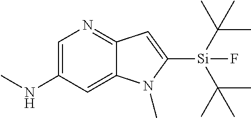 [SMILES: CNc1cnc2cc([Si](F)(C(C)(C)C)C(C)(C)C)n(C)c2c1]

di(tert-butyl)(fluoro)[1-(2-methoxyethyl)-1H-1,4-diazainden-2-yl]silane, with the following structure:

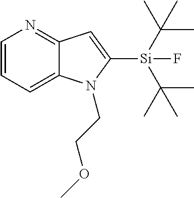 [SMILES: COCCn1c([Si](F)(C(C)(C)C)C(C)(C)C)cc2ncccc21]

di(tert-butyl)(fluoro)(5-methoxy-1-methyl-1H-1,7-diazainden-2-yl)silane, with the following structure:

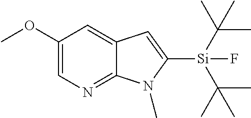 [SMILES: COc1cnc2c(c1)cc([Si](F)(C(C)(C)C)C(C)(C)C)n2C]

### Example 2: Theranostic HetSiFA Compounds

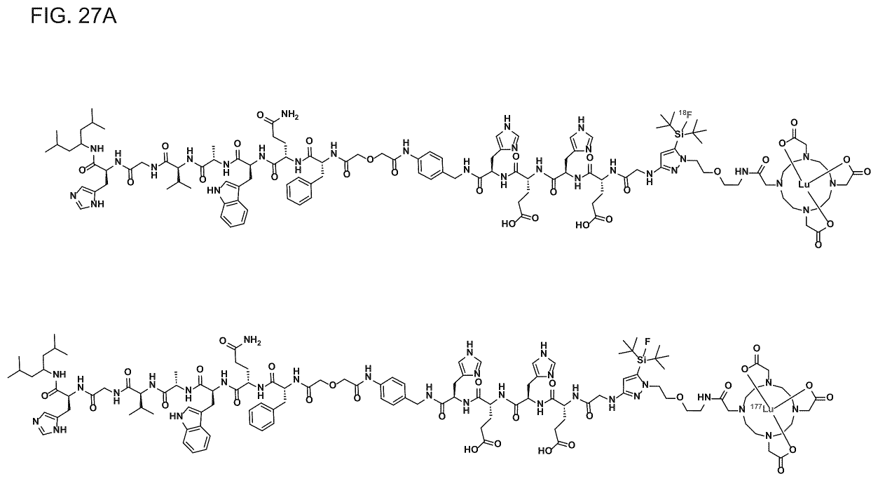

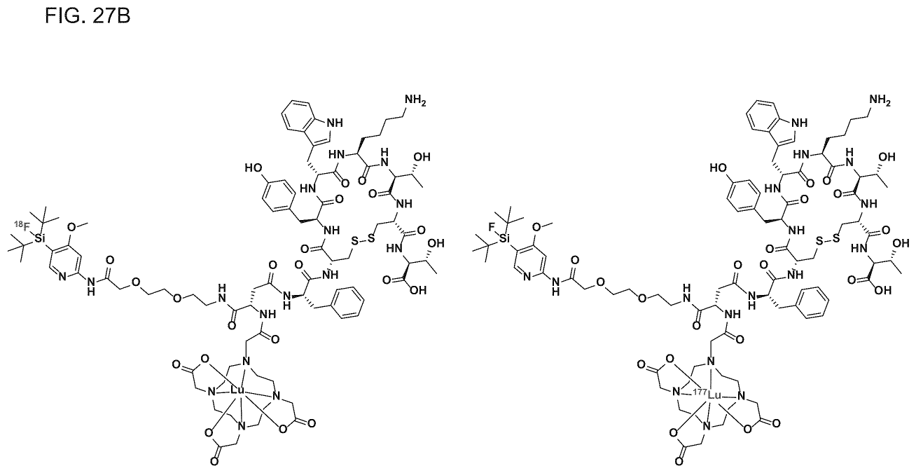

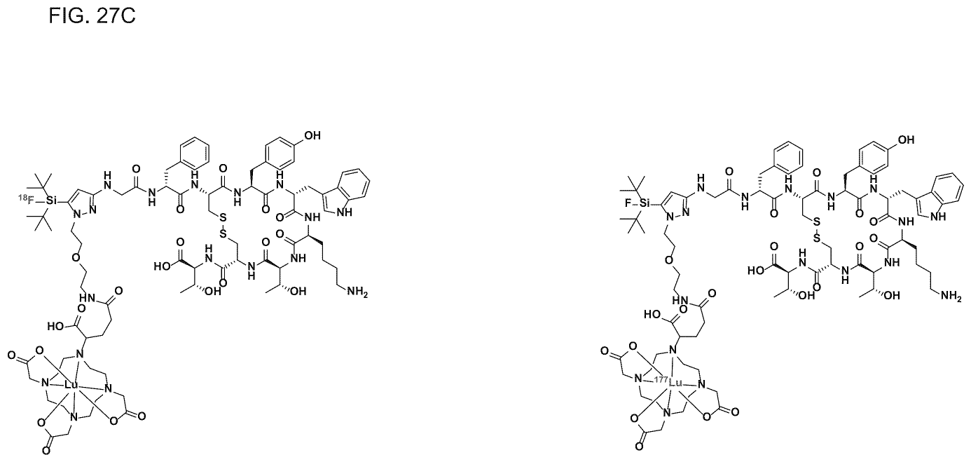

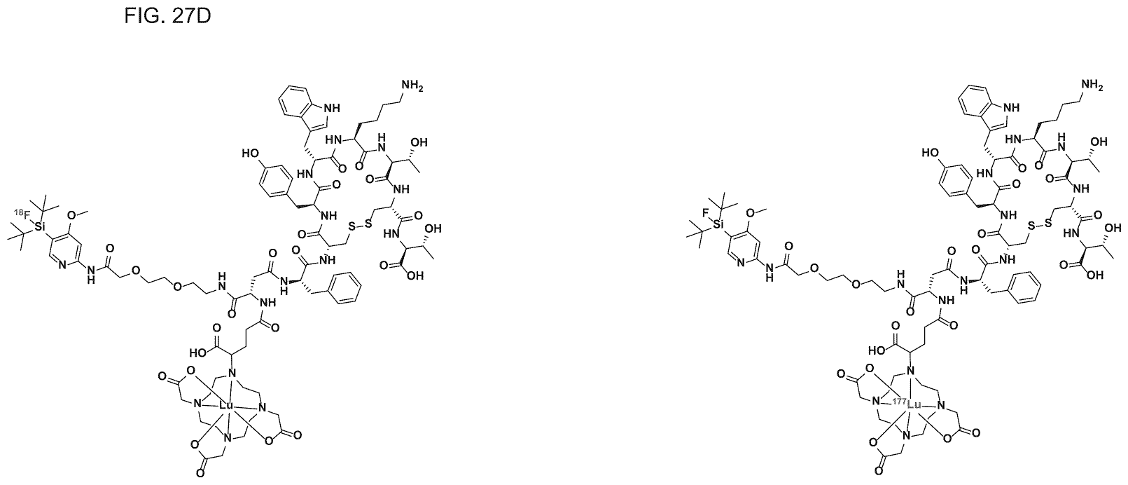

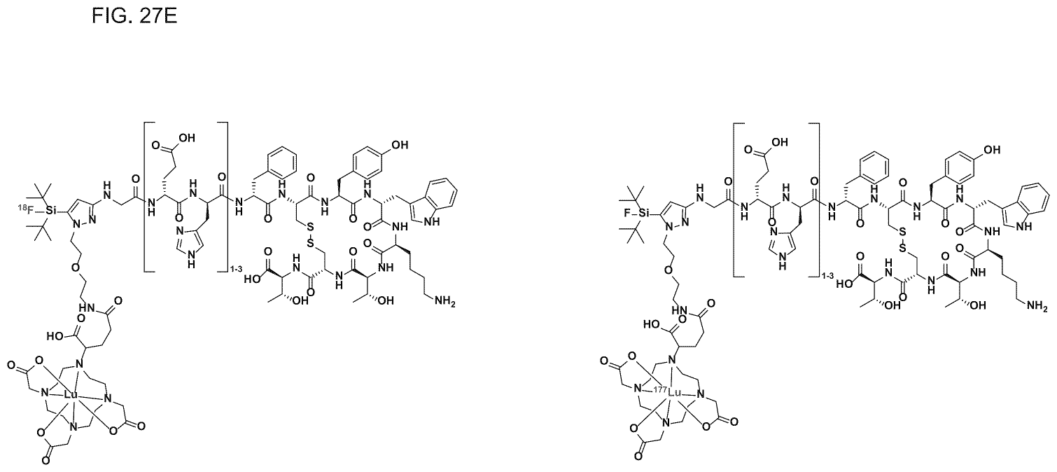

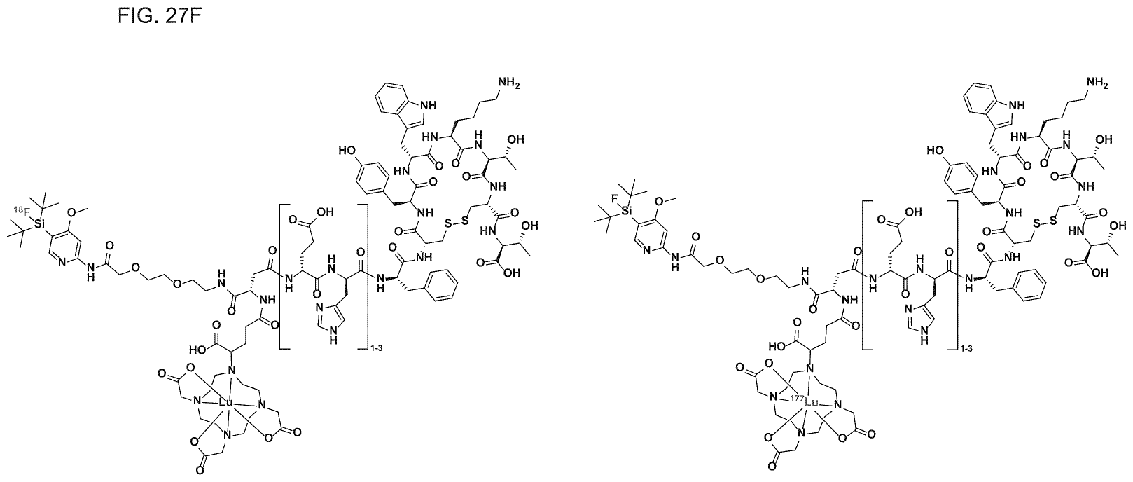

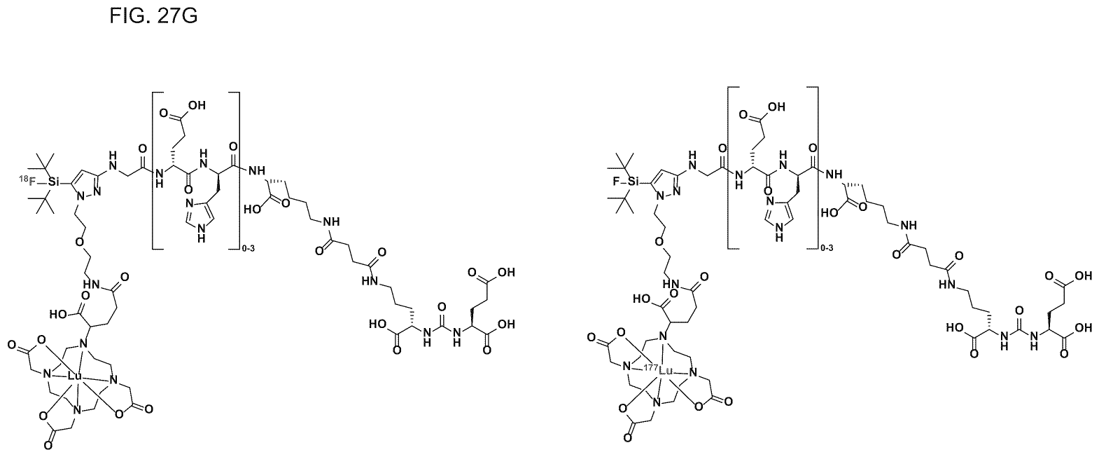

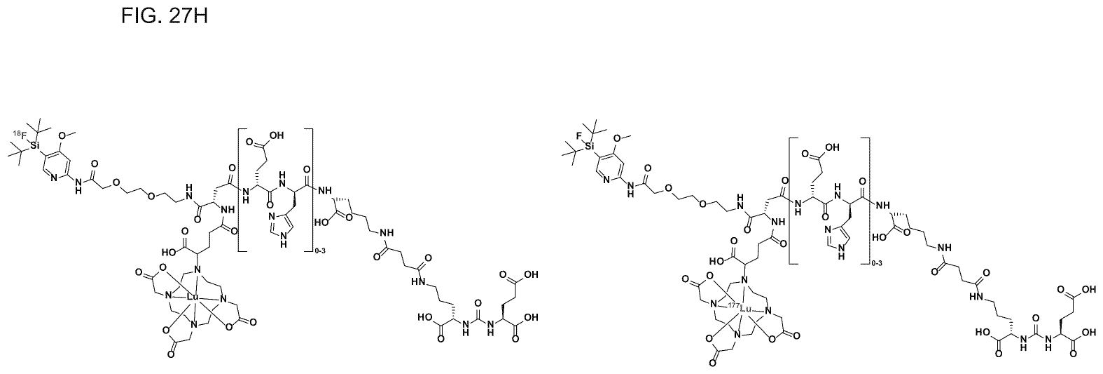

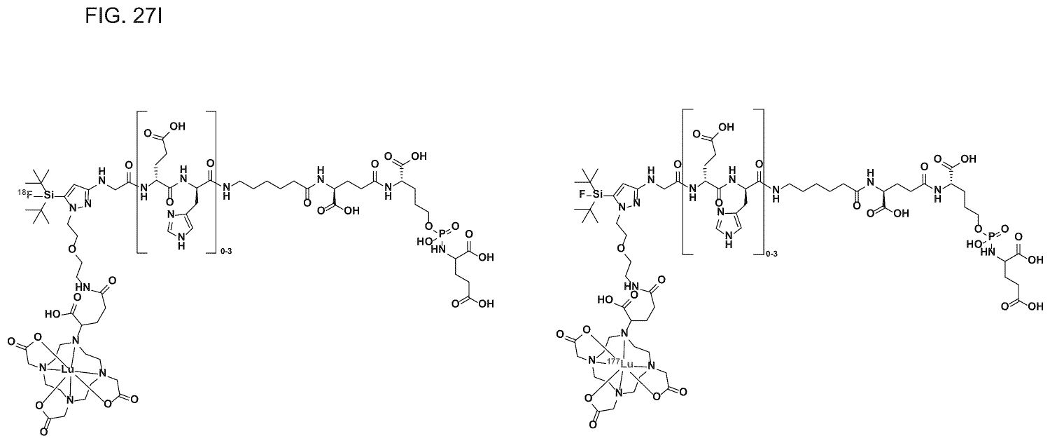

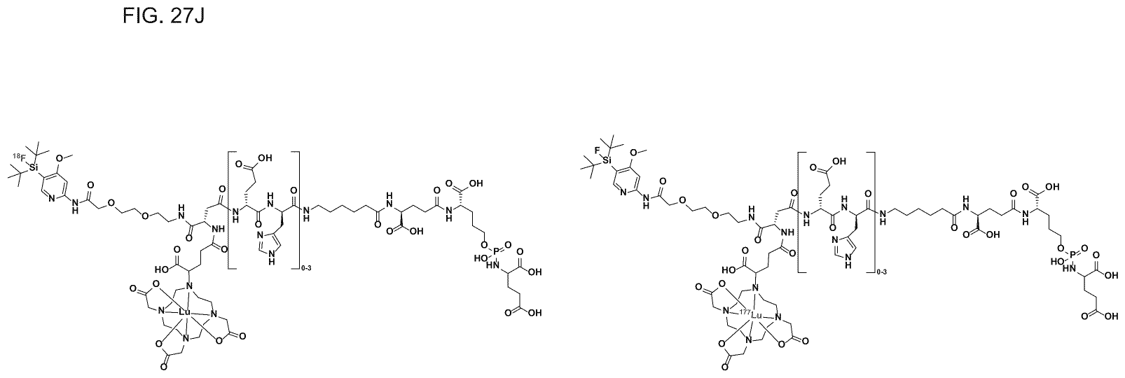

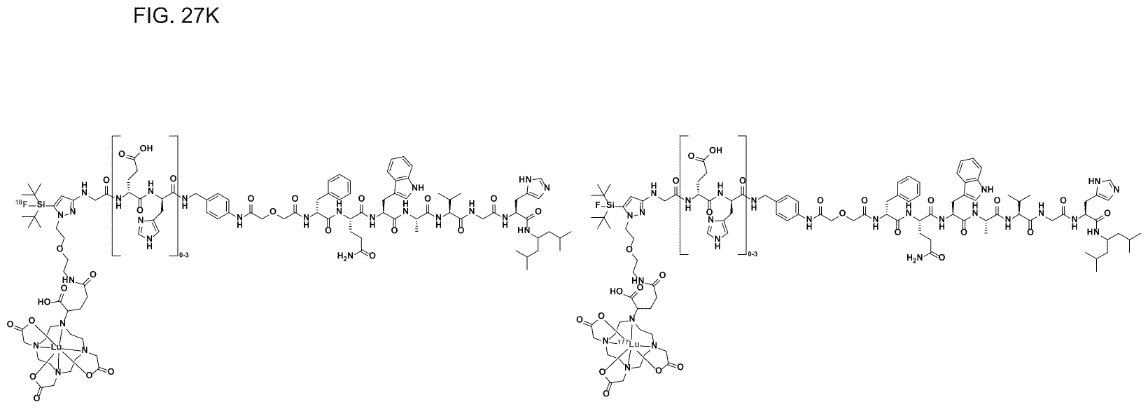

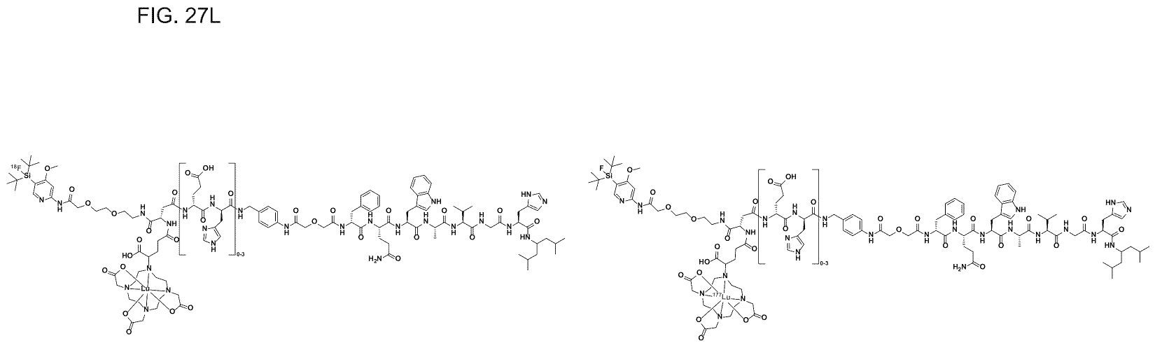

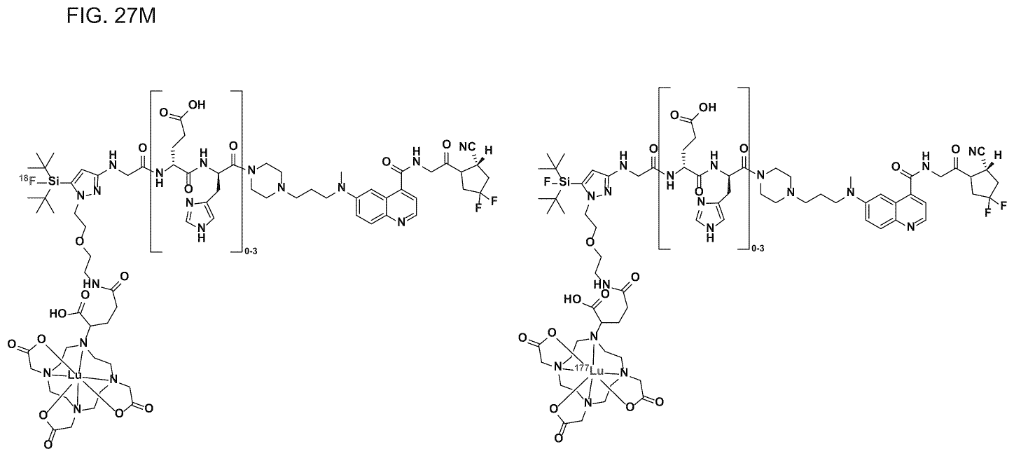

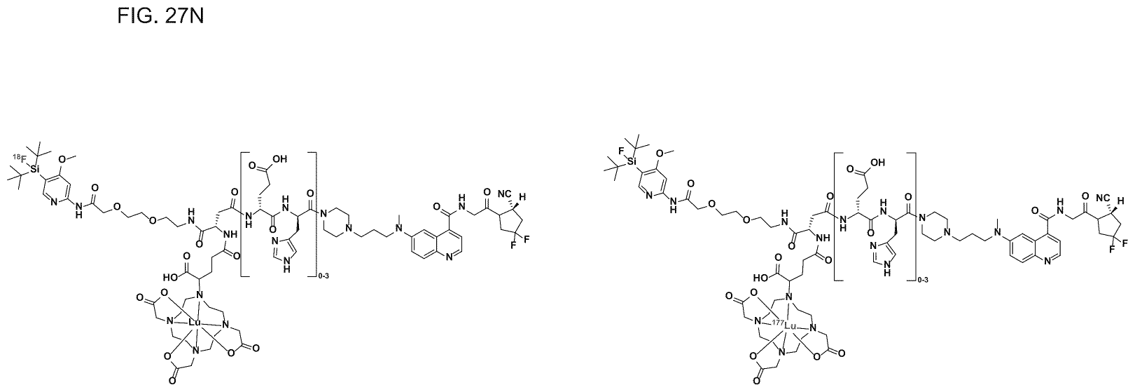

FIGS. 27A-27N illustrate theranostic HetSiFA pairs in accordance with an embodiment of the invention. FIG. 27A shows the structures of GRPR-targeting HetSiFA theranostics FTX-216 and FTX-219. FIG. 27B shows the structures of pyridine-HetSiFA-DOTA-TATE theranostic pairs. FIG. 27C shows the structures of pyrazole-HetSiFA-DOTAGA-TATE theranostic pairs. FIG. 27D shows the structures of pyridine-HetSiFA-DOTAGA-TATE theranostic pairs. FIG. 27E shows the structures of pyrazole-HetSiFA-DOTAGA-(HE)ᵢ-TATE (i=1-3) theranostic pairs. FIG. 27F shows the structures of pyridine-HetSiFA-DOTAGA-(HE)ᵢ-TATE (i=1-3) theranostic pairs. FIG. 27G shows the structures of pyrazole-HetSiFA-DOTAGA-(HE)ᵢ-PSMA (i=0-3) theranostic pairs. FIG. 27H shows the structures of pyridine-HetSiFA-DOTAGA-(HE)ᵢ-PSMA (i=0-3) theranostic pairs. FIG. 27I shows the structures of pyrazole-HetSiFA-DOTAGA-(HE)ᵢ-P-PSMA (i=0-3) theranostic pairs. FIG. 27J shows the structures of pyridine-HetSiFA-DOTAGA-(HE)ᵢ-P-PSMA (i=0-3) theranostic pairs. FIG. 27K shows the structures of pyrazole-HetSiFA-DOTAGA-(HE)ᵢ-GRPR (i=0-3) theranostic pairs. FIG. 27L shows the structures of pyridine-HetSiFA-DOTAGA-(HE)ᵢ-GRPR (i=0-3) theranostic pairs. FIG. 27M shows the structures of pyrazole-HetSiFA-DOTAGA-(HE)ᵢ-FAP (i=0-3) theranostic pairs. FIG. 27N shows the structures of pyridine-HetSiFA-DOTAGA-(HE)ᵢ-FAP (i=0-3) theranostic pairs.

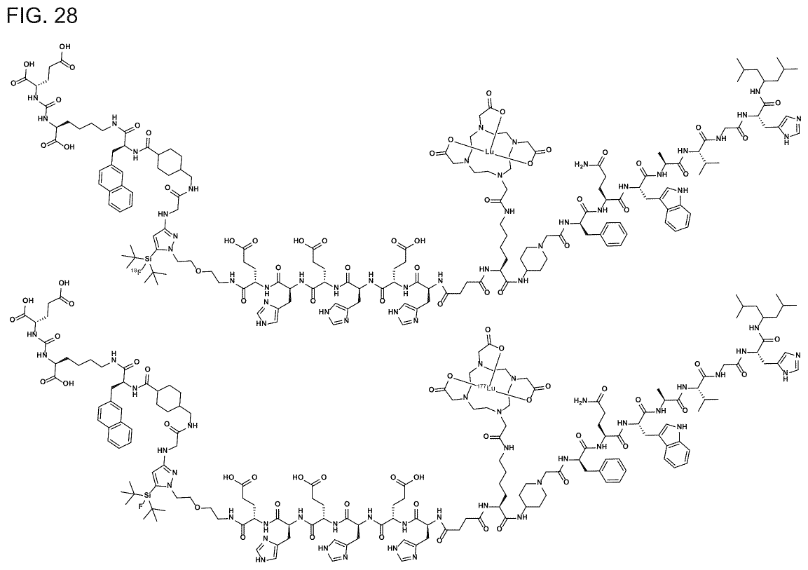

FIG. 28 illustrates a dual-targeting theranostic HetSiFA pair in accordance with an embodiment of the invention.

### Example 3: ¹⁸F-Radiolabeling Procedure for HetSiFA Compounds

Although the following describes a general ¹⁸F-radiolabeling procedure for HetSiFA compounds, as can be readily appreciated, any radiolabeling procedure to radiofluorinate HetSiFA precursor molecules, such as methods relying on azeotropic drying of [¹⁸F]fluoride may be used. Before the start of the synthesis, the reaction vessel is loaded with a solution of HetSiFA precursor (150 nmol) in anhydrous dimethyl sulfoxide (150 μL) and a 1 M solution (30 μL) of oxalic acid in anhydrous acetonitrile (MeCN). The solution must be freshly prepared by dissolving oxalic acid (9 mg) in anhydrous MeCN (0.1 mL). [¹⁸F]fluoride is trapped on a QMA cartridge (Sep-Pak Accell Plus QMA Carbonate Plus Light cartridge, 46 mg, 40 μm, Waters), preconditioned with 10 mL ultrapure water. The cartridge is dried by a flow of nitrogen, rinsed with MeCN (10 mL), and again dried by a flow of nitrogen. The activity is eluted from the QMA cartridge into the preloaded reactor with a solution of [K+œ2.2.2]OH⁻-cryptate in anhydrous MeCN (elution cocktail). For the preparation of lyophilized [K+œ2.2.2]OH⁻-cryptate, Kryptofix® 222 (343 mg, 910 μmol, 1.1 eq., Sigma-Aldrich) and 1 M KOH (830 μL, 830 μmol, 1.0 eq., 99.99% semiconductor grade, Sigma Aldrich) are dissolved in water (10 mL) and aliquoted into Eppendorf vials (1 mL or 1.5 mL each). The content of each vial is lyophilized, resulting in individual doses of [K+œ2.2.2]OH⁻ complex as an off-white solid. The lower-content vials will contain 91 μmol K222 and 83 μmol KOH and are typically used in manual radiolabeling experiments where the liquid losses are minimal. The higher-content vials will contain 137 μmol K222 and 125 μmol KOH and are typically used in automated radiolabeling experiments where liquid losses in the delivery lines are expected. Lyophilized [K+œ2.2.2]OH⁻-cryptate must be dissolved in anhydrous MeCN immediately before the start of the synthesis because decomposition occurs in MeCN at room temperature. The reaction is kept for 10 min at room temperature. Formulation buffer (10 mL phosphate buffered saline (PBS) pH 7.4 with 0.5% (w/v) Na-ascorbate) is added to the reactor, and the resulting mixture is transferred through an HLB cartridge (Oasis HLB cartridge Plus Short, 225 mg sorbent, 60 μm particle size, Waters), preconditioned with 10 mL water, into a waste container. The reactor is rinsed with additional formulation buffer (10 mL), and all contents are transferred through the HLB cartridge into the waste. The HLB cartridge is washed with formulation buffer (10 mL) into the waste. The product is eluted from the cartridge through a 0.22 Pm sterile filter into a sterile vial with ethanol (2 mL). Alternatively, aqueous ethanol (50% v/v, 4 mL) can be used. The product mixture is diluted with formulation buffer (20 mL). (See, e.g., Wurzer et al. EJNMMI radiopharm. chem. 6, 4 2021; the disclosure of which is herein incorporated by reference).

### Example 4: Synthesis of HetSiFA Compounds

The synthesis processes for pyrazole-HetSiFA compounds are described in detail below. As can be readily appreciated, various HetSiFA compounds can be synthesized in a similar process.

Commercially available reactants/reagents purchased from suppliers such as Sigma-Aldrich, TCI, Combi-Blocks, AK Scientific, Gelest, Ambeed, Macrocyclics, Chempep, or AAPPTec were used without further purification unless otherwise stated. Unless mentioned otherwise, all the reactions were set forth at room temperature and under positive pressure of nitrogen/argon in the air-dried (unless flame-dried is specified) reaction tubes/flasks. Reactions were monitored by using waters HPLC system equipped with diode array detector and ESI-MS detector. The reactions were performed in the commercially available reagent grade solvents without further purification or drying of solvent unless mentioned in the procedure. The crude products were worked-up before purification, if any.

Purification by reverse phase HPLC is carried out under standard conditions using Biotage® Isolera one high performance flash purification systems. Elution is performed using a linear gradient of acetonitrile in water (10 mM AmF. or AmBic in deionized water). Solvent system is tailored according to the polarity and stability of the compound, the flow rate used in the purification was under, or the suggested flow rate by the manufacturer, and varies according to the size of C18 column used. Compounds are collected either by UV or waters Mass Detector, ESI Positive Mode. Fractions containing the desired compound were combined, concentrated (rotary evaporator) to remove excess CH₃CN and the resulting aqueous solution is lyophilized to afford the desired material.

Nuclear magnetic resonance (NMR) spectra are recorded on JEOL 400 MHz spectrometer instrument. The residual solvent protons (¹H) were used as the chemical shift reference standard. The following solvents were used: CDCl₃, methanol-d₄, DMSO-d₆, acetone-d₆, and CD₃CN. ¹H-NMR data were presented as follows: chemical shift in ppm downfield from tetramethylsilane (multiplicity, coupling constant, integration). The following abbreviations are used in reporting NMR data: s, singlet; d, doublet; t, triplet; q, quartet; p, pentuplet; h, hextuplet; dd, doublet of doublets; ddd, doublet of doublets of doublets; dddd, doublet of doublets of doublets of doublets; dt, doublet of triplets; dtd, doublet of triplets of doublets; ddt, doublet of doublets of triplets; dq, doublet of quartets; dp, doublet of pentuplets; td, triplet of doublets; qd, quintet of doublets; m, multiplet. The following abbreviations are used in reporting NMR data: br, broad; app, apparent, to describe some singlet, or some multiplicity, whenever appropriate. First order coupling constants were reported using Hz as the unit of frequency, and was measured using the distance between the center of one peak to the center of the other peak.

Unless otherwise stated, purification by normal-phase flash column chromatography on silica gel is carried out under standard conditions using, but not restricted to, either of the following instruments and supplies: Biotage® SP1 or SP2 purification system with Biotage® SNAP Cartridge KP-Sil column 10g, 25g, 50g, 100g or 340g and, CombiFlash®Rf Teledyne Isco purification system with Silica RediSep®Rf normal phase column 12g, 24g, 40g, 80g, 120g, 220g or 330g. In cases where the crude material was purified by vacuum-liquid chromatography (scale-up procedures), technical-grade Supelco (Sigma Aldrich) Silica was used as the stationary phase, and conditioned with a generous amount of hexanes prior to use. Solvent system is tailored according to the polarity and stability (reversed phase) of the compound. Fractions containing the desired compound were combined and concentrated (rotary evaporator) to remove the solvent and to afford the desired material (mostly for normal phase purification), or lyophilized after the fractions were concentrated under reduced pressure (mostly for reverse phase purification to remove CH₃CN), or the residue from normal phase separation (after reduced pressure evaporation) was suspended in CH₃CN (drops)/water mixture and then lyophilized, whichever is deemed more appropriate to yield a better physical aspect of the desired compound. The LCMS methods are as mentioned below.

LCMS (method 8B): Column: Waters Acquity UPLC CSH C18, 1.8 μm, 2.1×30 mm at 40° C.; Gradient: 5% to 100% B in 5.2 minutes; hold 100% B for 1.8 minute; run time=7.0 min; flow=0.9 mL/min; Eluents: A=Milli-Q H2O+10 mM Ammonium formate pH=3.8 (Am.F); Eluent B: Acetonitrile (no additive).

LCMS (method 10A): Column: Waters Acquity UPLC CSH C18, 1.8 μm, 2.1×30 mm at 40° C.; Gradient: 5% to 100% B in 2.0 minutes; hold 100% B for 0.7 minute; run time=2.7 minutes; flow=0.9 mL/min; Eluents: A=Milli-Q H2O+10 mM Ammonium formate pH=3.8 (Am.F); Eluent B: Acetonitrile (no additive).

### Synthesis of 1-(2-(2-azidoethoxy)ethyl)-3-nitro-1H-pyrazole (5a) and 1-(2-(2-azidoethoxy)ethyl)-5-nitro-1H-pyrazole (5b)

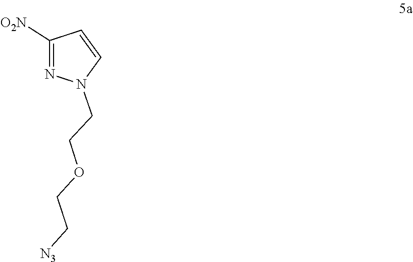 [SMILES: [N-]=[N+]=NCCOCCn1ccc([N+](=O)[O-])n1]

To a flame-dried round bottom flask was added 3-nitro-1H-pyrazole (4, 2.00 g, 17.3 mmol) and DMF (40.0 mL). Cooled the solution to 0° C., then added sodium hydride 60% in dispersion in mineral oil (797 mg, 19.9 mmol) portionwise. The mixture was stirred at 0° C. for 30 minutes, then 2-bromoethyl ether (3.44 mL, 26.0 mmol) was added dropwise. The resulting mixture was then warmed to room temperature.

After stirring at room temperature for 2 hours, the reaction was cooled to 0° C. and was slowly quenched with saturated aqueous ammonium chloride and extracted the mixture with ethyl acetate. The aqueous phase was extracted two more times with ethyl acetate. The combined organic layers were washed with brine, dried over Na₂SO₄, filtered and concentrated under reduced pressure to afford the crude alkylated product. To the crude material obtained from the first step was added DMF (20.0 mL) and sodium azide (2.38 g, 36.4 mmol). The resulting mixture was heated to 80° C. After stirring at 80° C. for 2 hours, the mixture was cooled to room temperature, and was then poured into water and extracted with ethyl acetate. Extracted the aqueous layer three more times with ethyl acetate, and combined organic layers were washed with saturated aqueous ammonium chloride, dried over Na₂SO₄, filtered and concentrated under reduced pressure. The crude oil was purified by normal phase chromatography using a 25g SiO₂ column and eluting with ethyl acetate/hexanes gradient. The product eluted at 35% ethyl acetate and was concentrated to afford 1-(2-(2-azidoethoxy)ethyl)-3-nitro-1H-pyrazole (5a, 2.11 g, 54%) as a clear liquid. ¹H-NMR (400 MHz, CDCl₃) δ 7.56 (d, J=2.5 Hz, 1H), 6.88 (d, J=2.5 Hz, 1H), 4.38 (t, J=4.8 Hz, 2H), 3.87 (t, 4.8 Hz), 3.60 (t, J=4.7 Hz, 2H), 3.32 (t, J=4.7 Hz, 2H). LCMS (method 10A): Calc. for C₇H₁₀N₆O₃Na⁺ [M+H]⁺: 249.1, found: 249.2. Rt=0.80 min.

Scale up. To a flame-dried, three-neck round bottom flask equipped with a mechanical stirrer, a condenser, and a temperature probe was charged 2-(2-azidoethoxy)ethyl 4-methylbenzenesulfonate (3, 63.9 g, 224 mmol) (Delmas, A. F. et. al., Angew. Chem. Int. Ed., 2012, 51, 11320-11324) in DMF (290 mL), and 3-nitro-1H-pyrazole (4, 26.6 g, 231 mmol) and potassium carbonate (63.2 g, 448 mmol) were subsequently added at room temperature. The resulting suspension was stirred and heated at 60° C. (external), maintaining internal temperature (˜55° C.), for 90 min.

The reaction mixture was cooled to room temperature, and the mixture was diluted with ethyl acetate (400 mL). This dilution was washed three times with water (150 mL×3) and partitioned. The combined aqueous layer was extracted with ethyl acetate (400 mL×3), then combined organic layer was pre-dried with brine, dried over Na₂SO₄, filtered and concentrated under reduced pressure to furnish 60.4 g of crude light yellow liquid (r.r. ˜8:1, in favor of the desired N-alkylated regioisomer). The crude liquid was purified by normal phase chromatography using a ˜1.4 kg SiO₂ (using a vacuum-liquid chromatography set up) column and the desired regioisomer, 1-(2-(2-azidoethoxy)ethyl)-3-nitro-1H-pyrazole (5a) was eluted with ethyl acetate/hexanes gradient. The desired regioisomer eluted at ˜50% ethyl acetate in hexanes to furnish the desired pure regioisomer (11.5 g, 23%) as a clear colourless liquid, and another fraction containing both regioisomers in ˜85:15 ratio of major:minor regioisomers (36.6 g, 72% combined). Note: we found that the undesired regioisomer, in a separate parallel reaction, does not react in the subsequent reaction step (silylation), and better separation can be done at the silylation stage; if desired however, the impure fraction can be purified again using the same gradient to furnish more of the desired regioisomer, 1-(2-(2-azidoethoxy)ethyl)-3-nitro-1H-pyrazole (5a). The characterization of pure 1-(2-(2-azidoethoxy)ethyl)-3-nitro-1H-pyrazole (5a) obtained here are in agreement with the previous one (small scale, vide supra).

### Synthesis of 1-(2-(2-azidoethoxy)ethyl)-5-(di-tert-butylsilyl)-3-nitro-1H-pyrazole (6)

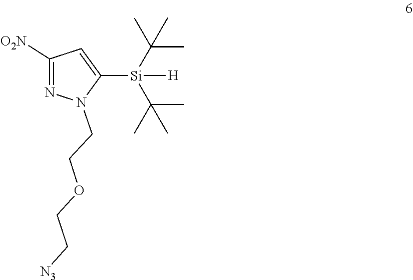 [SMILES: CC(C)(C)[SiH](c1cc([N+](=O)[O-])nn1CCOCCN=[N+]=[N-])C(C)(C)C]

To a flame dried round bottom flask was added diisopropylamine (1.37 mL, 9.75 mmol) and THF (15.0 mL). The solution was cooled to 0° C. and n-butyllithium (2.5 M in hexanes, 3.57 mL, 8.93 mmol) was slowly added. The solution was stirred at 0° C. for 30 minutes before it was added dropwise to a solution of 1-(2-(2-azidoethoxy)ethyl)-3-nitro-1H-pyrazole (5a, 1.68 g, 7.44 mmol) in THF (30.0 mL) at −78° C. The resulting mixture was stirred at −78° C. for 1 hour before adding di-tert-butylchlorosilane (1.67 mL, 8.18 mmol) at the same temperature. Removed the cold bath and allowed the mixture to gradually warm to room temperature. After stirring for 21 hours, the mixture was cooled to 0° C. and was quenched with saturated ammonium chloride. Extracted with ethyl acetate, washed with brine, dried over Na₂SO₄, filtered and concentrated under reduced pressure. The crude oil was purified by normal phase chromatography using a 25g SiO₂ column and eluting with ethyl acetate/hexanes gradient. The product eluted at 15% ethyl acetate where it was then concentrated to afford 1-(2-(2-azidoethoxy)ethyl)-5-(di-tert-butylsilyl)-3-nitro-1H-pyrazole (6, 1.97 g, 72%) as a light yellow solid. ¹H-NMR (400 MHz, CDCl₃) δ 7.03 (s, 1H), 4.52 (t, J=5.8 Hz. 2H), 4.14 (s, 1H), 3.98 (t, J=5.8 Hz, 2H), 3.62-3.57 (m, 2H), 3.33-3.26 (m, 2H), 1.06 (s, 18H). LCMS (method 10A): Calc. for C₁₅H₂₉N₆O₃Si⁺ [M+H]⁺: 369.2, found: 369.3. Rt=1.73 min.

Scale up. To a flame dried round bottom flask equipped with a stir bar was added 1-(2-(2-azidoethoxy)ethyl)-3-nitro-1H-pyrazole (5a, 14.3 g, 56.9 mmol) in THF (330 mL). The solution was cooled to −78° C. and lithium diisopropylamide (57.5 mL, 57.5 mmol) was slowly added over 40 minutes. The resulting mixture was stirred at -78° C. for 1 hour before adding di-tert-butylchlorosilane (14.2 mL, 68.3 mmol) dropwise over 30 minutes at the same temperature. The reaction mixture was allowed to reach room temperature over the course of the reaction overnight (16 h), by removal of the cooling bath.

The mixture was cooled to 0° C. and was quenched with saturated ammonium chloride. Extracted with ethyl acetate, pre-dried with brine, dried over Na₂SO₄, filtered and concentrated under reduced pressure. The crude oil was purified by normal phase chromatography using a SiO₂ column (vacuum-liquid chromatography set up) and eluting with ethyl acetate/hexanes gradient (10-55%). The desired product eluted at ˜45% ethyl acetate, where fractions containing the desired compound were concentrated under reduced pressure (solution immersed at ˜75° C. water bath) until constant weight of the oil was observed, the desired compound eventually solidified upon standing at room temperature. Then finally, the desired compound was resuspended in ACN/water mixture and lyophilized to afford 1-(2-(2-azidoethoxy)ethyl)-5-(di-tert-butylsilyl)-3-nitro-1H-pyrazole (6, 7.60 g, 34%) as a yellow powder. The characterization of the desired compound obtained here are in agreement with the previous one (small scale, vide supra).

### Synthesis of tert-butyl (2-(2-(5-(di-tert-butylsilyl)-3-nitro-1H-pyrazol-1-yl)ethoxy)ethyl)carbamate (7)

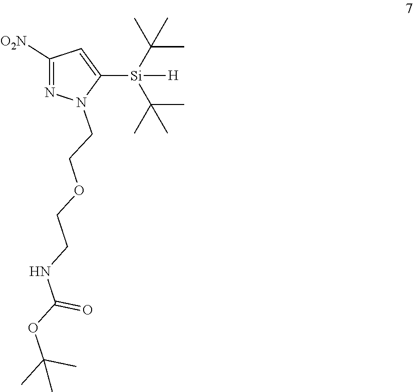 [SMILES: CC(C)(C)OC(=O)NCCOCCn1nc([N+](=O)[O-])cc1[SiH](C(C)(C)C)C(C)(C)C]

To a stirring solution of 1-(2-(2-azidoethoxy)ethyl)-5-(di-tert-butylsilyl)-3-nitro-1H-pyrazole (6, 1.97 g, 5.35 mmol) in THF (20.0 mL) was dropwise added trimethylphosphine (1 M in THF, 8.02 mL, 8.02 mmol). The mixture was stirred at room temperature for 45 minutes, then water (5.00 mL) was slowly added. The reaction was stirred at room temperature for another 30 minutes, then sodium carbonate (1.14 g, 10.7 mmol) and di-tert-butyl dicarbonate (1.84 mL, 8.02 mmol) were added to the mixture at room temperature. (Caution: vigorous bubbling is observed upon addition of trimethylphosphine. The reaction is exothermic upon addition of water). After stirring the resulting mixture at room temperature for 30 minutes, the mixture was extracted with ethyl acetate, washed with brine, dried over Na₂SO₄, filtered and concentrated under reduced pressure. The crude oil was purified by normal phase chromatography using a 25g SiO₂ column and eluting with ethyl acetate/hexanes gradient. The product eluted at 15% ethyl acetate where it was then concentrated to afford tert-butyl (2-(2-(5-(di-tert-butylsilyl)-3-nitro-1H-pyrazol-1-yl)ethoxy)ethyl)carbamate (7, 2.01 g, 85%) as a yellow oil. ¹H-NMR (400 MHz, CDCl₃) δ 7.03 (s, 1H), 4.69 (br s, 1H), 4.49 (t, J=5.7 Hz, 2H), 4.11 (s, 1H), 3.91 (t, J=5.7 Hz, 2H), 3.47 (t, J=5.2 Hz, 2H), 3.22 (dd, J=10.1, 5.0 Hz, 2H), 1.41 (s, 9H), 1.06 (s, 18H). LCMS (method 10A): Calc. for C₂₀H₃₉N₄O₅Si⁺ [M+H]⁺: 443.3, found: 443.4. Rt=1.75 min.

Scale up. To a round bottom flask equipped with a stir bar, a solution of 1-(2-(2-azidoethoxy)ethyl)-5-(di-tert-butylsilyl)-3-nitro-1H-pyrazole (6, 27.0 g, 73.3 mmol) in THF (200 mL) was added a solution of trimethylphosphine (1 M, 110 mL, 110 mmol) in THF, at 0° C. dropwise. The reaction mixture was stirred at 0° C. for ˜5 minutes more after the conclusion of the addition or until bubbling has ceased, then at room temperature for 1 hour. The reaction mixture was cooled to 0° C., H₂O (83.5 mL) was introduced slowly to the reaction mixture, and then stirred at room temperature for another 30 minutes before beginning the next step. The reaction mixture was added sodium carbonate (16.2 g, 152 mmol) and di-tert-butyl dicarbonate (16.8 mL, 73.3 mmol) at room temperature. After stirring at room temperature overnight, the reaction mixture was added generous amount ethyl acetate/water biphasic mixture until all sodium carbonate has dissolved, the reaction mixture was partitioned, and the organic layer washed generously with water, and then pre-dried with brine, dried over Na₂SO₄, filtered and concentrated under reduced pressure until constant weight, and then dried under high vacuum overnight to afford tert-butyl (2-(2-(5-(di-tert-butylsilyl)-3-nitro-1H-pyrazol-1-yl)ethoxy)ethyl)carbamate (7, 32.4 g, 100%, 96% purity by LCMS, method 10A)) as a light orange oil (quantitative), deemed pure enough for the next stage. This oil was carried forward onto the next step without purification. The LCMS characterization of the desired compound obtained here are in agreement with the previous one (small scale, vide supra).

### Synthesis of tert-butyl (2-(2-(3-amino-5-(di-tert-butylsilyl)-1H-pyrazol-1-yl)ethoxy)ethyl)carbamate (8)

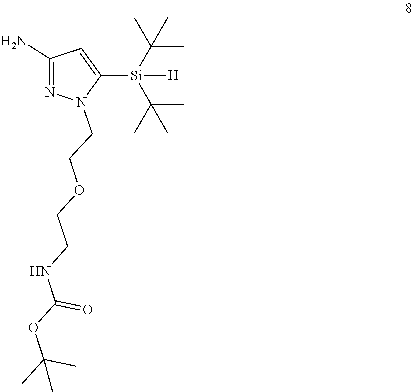 [SMILES: CC(C)(C)OC(=O)NCCOCCn1nc(N)cc1[SiH](C(C)(C)C)C(C)(C)C]

To a round bottom flask containing tert-butyl (2-(2-(5-(di-tert-butylsilyl)-3-nitro-1H-pyrazol-1-yl)ethoxy)ethyl)carbamate (7, 2.01 g, 4.53 mmol) was added MeOH (30.0 mL) and palladium (10% on activated carbon wet, 48.2 mg, 45.3 μmol). The mixture was flushed with nitrogen before attaching a hydrogen balloon. The reaction mixture was stirred at room temperature under hydrogen atmosphere. After stirring at room temperature for 19 hours, the reaction mixture was filtered through a Celite pad, and the filter cake was rinsed three more times with methanol. The filtrate was concentrated to afford tert-butyl (2-(2-(3-amino-5-(di-tert-butylsilyl)-1H-pyrazol-1-yl)ethoxy)ethyl)carbamate (8, 1.87 g, 92%) as a light green oil. LCMS (method 10A): Calc. for C₂₀H₄₁N₄O₃Si⁺ [M+H]⁺: 413.4, found: 413.6. Rt=1.54 min.

Scale up. To a round bottom flask equipped with a stir bar was added tert-butyl (2-(2-(5-(di-tert-butylsilyl)-3-nitro-1H-pyrazol-1-yl)ethoxy)ethyl)carbamate (7, 32.4 g, 73.2 mmol) in MeOH (500 mL). The solution was bubbled with nitrogen gas for 30 min, one good balloon (1 atm), then palladium (10% on activated carbon, 849 mg, 798 μmol) was added to the reaction mixture in one portion. Nitrogen gas atmosphere and saturation was replaced with hydrogen atmosphere by attaching a two-skinned hydrogen balloon (1 atm), then placing the reaction mixture under vacuum (hydrogen balloon off), followed by flushing with hydrogen (vacuum off), and this cycle was repeated multiple times. After ˜6 h, another hydrogen balloon was attached to the reaction mixture to make it into a two (two-skinned) hydrogen balloon. The reaction mixture was stirred at room temperature under hydrogen atmosphere for 36 h. Following completion of the reaction after 36 h, the hydrogen gas atmosphere of the reaction mixture was replaced with nitrogen gas, then it was placed under vacuum (nitrogen off), and then flushed with nitrogen gas (vacuum off). The cycle was repeated multiple times. The reaction mixture was filtered through a Celite pad, and the filter cake was rinsed three more times with generous amount of methanol. The filtrate containing the desired product was concentrated under reduced pressure to afford a crude tert-butyl (2-(2-(3-amino-5-(di-tert-butylsilyl)-1H-pyrazol-1-yl)ethoxy)ethyl)carbamate (8, 30.2 g, 95%, 95% purity by LCMS, method 10A)) as a yellow oil, this was used in the next step without purification. The LCMS characterization of the desired compound obtained here are in agreement with the previous one (small scale, vide supra).

### Synthesis of (1-(2-(2-((tert-butoxycarbonyl)amino)ethoxy)ethyl)-5-(di-tert-butylsilyl)-1H-pyrazol-3-yl)glycine (9)

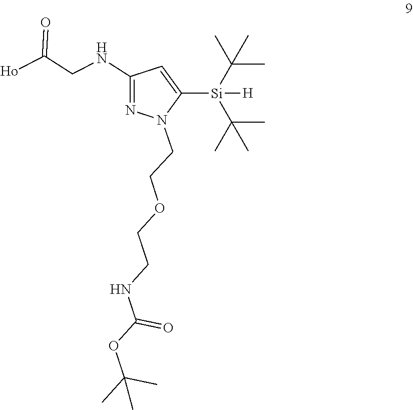

To a solution of tert-butyl (2-(2-(3-amino-5-(di-tert-butylsilyl)-1H-pyrazol-1-yl)ethoxy)ethyl)carbamate (8, 1.87 g, 4.17 mmol) in MeOH (30.0 mL) was added acetic acid (482 μL, 8.34 mmol) and glyoxylic acid monohydrate (392 mg, 4.17 mmol) at 0° C. The mixture was stirred at 0° C. for 30 minutes before adding sodium cyanoborohydride (276 mg, 4.17 mmol) and the reaction mixture was then warmed to room temperature. After stirring at room temperature for 1 hour, the reaction mixture was concentrated under reduced pressure, and the crude residue was subjected to purification by reverse phase chromatography using a 40 g C18 column and eluting with 10 mM ammonium formate/acetonitrile gradient. The product eluted at 55% ACN where it was concentrated down to a suspension, diluted with water then diluted with water and lyophilized over a weekend to afford (1-(2-(2-((tert-butoxycarbonyl)amino)ethoxy)ethyl)-5-(di-tert-butylsilyl)-1H-pyrazol-3-yl)glycine (9, 1.54 g, 69%) as a white powder. ¹H-NMR (400 MHz, CD₃CN) 5 5.76 (s, 1H), 5.37 (br s, 1H), 4.49 (t, J=6.0 Hz, 2H), 4.17 (t, J=5.6 Hz, 2H), 3.98 (s, 1H), 3.83 (s, 2H), 3.70 (t, J=5.6 Hz, 2H), 3.36 (t, J=5.5 Hz, 2H), 3.08 (q, J=5.6 Hz, 2H), 1.37 (s, 9H), 1.01 (s, 18H), COOH and NHBoc proton signals weak. LCMS (method 10A): Calc. for C₂₂H₄₃N₄O₅Si⁺ [M+H]⁺: 471.3, found: 471.4. Rt=1.46 min.

Scale up. To a solution of tert-butyl (2-(2-(3-amino-5-(di-tert-butylsilyl)-1H-pyrazol-1-yl)ethoxy)ethyl)carbamate (8, 30.2 g, 65.9 mmol) in MeOH (508 mL) was added acetic acid (7.54 mL, 132 mmol) and glyoxylic acid monohydrate (6.38 g, 65.9 mmol) at 0° C. The mixture was stirred at 0° C. for 1 h, before adding sodium cyanoborohydride (2.40 g, 36.2 mmol) portionwise (˜500 mg at a time) (Caution). At the conclusion of the addition, the mixture was left stirring at 0° C. for 30 min more. The reaction mixture was added H₂O (150 mL) slowly, and as much methanol as possible was removed under reduced pressure (for eased in partitioning). The crude solution was partitioned between generous amount of ethyl acetate and water, and the organic layer was pre-dried with brine. The aqueous layer was back-extracted two more times with ethyl acetate. (Note: the aqueous layer obtained after the extraction, and the methanol-rich fraction after the reduced pressure evaporation was treated with bleach, before disposal). Combined organic layers were dried over Na₂SO₄, filtered and concentrated under reduced pressure to furnish (1-(2-(2-((tert-butoxycarbonyl)amino)ethoxy)ethyl)-5-(di-tert-butylsilyl)-1H-pyrazol-3-yl)glycine (9, 33.0 g, 82% purity by LC10 3-min run) as a white foam, this was carried forward onto the next step without purification. The characterization of the desired compound obtained here are in agreement with the previous one (small scale, vide supra).

### Synthesis of (1-(2-(2-((tert-butoxycarbonyl)amino)ethoxy)ethyl)-5-(di-tert-butylfluorosilyl)-1H-pyrazol-3-yl)glycine (PPI-24068)

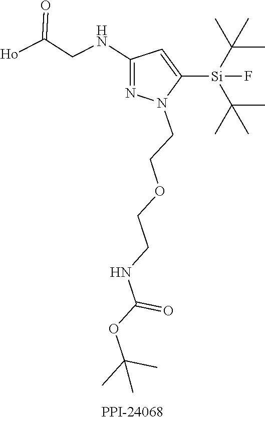

To round bottom flask containing (1-(2-(2-((tert-butoxycarbonyl)amino)ethoxy)ethyl)-5-(di-tert-butylsilyl)-1H-pyrazol-3-yl)glycine (9, 1.54 g, 2.88 mmol) was added potassium fluoride (253 mg, 4.32 mmol), 18-crown-6 (1.16 g, 4.32 mmol) and THF (30.0 mL). Acetic acid (499 μL, 8.64 mmol) was then added and the mixture was heated to 40° C. After 4 hours at 40° C., the mixture was then concentrated under reduced pressure, dissolved in minimal DMF and subjected to reverse phase chromatography (25g C18 column, eluting with 10 mM ammonium formate/acetonitrile). The product eluted at 67% ACN where it was concentrated down to a suspension, diluted with minimal ACN and was lyophilized overnight to afford (1-(2-(2-((tert-butoxycarbonyl)amino)ethoxy)ethyl)-5-(di-tert-butylfluorosilyl)-1H-pyrazol-3-yl)glycine (PPI-24068, 1.07 g, 76%) as a white powder. ¹H-NMR (400 MHz, CD₃CN) δ 5.77 (s, 1H), 5.39 (br s, 1H), 4.54 (br s, 1H), 4.14 (t, J=5.5 Hz, 2H), 3.81 (s, 2H), 3.70 (t, J=5.4 Hz, 2H), 3.36 (t, J=5.4 Hz, 2H), 3.08 (q, J=5.6 Hz, 2H), 1.37 (s, 9H), 1.02 (s, 18H), COOH proton signal weak, NH proton at 4.54 ppm very broad. ¹⁹F-NMR (376 MHz, CD₃CN) 5-181.96 (s). LCMS (method 10A): Calc. for C₂₂H₄₂FN₄O₅Si⁺ [M+H]⁺: 489.3, found: 489.4. Calc. for C₂₂H₄₀FN₄O₅Si⁻ [M−H]⁻: 487.3, found: 487.4. Rt=1.43 min.

Scale up. To round bottom flask containing (1-(2-(2-((tert-butoxycarbonyl)amino)ethoxy)ethyl)-5-(di-tert-butylsilyl)-1H-pyrazol-3-yl)glycine (9, 33.0 g, 70.1 mmol) and THF (275 mL) was added potassium fluoride (6.17 g, 105 mmol), and 18-CROWN-6 (28.4 g, 105 mmol). Effervescence and exotherm (˜5° C. from rt) was observed after addition of potassium fluoride and crown ether.

After stirring at room temperature for 2 hr, the reaction was deemed complete. The reaction mixture was concentrated under reduced pressure, and the crude residue was partitioned between generous amount of ethyl acetate and water. The aqueous layer was extracted two more times with ethyl acetate and the combined organic layers were dried over Na₂SO₄, filtered and concentrated under reduced pressure. The crude residue was purified by reverse phase chromatography using a 400 g C18 column and the desired compound was eluted with 10 mM ammonium formate/acetonitrile gradient (0-55%). The product eluted at ˜45% ammonium formate. Fractions of interest were pooled and concentrated to remove as much ACN/water as possible, and was then divided according to purity (two fractions). The small second fraction (˜5 g, ˜80% purity) was re-purified using the same gradient, but slower pace of elution, and using 120 g of C18 column in place of the 400 g C18 column. In total, after two dissected purifications, (1-(2-(2-((tert-butoxycarbonyl)amino)ethoxy)ethyl)-5-(di-tert-butylfluorosilyl)-1H-pyrazol-3-yl)glycine (PPI-24068, 21 g, ˜30% over 4 steps, ˜93% purity by LC10 at 3-min run) was obtained as a white powder after lyophilisation. The characterization of the desired compound obtained here are in agreement with the previous one (small scale, vide supra). Note: the compound show some instability after prolong storage, and under basic medium, attempts to purify title the compound using 10 mM AmB in deionized water and ACN gradient were not fruitful.

### Synthesis of N-(((9H-fluoren-9-yl)methoxy)carbonyl)-N-(1-(2-(2-((((9H-fluoren-9-yl)methoxy)carbonyl)amino)ethoxy)ethyl)-5-(di-tert-butylfluorosilyl)-1H-pyrazol-3-yl)glycine (PPI-24069)

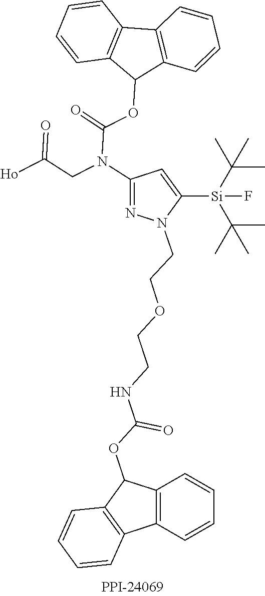

To a round bottom flask equipped with a stir bar, (1-(2-(2-((tert-butoxycarbonyl)amino)ethoxy)ethyl)-5-(di-tert-butylfluorosilyl)-1H-pyrazol-3-yl)glycine (PPI-24068, 3.19 g, 5.87 mmol) was dissolved in DCM (70.0 mL), then trifluoroacetic acid (TFA) (5.72 mL, 74.0 mmol) was added dropwise at room temperature, and the reaction mixture was continuously stirred at 40° C. for 90 min. After 90 minutes, the reaction mixture was cooled down to room temperature, and then concentrated to afford the Boc-deprotected intermediate (not shown). The reaction was concentrated under reduced pressure and the residue was resuspended in ˜50 mL DCM, sonicated and then concentrated under reduced pressure, this cycle was repeated three more times to furnish a colorless gum, and this gum was dried further under vacuum overnight and then used in the next step (Fmoc coupling) without purification. The gum was dissolved in DCM (70.0 mL), Fmoc chloride (3.10 g, 11.7 mmol) was added in two equal portions (one equivalent before DIPEA was added and another equivalent after ˜4 equivalent of DIPEA was added, or halfway through completion) and N,N-Diisopropylethylamine (DIPEA) (8.00 mL, 45.5 mmol) (˜1.0 mL every ˜15 min to 30 min) in portions every 15 mins to 30 min, at 0° C. The course of reaction was followed using LC10 until the SM have been fully consumed.

After ˜3 h at 0° C., the reaction mixture was diluted with generous amount of DCM and washed with saturated aqueous ammonium chloride. The organic layer was dried over Na₂SO₄, filtered and concentrated under reduced pressure. The crude residue was purified by reverse phase chromatography (C18, 120 g) and eluting with 10 mM ammonium formate/acetonitrile gradient, 50-100%. The title compound eluted at 90-100% ACN, fractions were then concentrated and lyophilized to afford furnish N-(((9H-fluoren-9-yl)methoxy)carbonyl)-N-(1-(2-(2-((((9H-fluoren-9-yl)methoxy)carbonyl)amino)ethoxy)ethyl)-5-(di-tert-butylfluorosilyl)-1H-pyrazol-3-yl)glycine (PPI-24069, 2.90 g, 56%, ˜95% purity by LCMS, method 10A) as a white powder. This was used in the peptide coupling without further purification. LCMS (method 10A): Calc. for C₄₇H₅₄FN₄O₇Si⁺ [M+H]⁺: 833.4, found: 833.6. Rt=2.01 min.

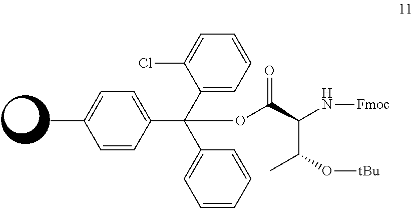

Resin activation and first amino acid coupling. PPI-24073 was synthesized on solid-phase using Fmoc-based approach. In a glass reactor with a frit, 2-chlorotrityl chloride resin (10, 1.2 mmol/g, 100-200 mesh, 12 mmol) was swelled in dichloromethane, drained, and activated by shaking for 10 min in 100 mL of dichloromethane and drained again. Fmoc-Thr(tBu)-OH (8.60 g, 21.0 mmol) was dissolved in dichloromethane (80 mL) in a separate flask, DIPEA (12.6 mL, 72 mmol) was added, and the solution was mixed for 5 minutes (sonication was performed to aid dissolution). It was then poured in to an activated 2-chlorotrityl chloride resin (10) and reacted by shaking for 4.5 h. Then methanol (20 mL, HPLC grade) was added, and the mixture was Shaken for 30 minutes (this step is performed to cap unreacted resin, if any). The reaction vessel was drained, and the resin was washed with dichloromethane (2×120 mL), DMF (2×120 mL), methanol (2×120 mL) and again dichloromethane (2×120 mL). Resin 11 was used in next step.

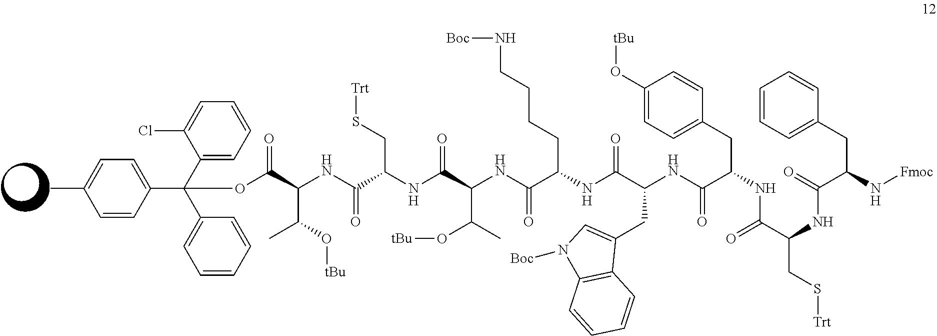

Peptide chain elongation. Resin 11 was treated twice with a mixture of piperidine and DMF (1:2 ratio) for 45 minutes each to deprotect Fmoc-group. Fmoc removal was confirmed by Kaiser test displaying positive test. After successive washing of the resin with DMF (2×120 mL), methanol (2×120 mL), and dichloromethane (2×120 mL). Fmoc-Cys(Trt)-OH (1.5 equiv) was preactivated with HATU (1.5 equiv), HOAt (1.5 equiv) and DIPEA (4 equiv) in DMF (100 mL) and the resulting solution was then added to the reaction vessel containing resin and shaken for at least 4 h at room temperature for two reaction cycles. The amino acid coupling was confirmed by negative Kaiser test. Using a similar procedure, Fmoc-Thr(tBu)-OH, Fmoc-Lys(Boc)-OH, Fmoc-D-Trp(Boc)-OH, Fmoc-L-Tyr(tBu)-OH, Fmoc-Cys(Trt)-OH, and Fmoc-D-Phe-OH were subsequently coupled to the peptide sequence to obtain 12. The resin was washed subsequently with DMF (2×120 mL), methanol (2×120 mL), and dichloromethane (2×120 mL). If required, the reaction was paused after an amino acid coupling with air dry resin was stored in refrigerator. Sequence was resumed after resin activation using DCM for 30 min.

Kaiser test solution recipe. [(0.5 g ninhydrin in 10 mL ethanol)+0.4 mL, 0.001 M aqueous KCN in 20 mL pyridine]. These two solutions were mixed in 1:1 ratio, resin was suspended and heated to observe color of resin.

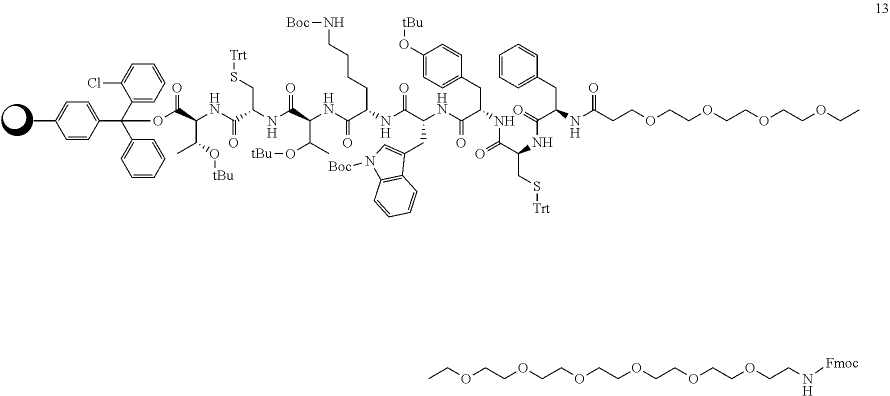

PEG12-linker coupling. Resin 12 (1.2 mmol/g, 0.4 mmol) was treated twice with 1:2 mixture of piperidine and DMF (3 mL) to deprotect Fmoc-group followed by subsequent washings with DMF, methanol and dichloromethane (positive Kaiser test). Fmoc-NH-PEG(12)-GOGH (1.0 equiv) was preactivated with HATU (1.0 equiv), HOAt (1.0 equiv) and DIPEA (2.7 equiv) in DMF (3 mL) and the resulting solution was then added to the reaction vessel containing resin and shaken for overnight at room temperature. The resin was washed subsequently with DMF (2×120 mL), methanol (2×120 mL), and dichloromethane (2×120 mL). The completion of reaction was confirmed by negative Kaiser test.

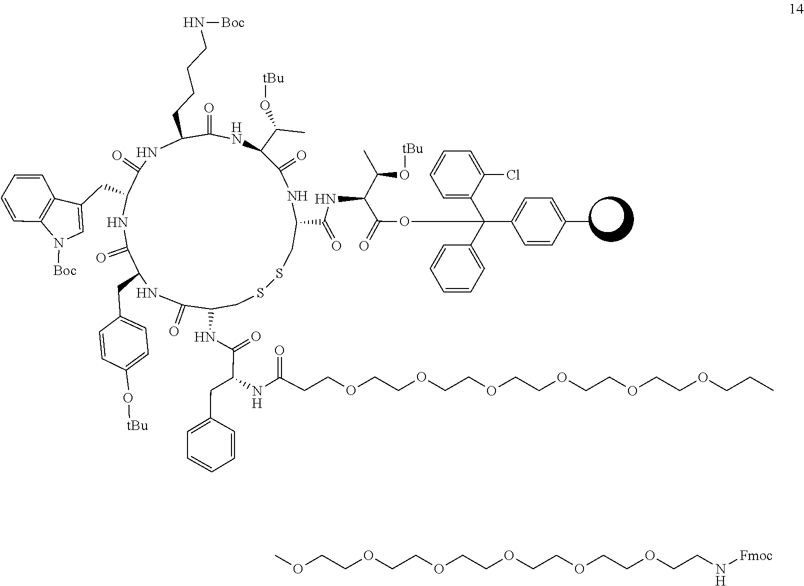

Peptide bridge formation. To a stirred solution of iodine (500 mg, 1.97 mmol) in DMF (40.0 mL) in a glass reactor was added resin 13 (1.2 mmol/g, 0.4 mmol) suspended in DMF (20.0 mL) in portion over 30 min. The whole mixture was shaken gently over a period of 4 h at room temperature. The resin was then drained, and excess iodine was washed out using DMF (3×20 mL) and DCM (3×20 mL). A small amount of resin was analyzed using LCMS after treatment of small amount of deprotection cocktail containing TFA for overnight.

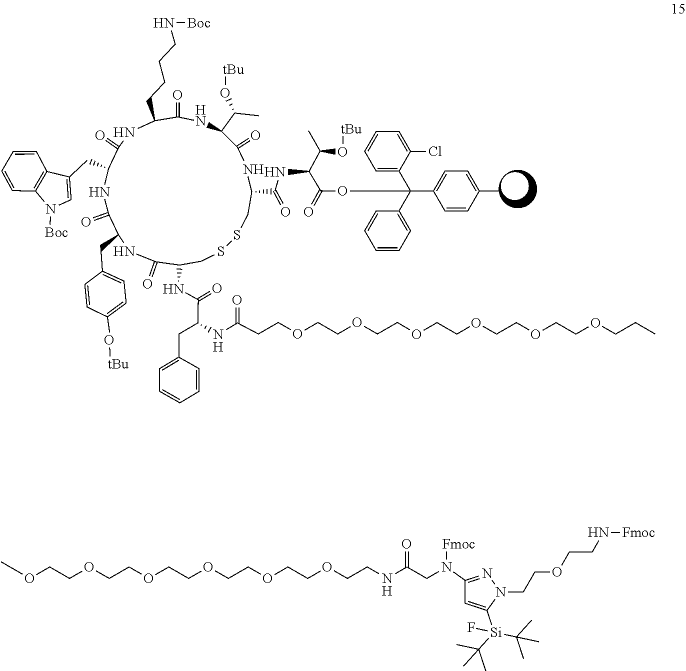

Coupling of N-(((9H-fluoren-9-yl)methoxy)carbonyl)-N-(1-(2-(2-((((9H-fluoren-9-yl)methoxy)carbonyl)amino)ethoxy)ethyl)-5-(di-tert-butylfluorosilyl)-1H-pyrazol-3-yl)glycine (PPI-24069).

Resin 14 (1.2 mmol/g, 0.4 mmol) was treated twice with 1:2 mixture of piperidine and DMF (3 mL) to deprotect Fmoc-group followed by subsequent washings with DMF, methanol and dichloromethane (positive Kaiser test). PPI-24069 (1.0 equiv) was preactivated with HATU (1.0 equiv), HOAt (1.0 equiv) and DIPEA (2.7 equiv) in DMF (3 mL) and the resulting solution was then added to the reaction vessel containing resin and shaken for overnight at room temperature. The resin 15 was washed subsequently with DMF (2×10 mL), methanol (2×10 mL), and dichloromethane (2×10 mL). The completion of reaction was confirmed by negative Kaiser test.

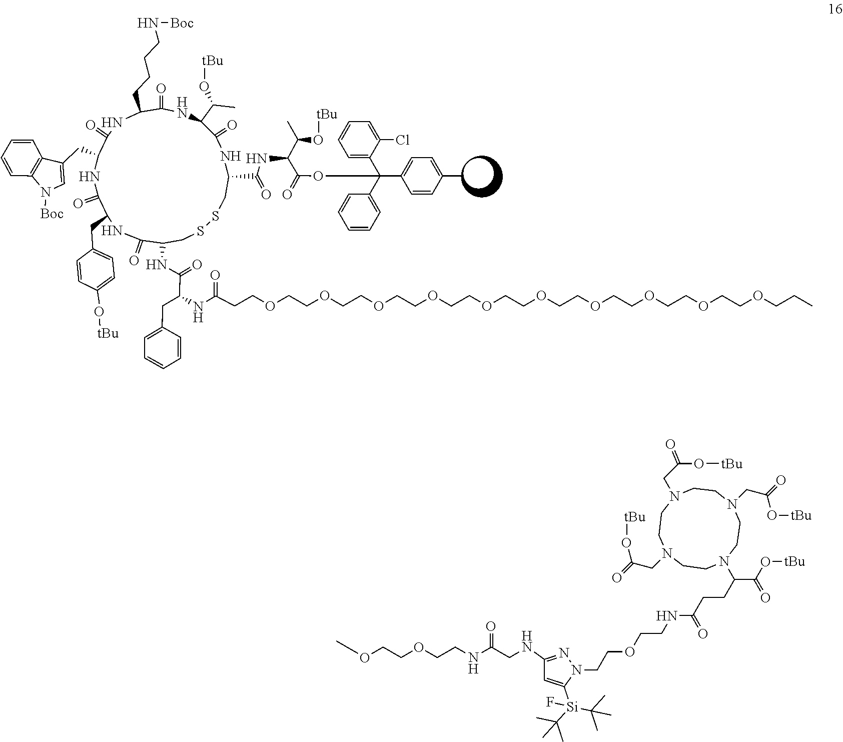

DOTAGA-tetra(t-Bu)-COOH coupling. Resin 15 (1.2 mmol/g, 0.4 mmol) was treated twice with 1:2 mixture of piperidine and DMF (3 mL) to deprotect Fmoc-group followed by subsequent washings with DMF, methanol and dichloromethane (positive Kaiser test). DOTAGA-tetra(t-Bu)-COOH (1.4 equiv) was preactivated with HATU (1.4 equiv), HOAt (1.4 equiv) and DIPEA (3.8 equiv) in DMF (3 mL) and the resulting solution was then added to the reaction vessel containing resin and shaken for overnight at room temperature. The resin 16 was washed subsequently with DMF (2×10 mL), methanol (2×10 mL), and dichloromethane (2×10 mL). The completion of reaction was confirmed by negative Kaiser test.

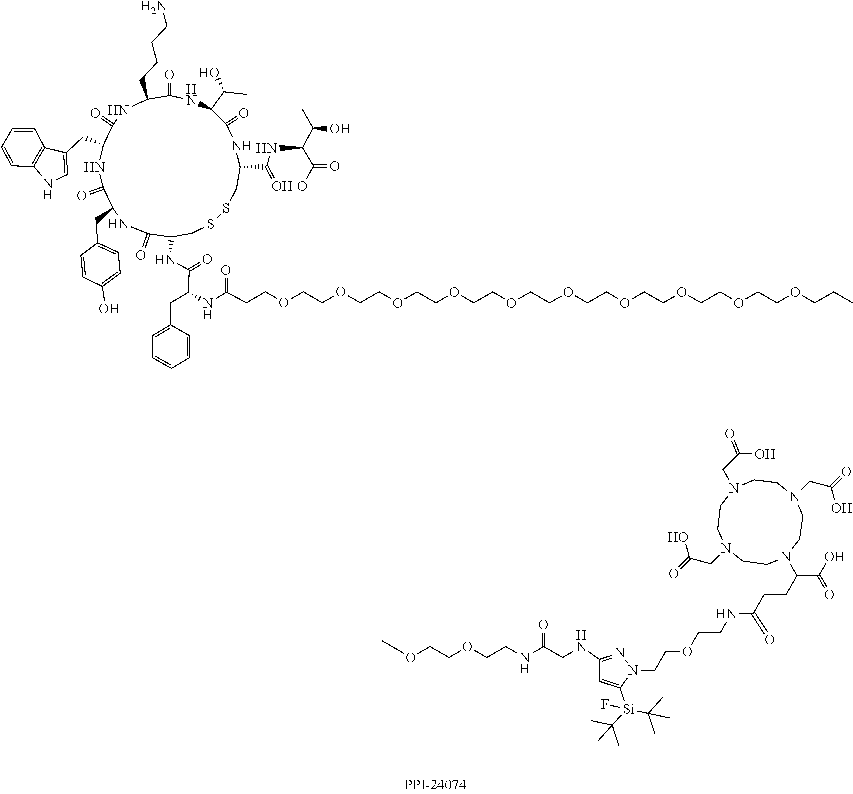

Peptide deprotection and removal from resin. Synthesis of 2,2′,2″-(10-(4-((2-(2-(3-(((R)-45-(((4R,7S,10S,13R,16S,19R)-13-((1H-indol-3-yl)methyl)-10-(4-aminobutyl)-4-(((1 S,2R)-1-carboxy-2-hydroxypropyl)carbamoyl)-16-(4-hydroxybenzyl)-7-((R)-1-hydroxyethyl)-6,9,12,15,18-pentaoxo-1,2-dithia-5,8,11,14,17-pentaazacycloicosan-19-yl)amino)-44-benzyl-2,42,45-trioxo-6,9,12,15,18,21,24,27,30,33,36,39-dodecaoxa-3,43-diazapentatetracontyl)amino)-5-(di-tert-butylfluorosilyl)-1H-pyrazol-1-yl)ethoxy)ethyl)amino)-1-carboxy-4-oxobutyl)-1,4,7,10-tetraazacyclododecane-1,4,7-triyl)triacetic acid (PPI-24074).

Resin 16 was treated overnight at room temperature with premade deprotection cocktail (15 mL, 446 μmol) as below. The solvent was collected by filtration and its amount was reduced on rotavapor. The peptide was precipitated by adding cold diethyl ether, centrifuged and the solid was washed twice with cold diethyl ether to obtain desired peptide product PPI-24074 (650 mg, 59% yield). The obtained solid was analyzed by using LCMS, and it was used as such for next step.

LCMS (method 8B): EM=C₁₁₂H₁₇₈FN₁₉O₃₆S₂Si; Calc. for [M+2H]²⁺=1239.1, found=1239.5; Calc. for [M+3H]³⁺=826.4, found=826.4; Calc. for [M−2H]²⁻=1237.1, found=1237.5; Rt=2.02 min.

Deprotection cocktail: [88% TFA+5% water+5% Phenol+2% Triisopropylsilane].

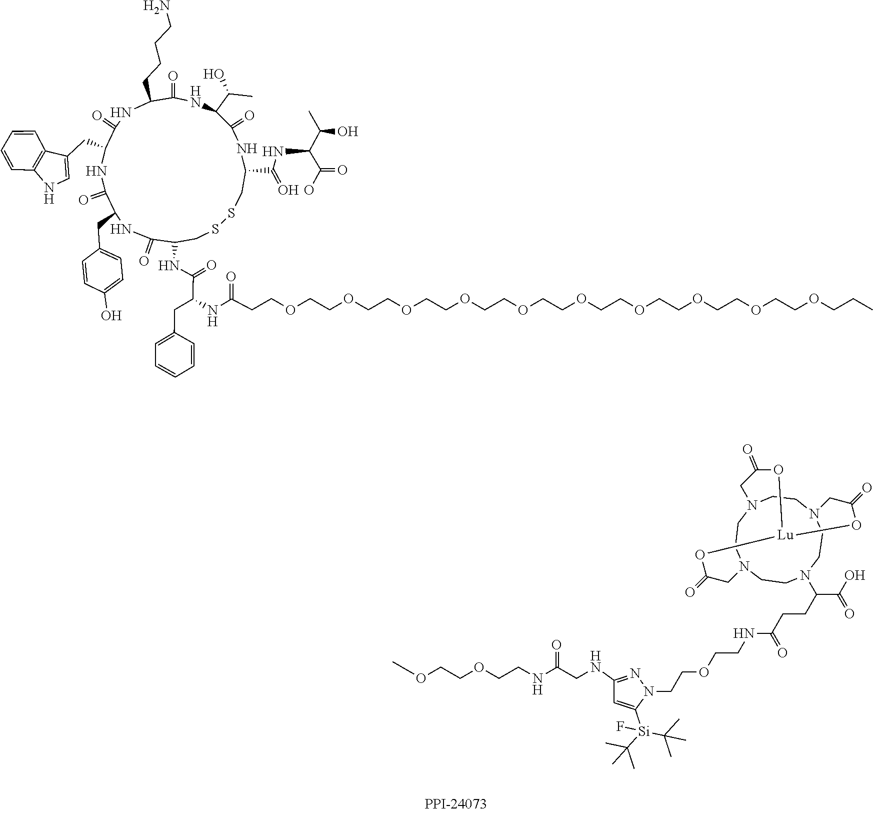

Formation of Lutetium chelate (PPI-24073). To a solution of PPI-24074 (50.0 mg, 20.2 μmol) in DMSO (1.00 mL) was added aqueous LuCl₃ (250 PL, 200 mM) and the mixture was stirred at 90° C. for 1 h. Reaction was then stopped, cooled to room temperature, and directly purified using reverse phase column (C18, 30 g) eluting with 10 mM ammonium formate (pH 3.8) and acetonitrile gradient. Pure product fractions collected in 36% acetonitrile were combined and lyophilized overnight to obtain desired product PPI-24073 (7 mg, 13% yield).

LCMS (method 8B): EM=C₁₁₂H₁₇₅FLuN₁₉O₃₆S₂Si; Calc. for [M+2H]²⁺=1325.1, found=1325.2; Calc. for [M+3H]³⁺=883.7, found=883.7; Rt=2.05 min.

HRMS: Mass observed for [M+2Na−2H] 2692.0625, calc. 2692.0689. Mass observed for [M+3Na−3H] 2714.0625, calc. 2714.0508. Mass observed for [M+4Na−4H] 2736.0313, calc. 2736.0327.

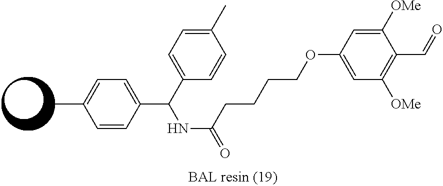 [SMILES: COc1cc(OCCCCC(=O)NC(c2ccc(C)cc2)c2ccc(C)cc2)cc(OC)c1C=O]

BAL resin synthesis. MBHA resin (17, 0.8-1.6 mmol/g, 3.50 g, 4.20 mmol) was activated in dichloromethane (50 mL) for 30 min. in a glass reactor with fret and drained. The resin showed a positive Kaiser test. In a separate flask, 5-(4-formyl-3,5-dimethoxyphenoxy)pentanoic acid (18, 1.5 equiv), DIC (1.5 equiv) and HOBt (1.5 equiv) were mixed in DMF (25.0 mL) for 5 min and the solution was added to the resin. The resin was shaken for 4 h at room temperature, drained and resin was treated again with fresh reagents as above. The solvent was drained, and the obtained BAL resin 19 was washed subsequently with DMF (3×25 mL), methanol (3×25 mL), and dichloromethane (3×25 mL). Completion of the reaction was confirmed by a negative Kaiser test.

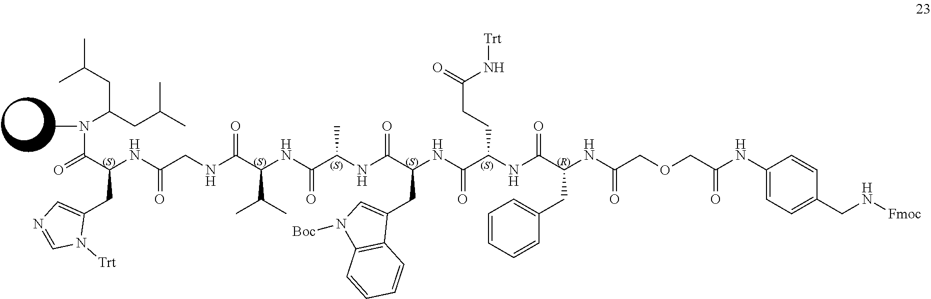

Synthesis of product resin 23. Synthesis of product resin 23 from Bal resin 19 was achieved using the protocol described in the reference paper (ACS Omega 2019, 4, 1470-1478). After coupling of the Fmoc-protected pABzA-DIG linker, the obtained resin 23 was used for further functionalization.

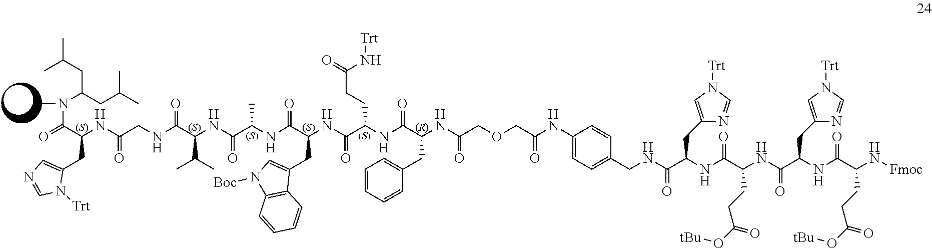

Synthesis of product resin 24. Fmoc-L-His(Trt)-OH (3 equiv) preactivated with HATU (3 equiv), HOAt (3 equiv) and DIEA (8 equiv) in DMF (6 mL) was then added to the reaction vessel containing resin 23 in two equal portions and shaken for 4 h and overnight respectively at room temperature. Fmoc-deprotection was performed using piperidine/DMF (1:2) solution over two reaction cycles for total 1.5 h. Using a similar procedure, Fmoc-L-Glu(tBu)-OH, Fmoc-L-His(Trt)-OH and again Fmoc-L-Glu(tBu)-OH were subsequently coupled to the peptide sequence. Formation of product resin 24 was confirmed by Kaiser test.

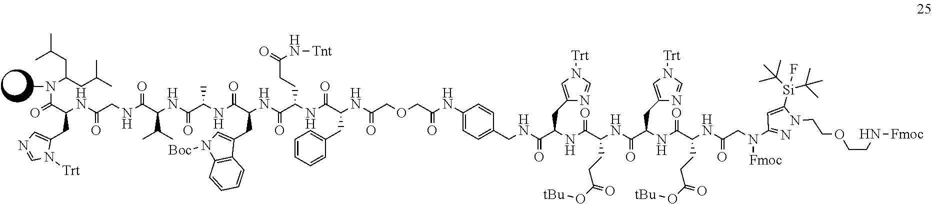

Coupling of N-(((9H-fluoren-9-yl)methoxy)carbonyl)-N-(1-(2-(2-((((9H-fluoren-9-yl)methoxy)carbonyl)amino)ethoxy)ethyl)-5-(di-tert-butylfluorosilyl)-1H-pyrazol-3-yl)glycine (PPI-24069).

Resin 24 (1.2 mmol/g, 0.65 mmol) was treated twice with 1:2 mixture of piperidine and DMF (3 mL) to deprotect Fmoc-group followed by subsequent washings with DMF, methanol and dichloromethane (positive Kaiser test). PPI-24069 (1.5 equiv) was preactivated with HATU (1.0 equiv), HOAt (1.5 equiv) and DIPEA (4 equiv) in DMF (3 mL) and the resulting solution was then added to the reaction vessel containing resin and shaken for overnight at room temperature. The resin 25 was washed subsequently with DMF (2×10 mL), methanol (2×10 mL), and dichloromethane (2×10 mL). The completion of reaction was confirmed by negative Kaiser test.

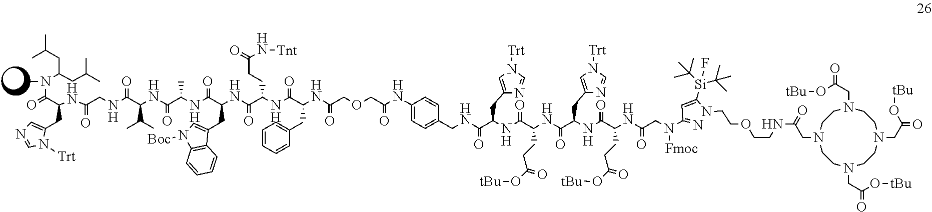

DOTA-tris(t-Bu)-COOH coupling. Resin 25 (1.2 mmol/g, 0.65 mmol) was treated twice with 1:2 mixture of piperidine and DMF (3 mL) to deprotect Fmoc-group followed by subsequent washings with DMF, methanol and dichloromethane (positive Kaiser test). DOTA-tris(t-Bu)-COOH (1.5 equiv) was preactivated with HATU (1.5 equiv), HOAt (1.5 equiv) and DIPEA (4 equiv) in DMF (3 mL) and the resulting solution was then added to the reaction vessel containing resin and shaken for overnight at room temperature. The obtained resin 26 was washed subsequently with DMF (2×10 mL), methanol (2×10 mL), and dichloromethane (2×10 mL). The completion of reaction was confirmed by negative Kaiser test.

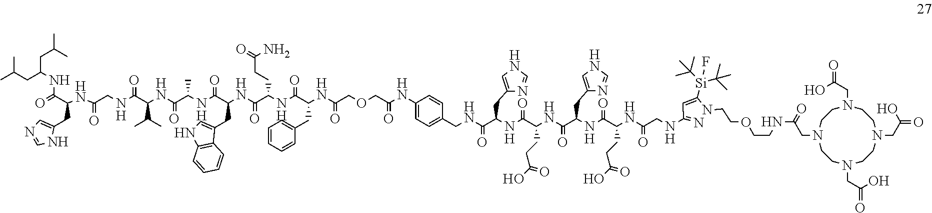 [SMILES: CC(C)CC(CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)COCC(=O)Nc1ccc(CNC(=O)[C@@H](Cc2c[nH]cn2)NC(=O)[C@@H](CCC(=O)O)NC(=O)[C@@H](Cc2c[nH]cn2)NC(=O)[C@@H](CCC(=O)O)NC(=O)CNc2cc([Si](F)(C(C)(C)C)C(C)(C)C)n(CCOCCNC(=O)CN3CCN(CC(=O)O)CCN(CC(=O)O)CCN(CC(=O)O)CC3)n2)cc1)C(C)C]

Peptide deprotection and removal from resin. Synthesis of 2,2′,2″-(10-(2-((2-(2-(3-(((4R,7R,10R,13R)-4,10-bis((1H-imidazol-4-yl)methyl)-1-(4-((7R,10S,13S,16S,19S,25S)-25-((1H-imidazol-5-yl)methyl)-13-((1H-indol-3-yl)methyl)-10-(3-amino-3-oxopropyl)-7-benzyl-28-isobutyl-19-isopropyl-16,30-dimethyl-5,8,11,14,17,20,23,26-octaoxo-3-oxa-6,9,12,15,18,21,24,27-octaazahentriacontanamido)phenyl)-7,13-bis(2-carboxyethyl)-3,6,9,12,15-pentaoxo-2,5,8,11,14-pentaazahexadecan-16-yl)amino)-5-(di-tert-butylfluorosilyl)-1H-pyrazol-1-yl)ethoxy)ethyl)amino)-2-oxoethyl)-1,4,7,10-tetraazacyclododecane-1,4,7-triyl)triacetic acid (27).

Resin 26 from previous step was treated overnight at room temperature with premade deprotection cocktail (15 mL, 446 μmol) as below. The solvent was collected by filtration and its amount was reduced on rotavapor. The peptide was precipitated by adding cold diethyl ether, centrifuged and the solid was washed twice with cold diethyl ether to obtain desired product 27. It was used as such for next step.

Deprotection cocktail was prepared by mixing following reagents: [88% TFA+5% water+5% Phenol+2% Triisopropylsilane].

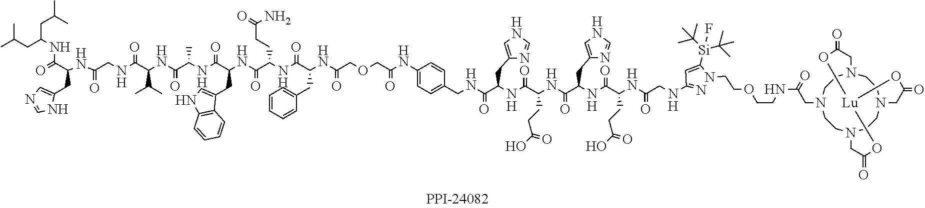

Formation of Lutetium chelate (PPI-24082). To a solution of 27 (69.0 mg, 20.2 μmol) in DMSO (1.38 mL) was added aqueous LuCl₃ (552 μL, 200 mM) and the mixture was stirred at 90° C. for 1 h. Reaction was then stopped, cooled to room temperature, and directly purified using preparative HPLC eluting with 0.1% TFA and acetonitrile gradient. Pure product fractions collected in 30% acetonitrile were combined and lyophilized overnight to obtain desired product PPI24082 (1.04 mg, yield to be optimized).

LCMS (method 8B): EM=C₁₁₆H₁₆₆FLuN₃₀O₂₈Si; Calc. for [M+2H]²⁺=1325.6, found=1325.5; Rt=2.79 min.

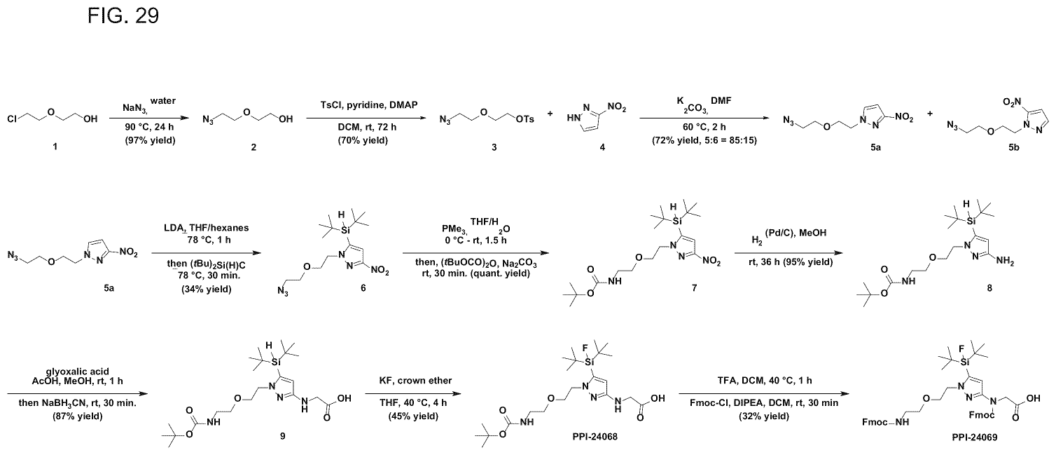

FIG. 29 illustrates a synthesis scheme of PPI-24069 in accordance with an embodiment of the invention.

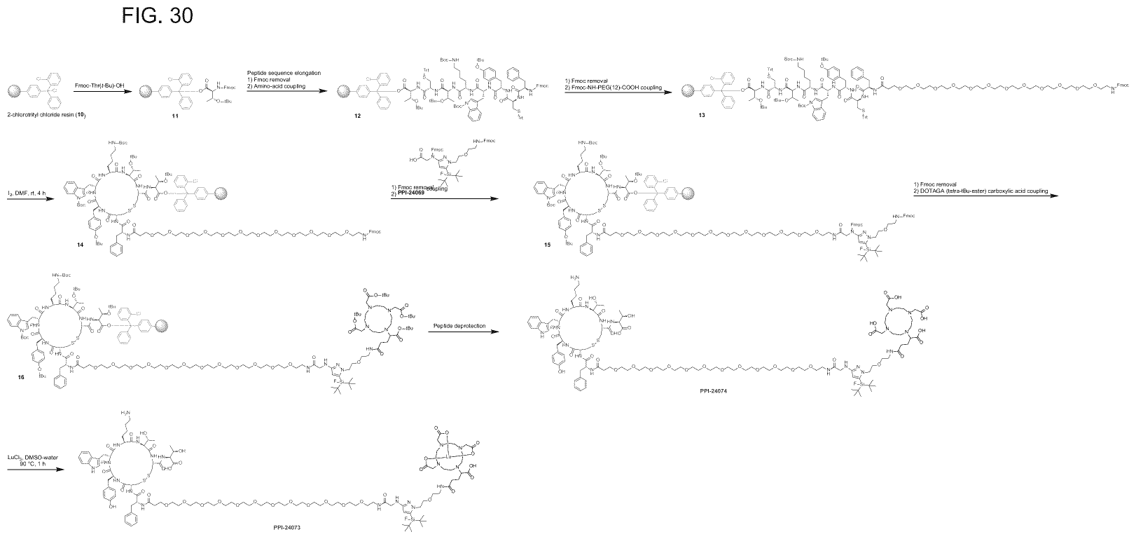

FIG. 30 illustrates a synthesis scheme of PPI-24073/FTX-181 in accordance with an embodiment of the invention.

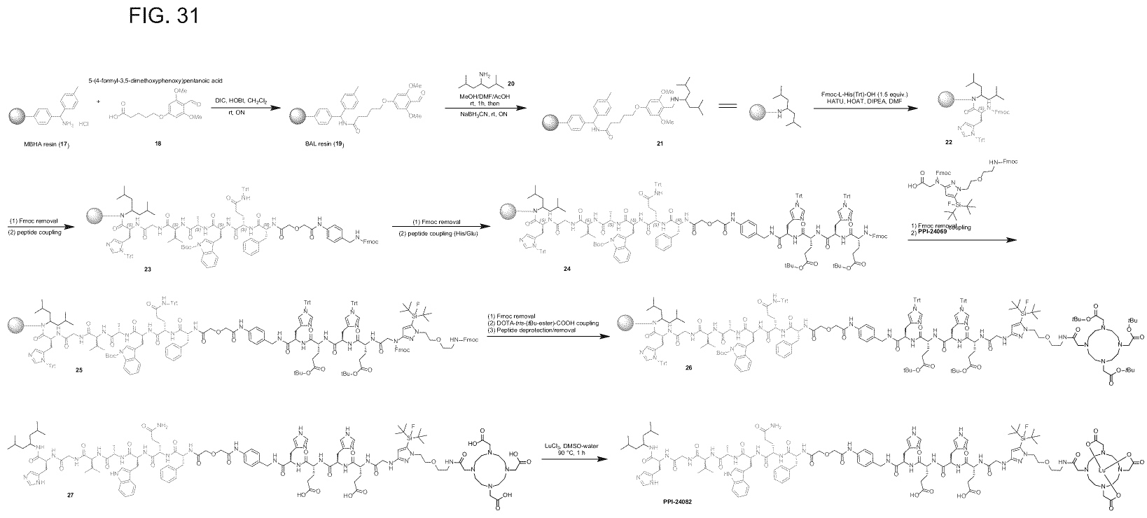

FIG. 31 illustrates a synthesis scheme of PPI-24082/FTX-219 in accordance with an embodiment of the invention.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A compound comprising:
a silicon-fluoride acceptor; and
a heteroaromatic ring;
wherein the heteroaromatic ring is selected from the group consisting of: pyridine, pyridine oxide, pyridinium, pyrazole, fused pyrazole derivative, benzofuran, benzothiophene, indole, azaindole, imidazole, and pyrimidine;
wherein the pyridine, pyridine oxide, or pyridinium compound has a formula I of: 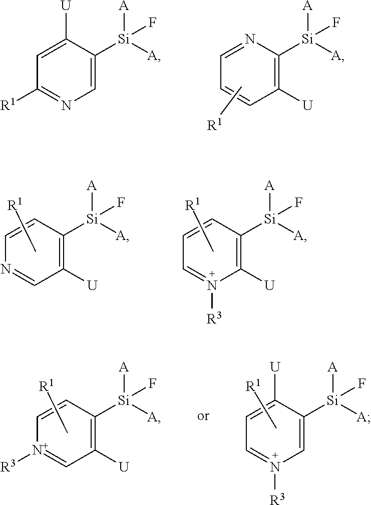
wherein the pyrazole compound has a formula II of: 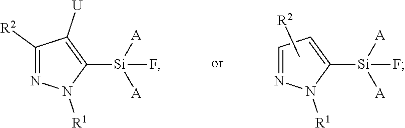
wherein the fused pyrazole derivative compound has a formula III of: 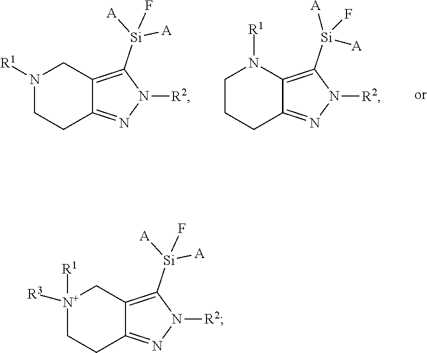
wherein the benzofuran, benzothiophene, indole, or azaindole compound has a formula IV of: 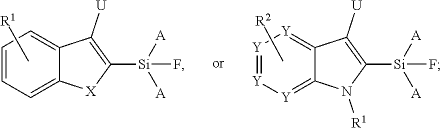
wherein the imidazole compound as a formula of: 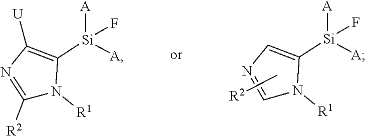
wherein the pyrimidine compound has a formula VI of 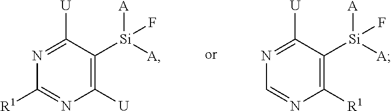
wherein:
each F is independently: F, or ¹⁸F, or ¹⁹F;
each A is independently: H, CH₃, CH₂—CH₃, CH₃—CH₂—CH(CH₃), CH(CH₃)₂, and C(CH₃)₃;
each U is independently: O—CH₃, CH₃, CH₂CH₃, H, I, Br, Cl, F, N(CH₃)₂ and CH₂CH(NH₂)CO₂H;
each X is independently: O, S, and N;
each Y is independently: C and N;
each R¹ or R² or R³ is independently: CH₃, CH₂—CH₃, H, L¹-CH₂—C≡C, L¹-CH₃, L¹-G, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²-L³-L⁴-G, L¹-L²-L³-L⁴-H, L¹-L²-L³-L⁴-Q-L⁵-G, L¹-L²-L³-Q, L¹-L²-L³-Q-L⁴-G, L¹-L²-N₃, L¹-L²-OH, L¹-L²-Q, L¹-L²-Q-G, L¹-L²-Q-L³-G, L¹-L²-Q-L³-L⁴-G, L¹-L²-Q-L³-L⁴-H, L¹-OH, L¹-Q, L¹-Q-L²-L³-L⁴-G, L¹-Q-L²-G, NH₂, O⁻, O—CH₃, OH, 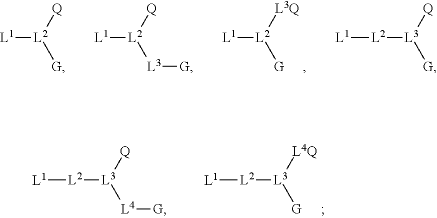
each L¹ or L² or L³ or L⁴ or L⁵ is independently: —(O—CH₂—CH₂)ₚ—, —(CH₂—CH₂—O)ₚ—, -(Glu-His)ₚ-, -(His-Glu)ₚ-, -(Glu-Trp)ₚ-, -(Trp-Glu)ₚ-, —NH—CH₂—C₆H₄—NH—(C═O)—CH₂—O—CH₂—(C═O)—, —(C═O)—CH₂—O—CH₂—(C═O)—NH—C₆H₄—CH₂—NH—, —(C═O)—CH₂—CH₂—(C═O)—, —NH—C₅H₉N—CH₂—(C═O)—, —(C═O)—CH₂—NC₅H₉—NH—, -(Gly)ₚ-, —CH₂—CH₂—NH—, —NH—CH₂—(C═O)—, -(Glu)-, —NH—CH₂—CH₂—, —(C═O)—, NH—CH₂—CH₂—(O—CH₂—CH₂)ₚ—(C═O)—, —(C═O)—(CH₂—CH₂—O)ₚ—CH₂—CH₂—NH—, —NH—(C═O)—CH₂—, —NH—(C═O)—NH—CH₂—, —NH—(C═S)—NH—CH₂—, —NH—(CH₂—CH₂—O)ₚ—CH₂—(C═O)—, —(C═O)—CH₂—(O—CH₂—CH₂)ₚ—NH—, -Asp-, —NH—, —NH—(CH₂—CH₂—O)ₚ—CH₂—CH₂—(C═O)—, —(C═O)—CH₂—CH₂—(O—CH₂—CH₂)ₚ—NH—, —CH₂—(C═O)—, —(C═O)—CH₂—, —O—(C═O)—, —(C═O)—O—, —CH₂—O—, —NH—(C═O)—O—, —O—(C═O)—NH—, —(C═O)—CH₂—(O—CH₂—CH₂)ₚ—O—CH₂—(C═O)—, —NH—(C═O)—NH—, —NH—(C═S)—NH—, —CH₂—N(CH₃)—CH₂—, —N(CH₃)—, —(C═O)—C₆H₄—(C═O)—, —C₆H₄—(C═O)—, —(C═O)—C₆H₄—, —O—CH₂—(C═O)—, —(C═O)—CH₂—O—, —CH₂—C₂N₃—CH₂—, —CH₂—CH₂—(C═O)—, —(C═O)—CH₂—CH₂—, —CH₂—N⁺(CH₃)₂—CH₂—, —CH₂—N(CH₃)—, —(C═O)—CH₂—(O—CH₂—CH₂)ₚ—, —(CH₂—CH₂—O)ₚ—CH₂—CH₂—NH—, or 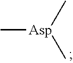
p=0 to 12;
each G is a disease binding ligand, wherein G is independently: a somatostatin receptor type 2 (SSTR2) binding ligand, a gastrin releasing peptide receptor (GRPR) binding ligand, a prostate specific membrane antigen (PSMA) binding ligand, a fibroblast activation protein (FAP) binding ligand, or a C-X-C chemokine receptor type 4 (CXCR-4) binding ligand; and
each Q is a chelator.

2. The compound of claim 1, wherein each Q is independently: -DOTA, -DOTAGA, -Dap(DOTA), -Lys(DOTA), -3p-C-NETA, -bis-thioseminarabazones, -EDTA, -CHX-A″-EDTA, -DTPA, -p-SCN-DPTA, -CHX-A″-DTPA, -p-SCN-Bz-Mx-DTPA, -NOTA, -TETA, -CB-TE2A, -p-SCN-NOTA (cNOTA), -nNOTA, -NODAGA, -p-SCN-DOTA (cDOTA), -2-cTETA, -6-cTETA, -BAT, -Diamsa, -SarAr, -PCTA, -NODIA-Me, -TRAP, -pycup1A1B, -p-SCN-DTPA, -Desferrioxamine B(DFO) Mesylate, -Desferrioxamine-p-SCN, -DFO-Star (DFO*), -L5, -Orn3hx-NCS, -Orn4hx-NCS, -p-SCN-Bn-HOPO, -2,3-HOPO-p-Bn-NCS, -YM103, -Tc(V)oxo, -Tc(V)nitride, -Tc(V)HYNIC, -Tc(I)-fac-tricarbonyl, -Tc(VII)trioxo, -3p-C-NETA-NCS, -3p-C-DEPA-NCS, -TCMC, -p-SCN-Bn-H₄octapa, -HEHA-NCS, or -Macropa-NCS.

3. The compound of claim 1, wherein Q is unchelated or optionally chelated with M, wherein M is a cation of a metal selected from the group consisting of: ⁴³Sc, ⁴⁴Sc, ⁴⁵Sc, ⁴⁷Sc, ⁵¹Cr, ^{52m}Mn, ⁶⁸Co, ⁵²Fe, ⁵⁶Ni, ⁵⁷Ni, ⁶¹Cu, ⁶²Cu, ⁶³Cu, ⁶⁴Cu, ⁶⁵Cu, ⁶⁷Cu, ⁶⁶Ga, ⁶⁷Ga, ⁶⁸Ga, ⁶⁹Ga, ⁷¹Ga, ^{nat}Ga, ⁹⁰Zr, ⁹¹Zr, ⁹²Zr, ⁸⁹Zr, ⁸⁶Y, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹I, ¹¹³In, ^{133m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵⁵Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁶Lu, ¹⁷⁷Lu, ^{177m}Lu, ^{nat}Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²⁰⁴Pb, ²⁰⁶Pb, ²⁰⁷Pb, ²⁰⁸Pb, ²¹¹At, ²⁰⁹Bi, ²¹²Bi, ²¹³Bi, ²²³Ra, ²²⁵Ac, ²²⁷Th, ²³²Th, and a cationic molecule comprising ¹⁸F, and ¹⁸F[AlF]²⁺.

4. The compound of claim 1, wherein A is C(CH₃)₃.

5. The compound of claim 1, wherein G has a formula selected from the group consisting of: 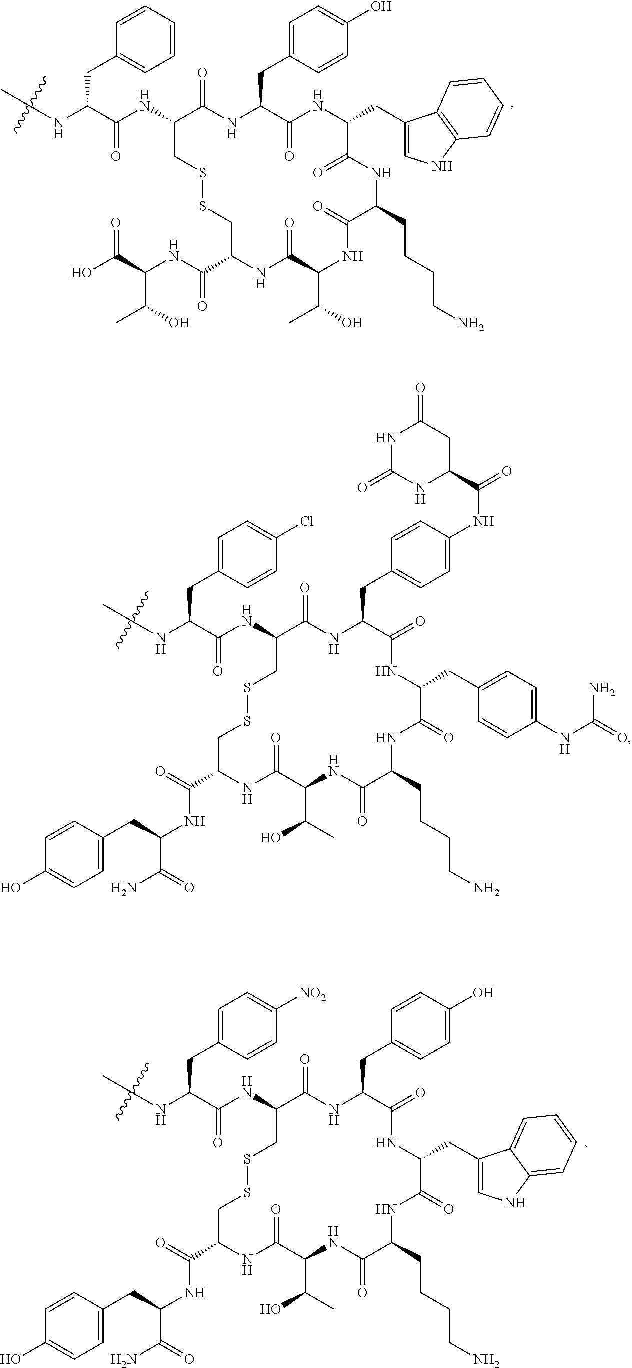 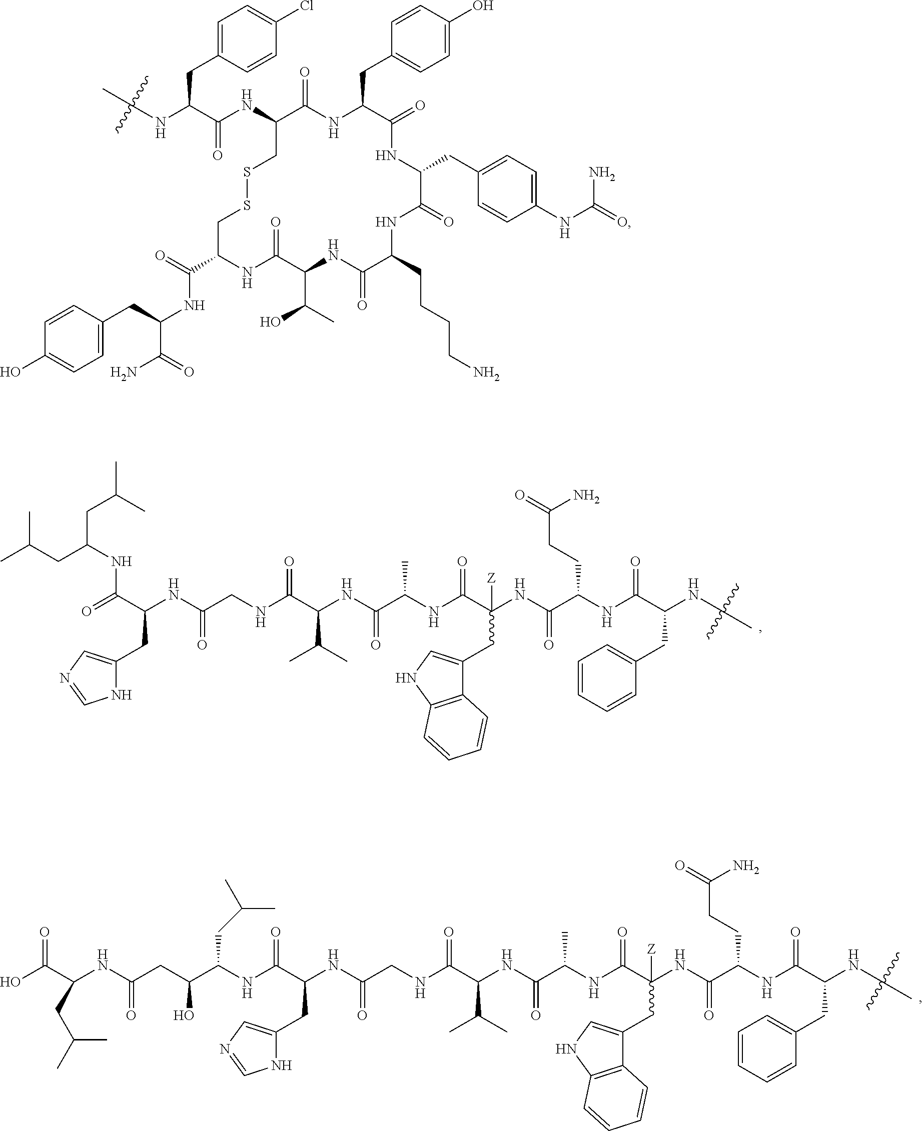 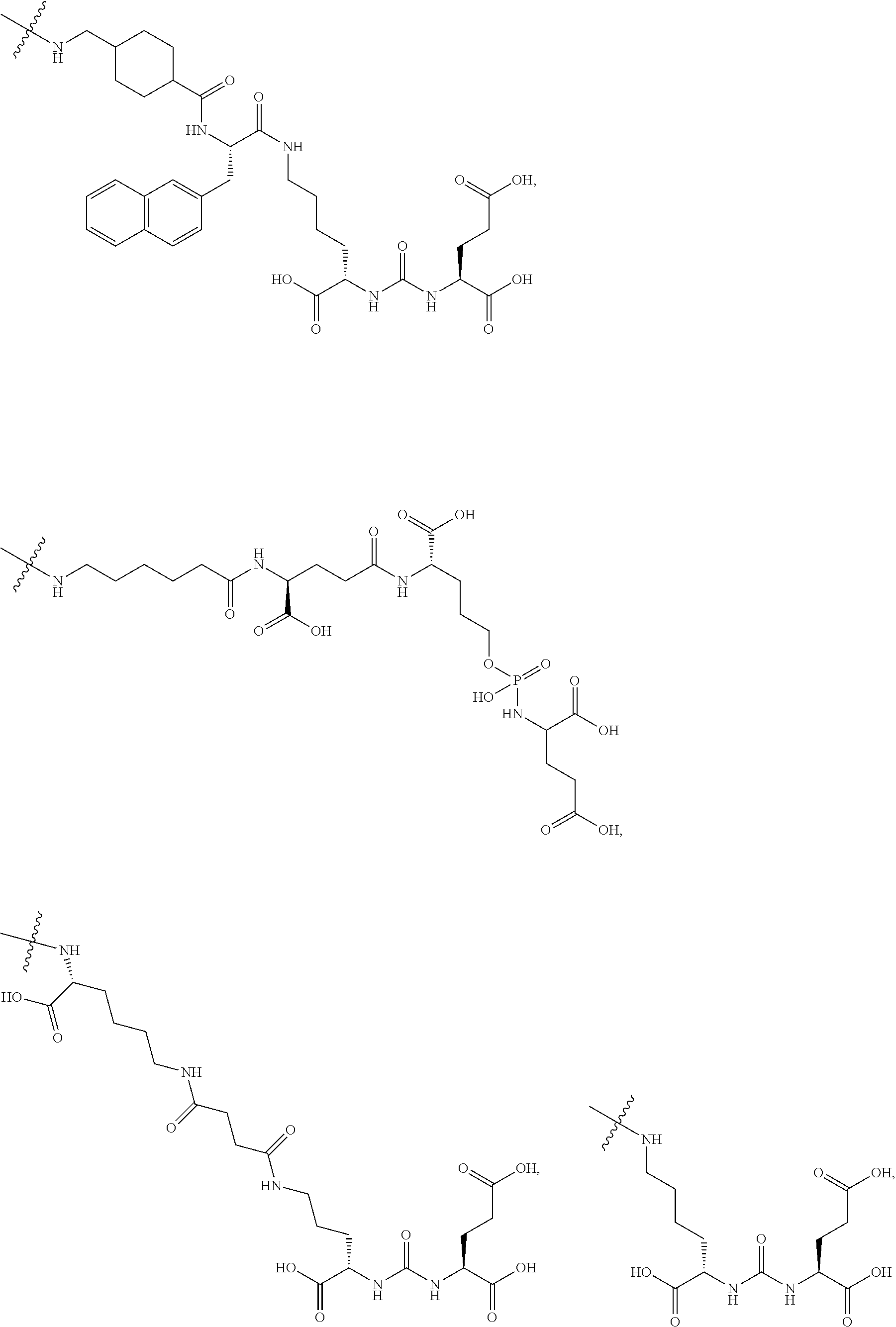 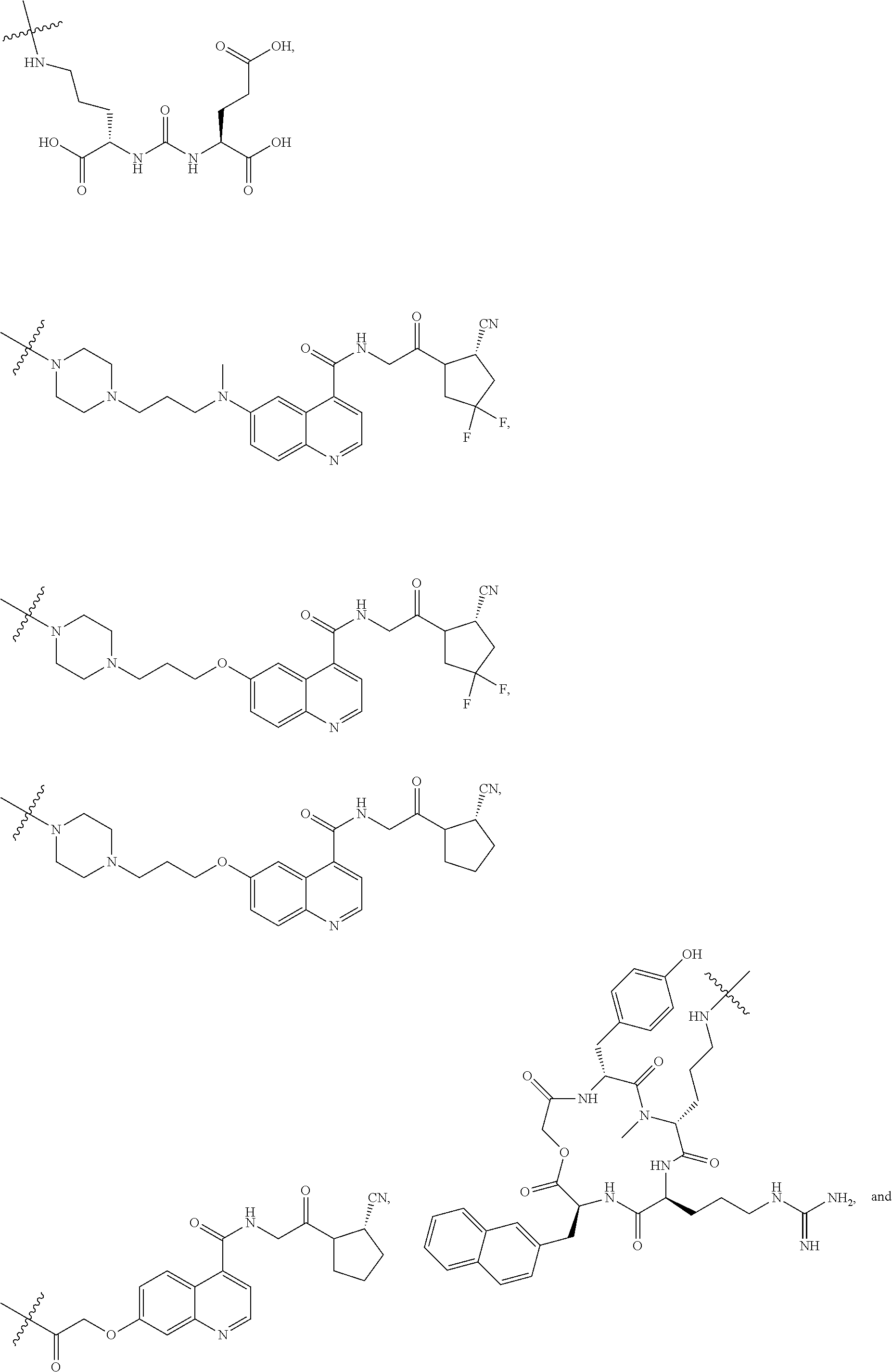 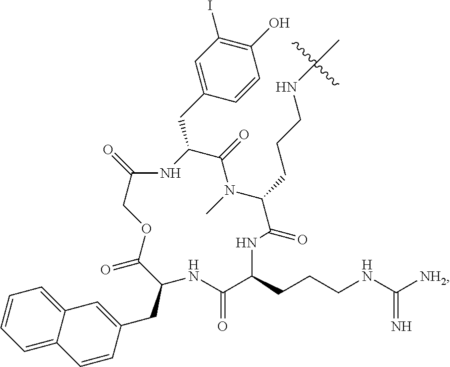
wherein Z is H, CH₃ or CF₃.

6. The compound of claim 1, wherein:
R¹ is independently: CH₃, H, L¹-CH₂—C≡C, L¹-CH₃, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²-L³-L⁴-Q-L⁵-G, L¹-L²-L³-Q, L¹-L²-L³-Q-L⁴-G, L¹-L²-N₃, L¹-L²-OH, L¹-L²-Q, L¹-L²-Q-L³-L⁴-G, L¹-OH, L¹-Q, L¹-Q-L²-L³-L⁴-G, NH₂, O—CH₃, OH, 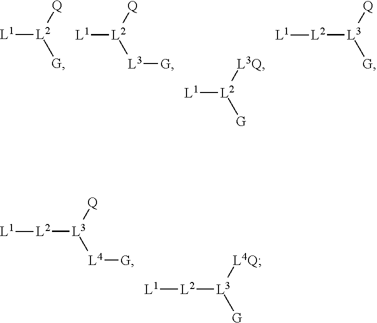
R² is independently: CH₃, H, L¹-CH₂—C≡C, L¹-G, L¹-H, L¹-L²-CH₂—C≡C, L¹-L²-G, L¹-L²-L³-G, L¹-L²-L³-L⁴-G, L¹-L²-L³-Q, L¹-L²-L³-Q-L⁴-G, L¹-L²-N₃, L¹-L²-Q, L¹-L²-Q-G, L¹-L²-Q-L³-G, L¹-OH, L¹-Q, L¹-Q-L²-G, NH₂, O⁻, or O—CH₃; and
R³ is independently: CH₃, CH₂—CH₃, or O⁻.

7. The compound of claim 1, wherein the compound is a small molecule with a formula selected from the group consisting of:

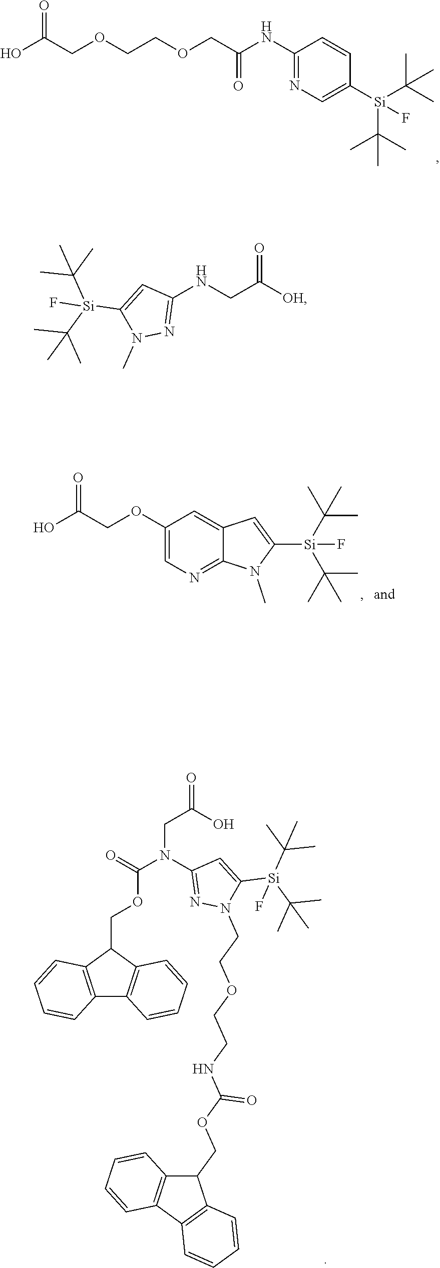

8. The compound of claim 1, wherein the compound is for positron emission tomography (PET) imaging and has a formula of:

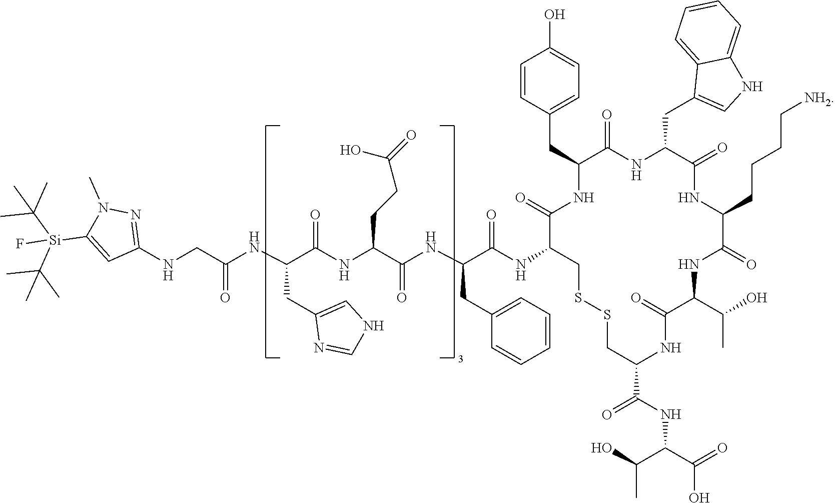

9. The compound of claim 1, wherein the compound is a theranostic compound with a formula selected from the group consisting of:

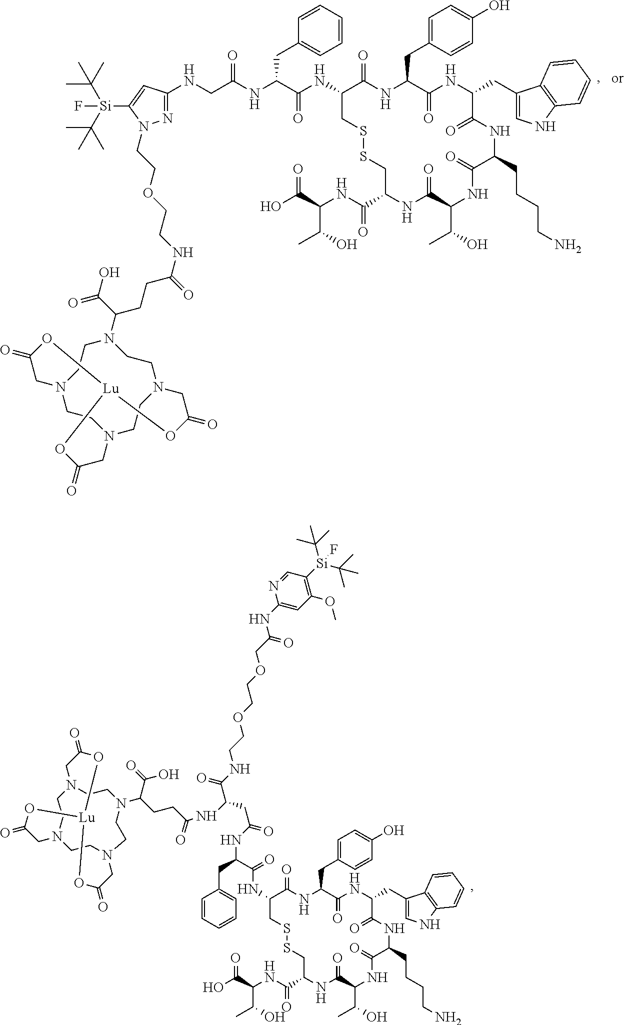

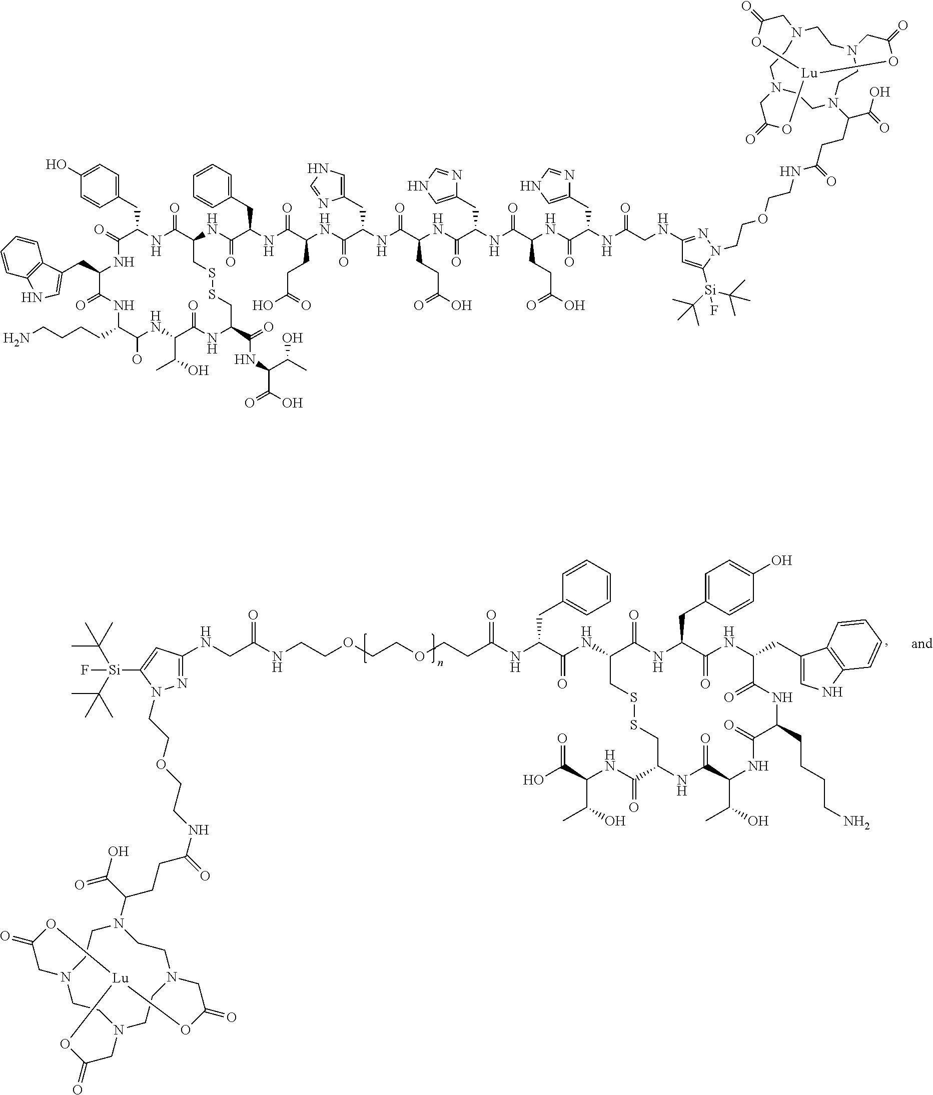

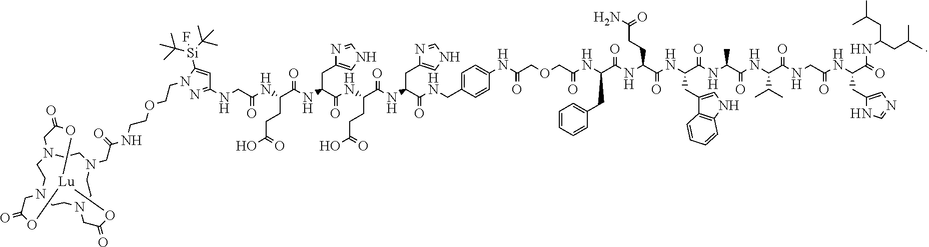

10. The compound of claim 1, wherein the compound is a dual targeting theranostic compound with a formula of:

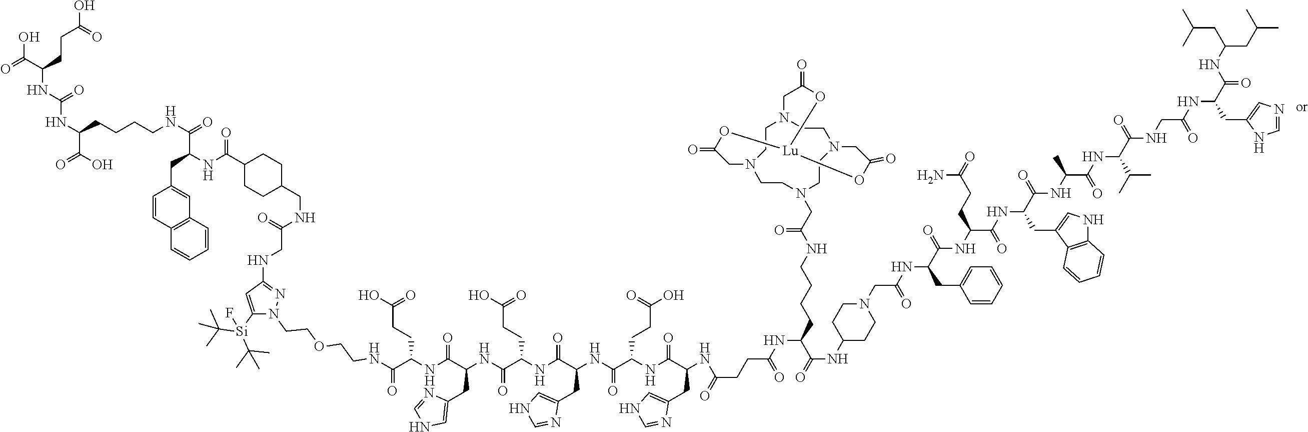

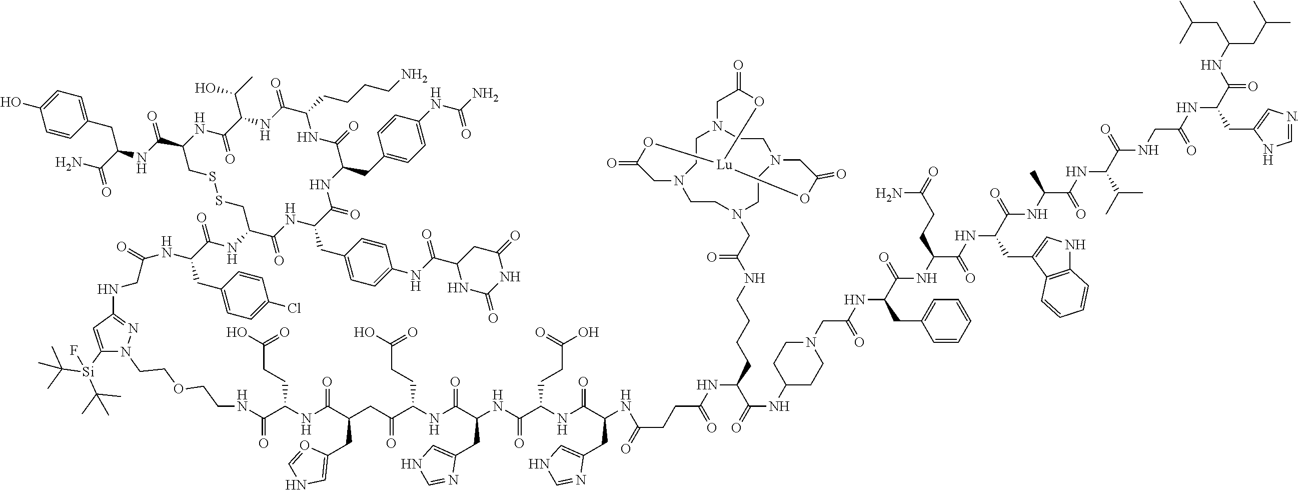

11. The compound of claim 1, wherein the compound is configured for PET imaging and therapeutic radioligand therapy.

12. The compound of claim 1, wherein the disease binding ligand G is conjugated to the compound via a process selected from the group consisting of: coupling chemistry, peptide coupling chemistry, copper-catalyzed azide-alkyne cycloaddition (CuAAC), and solid-phase peptide synthesis (SPPS).

13. The compound of claim 2, wherein:
3p-C-NETA is configured to chelate with ¹⁷⁷Lu for therapeutics;
DOTA and DOTAGA are configured to chelate with ¹⁷⁷Lu or ²²⁵Ac for therapeutics; and
DOTA and DOTAGA are configured to chelate with ⁶⁸Ga, ⁸⁹Zr, or ⁶⁴Cu for PET imaging.

14. A method for synthesizing a radiopharmaceutical compound comprising:
providing a silicon fluoride compound comprising a heteroaromatic ring;
conjugating a disease binding ligand G to the silicon fluoride compound to form the radiopharmaceutical compound;
wherein the heteroaromatic ring is selected from the group consisting of: pyridine, pyridine oxide, pyridinium, pyrazole, fused pyrazole derivative, benzofuran, benzothiophene, indole, azaindole, imidazole, and pyrimidine;
wherein the pyridine, pyridine oxide, or pyridinium compound has a formula I of: 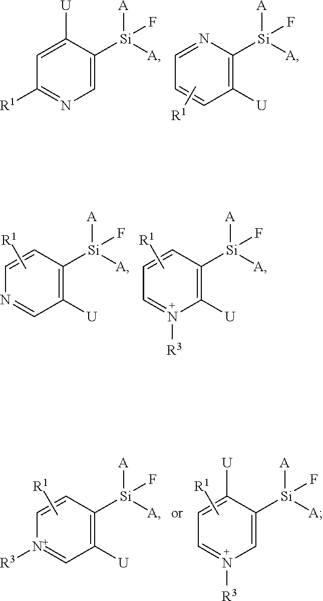
wherein the pyrazole compound has a formula II of: 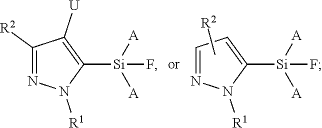
wherein the fused pyrazole derivative compound has a formula III of: 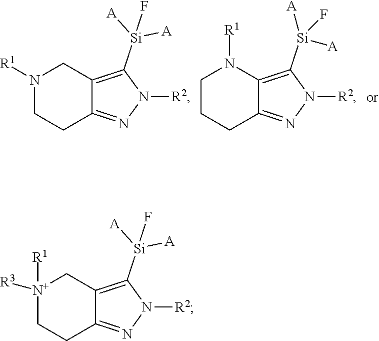
wherein the benzofuran, benzothiophene, indole, or azaindole compound has a formula IV of: 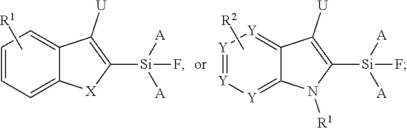
wherein the imidazole compound has a formula V of: 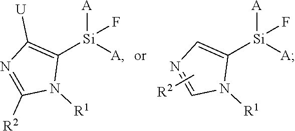
wherein the pyrimidine compound has a formula VI of: 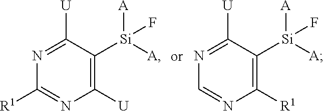
wherein:
each F is independently: F, or ¹⁸F, or ¹⁹F;
each A is independently: H, CH₃, CH₂—CH₃, CH₃—CH₂—CH(CH₃), CH(CH₃)₂, and C(CH₃)₃;
each U is independently: O—CH₃, CH₃, CH₂CH₃, H, I, Br, Cl, F, N(CH₃)₂ and CH₂CH(NH₂)CO₂H;
each X is independently: O, S, and N;
each Y is independently: C and N;
each R¹ or R² or R³ is independently: CH₃, CH₂—CH₃, H, L¹-CH₂—C≡C, L¹-CH₃, L¹-G, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²-L³-L⁴-G, L¹-L²-L³-L⁴-H, L¹-L²-L³-L⁴-Q-L⁵-G, L¹-L²-L³-Q, L¹-L²-L³-Q-L⁴-G, L¹-L²-N₃, L¹-L²-OH, L¹-L²-Q, L¹-L²-Q-G, L¹-L²-Q-L³-G, L¹-L²-Q-L³-L⁴-G, L¹-L²-Q-L³-L⁴-H, L¹-OH, L¹-Q, L¹-Q-L²-L³-L⁴-G, L¹-Q-L²-G, NH₂, O⁻, O—CH₃, OH, 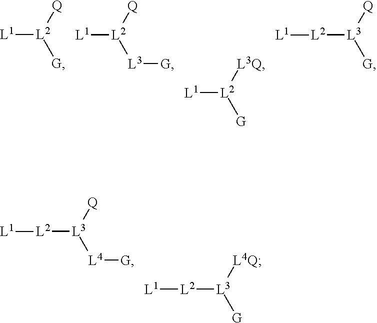
each L¹ or L² or L³ or L⁴ or L⁵ is independently: —(O—CH₂—CH₂)ₚ—, —(CH₂—CH₂—O)ₚ—, (Glu-His)ₚ-, -(His-Glu)ₚ-, -(Glu-Trp)ₚ-, -(Trp-Glu)ₚ-, —NH—CH₂—C₆H₄—NH—(C═O)—CH₂—O—CH₂—(C═O)—, —(C═O)—CH₂—O—CH₂—(C═O)—NH—C₆H₄—CH₂—NH—, —(C═O)—CH₂—CH₂—(C═O)—, —NH—C₅H₉N—CH₂—(C═O)—, —(C═O)—CH₂—NC₅H₉—NH—, -(Gly)ₚ-, —CH₂—CH₂—NH—, —NH—CH₂—(C═O)—, -(Glu)-, —NH—CH₂—CH₂—, —(C═O)—, NH—CH₂—CH₂—(O—CH₂—CH₂)ₚ—(C═O)—, —(C═O)—(CH₂—CH₂—O)ₚ—CH₂—CH₂—NH—, —NH—(C═O)—CH₂—, —NH—(C═O)—NH—CH₂—, —NH—(C═S)—NH—CH₂—, —NH—(CH₂—CH₂—O)ₚ—CH₂—(C═O)—, —(C═O)—CH₂—(O—CH₂—CH₂)ₚ—NH—, -Asp-, —NH—, —NH—(CH₂—CH₂—O)ₚ—CH₂—CH₂—(C═O)—, —(C═O)—CH₂—CH₂—(O—CH₂—CH₂)ₚ—NH—, —CH₂—(C═O)—, —(C═O)—CH₂—, —O—(C═O)—, —(C═O)—O—, —CH₂—O—, —NH—(C═O)—O—, —O—(C═O)—NH—, —(C═O)—CH₂—(O—CH₂—CH₂)ₚ—O—CH₂—(C═O)—, —NH—(C═O)—NH—, —NH—(C═S)—NH—, —CH₂—N(CH₃)—CH₂—, —N(CH₃)—, —(C═O)—C₆H₄—(C═O)—, —C₆H₄—(C═O)—, —(C═O)—C₆H₄—, —O—CH₂—(C═O)—, —(C═O)—CH₂—O—, —CH₂—C₂N₃—CH₂—, —CH₂—CH₂—(C═O)—, —(C═O)—CH₂— CH₂—, —CH₂—N⁺(CH₃)₂—CH₂—, —CH₂—N(CH₃)—, —(C═O)—CH₂—(O—CH₂—CH₂)ₚ—, —(CH₂—CH₂—O)ₚ—CH₂—CH₂—NH—, or 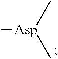
p=0 to 12;
each G is independently: a somatostatin receptor type 2 (SSTR2) binding ligand, a gastrin releasing peptide receptor (GRPR) binding ligand, a prostate specific membrane antigen (PSMA) binding ligand, a fibroblast activation protein (FAP) binding ligand, or a C-X-C chemokine receptor type 4 (CXCR-4) binding ligand; and
each Q is a chelator.

15. The method of claim 14, wherein each Q is independently: -DOTA, -DOTAGA, -Dap(DOTA), -Lys(DOTA), -3p-C-NETA, -bis-thioseminarabazones, -EDTA, -CHX-A″-EDTA, -DTPA, -p-SCN-DPTA, -CHX-A″-DTPA, -p-SCN-Bz-Mx-DTPA, -NOTA, -TETA, -CB-TE2A, -p-SCN-NOTA (cNOTA), -nNOTA, -NODAGA, -p-SCN-DOTA (cDOTA), -2-cTETA, -6-cTETA, -BAT, -Diamsa, -SarAr, -PCTA, -NODIA-Me, -TRAP, -pycup1A1B, -p-SCN-DTPA, -Desferrioxamine B(DFO) Mesylate, -Desferrioxamine-p-SCN, -DFO-Star (DFO*), -L5, -Orn3hx-NCS, -Orn4hx-NCS, -p-SCN-Bn-HOPO, -2,3-HOPO-p-Bn-NCS, -YM103, -Tc(V)oxo, -Tc(V)nitride, -Tc(V)HYNIC, -Tc(I)-fac-tricarbonyl, -Tc(VII)trioxo, -3p-C-NETA-NCS, -3p-C-DEPA-NCS, -TCMC, -p-SCN-Bn-H₄octapa, -HEHA-NCS, or -Macropa-NCS.

16. The method of claim 14, further comprising chelating Q with M, wherein M is a cation of a metal selected from the group consisting of: ⁴³Sc, ⁴⁴Sc, ⁴⁵Sc, ⁴⁷Sc, ⁵¹Cr, ^{52m}Mn, ⁶⁸Co, ⁵²Fe, ⁵⁶Ni, ⁵⁷Ni, ⁶¹Cu, ⁶²Cu, ⁶³Cu, ⁶⁴Cu, ⁶⁵Cu, ⁶⁷Cu, ⁶⁶Ga, ⁶⁷Ga, ⁶⁸Ga, ⁶⁹Ga, ⁷¹Ga, ^{nat}Ga, ⁹⁰Zr, ⁹¹Zr, ⁹²Zr, ⁸⁹Zr, ⁸⁶Y, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹I, ¹¹³In, ^{133m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵⁵Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁶Lu, ¹⁷⁷Lu, ^{177m}Lu, ^{nat}Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²⁰⁴Pb, ²⁰⁶Pb, ²⁰⁷Pb, ²⁰⁸Pb, ²¹¹At, ²⁰⁹Bi, ²¹²Bi, ²¹³Bi, ²²³Ba, ²²⁵Ac, ²²⁷Th, ²³²Th, and a cationic molecule comprising ¹⁸F, and ¹⁸F[AlF]²⁺.

17. The method of claim 14, wherein A is C(CH₃)₃.

18. The method of claim 14, wherein G has a formula selected from the group consisting of: 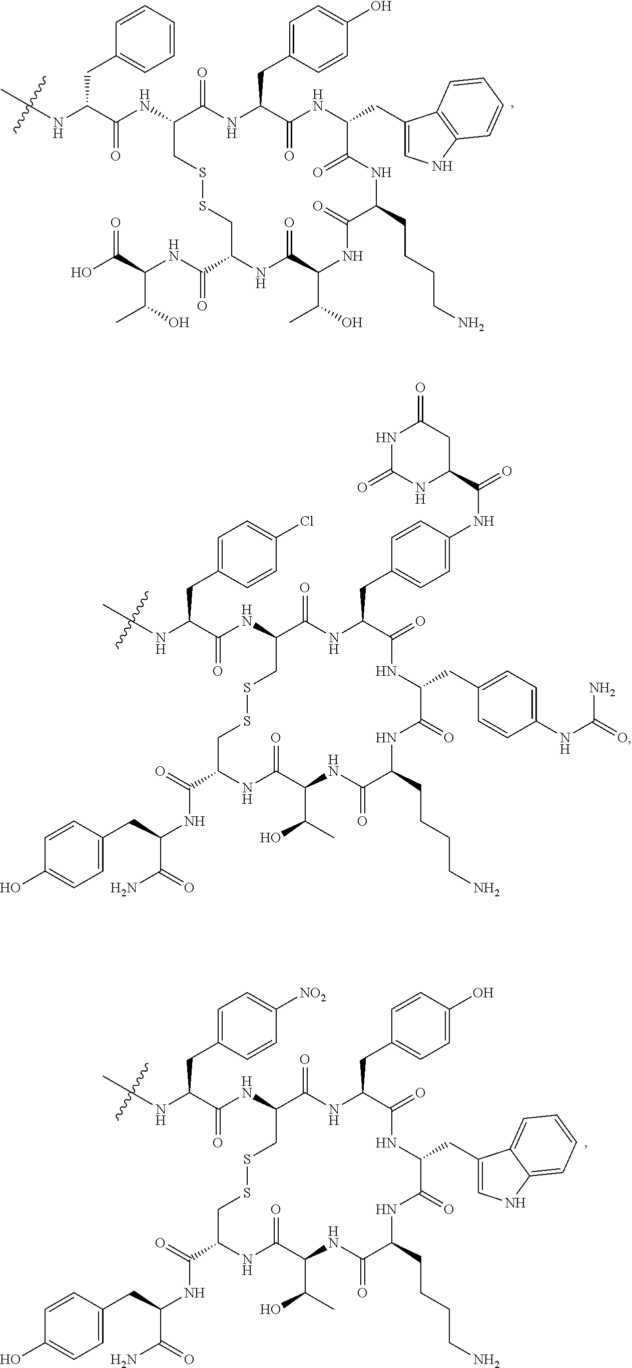 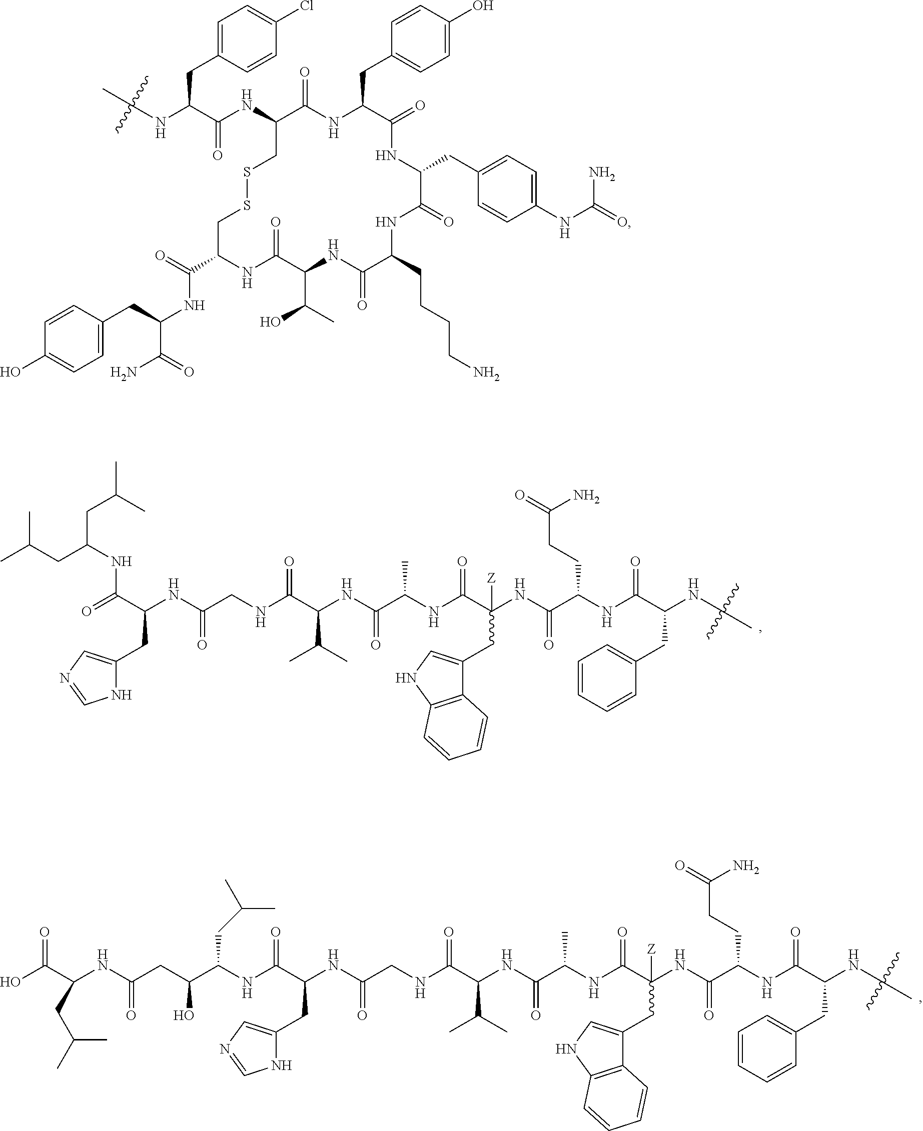 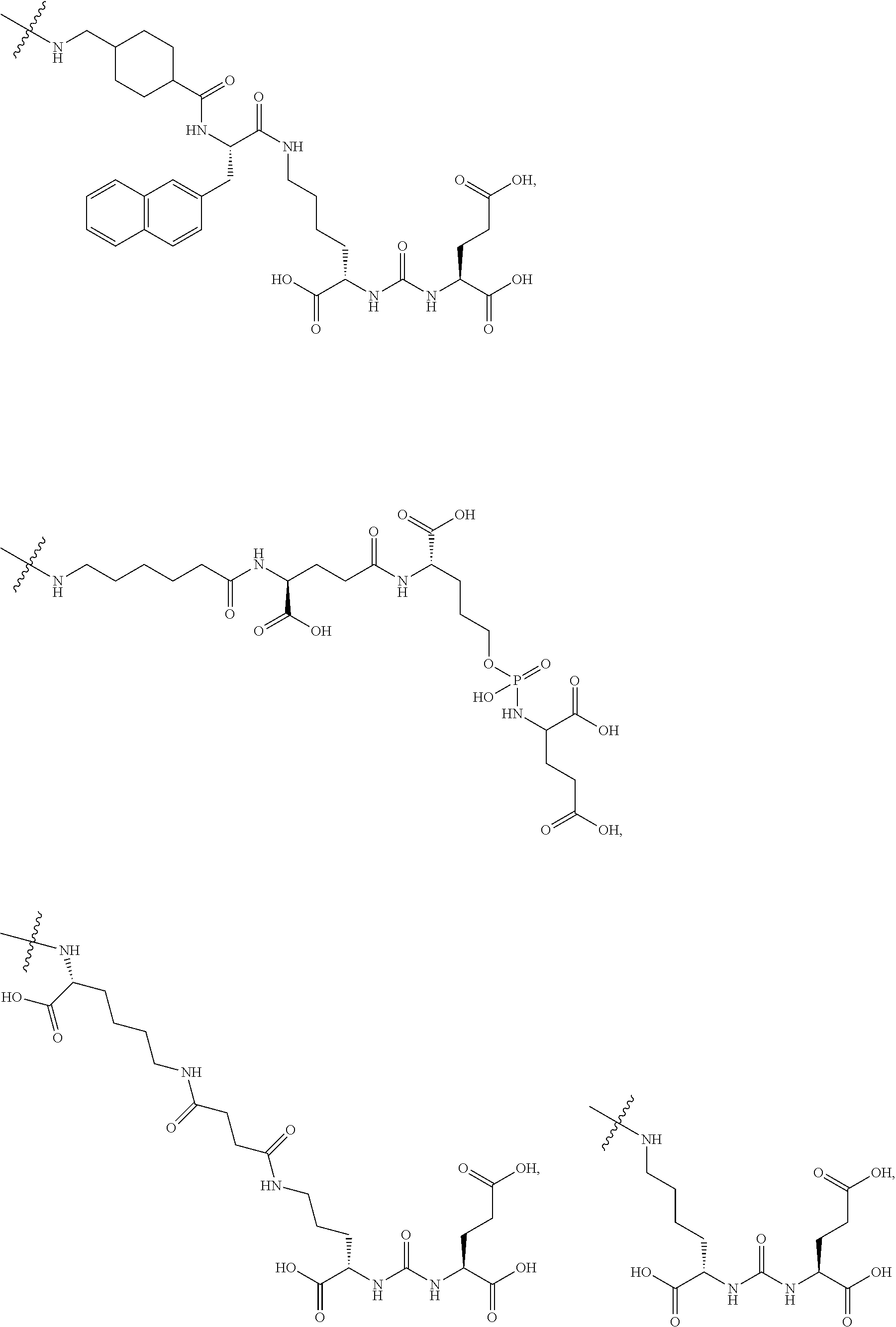 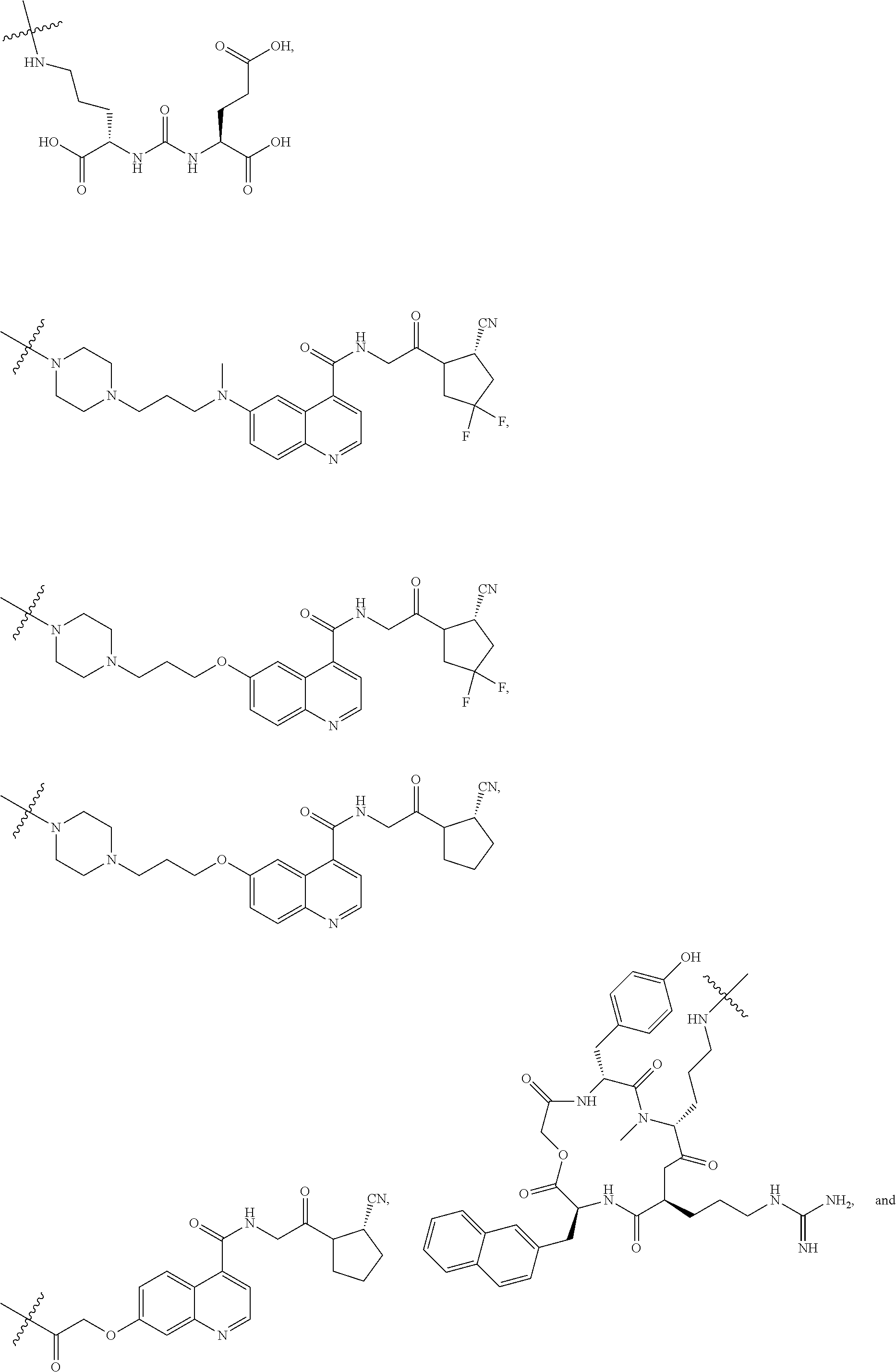 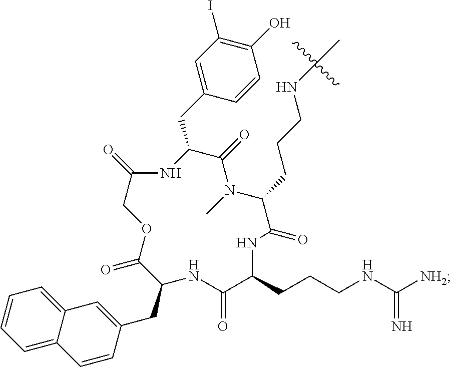
wherein Z is H, CH₃ or CF₃.

19. The method of claim 14, wherein:
R¹ is independently: CH₃, H, L¹-CH₂—C≡C, L¹-CH₃, L¹-H, L¹-L²-CH₂—C≡C, L¹-L²-CH₃, L¹-L²-G, L¹-L²-H, L¹-L²-H₂, L¹-L²-L³-G, L¹-L²-L³-H, L¹-L²-L³-L⁴-Q-L⁵-G, L¹-L²-L³-Q, L¹-L²-L³-Q-L⁴-G, L¹-L²-N₃, L¹-L²-OH, L¹-L²-Q, L¹-L²-Q-L³-L⁴-G, L¹-OH, L¹-Q, L¹-Q-L²-L³-L⁴-G, NH₂, O—CH₃, OH, 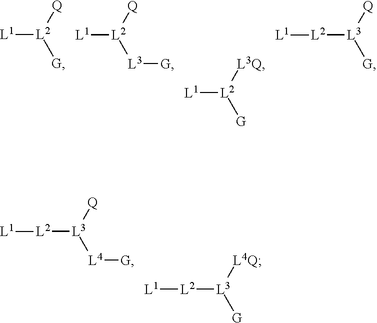
R² is independently: CH₃, H, L¹-CH₂—CC, L¹-G, L¹-H, L¹-L²-CH₂—C≡C, L¹-L²-G, L¹-L²-L³-G, L¹-L²-L³-L⁴-G, L¹-L²-L³-Q, L¹-L²-L³-Q-L⁴-G, L¹-L²-N₃, L¹-L²-Q, L¹-L²-Q-G, L¹-L²-Q-L³-G, L¹-OH, L¹-Q, L¹-Q-L²-G, NH₂, O⁻, or O—CH₃; and
R³ is independently: CH₃, CH₂—CH₃, or O⁻.

20. The method of claim 14, wherein the radiopharmaceutical compound is a small molecule with a formula selected from the group consisting of:

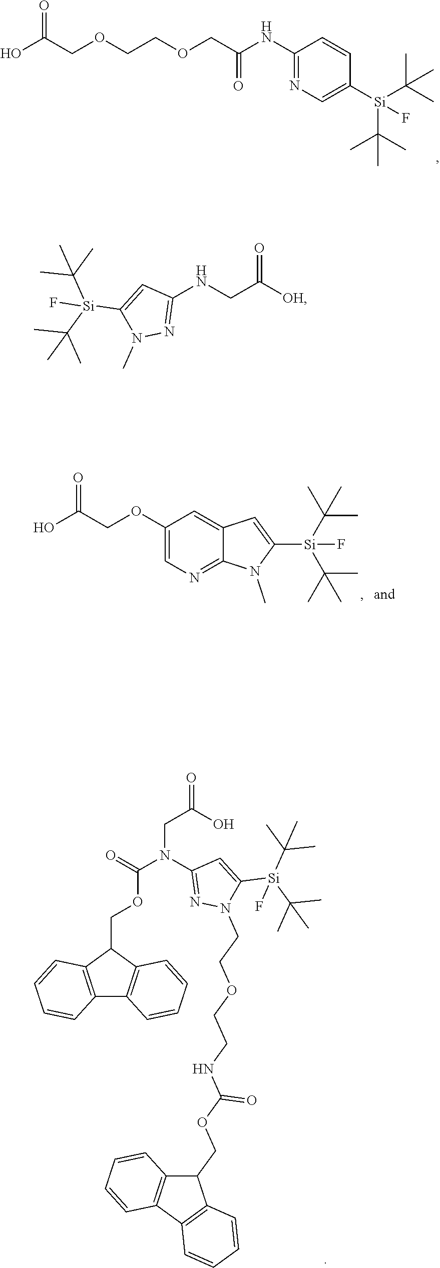

21. The method of claim 14, wherein the radiopharmaceutical compound is for positron emission tomography (PET) imaging and has a formula of:

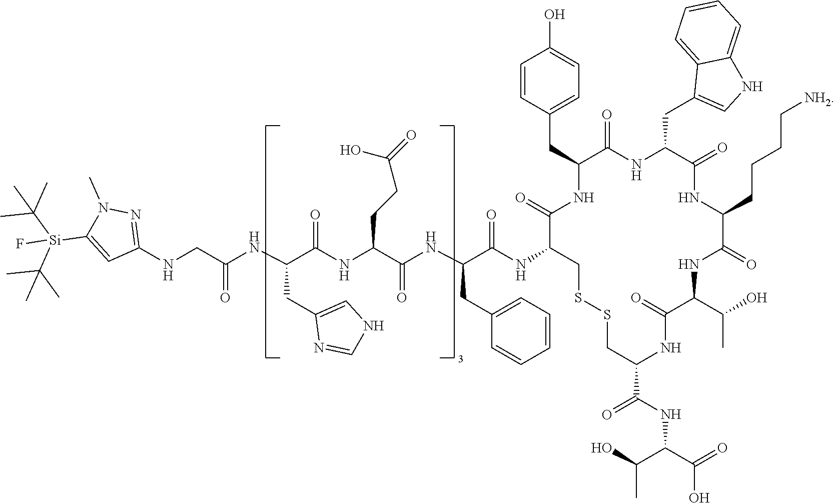

22. The method of claim 14, wherein the radiopharmaceutical compound is a theranostic compound with a formula selected from the group consisting of:

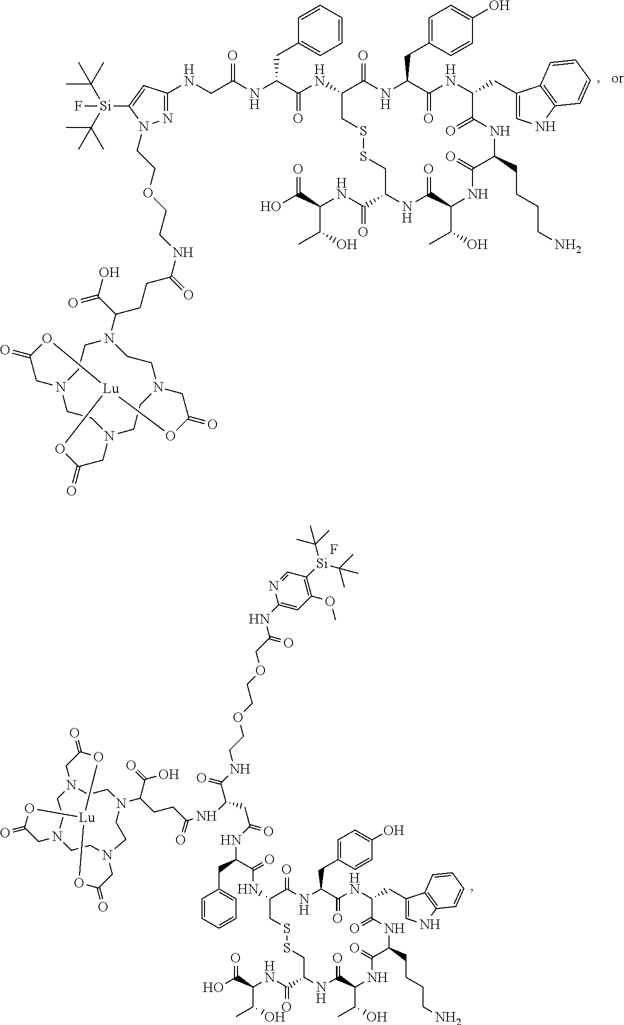

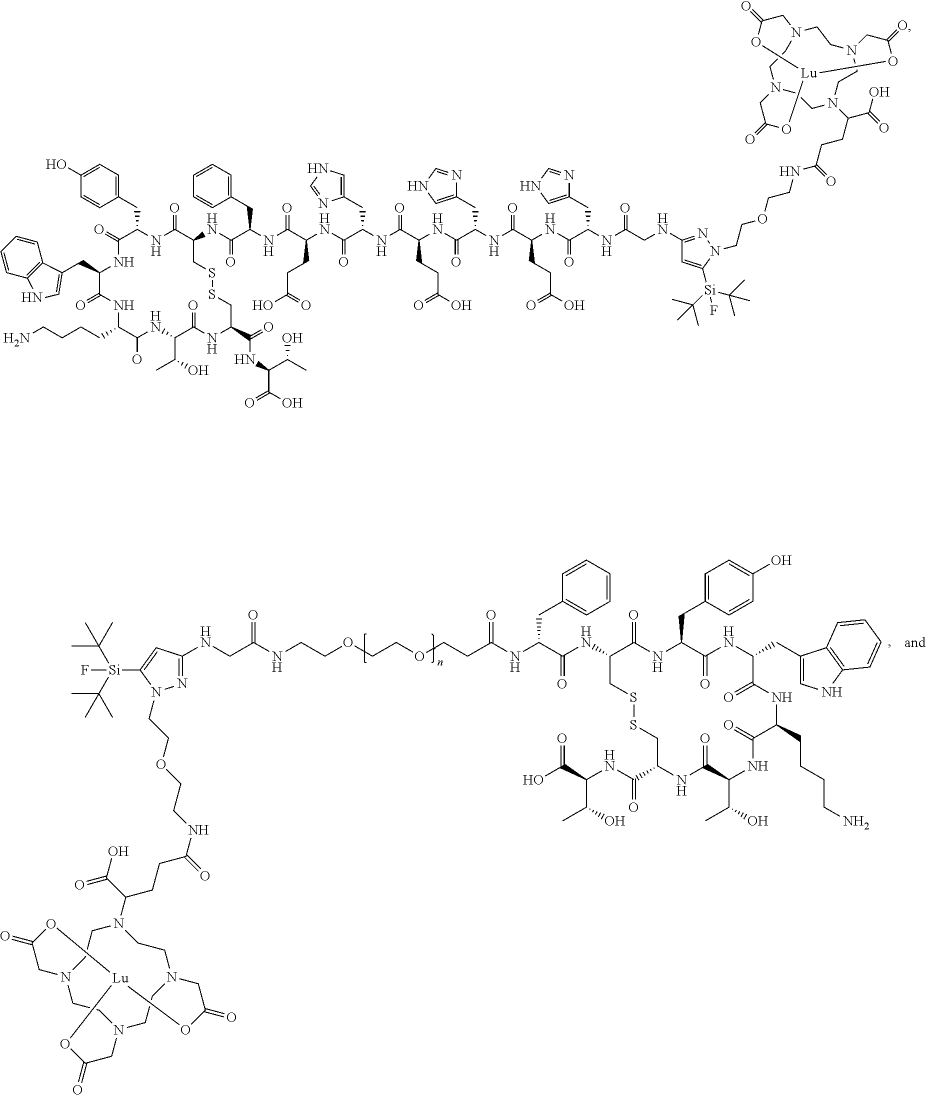

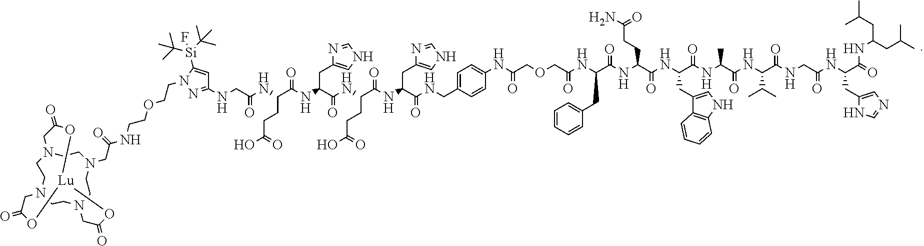

23. The method of claim 14, wherein the radiopharmaceutical compound is a dual targeting theranostic compound with a formula of:

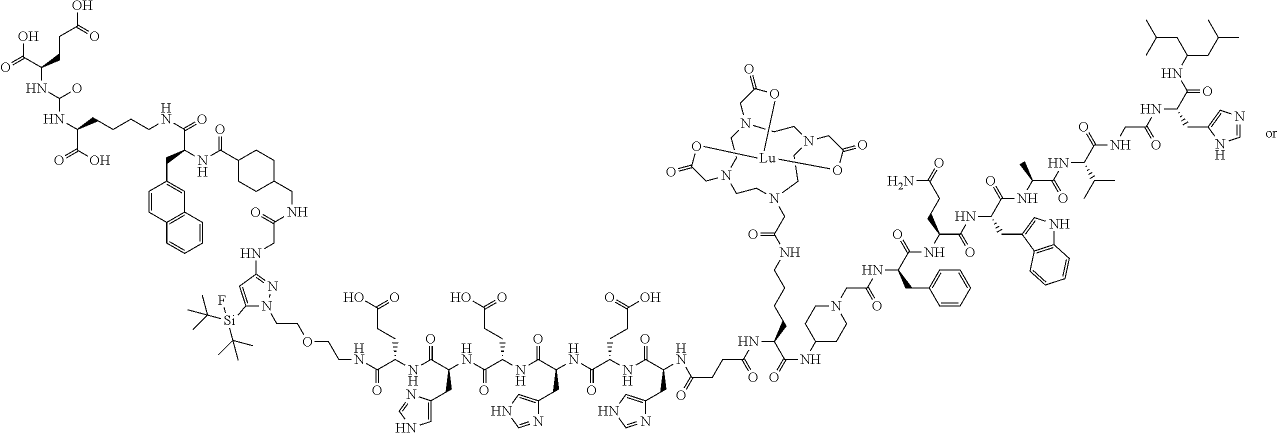

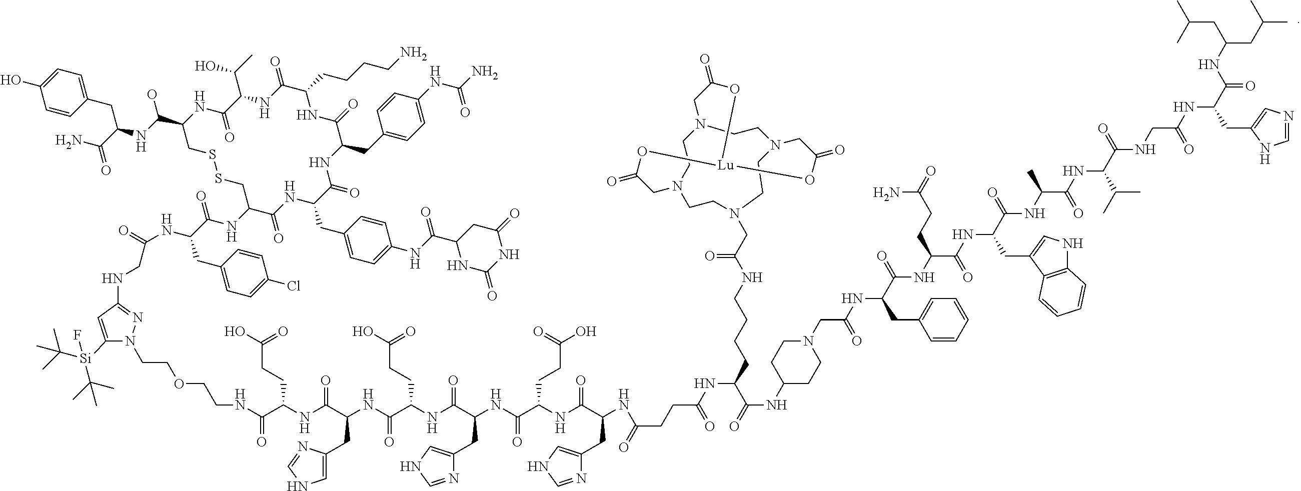

24. The method of claim 14, wherein the radiopharmaceutical compound is configured for PET imaging and therapeutic radioligand therapy.

25. The method of claim 14, wherein the conjugation is via a process selected from the group consisting of: coupling chemistry, peptide coupling chemistry, copper-catalyzed azide-alkyne cycloaddition (CuAAC), and solid-phase peptide synthesis (SPPS).

26. The method of claim 15, further comprising:
chelating 3p-C-NETA with ¹⁷⁷Lu for therapeutics;
chelating DOTA and DOTAGA with ¹⁷⁷Lu or ²²⁵Ac for therapeutics; and
chelating DOTA and DOTAGA with one of: ⁶⁸Ga, ⁸⁹Zr, and ⁶⁴Cu for PET imaging.
